SEQUENCE LISTING (6 sequences)

SEQ ID NO: 1 - protein, 391 aa, Homo sapiens
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 2 - protein, 391 aa, Homo sapiens; identical to SEQ ID NO: 1

SEQ ID NO: 3 - protein, 255 aa, Homo sapiens
MYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 4 - protein, 10 aa, Artificial Sequence
  CK2 Assay substrate peptide
RRRDDDSDDD

SEQ ID NO: 5 - protein, 9 aa, Artificial Sequence
  Pim-1 Assay substrate peptide
KKRNRTLTK

SEQ ID NO: 6 - protein, 10 aa, Artificial Sequence
  PIM-2 Assay substrate peptide
RSRSSYPAGT

(19) United States
(12) Patent Application Publication
(10) Pub. No.: US 2011/0071115 A1
(43) Pub. Date: Mar. 24, 2011

(54) PHARMACEUTICALLY USEFUL HETEROCYCLE-SUBSTITUTED LACTAMS

(75) Inventors: MUSTAPHA HADDACH, SAN DIEGO, CA (US); DAVID M. RYCKMAN, SAN DIEGO, CA (US); NICHOLAS RAFFAELE, SAN DIEGO, CA (US)

(73) Assignee: CYLENE PHARMACEUTICALS, INC., SAN DIEGO, CA (US)

(21) Appl. No.: 12/879,851

(22) Filed: Sep. 10, 2010

Related U.S. Application Data
(60) Provisional application No. 61/241,806, filed on Sep. 11, 2009.
(60) Provisional application No. 61/371,147, filed on Aug. 5, 2010.

(51) Int. Cl. C07D 487/04; C07F 9/6561; A61K 31/519; C12N 5/09; A61K 31/5513; A61K 31/5377; A61K 31/553; A61K 31/53; A61K 31/5025; A61K 31/675; A61P 35/00; A61P 29/00; A61P 9/00; A61P 31/00; A61P 37/00

(52) U.S. Cl. 514/81; 544/281; 540/575; 544/117; 540/544; 544/212; 544/236; 544/113; 544/244; 514/259.3; 435/375; 514/252.16; 514/218; 514/233.2; 514/255.05; 514/211.15; 514/245; 514/248

(57) ABSTRACT

The invention provides compounds that inhibit CK2 and/or Pim kinases and compositions containing such compounds. These compounds and compositions are useful for treating proliferative disorders such as cancer, as well as other kinase-associated conditions including inflammation, pain, infections, and certain immunological disorders.

### CROSS REFERENCE TO RELATED APPLICATIONS

This application claims the benefit of U.S. Provisional Application No. 61/241,806, filed on Sep. 11, 2009 and entitled “PHARMACEUTICALLY USEFUL HETEROCYCLE-SUBSTITUTED LACTAMS” and U.S. Provisional Application No. 61/371,147, filed on Aug. 5, 2010 and entitled “PHARMACEUTICALLY USEFUL HETEROCYCLE-SUBSTITUTED LACTAMS”, the content of which are incorporated by reference in their entirety for all purposes.

### FIELD OF THE INVENTION

The invention relates in part to molecules having certain biological activities that include, but are not limited to, inhibiting cell proliferation, and modulating certain protein kinase activities. Molecules of the invention can modulate protein kinase CK2 activity formely known as casein kinase activity and/or Pim kinase activity (e.g., Pim-1 activity), and are useful to treat cancers and inflammatory conditions as well as certain infectious disorders. The invention also relates in part to methods for using such compounds, and pharmaceutical compositions containing these compounds.

### BACKGROUND OF THE INVENTION

Protein kinase CK2 (formerly called Casein kinase II, referred to herein as “CK2”) is a ubiquitous and highly conserved protein serine/threonine kinase. The holoenzyme is typically found in tetrameric complexes consisting of two catalytic (alpha and/or alpha') subunits and two regulatory (beta) subunits. CK2 has a number of physiological targets and participates in a complex series of cellular functions including the maintenance of cell viability. The level of CK2 in normal cells is tightly regulated, and it has long been considered to play a role in cell growth and proliferation. Inhibitors of CK2 that are useful for treating certain types of cancers are described in PCT/US2007/077464, PCT/US2008/074820, PCT/US2009/35609.

Both the prevalence and the importance of CK2 suggest it is an ancient enzyme on the evolutionary scale, as does an evolutionary analysis of its sequence; its longevity may explain why it has become important in so many biochemical processes, and why CK2 from hosts have even been co-opted by infectious pathogens (e.g., viruses, protozoa) as an integral part of their survival and life cycle biochemical systems. These same characteristics explain why inhibitors of CK2 are believed to be useful in a variety of medical treatments as discussed herein. Because it is central to many biological processes, as summarized by Guerra & Issinger, Curr. Med. Chem., 2008, 15:1870-1886, inhibitors of CK2, including the compounds described herein, should be useful in the treatment of a variety of diseases and disorders.

Cancerous cells show an elevation of CK2, and recent evidence suggests that CK2 exerts potent suppression of apoptosis in cells by protecting regulatory proteins from caspase-mediated degradation. The anti-apoptotic function of CK2 may contribute to its ability to participate in transformation and tumorigenesis. In particular, CK2 has been shown to be associated with acute and chronic myelogenous leukemia, lymphoma and multiple myeloma. In addition, enhanced CK2 activity has been observed in solid tumors of the colon, rectum and breast, squamous cell carcinomas of the lung and of the head and neck (SCCHN), adenocarcinomas of the lung, colon, rectum, kidney, breast, and prostate. Inhibition of CK2 by a small molecule is reported to induce apoptosis of pancreatic cancer cells, and hepatocellular carcinoma cells (HegG2, Hep3, HeLa cancer cell lines); and CK2 inhibitors dramatically sensitized RMS (Rhabdomyosarcoma) tumors toward apoptosis induced by TRAIL. Thus an inhibitor of CK2 alone, or in combination with TRAIL or a ligand for the TRAIL receptor, would be useful to treat RMS, the most common soft-tissue sarcoma in children. In addition, elevated CK2 has been found to be highly correlated with aggressiveness of neoplasias, and treatment with a CK2 inhibitor of the invention should thus reduce tendency of benign lesions to advance into malignant ones, or for malignant ones to metastasize.

Unlike other kinases and signaling pathways, where mutations are often associated with structural changes that cause loss of regulatory control, increased CK2 activity level appears to be generally caused by upregulation or overexpression of the active protein rather than by changes that affect activation levels. Guerra and Issinger postulate this may be due to regulation by aggregation, since activity levels do not correlate well with mRNA levels. Excessive activity of CK2 has been shown in many cancers, including SCCHN tumors, lung tumors, breast tumors, and others. Id.

Elevated CK2 activity in colorectal carcinomas was shown to correlate with increased malignancy. Aberrant expression and activity of CK2 have been reported to promote increase nuclear levels of NF-kappaB in breast cancer cells. CK2 activity is markedly increased in patients with AML and CML during blast crisis, indicating that an inhibitor of CK2 should be particularly effective in these conditions. Multiple myeloma cell survival has been shown to rely on high activity of CK2, and inhibitors of CK2 were cytotoxic to MM cells. Similarly, a CK2 inhibitor inhibited growth of murine p190 lymphoma cells. Its interaction with Bcr/Abl has been reported to play an important role in proliferation of Bcr/Abl expressing cells, indicating inhibitors of CK2 may be useful in treatment of Bcr/Abl-positive leukemias. Inhibitors of CK2 have been shown to inhibit progression of skin papillomas, prostate and breast cancer xenografts in mice, and to prolong survival of transgenic mice that express prostate-promoters. Id. The role of CK2 in various non-cancer disease processes has been recently reviewed. See Guerra & Issinger, Curr. Med. Chem., 2008, 15:1870-1886. Increasing evidence indicates that CK2 is involved in critical diseases of the central nervous system, including, for example, Alzheimer's disease, Parkinson's disease, and rare neurodegenerative disorders such as Guam-Parkinson dementia, chromosome 18 deletion syndrome, progressive supranuclear palsy, Kuf's disease, or Pick's disease. It is suggested that selective CK2-mediated phosphorylation of tau proteins may be involved in progressive neurodegeneration of Alzheimer's. In addition, recent studies suggest that CK2 plays a role in memory impairment and brain ischemia, the latter effect apparently being mediated by CK2's regulatory effect on the PI3K survival pathways.

CK2 has also been shown to be involved in the modulation of inflammatory disorders, for example, acute or chronic inflammatory pain, glomerulonephritis, and autoimmune diseases, including, e.g., multiple sclerosis (MS), systemic lupus erythematosus, rheumatoid arthritis, and juvenile arthritis. It positively regulates the function of the serotonin 5-HT3 receptor channel, activates heme oxygenase type 2, and enhances the activity of neuronal nitric oxide synthase. A selective CK2 inhibitor was reported to strongly reduce pain response of mice when administered to spinal cord tissue prior to pain testing. It phosphorylates secretory type HA phospholipase A2 from synovial fluid of RA patients, and modulates secretion of DEK (a nuclear DNA-binding protein), which is a proinflammatory molecule found in synovial fluid of patients with juvenile arthritis. Thus inhibition of CK2 is expected to control progression of inflammatory pathologies such as those described here, and the inhibitors disclosed herein have been shown to effectively treat pain in animal models.

Protein kinase CK2 has also been shown to play a role in disorders of the vascular system, such as, e.g., atherosclerosis, laminar shear stress, and hypoxia. CK2 has also been shown to play a role in disorders of skeletal muscle and bone tissue, such as cardiomyocyte hypertrophy, impaired insulin signaling and bone tissue mineralization. In one study, inhibitors of CK2 were effective at slowing angiogenesis induced by growth factor in cultured cells. Moreover, in a retinopathy model, a CK2 inhibitor combined with octreotide (a somatostatin analog) reduced neovascular tufts; thus the CK2 inhibitors described herein would be effective in combination with a somatostatin analog to treat retinopathy.

CK2 has also been shown to phosphorylate GSK, troponin and myosin light chain; thus it is important in skeletal muscle and bone tissue physiology, and is linked to diseases affecting muscle tissue.

Evidence suggests that CK2 is also involved in the development and life cycle regulation of protozoal parasites, such as, for example, Theileria parva, Trypanosoma cruzi, Leishmania donovani, Herpetomonas muscarum muscarum, Plasmodium falciparum, Trypanosoma brucei, Toxoplasma gondii and Schistosoma mansoni. Numerous studies have confirmed the role of CK2 in regulation of cellular motility of protozoan parasites, essential to invasion of host cells. Activation of CK2 or excessive activity of CK2 has been shown to occur in hosts infected with Leishmania donovani, Herpetomonas muscarum muscarum, Plasmodium falciparum, Trypanosoma brucei, Toxoplasma gondii and Schistosoma mansoni. Indeed, inhibition of CK2 has been shown to block infection by T. cruzi.

CK2 has also been shown to interact with and/or phosphorylate viral proteins associated with human immunodeficiency virus type 1 (HIV-1), human papilloma virus, and herpes simplex virus, in addition to other virus types (e.g. human cytomegalovirus, hepatitis C and B viruses, Borna disease virus, adenovirus, coxsackievirus, coronavirus, influenza, and varicella zoster virus). CK2 phosphorylates and activates HIV-1 reverse transcriptase and proteases in vitro and in vivo, and promotes pathogenicity of simian-human immunodeficiency virus (SHIV), a model for HIV. Inhibitors of CK2 are thus able to reduce reduce pathogenic effects of a model of HIV infection. CK2 also phosphorylates numerous proteins in herpes simplex virus and numerous other viruses, and some evidence suggests viruses have adopted CK2 as a phosphorylating enzyme for their essential life cycle proteins. Inhibition of CK2 is thus expected to deter infection and progression of viral infections, which rely upon the host's CK2 for their own life cycles.

CK2 is unusual in the diversity of biological processes that it affects, and it differs from most kinases in other ways as well: it is constitutively active, it can use ATP or GTP, and it is elevated in most tumors and rapidly proliferating tissues. It also has unusual structural features that may distinguish it from most kinases, too, enabling its inhibitors to be highly specific for CK2 while many kinase inhibitors affect multiple kinases, increasing the likelihood of off-target effects, or variability between individual subjects. For all of these reasons, CK2 is a particularly interesting target for drug development, and the invention provides highly effective inhibitors of CK2 that are useful in treating a variety of different diseases and disorders mediated by or associated with excessive, aberrant or undesired levels of CK2 activity.

The PIM protein kinases which include the closely related Pim-1, -2, and -3, have been implicated in diverse biological processes such as cell survival, proliferation, and differentiation. Pim-1 is involved in a number of signaling pathways that are highly relevant to tumorigenesis [reviewed in Bachmann & Moroy, Internat. J. Biochem. Cell Biol., 37, 726-730 (2005)]. Many of these are involved in cell cycle progression and apoptosis. It has been shown that Pim-1 acts as an anti-apoptotic factor via inactivation of the pro-apoptotic factor BAD (Bcl2 associated death promoter, an apoptosis initiator). This finding suggested a direct role of Aim-1 in preventing cell death, since the inactivation of BAD can enhance Bcl-2 activity and can thereby promote cell survival [Aho et al., FEBS Letters, 571, 43-49 (2004)]. Pim-1 has also been recognized as a positive regulator of cell cycle progression. Pim-1 binds and phosphorylates Cdc25A, which leads to an increase in its phosphatase activity and promotion of G1/S transition [reviewed in Losman et al., JBC, 278, 4800-4805 (1999)]. In addition, the cyclin kinase inhibitor p21^{Waf} which inhibits G1/S progression, was found to be inactivated by Pim-1 [Wang et al., Biochim. Biophys. Acta. 1593, 45-55 (2002)]. Furthermore, by means of phosphorylation, Pim-1 inactivates C-TAKl and activates Cdc25C which results in acceleration of G2/M transition [Bachman et al., JBC, 279, 48319-48 (2004)].

Pim-1 appears to be an essential player in hematopoietic proliferation. Kinase active Pim-1 is required for the gp130-mediated STAT3 proliferation signal [Hirano et. al., Oncogene 19, 2548-2556, (2000)]. Pim-1 is overexpressed or even mutated in a number of tumors and different types of tumor cell lines and leads to genomic instability. Fedorov, et al., concluded that a Phase III compound in development for treating leukemia, LY333′531, is a selective Pim-1 inhibitor. O. Fedorov, et al., PNAS 104(51), 20523-28 (December 2007). Evidence has been published to show that Pim-1 is involved in human tumors including prostate cancer, oral cancer, and Burkitt lymphoma (Gaidano & Dalla Faver, 1993). All these findings point to an important role of Pim-1 in the initiation and progression of human cancers, including various tumors and hematopoietic cancers, thus small molecule inhibitors of Pim-1 activity are a promising therapeutic strategy.

Additionally, Pim-2 and Pim-3 have overlapping functions with Pim-1 and inhibition of more than one isoform may provide additional therapeutic benefits. However, it is sometimes preferable for inhibitors of PIM to have little or no in vivo impact through their inhibition of various other kinases, since such effects are likely to cause side effects or unpredictable results. See, e.g., O. Fedorov, et al., PNAS 104(51), 20523-28 (December 2007), discussing the effects that non-specific kinase inhibitors can produce. Accordingly, in some embodiments, the invention provides compounds that are selective inhibitors of at least one of Pim-1, Pim-2, and Pim-3, or some combination of these, while having substantially less activity on certain other human kinases, as described further herein, although the compounds of Formula (I) are typically active on CK2 as well as one or more Pim proteins.

The implication of a role for PIM-3 in cancer was first suggested by transcriptional profiling experiments showing that PIM3 gene transcription was upregulated in EWS/ETS-induced malignant transformation of NIH 3T3 cells. These results were extended to show that PIM-3 is selectively expressed in human and mouse hepatocellular and pancreatic carcinomas but not in normal liver or pancreatic tissues. In addition, PIM-3 mRNA and protein are constitutively expressed in multiple human pancreatic and hepatocellular cancer cell lines.

The link between PIM-3 overexpression and a functional role in promoting tumorigenesis came from RNAi studies in human pancreatic and hepatocellular cancer cell lines overexpressing PIM-3. In these studies the ablation of endogenous PIM-3 protein promoted apoptosis of these cells. The molecular mechanism by which PIM-3 suppresses apoptosis is in part carried out through the modulation of phosphorylation of the pro-apoptotic protein BAD. Similar to both Pim-1 & 2 which phosphorylate BAD protein, the knockdown of PIM-3 protein by siRNA results in a decrease in BAD phosphorylation at Ser112. Thus, similar to Pim-1 and 2, Pim-3 acts a suppressor of apoptosis in cancers of endodermal origin, e.g., pancreatic and liver cancers. Moreover, as conventional therapies in pancreatic cancer have a poor clinical outcome, PIM-3 could represent a new important molecular target towards successful control of this incurable disease.

At the 2008 AACR Annual Meeting, SuperGen announced that it has identified a lead PIM kinase inhibitor, SGI-1776, that causes tumor regression in acute myelogenous leukemia (AML) xenograft models (Abstract No. 4974). In an oral presentation entitled, “A potent small molecule PIM kinase inhibitor with activity in cell lines from hematological and solid malignancies,” Dr. Steven Warner detailed how scientists used SuperGen's CLIMB™ technology to build a model that allowed for the creation of small molecule PIM kinase inhibitors. SGI-1776 was identified as a potent and selective inhibitor of the NM kinases, inducing apoptosis and cell cycle arrest, thereby causing a reduction in phospho-BAD levels and enhancement of mTOR inhibition in vitro. Most notably, SGI-1776 induced significant tumor regression in MV-4-11 (AML) and MOLM-13 (AML) xenograft models. This demonstrates that inhibitors of PIM kinases can be used to treat leukemias.

Fedorov, et al., in PNAS vol. 104(51), 20523-28, showed that a selective inhibitor of Pim-1 kinase (Ly5333′531) suppressed cell growth and induced cell death in leukemic cells from AML patients. Pim-3 has been shown to be expressed in pancreatic cancer cells, while it is not expressed in normal pancreas cells, demonstrating that it should be a good target for pancreatic cancer. Li, et al., Cancer Res. 66(13), 6741-47 (2006).

Because these two protein kinases have important functions in biochemical pathways associated with cancer and inflammation, and are also important in pathogenicity of many microorganisms, inhibitors of their activity have many medicinal applications. The present invention provides novel compounds that inhibit CK2 or PIM or both, as well as compositions and methods of use utilizing these compounds.

### DISCLOSURE OF THE INVENTION

The present invention in part provides chemical compounds having certain biological activities that include, but are not limited to, inhibiting cell proliferation, inhibiting angiogenesis, and modulating protein kinase activities. These compounds modulate casein kinase 2 (CK2) activity and/or Pim kinase activity, and thus affect biological functions that include but are not limited to, inhibiting gamma phosphate transfer from ATP to a protein or peptide substrate, inhibiting angiogenesis, inhibiting cell proliferation, and inducing cell apoptosis, for example. Also provided are compositions comprising the present compounds, alone or in combination with other materials including inert excipients and/or other therapeutic agents. The present invention also in part provides methods for preparing these compounds and compositions comprising them, and methods of using these compounds and compositions comprising them.

The compounds of the invention have the general formula (I):

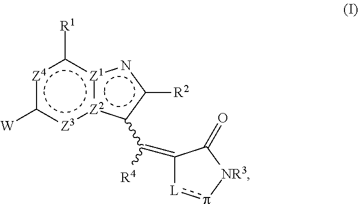

or a pharmaceutically acceptable salt, solvate, and/or prodrug thereof,
wherein:

the bicyclic ring system containing Z¹-Z⁴ is aromatic;

one of Z¹ and Z² is C, the other of Z¹ and Z² is N;

Z³ and Z⁴ are independently CR^{1a} or N,

R¹ and R^{1a} are independently H, halo, CN, optionally substituted C1-C4 alkyl, optionally substituted C2-C4 alkenyl, optionally substituted C2-C4 alkynyl, optionally substituted C1-C4 alkoxy, or —NR⁷R⁸;

R² is H, halo, CN, or an optionally substituted group selected from C1-C4 alkyl, C2-C4 alkenyl, and C2-C4 alkynyl;

R³ and R⁴ are independently selected from H and optionally substituted C1-C10 alkyl;

π is sp²-hybridized C or N;

the bond shown with a dotted line is a single bond if π is C═Y, where Y is O or S,

or the bond shown with a dotted line is a double bond if π is N or CR¹;

L is a one-carbon or two-carbon linker;

or L and π taken together form an additional 6-membered ring fused onto the ring containing the N of NR³, wherein the 6-membered ring optionally contains up to two heteroatoms selected from N, O and S as ring members;

W is halo, —OR⁷, —NR⁷R⁸, —S(O)ₙR⁷, —C(O)OR⁷, optionally substituted aryl, optionally substituted heteroaryl, optionally substituted heterocyclyl, optionally substituted C3-C8 cycloalkyl, or CR⁷R⁸R⁹,

wherein n is 0, 1 or 2,

each R⁷ and R⁸ and R⁹ is independently selected from H, optionally substituted C1-C10 alkyl, optionally substituted aryl, optionally substituted arylalkyl, optionally substituted heteroaryl, optionally substituted heteroarylalkyl, and optionally substituted heterocyclyl; or alternatively, R⁷ and R⁸ in NR⁷R⁸, taken together with the nitrogen atom to which they are attached, form a 5 to 8 membered ring that is optionally substituted and optionally contain an additional heteroatom selected from N, O and S as a ring member.

The invention also includes the pharmaceutically acceptable salts, solvates, and/or prodrugs of compounds of formula (I).

In certain embodiments, the invention provides compounds of Formula (Ia) or Formula (Ib):

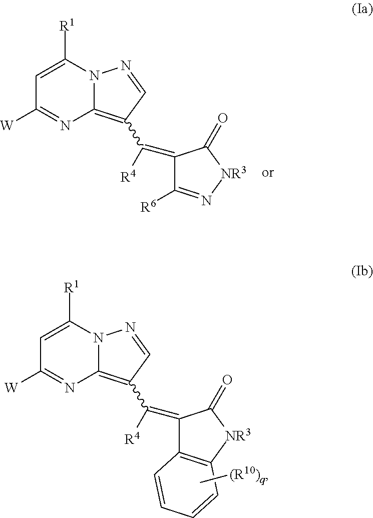

or a pharmaceutically acceptable salt, solvate, and/or prodrug thereof,

wherein q is 0, 1, or 2; each R¹⁰ is independently selected from halogen, cyano, R″, OR″, NR″R″, CONR″R″, and SO₂NR″R″, wherein each R″ is independently H or C1-C4 alkyl; and R⁶ is H or an optionally substituted C1-C10 alkyl.

In certain embodiments, the invention provides compounds of Formula (Ic) or Formula (Id):

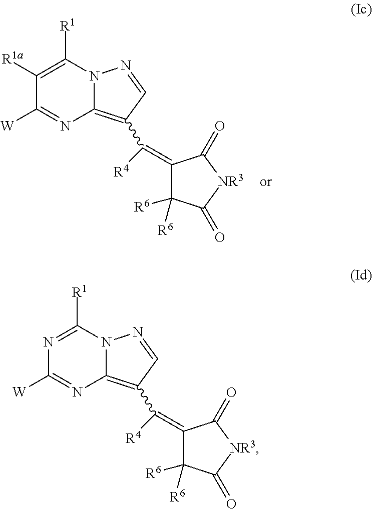

or a pharmaceutically acceptable salt, solvate, and/or prodrug thereof,

wherein R^{1a} is H or C1-C4 alkyl; R¹ is —NR⁷R⁸; and each R⁶ is H or an optionally substituted C1-C10 alkyl.

In certain embodiments, the present compounds may be in a prodrug form, such as compounds represented by Formula (Ie):

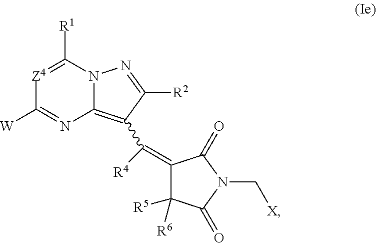

or a pharmaceutically acceptable salt and/or solvate thereof;
wherein,

Z⁴ are independently CR^{1a} or N,

R¹ and R^{1a} are independently H, halo, CN, optionally substituted C1-C4 alkyl, optionally substituted C2-C4 alkenyl, optionally substituted C2-C4 alkynyl, optionally substituted C1-C4 alkoxy, or —NR⁷R⁸;

R² is H, halo, CN, or an optionally substituted group selected from C1-C4 alkyl, C2-C4 alkenyl, and C2-C4 alkynyl;

R⁴ is H or optionally substituted C1-C10 alkyl;

each R⁶ is independently H or optionally substituted C1-C10 alkyl

W is halo, —OR⁷, —NR⁷R⁸, —S(O)ₙR⁷, —C(O)OR⁷, optionally substituted aryl, optionally substituted heteroaryl, optionally substituted heterocyclyl, optionally substituted C3-C8 cycloalkyl, or CR⁷R⁸R⁹,

wherein n is 0, 1 or 2,

each R⁷, R⁸, and R⁹ is independently selected from H, optionally substituted C1-C10 alkyl, optionally substituted aryl, optionally substituted arylalkyl, optionally substituted heteroaryl, optionally substituted heteroarylalkyl, and optionally substituted heterocyclyl; or alternatively, R⁷ and R⁸ in NR⁷R⁸, taken together with the nitrogen atom to which they are attached, form a 5 to 8 membered ring that is optionally substituted and optionally contain an additional heteroatom selected from N, O and S as a ring member;

X is hydroxyl or a group having structural formula (II), (III), (IV), or (V):

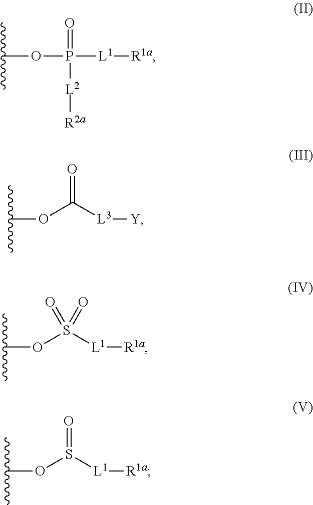

L′ and L² are each independently a covalent bond, —O—, or —NR^{3a}—;

R^{1a} and R^{2a} are each independently hydrogen, alkyl, heteroalkyl, heteroaryl, heterocyclyl, alkenyl, alkynyl, arylalkyl, heteroarylalkyl, heterocyclylalkyl, -alkylene-C(O)—O—R^{4a}, or -alkylene-O—C(O)—O—R^{4a}; and

R^{3a} and R^{4a} are each independently hydrogen, alkyl, heteroalkyl, cyclylalkyl, heterocyclyl, aryl, heteroaryl, alkenyl, alkynyl, arylalkyl, heterocyclylalkyl, or heteroarylalkyl;

L³ is a covalent bond or alkylene;

Y is OR^{5a}, NR^{5a}R^{6a}, or C(O)OR^{7a}, provided that when Y is C(O)OR^{7a}, then L³ is not a covalent bond; and

R^{5a}, R^{6a}, and R^{7a} are each independently hydrogen, alkyl, arylalkyl, aryl, heteroalkyl, alkylheteroaryl, heterocyclyl, or heteroaryl; or alternatively, R^{5a} and R^{6a}, taken together with the nitrogen atom to which they are attached, form a heterocyclyl ring optionally containing one or more additional heteroatom independently selected from N, O, and S.

The invention also provides pharmaceutical compositions containing the present compounds plus one or more pharmaceutically acceptable carriers or excipients; and methods of using these compounds and compositions for the treatment of certain conditions or diseases as further described herein.

The present compounds bind to certain kinase proteins, which are believed to be the basis for their pharmaceutical activity. In certain embodiments, the protein is a CK2 protein, such as a CK2 protein comprising the amino acid sequence of SEQ ID NO: 1, 2 or 3 or a -substantially identical variant thereof, for example.

| SEQ ID NO: 1 (NP_001886; casein kinase II alpha 1 subunit isoform a [Homo sapiens]) | |
|---|---|
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| SEQ ID NO: 2 (NP_808227; casein kinase II alpha 1 subunit isoform a [Homo sapiens]) | |
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| SEQ ID NO: 3 (NP_808228; casein kinase II alpha 1 subunit isoform b [Homo sapiens]) | |
| | myeilkaldy chsmgimhrd vkphnvmidh ehrklrlidw glaefyhpgq eynvrvasry |
| | fkgpellvdy qmydysldmw slgcmlasmi frkepffhgh dnydqlvria kvlgtedlyd |
| 121 | yidkynield prfndilgrh srkrwerfvh senqhlvspe aldfldkllr ydhqsrltar |
| 181 | eamehpyfyt vvkdqarmgs ssmpggstpv ssanmmsgis svptpsplgp lagspviaaa |
| 241 | nplgmpvpaa agaqq |

Substantially identical variants of these include proteins having at least 90% sequence homology with one of these, preferably at least 90% sequence identity; and having at least 50% of the level of in vitro kinase activity of the specified sequence under typical assay conditions.

The invention includes methods to modulate the activity of CK2 protein, either in vitro, in vivo, or ex vivo. Suitable methods comprise contacting a system comprising the protein with a compound described herein in an amount effective for modulating the activity of the protein. In certain embodiments the activity of the protein is inhibited, and sometimes the protein is a CK2 protein comprising the amino acid sequence of SEQ ID NO: 1, 2 or 3 or a substantially identical variant thereof, for example. In certain embodiments the system is a cell or tissue; in other embodiments, it can be in a cell-free system.

Also provided are methods for modulating the activity of a Pim protein, which comprise contacting a system comprising the protein with a compound described herein in an amount effective for modulating the activity of the protein. In certain embodiments, the system is a cell or tissue, and in other embodiments the system is a cell-free system. In certain embodiments, the activity of the Pim protein is inhibited.

Provided also are methods for inhibiting cell proliferation, which comprise contacting cells with a compound described herein in an amount effective to inhibit proliferation of the cells. The cells sometimes are in a cell line, such as a cancer cell line (e.g., breast cancer, prostate cancer, pancreatic cancer, lung cancer, hemopoietic cancer, colorectal cancer, skin cancer, ovary cancer cell line), for example. In some embodiments, the cancer cell line is a breast cancer, prostate cancer or pancreatic cancer cell line. The cells sometimes are in a tissue, can be in a subject, at times are in a tumor, and sometimes are in a tumor in a subject. In certain embodiments, the method further comprises inducing cell apoptosis. Cells sometimes are from a subject having macular degeneration.

Also provided are methods for treating a condition related to aberrant cell proliferation, which comprise administering a compound described herein to a subject in need thereof in an amount effective to treat the cell proliferative condition. In certain embodiments the cell proliferative condition is a tumor-associated cancer. The cancer sometimes is cancer of the breast, prostate, pancreas, lung, colorectum, skin, or ovary. In some embodiments, the cell proliferative condition is a non-tumor cancer, such as a hematopoietic cancer, for example, including leukemias and lymphomas. The cell proliferative condition is macular degeneration in some embodiments.

The invention also includes methods for treating cancer or an inflammatory disorder in a subject in need of such treatment, comprising: administering to the subject a therapeutically effective amount of a therapeutic agent useful for treating such disorder; and administering to the subject a molecule that inhibits CK2 and/or Pim in an amount that is effective to enhance a desired effect of the therapeutic agent. In certain embodiments, the molecule that inhibits CK2 and/or Pim is a compound of Formula (I), including compounds of Formula (Ia), (Ib), (Ic), and (Id), or a pharmaceutically acceptable salt, solvate, and/or prodrug thereof. In certain embodiments, the desired effect of the therapeutic agent that is enhanced by the molecule that inhibits CK2 and/or Pim is an increase in apoptosis in at least one type of cell.

In some embodiments, the present compound and at least one additional therapeutic agent are co-administered to a patient. The at least one additional therapeutic agent and the present compound may be administered simultaneously, sequentially, or separately. The at least one additional therapeutic agent and the present compound can be combined into one pharmaceutical composition in certain embodiments; in other embodiments that are administered as separate compositions.

Also provided are compositions of matter comprising a compound described herein and an isolated protein. The protein sometimes is a CK2 protein, such as a CK2 protein comprising the amino acid sequence of SEQ ID NO: 1, 2 or 3 or a substantially identical variant thereof, for example. In some embodiments, the protein is a Pim protein. Certain compositions comprise a compound described herein in combination with a cell. The cell may be from a cell line, such as a cancer cell line. In the latter embodiments, the cancer cell line is sometimes a breast cancer, prostate cancer, pancreatic cancer, lung cancer, hematopoietic cancer, colorectal cancer, skin cancer, of ovary cancer cell line.

These and other embodiments of the invention are described in the description that follows.

### MODES OF CARRYING OUT THE INVENTION

Compounds of Formula (I) exert biological activities that include, but are not limited to, inhibiting cell proliferation, reducing angiogenesis, preventing or reducing inflammatory responses and pain, and modulating certain immune responses. Compounds of this Formula can modulate CK2 activity, Pim activity or both, as demonstrated by the data herein. Such compounds therefore can be utilized in multiple applications by a person of ordinary skill in the art. For example, compounds described herein can be used, for example, for (i) modulation of protein kinase activity (e.g., CK2 activity), (ii) modulation of Pim activity (e.g., Pim-1 activity), (iii) modulation of cell proliferation, (iv) modulation of apoptosis, and (v) treatments of cell proliferation related disorders (e.g., administration alone or co-administration with another molecule).

### DEFINITIONS

The terms “a” and “an” do not denote a limitation of quantity, but rather denote the presence of at least one of the referenced item. The terms “a” and “an” are used interchangeable with “one or more” or “at least one”. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

The terms “compound(s) of the invention”, “these compounds”, “such compound(s)”, “the compound(s)”, and “the present compound(s)” refer to compounds encompassed by structural formulae disclosed herein, e.g., Formula (I), (Ia), (Ib), (Ic), (Id), and (Ie), includes any specific compounds within these formulae whose structure is disclosed herein. Compounds may be identified either by their chemical structure and/or chemical name. When the chemical structure and chemical name conflict, the chemical structure is determinative of the identity of the compound. Furthermore, the present compounds can modulate, i.e., inhibit or enhance, the biological activity of a CK2 protein, a Pim protein or both, and thereby is also referred to herein as a “modulator(s)” or “CK2 and/or Pim modulator(s)”. Compounds of Formula (I), (Ia), (Ib), (Ic), (Id), and (Ie), including any specific compounds described herein are exemplary “modulators”.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms as well as mixtures of stereoisomers in varying degrees of chiral purity, including racemic mixtures and mixtures of diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. As a non-limiting example, the compounds of Formula (I) have a Carbon-Carbon double bond to which group R⁴ is attached. Because the four groups attached to the double bond are typically all different, the double bond can exist as distinct E and Z isomers. The Formula (I) s depicted to indicate it can represent either the E isomer or the Z isomer, or both. Other structures may appear to depict a specific isomer, but that is merely for convenience, and is not intended to limit the invention to the depicted olefin isomer.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound. In another example, a substituted 1,2,4-triazole derivative may exist in at least three tautomeric forms as shown below:

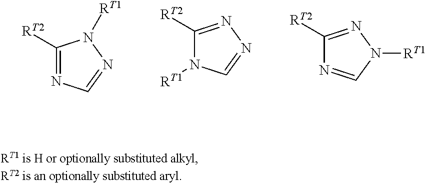

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases arc well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

“Solvate”, as used herein, means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate”. Examples of hydrate include, but are not limited to, hemihydrate, monohydrate, dihydrate, trihydrate, hexahydrate, etc. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

The term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolysable ester groups. That is, these esters are pharmaceutically acceptable and can be hydrolyzed to the corresponding carboxyl acid in vivo. These esters may be conventional ones, including lower alkanoyloxyalkyl esters, e.g. pivaloyloxymethyl and 1-pivaloyloxyethyl esters; lower alkoxycarbonylalkyl esters, e.g., methoxycarbonyloxymethyl, 1-ethoxycarbonyloxyethyl, and 1-isopropylcarbonyloxyethyl esters; lower alkoxymethyl esters, e.g., methoxymethyl esters, lactonyl esters, benzofuran keto esters, thiobenzofuran keto esters; lower alkanoylaminomethyl esters, e.g., acetylaminomethyl esters. Other esters can also be used, such as benzyl esters and cyano methyl esters. Other examples of these esters include: (2,2-dimethyl-1-oxypropyloxy)methyl esters; (1RS)-1-acetoxyethyl esters, 2-[(2-methylpropyloxy)carbonyl]-2-pentenyl esters, 1-[[(1-methylethoxy)carbonyl]-oxy]ethyl esters; isopropyloxycarbonyloxyethyl esters, (5-methyl-2-oxo-1,3-dioxole-4-yl)methyl esters, 1-[[(cyclohexyloxy)carbonyl]oxy]ethyl esters; 3,3-dimethyl-2-oxobutyl esters. It is obvious to those skilled in the art that hydrolysable esters of the compounds of the present invention can be formed at free carboxyls of said compounds by using conventional methods. Representative esters include pivaloyloxymethyl esters, isopropyloxycarbonyloxyethyl esters and (5-methyl-2-oxo-1,3-dioxole-4-yl)methyl esters.

The term “prodrug” refers to a precursor of a pharmaceutically active compound wherein the precursor itself may or may not be pharmaceutically active but, upon administration, will be converted, either metabolically or otherwise, into the pharmaceutically active compound or drug of interest. For example, prodrug can be an ester, ether, or amide form of a pharmaceutically active compound. Various types of prodrug have been prepared and disclosed for a variety of pharmaceuticals. See, for example, Bundgaard, H. and Moss, J., J. Pharm. Sci. 78: 122-126 (1989). Thus, one of ordinary skill in the art knows how to prepare these prodrugs with commonly employed techniques of organic synthesis.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Excipient” refers to a diluent, adjuvant, vehicle, or carrier with which a compound is administered.

An “effective amount” or “therapeutically effective amount” is the quantity of the present compound in which a beneficial outcome is achieved when the compound is administered to a patient or alternatively, the quantity of compound that possesses a desired activity in vivo or in vitro. In the case of proliferative disorders, a beneficial clinical outcome includes reduction in the extent or severity of the symptoms associated with the disease or disorder and/or an increase in the longevity and/or quality of life of the patient compared with the absence of the treatment. For example, for a subject with cancer, a “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the patient, such as general health, age, sex, body weight and tolerance to drugs. It will also depend on the degree, severity and type of proliferative disorder. The skilled artisan will be able to determine appropriate dosages depending on these and other factors.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C3-C8 heterocyclyl, C4-C10 heterocyclylalkyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C3-C8 heterocyclyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl, C3-C8 cycloalkyl, C3-C8 heterocyclyl, or C5-C10 heteroaryl, each of which can be substituted by the substituents that are appropriate for the particular group. Where a substituent group contains two R or R′ groups on the same or adjacent atoms (e.g., —NR₂, or —NR—C(O)R), the two R or R′ groups can optionally be taken together with the atoms in the substituent group to which they are attached to form a ring having 5-8 ring members, which can be substituted as allowed for the R or R′ itself, and can contain an additional heteroatom (N, O or S) as a ring member.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Substituted,” when used to modify a specified group or radical, means that one or more hydrogen atoms of the specified group or radical are each, independently of one another, replaced with the same or different substituent(s).

Substituent groups useful for substituting saturated carbon atoms in the specified group or radical include, but are not limited to —R^{a}, halo, —O⁻, ═O, —OR^{b}, —SR^{b}, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂—P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s may be taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. As another specific example, a substituted alkyl is meant to include -alkylene-O-alkyl, -alkylene-heteroaryl, -alkylene-cycloheteroalkyl, -alkylene-C(O)OR^{b}, -alkylene-C(O)NR^{b}R^{b}, and —CH₂—CH₂—(O)—CH₃. The one or more substituent groups, taken together with the atoms to which they are bonded, may form a cyclic ring including cycloalkyl and cycloheteroalkyl.

Similarly, substituent groups useful for substituting unsaturated carbon atoms in the specified group or radical include, but are not limited to, —R^{a}, halo, —O⁻, —OR^{b}, —SR^{b}, —S⁻, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —OR^{b}, —SR^{b}, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

“Acetylene” substituents are 2-10C alkynyl groups that are optionally substituted, and are of the formula —C≡C—R⁸, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and each R^{a} group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C═C—R^{a} is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Thus heteroacyl includes, for example, —C(═O)OR and —C(═O)NR₂ as well as —C(═O)— heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C1-C2 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCSNR₂, NRC(═NR)NR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C3-C8 heterocyclyl, C4-C10 heterocyclylalkyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups. Where a substituent group contains two R or R′ groups on the same or adjacent atoms (e.g., —NR2, or —NR—C(O)R), the two R or R′ groups can optionally be taken together with the atoms in the substituent group to which the are attached to form a ring having 5-8 ring members, which can be substituted as allowed for the R or R′ itself, and can contain an additional heteroatom (N, O or S) as a ring member.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups; where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” or “carbocyclic” refers to a cyclic ring containing only carbon atoms in the ring, whereas the term “heterocycle” or “heterocyclic” refers to a ring comprising a heteroatom. The carbocyclic and heterocyclic structures encompass compounds having monocyclic, bicyclic or multiple ring systems.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4 b]pyridine, piperazine, pyrazine, morpholine, thiomorpholine, imidazole, imidazolidine 2,4-dione, 1,3-dihydrobenzimidazol-2-one, indole, thiazole, benzothiazole, thiadiazole, thiophene, tetrahydro thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

The terms “treat” and “treating” as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganisms include but are not limited to virus, bacterium and fungus.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

### Embodiments of the Compounds

In one embodiment, the invention provides a compound having structural Formula (I):

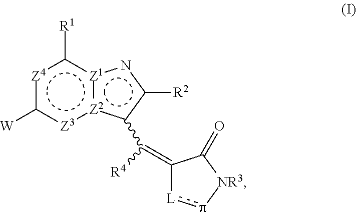

or a pharmaceutically acceptable salt, solvate, and/or prodrug thereof,
wherein:

the bicyclic ring system containing Z¹-Z⁴ is aromatic;

one of Z¹ and Z² is C, the other of Z¹ and Z² is N;

Z³ and Z⁴ are independently CR^{1a} or N,

R¹ and R^{1a} are independently H, halo, CN, optionally substituted C1-C4 alkyl, optionally substituted C2-C4 alkenyl, optionally substituted C2-C4 alkynyl, optionally substituted C1-C4 alkoxy, or —NR⁷R⁸;

R² is H, halo, CN, or an optionally substituted group selected from C1-C4 alkyl, C2-C4 alkenyl, and C2-C4 alkynyl;

R³ and R⁴ are independently selected from H and optionally substituted C1-C10 alkyl;

π is sp²-hybridized C or N;

the bond shown with a dotted line is a single bond if π is C═Y, where Y is O or S,

or the bond shown with a dotted line is a double bond if π N or CR¹;

L is a one-carbon or two-carbon linker;

or L and π taken together form an additional 6-membered ring fused onto the ring containing the N of NR³, wherein the 6-membered ring optionally contains up to two heteroatoms selected from N, O and S as ring members;

W is halo, —OR⁷, —NR⁷R⁸, —S(O)OR⁷, —C(O)OR⁷, optionally substituted aryl, optionally substituted heteroaryl, optionally substituted heterocyclyl, optionally substituted C3-C8 cycloalkyl, or CR⁷R⁸R⁹,

wherein n is 0, 1 or 2,

each R⁷, R⁸, and R⁹ is independently selected from H, optionally substituted C1-C10 alkyl, optionally substituted aryl, optionally substituted arylalkyl, optionally substituted heteroaryl, optionally substituted heteroarylalkyl, and optionally substituted heterocyclyl; or alternatively, R⁷ and R⁸ in NR⁷R⁸, taken together with the nitrogen atom to which they are attached, form a 5 to 8 membered ring that is optionally substituted and optionally contain an additional heteroatom selected from N, O and S as a ring member.

The compounds of the invention are characterized by a bicyclic aromatic heterocyclic ring system containing two or more nitrogen atoms: one N atom is shown, and one of Z¹ and Z² is also N. In certain embodiments of interest, Z¹ is N and Z² is C; in other embodiments, Z¹ is C and Z² is N.

Optionally, Z³ and/or Z⁴ can also be N. In certain embodiments, they are both CR¹; in other embodiments Z³ is N and Z⁴ is CR¹; and in other embodiments Z⁴ is N and Z³ is CR¹; while in other embodiments, Z³ and Z⁴ are both N.

In addition, the compounds of Formula (I) contain another heterocyclic group linked to the bicyclic group, and the additional heterocyclic group contains an amide linkage within the ring, and additional atoms forming a 5-6 membered ring. The additional atoms include a linker, L, which can comprise one or two carbon atoms as ring members, which can be substituted, e.g., L can be C(R⁶)₂ or C(R⁶)₂C(R⁶)₂. Alternatively, L can be CR⁶, when it is double-bonded to the adjacent center represented by rt. Each R⁶ can be same or different.

In the compounds of Formula (I), R⁶ can be H or an optionally substituted C1-C10 alkyl, independently at each occurrence.

π represents an sp² hybridized ring member, which can be C or N. When it represents N, it is double-bonded to the linker L. Thus in some embodiments, -L-π-NR³ is —CR⁶═N—NR³, and the ring becomes a pyrazolone ring. When it represents C, it can be either C═Y or CR¹, depending on the position of its double bond, which can be in the ring or exocyclic (i.e., it can be C═Y as explained below).

In some embodiments, π represents an sp² hybridized carbon atom such as C═Y; in these embodiments, Y is typically a heteroatom selected from N, O and S, and typically Y is O or S. Thus in such embodiments, -L-π-NR³ is often —C(R⁶)₂—C(═Y)—NR³ or —C(R⁶)₂—C(R⁶)₂—C(═Y)—NR³, In such embodiments, each R⁶ can be H or an optionally substituted alkyl; in specific embodiments, at least one R⁶ present is H. In certain embodiments, each R⁶ of the group represented by L is H.

In some embodiments of these compounds, Y is O and in some embodiments Y is S.

In still other embodiments, π represents an sp² hybridized carbon atom of the formula ═C(R¹)— (where the bond with a dotted line is a double bond, so the carbon atom is connected to one monovalent group R¹).

The additional heterocyclic group also contains NR³, and R³ in this group can be H or a small alkyl such as Me or ethyl, or cyclopropyl. In some embodiments, it is a substituted alkyl group such as formyl, acetyl, propionyl, benzoyl, and the like; these can be active on their own, or can function as prodrugs that become active when the acyl group is lost. Preferably, R³ is H.

The sp² carbon connecting the two heterocyclic groups is CR⁴, where R⁴ can be H or a small alkyl (Me, Et, iPr, tBu, cyclopropyl); in preferred embodiments, it is H.

The five-membered ring of the bicyclic group is substituted by R². This can be H, halo or a small alkyl, such as Me, Et, CF₃, —CH₂OMe, vinyl, or acetylene. In preferred embodiments, R² is H.

The six-membered ring of the bicyclic group is substituted by R and R¹ or R¹ only. This can be a variety of groups, including H, halo or an optionally substituted alkyl, amine or alkoxy group. In some embodiments, R and R¹ are independently selected from H, halo, and small alkyls, such as Me, Et, CF₃, —CH₂OMe, vinyl, or acetylene. In certain embodiments, R and R¹ are independently H, halo, Me, NHMe, NMe₂, CF₃, or CN.

The six-membered ring of the bicyclic group is also substituted by a group W. This can represent a range of different features while retaining the desired protein kinase modulatory activities. In certain embodiments, W is an optionally substituted aryl or heteroaryl group, often selected from phenyl, pyridyl, pyrimidinyl, and pyrazinyl. In particular, it can be an optionally substituted phenyl group. In specific embodiments, W is phenyl substituted with up to two substituents; in certain embodiments, the phenyl group is substituted by at least one group other than H, such as F, Cl, Me, CF₃, CN, OMe, COOH, or COOMe, at the ortho or meta position relative to the point at which the phenyl is connected to the bicyclic group.

Specific embodiments of the substituted phenyl that can be W include 3-chlorophenyl, 2-fluorophenyl, 3-fluorophenyl, 3-carboxyphenyl, and 3-(COOMe)-phenyl.

In other embodiments, W can be a group of the formula —NR⁷R⁸, where R⁷ and R⁸ are as described above. Typically, R⁷ and R⁸ are not both H. In certain of these embodiments, R⁷ is H, Me, or an acyl group such as formyl, acetyl, methoxyacetyl, benzoyl, or trifluoroacetyl; such acylated compounds may be active as kinase inhibitors, or they can serve as prodrugs for compounds wherein R⁷ is H. In these embodiments, R⁸ can be an optionally substituted alkyl group, or an aryl or heteroaryl group, such as phenyl, pyridinyl, pyrimidinyl, pyrazinyl, and the like, which can be optionally substituted. Suitable optionally substituted alkyl groups include C1-C6 alkyls, e.g., methyl, ethyl, butyl, propyl, isopropyl, t-butyl, fluoroethyl, methoxyethyo, isobutyl, and the like. In certain embodiments, the aryl or, heteroaryl group is substituted by at least one non-H substituent group. Some specific non-H substituents include halo (especially Cl or F), small alkyl groups (e.g., Me, Et, iPr, CF₃, cyclopropyl, and the like); C1-C4 alkoxy, CN, and the like, and can be at the position meta or para to the point where the aryl/phenyl ring connects to the nitrogen atom of NR⁷R⁸.

R⁸ can also be such an aryl or heteroaryl group that is connected to NR⁷ through a C1-C4 alkylene chain; e.g., it can be imidazolylmethyl, phenylethyl, and the like. In specific embodiments, the aryl is phenyl, and is substituted by at least one non-H substituent, often at the position that is meta or para to the point where the phenyl is connected to the N of NR⁷R⁸.

The substituent(s) on this aryl or heteroaryl group can be halo, C1-C4 alkyl, or C1-C4 alkoxy groups, or aryl or heteroaryl groups such as imidazole, phenyl, pyridyl, pyrazolyl, triazolyl, and the like; or they can be C5-C8 heterocyclic groups such as morpholine, piperidine, piperazine, and the like. In some embodiments, the aryl ring (e.g., phenyl) represented by R⁸ is substituted with a group of the formula R′₂N—(CH₂)ₚ-L-, where p is 0-3, L is a bond, O, S, or NR″ (R″ is H or C1-C4 alkyl), and each R′ is independently H or C1-C6 alkyl that is optionally substituted, and wherein the two R′ groups can optionally cyclize to form a ring, which can include an additional heteroatom (N, O or S) as a ring member. Representative examples of this version of R⁸ include dimethylamino; 4-methylpiperazinyl; 4-morpholinyl; 4-morpholinomethyl; 4-Me-piperazinoethyl; dimethylaminomethyl; diethylaminomethyl; dimethylaminoethoxy, and the like.

Alternatively, R⁸ can be an arylalkyl or heteroarylalkyl group, such as an optionally substituted benzyl group.

Alternatively, W can be NR⁷R⁸, where R⁷ and R⁸ taken together with N form a ring, which in some embodiments is a 5-8 membered ring that can optionally contain N, O or S as an additional ring member and can be substituted. Exemplary rings include piperidine, piperazine, homopiperazine, morpholine, thiomorpholine, pyrrolidine, pyrrolidinone, and the like.

In compounds of Formula (I), X and Y each represent a heteroatom, and they can be the same or they can be different. In some embodiments, Y is O, while X is S or NH or NMe or O; in other embodiments, Y is S, while X is S, or NH, or NMe or O. Where X is NR⁶, R⁶ can be H, methyl, ethyl, methoxyethyl, and the like; in preferred embodiments, R⁶ is H or it is Me.

The compounds of the invention include compounds of Formula (I) that contain the features specifically described below, or any combination of these features.

In certain embodiments of the compounds of Formula (I), Z¹ is N and Z² is C.

In certain embodiments of the compounds described above, Z³ is N.

In certain embodiments of the compounds described above, Z⁴ is N or CR^{1a}, wherein R^{1a} is H or C1-C4 alkyl.

In certain embodiments of the compounds described above, R² is H or Me.

In certain embodiments of the compounds described above, R³ and R⁴ are both H.

In certain embodiments of the compounds described above, R¹ is Me, halo, OMe, or CF₃.

In certain embodiments of the compounds described above, R¹ is H or —NR⁷R⁸.

In certain embodiments of the compounds described above, π is C═Y, where Y is O or S.

In certain embodiments of the compounds described above, L is C(R⁶)₂.

In certain embodiments of the compounds described above, -L-π-N(R³)— is —CR⁶═N—N(R³)—.

In certain embodiments of the compounds described above, R⁶ is H or optionally substituted C1-C10 alkyl.

In certain embodiments of the compounds described above, -L-π-N(R³)— is

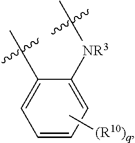

where R¹⁰ is selected from halogen, cyano, R″, OR″, NR″R″, CONR″R″, SO₂NR″R″, where each R″ is independently H or C1-C4 alkyl, and q is 0-2.

In certain embodiments of the compounds described above, W is —OR⁷ or —NR⁷R⁸.

In certain embodiments of the compounds described above, W is optionally substituted aryl or optionally substituted heteroaryl.

In certain embodiments of the compounds described above, W is optionally substituted phenyl.

In certain embodiments of the compounds described above, R⁸ is H, or alternatively, R⁷ and R⁸, taken together with the nitrogen atom, forms a 5 to 8 membered ring that is optionally substituted and optionally contains an additional heteroatom selected from N, O and S as a ring member.

In certain embodiments of the compounds described above, the compound is represented by Formula (Ic) or Formula (Id):

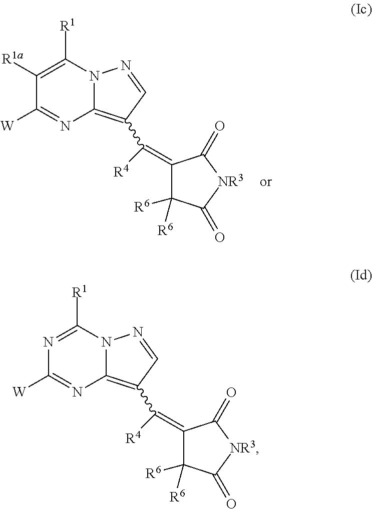

or a pharmaceutically acceptable salt, solvate, and/or prodrug thereof,
wherein R^{1a} is H or C1-C4 alkyl; R¹ is —NR⁷R⁸; and each R⁶ is H or an optionally substituted C1-C10 alkyl.

In certain embodiments of the compounds described above, the compound is represented by Formula (Ic) or Formula (Id):

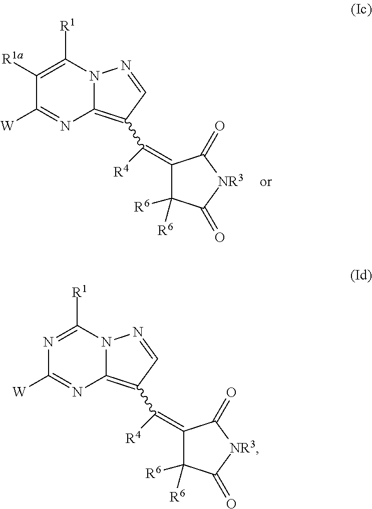

or a pharmaceutically acceptable salt, solvate, and/or prodrug thereof,
wherein R^{1a} is H or C1-C4 alkyl; R¹ is —NR⁷R⁸; and each R⁶ is H or an optionally substituted C1-C10 alkyl.

In certain embodiments of the compounds described above, W is —NH-A, wherein A is optionally substituted phenyl. In alternative embodiments of the above compounds, W is optionally substituted aryl or optionally substituted heteroaryl. In specific embodiments of this type, W can be optionally substituted phenyl. Suitable substitution patterns comprise up to three substituents, and in some embodiments, this phenyl has 1 or 2 substituents. The substituents are often attached at a carbon that is meta or para to the point where the phenyl attaches to nitrogen of —NR⁷R⁸.

In certain embodiments of these compounds, W is optionally substituted phenyl. In these embodiments, R³ and R⁴ arc in some instances, selected from H and Me, and preferably both R³ and R⁴ are H. In these embodiments, R¹ can be H, Me, CF₃, CN, NH₂, NHMe, NMe₂, OMe, or halo.

In Formula (Ia), R⁶ can be H or it can be a substituted C1-C10 alkyl. Where it represents an optionally substituted alkyl, it is often Me, Et, iPr, or cyclopropyl, or a substituted alkyl such as CF₃ or CH₂CF₃, or —CH₂OMe. In preferred embodiments, R⁶ is H or Me or CF₃.

In Formula (Ia), (Ib), (Ic) or (Id), W can be —NR⁷R⁸, where R⁸ can be an optionally substituted aryl or heteroaryl or arylalkyl or heteroarylalkyl group. In some embodiments, R⁸ is an optionally substituted phenyl pyridyl, pyrimidinyl, or pyrazinyl group, while R⁷ is H.

In Formula (Ib), q can be 0-2, and is often 0 or 1. Where one or more R¹⁰ groups are present (i.e., q is not 0), they are often selected from F, Cl, Me, OMe, CN, SMe, SO₂Me, COOMe, and CF₃.

In certain specific embodiments, the present invention provides compounds selected from the group consisting of

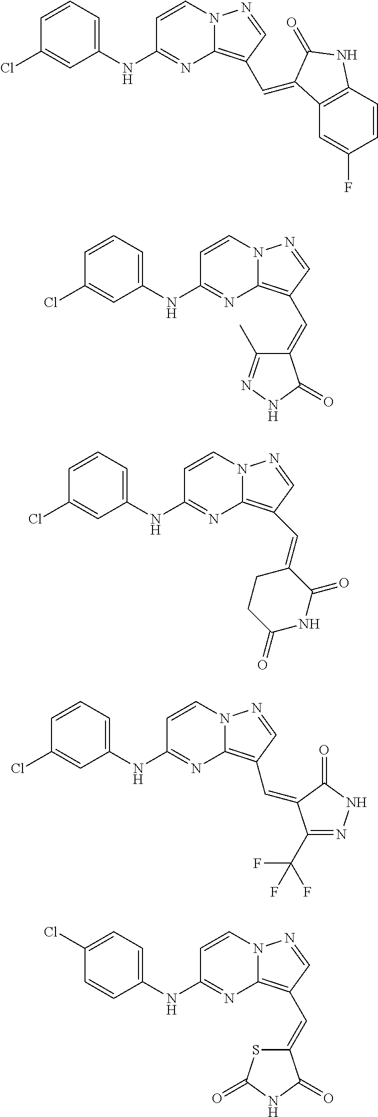

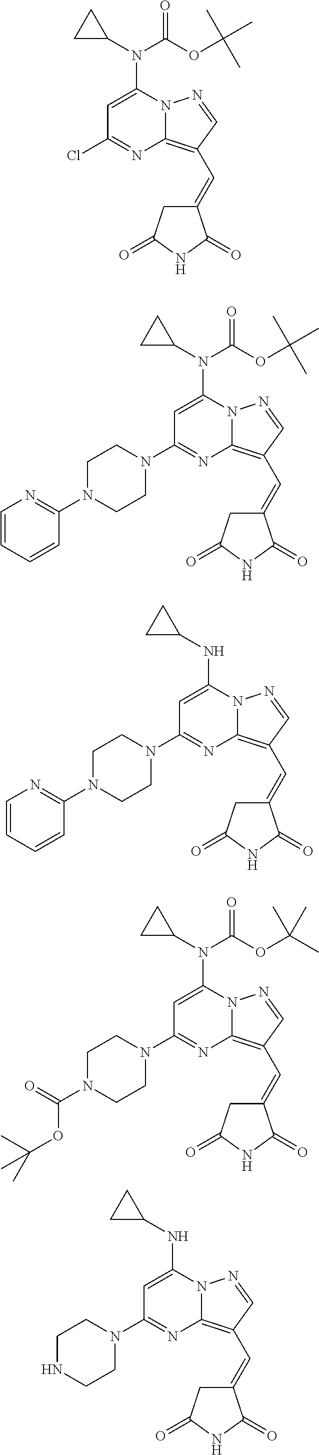

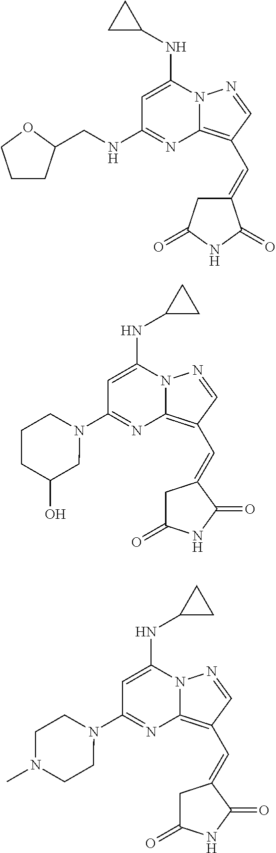

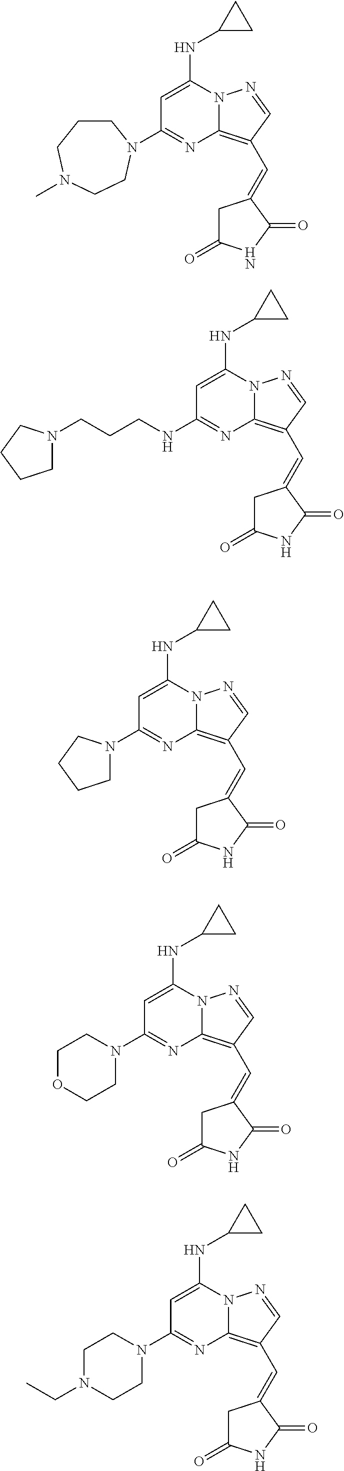

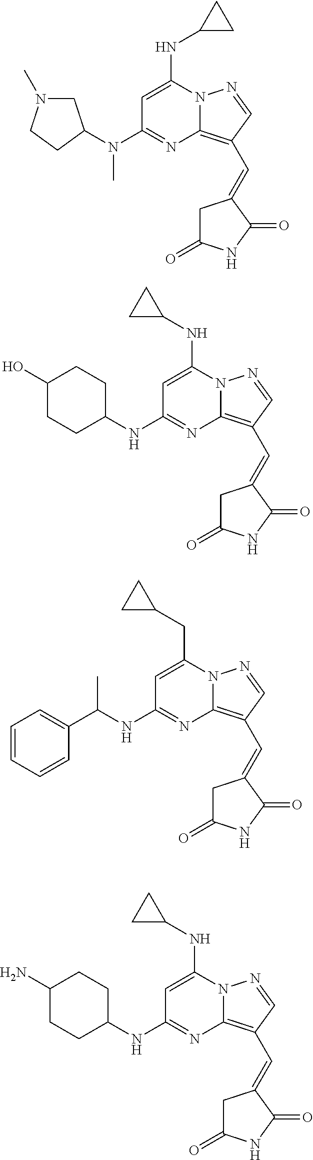

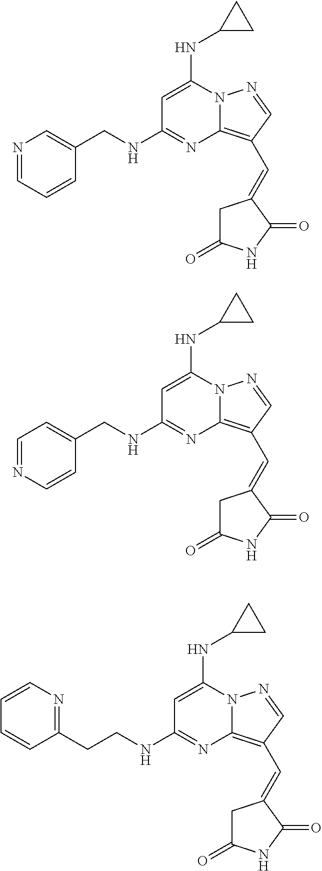

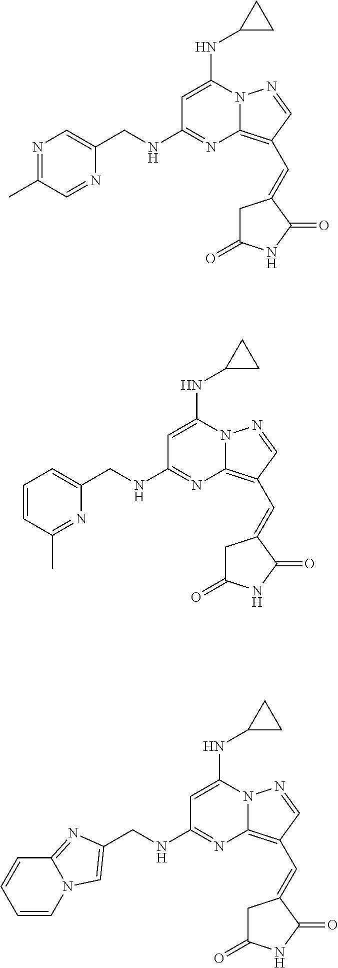

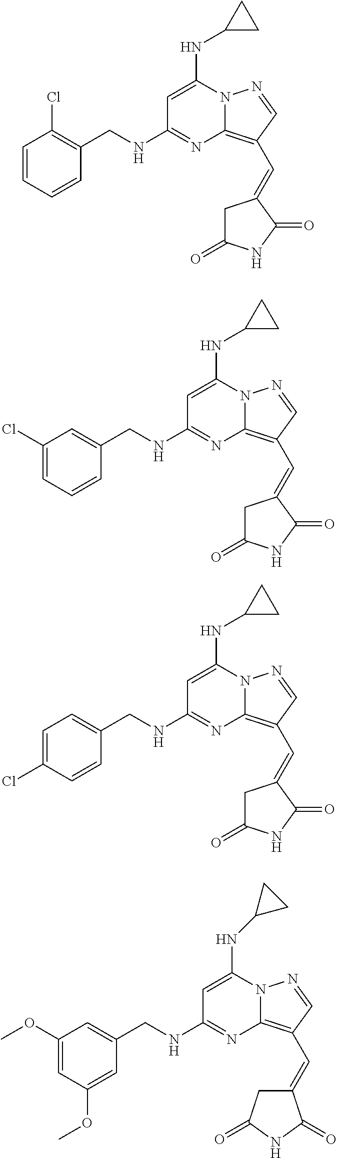

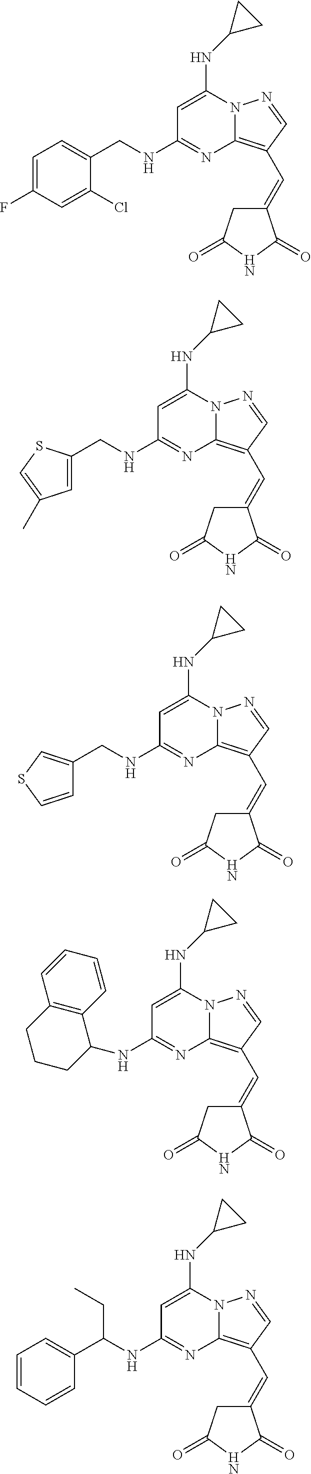

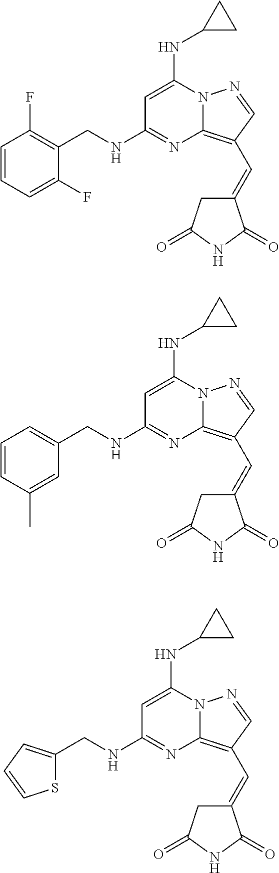

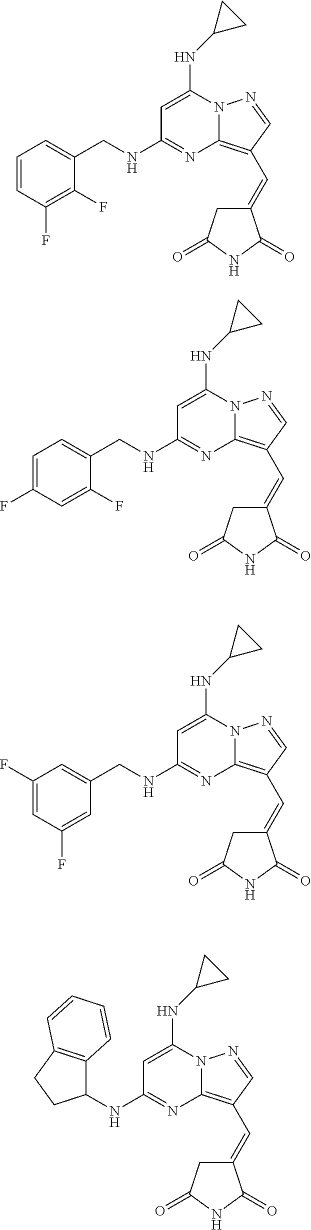

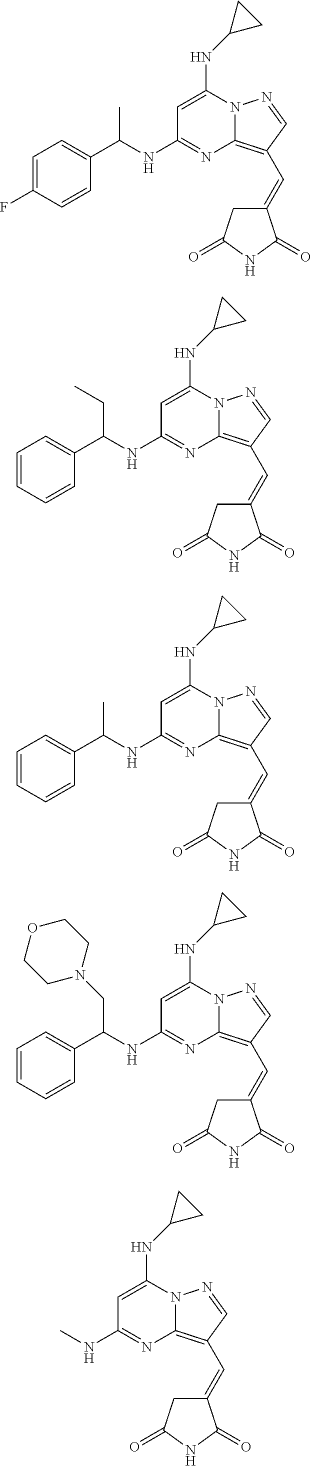

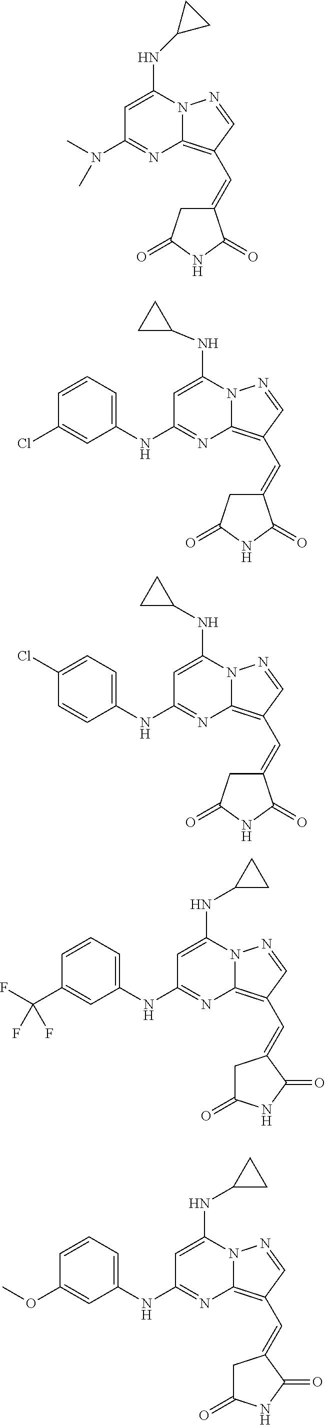

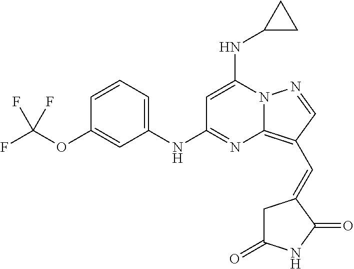

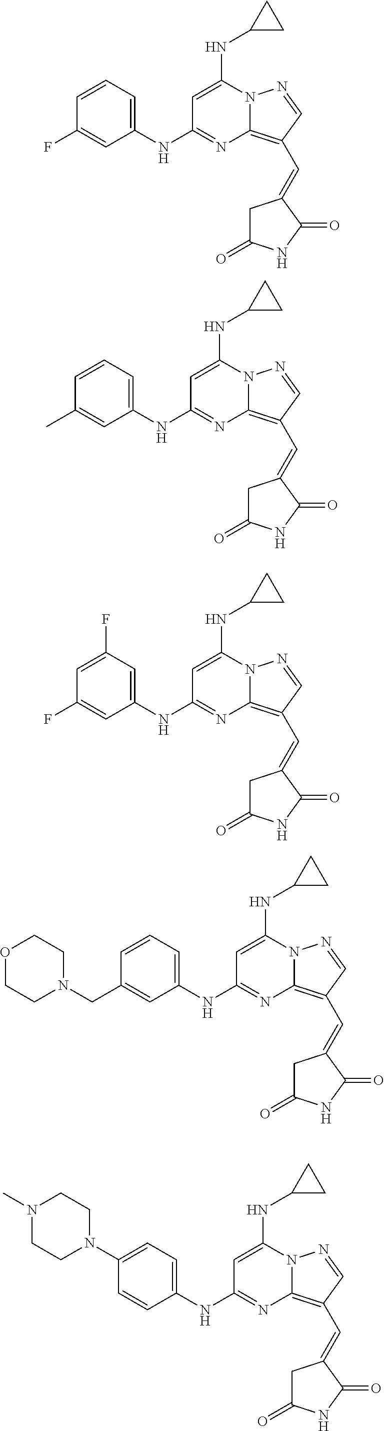

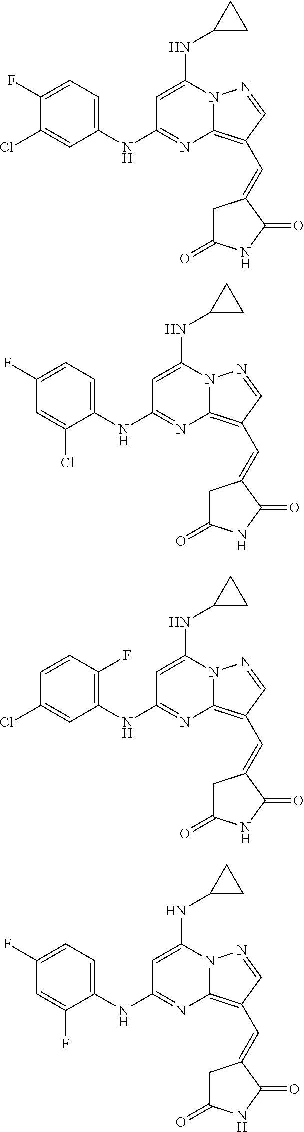

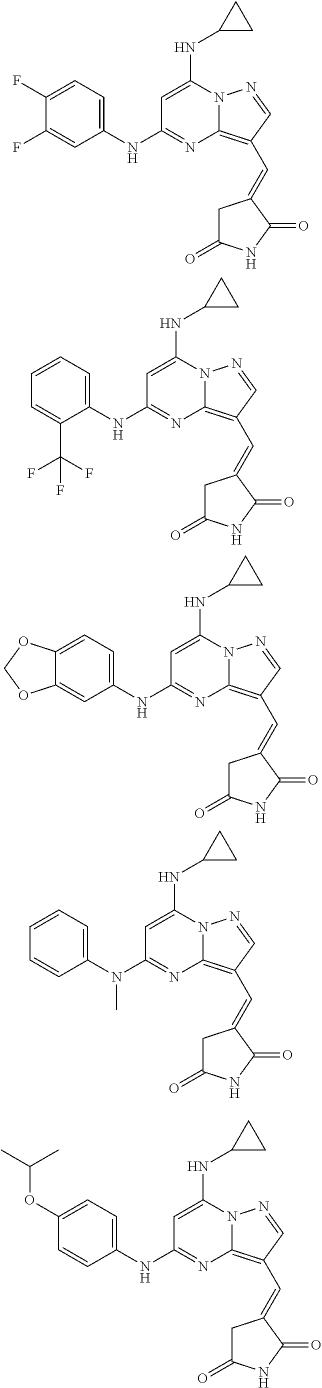

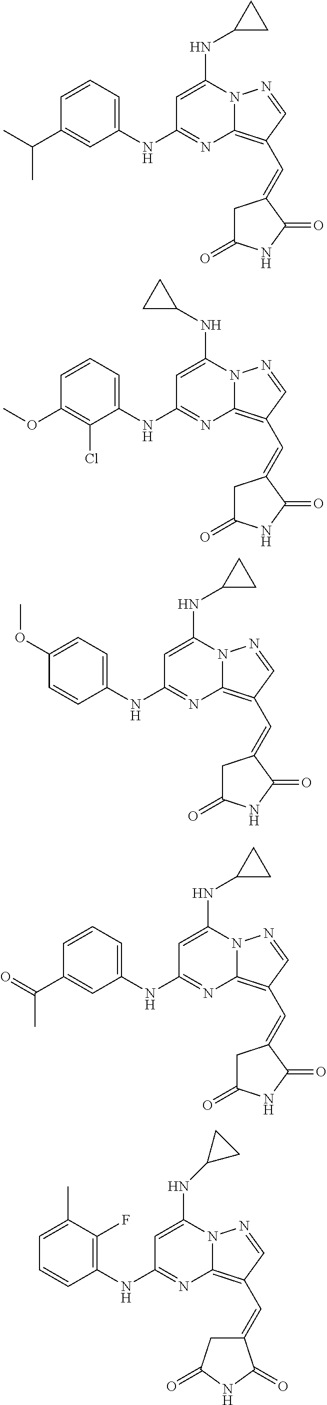

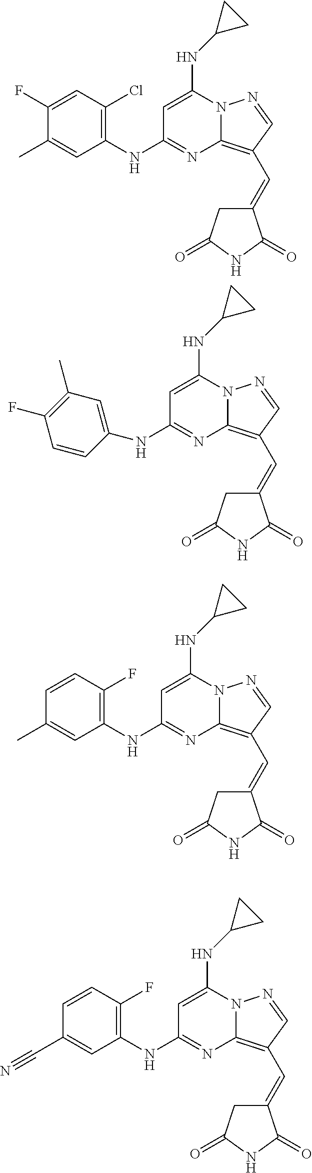

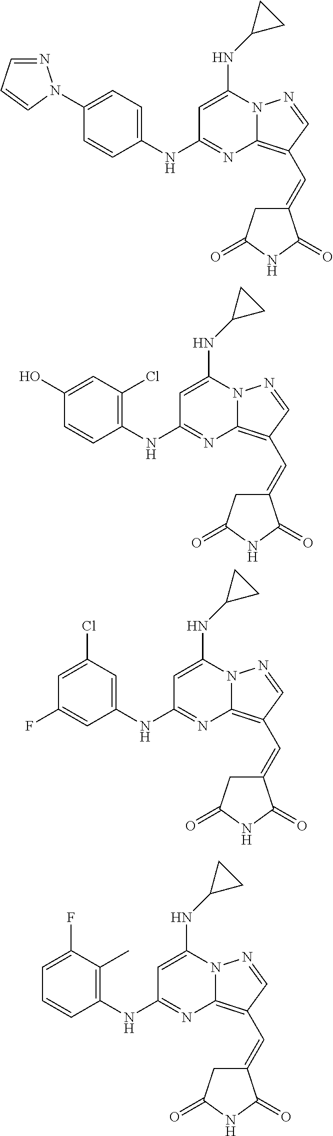

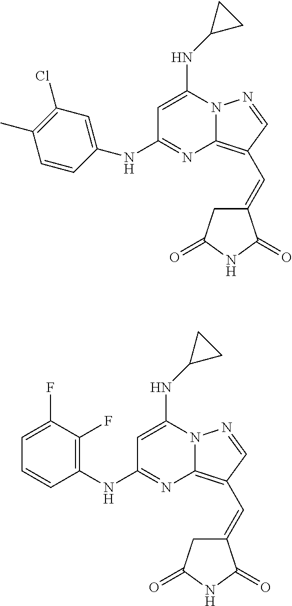

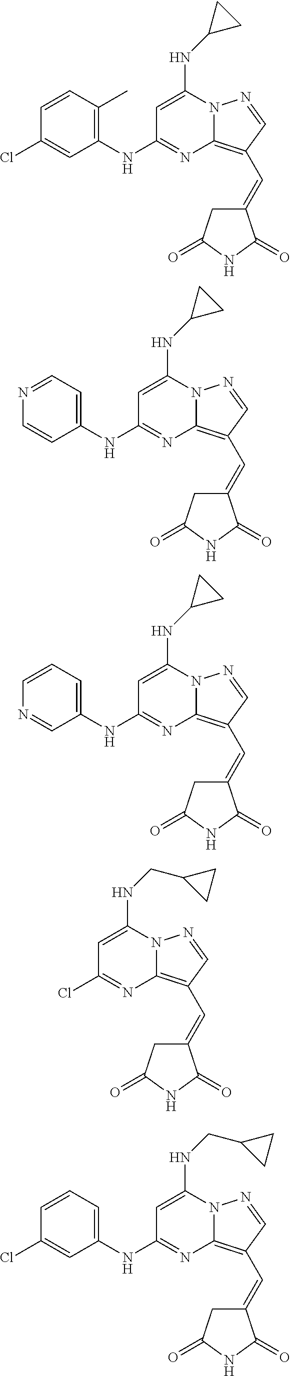

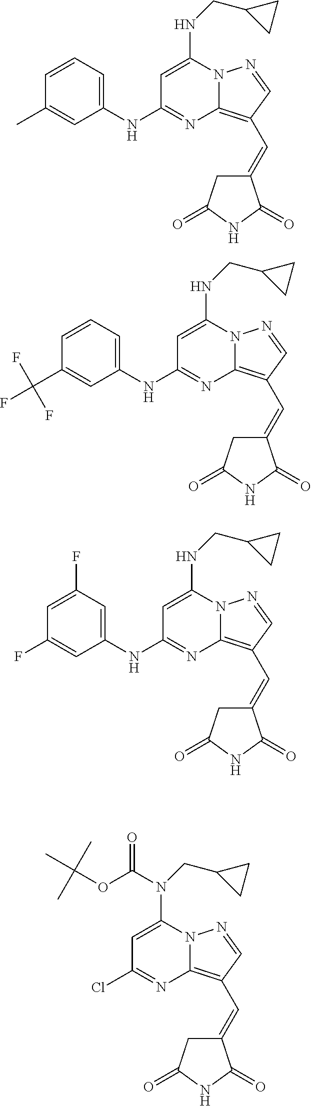

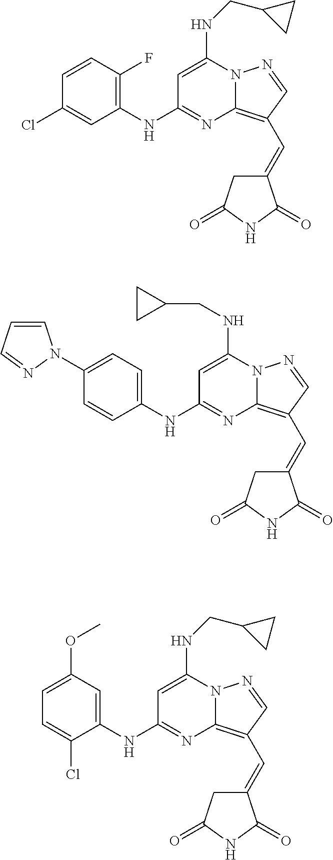

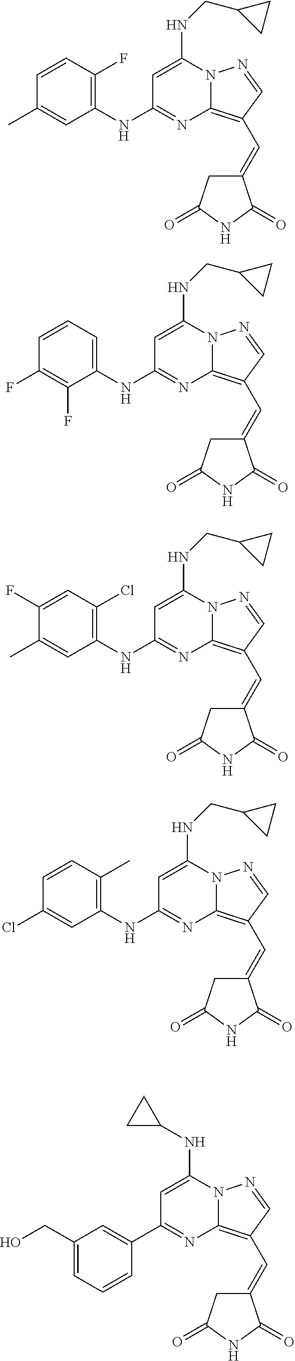

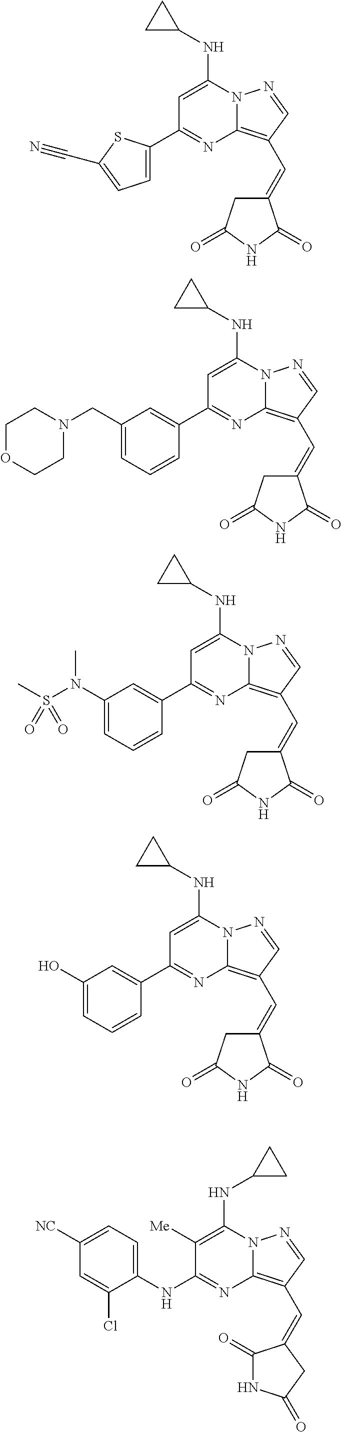

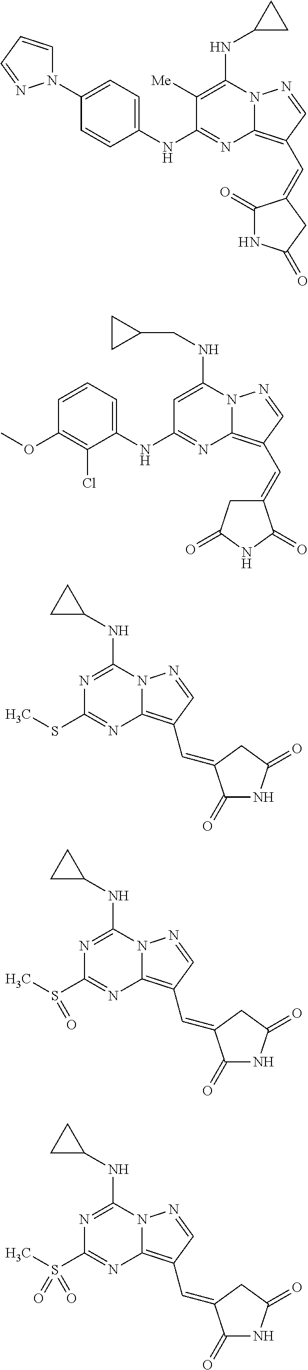

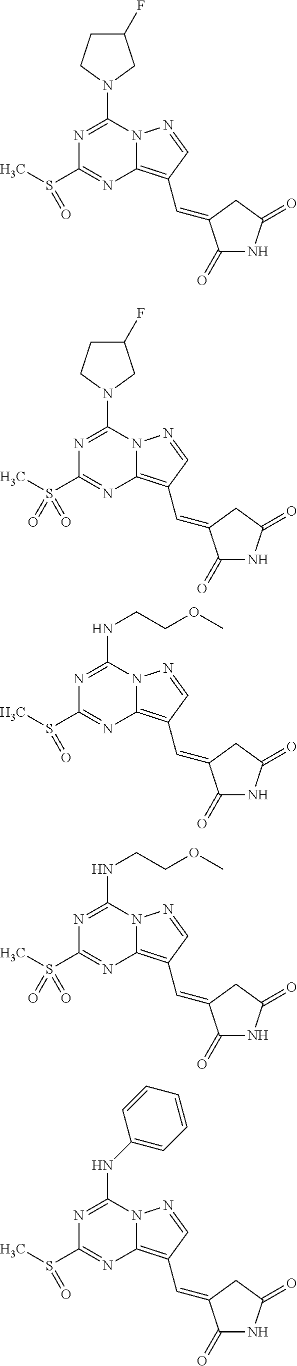

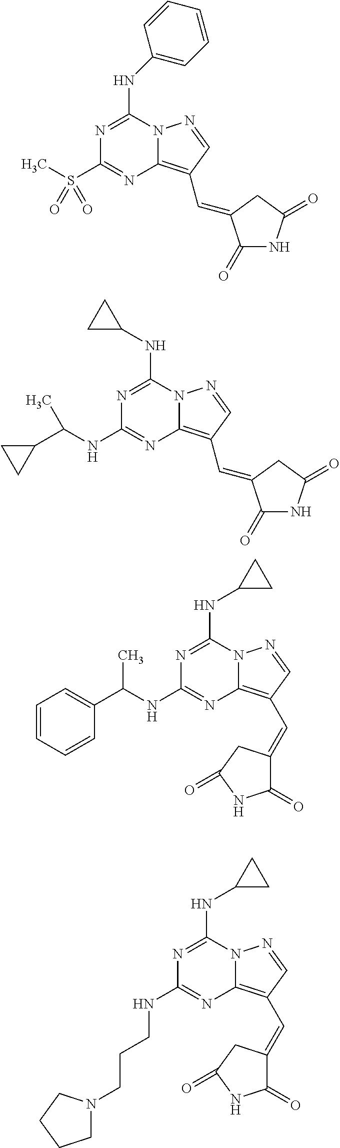

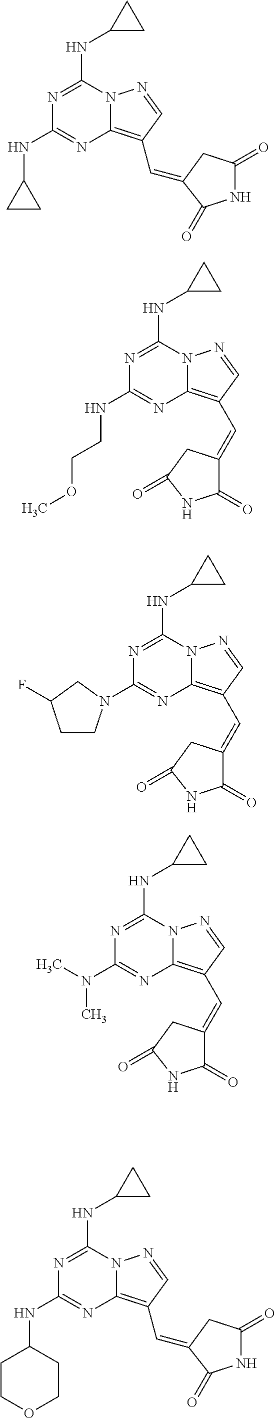

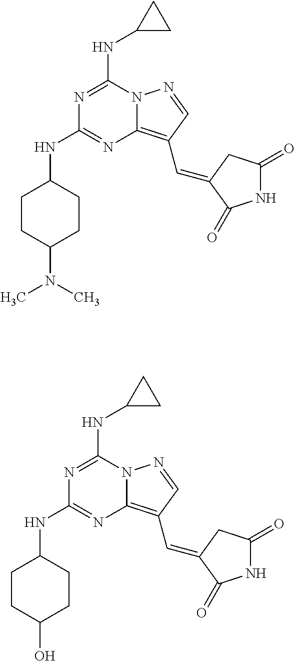

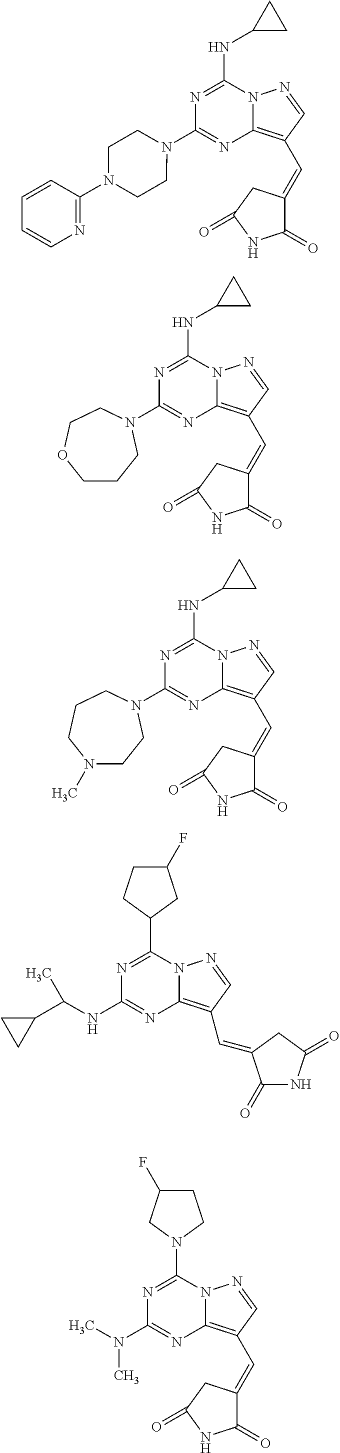

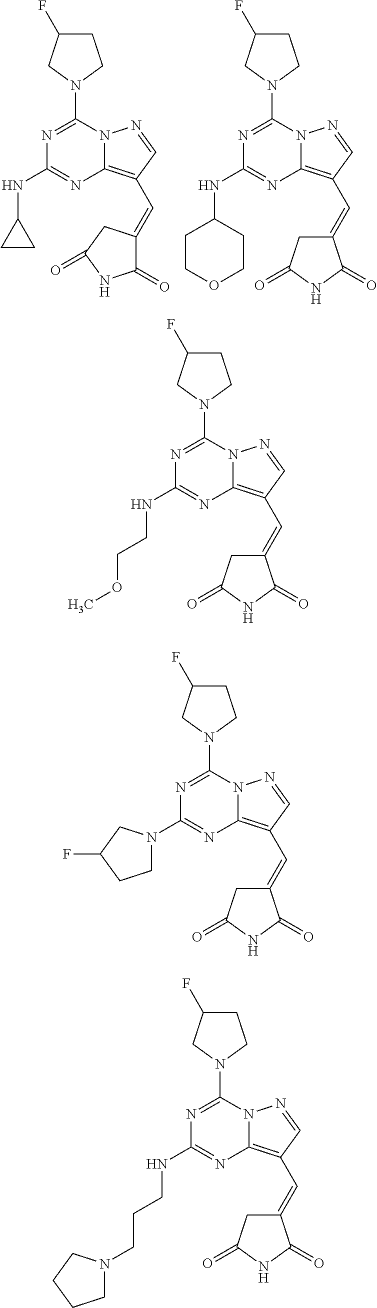

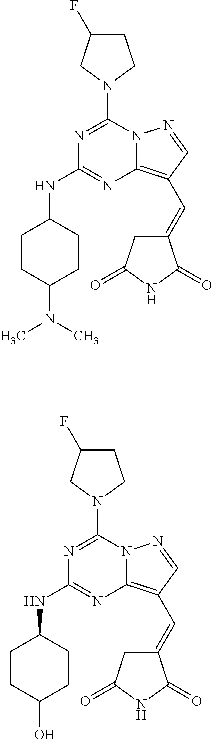

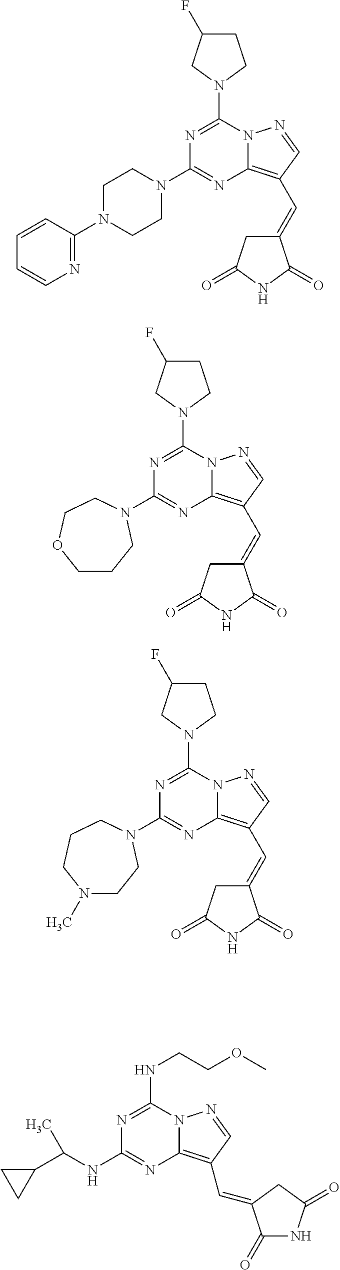

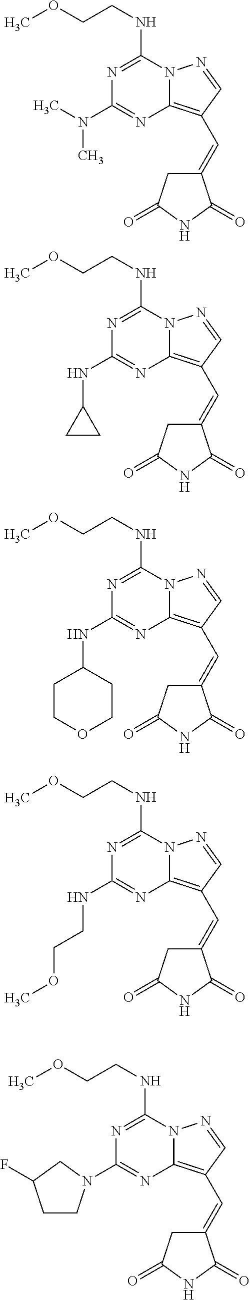

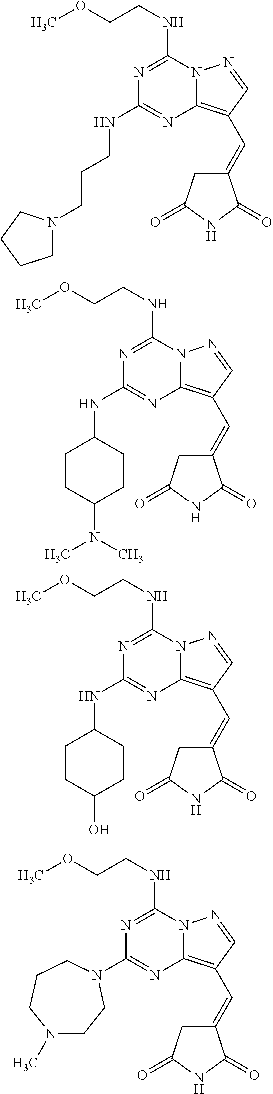

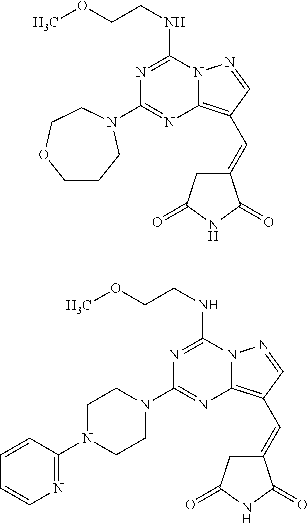

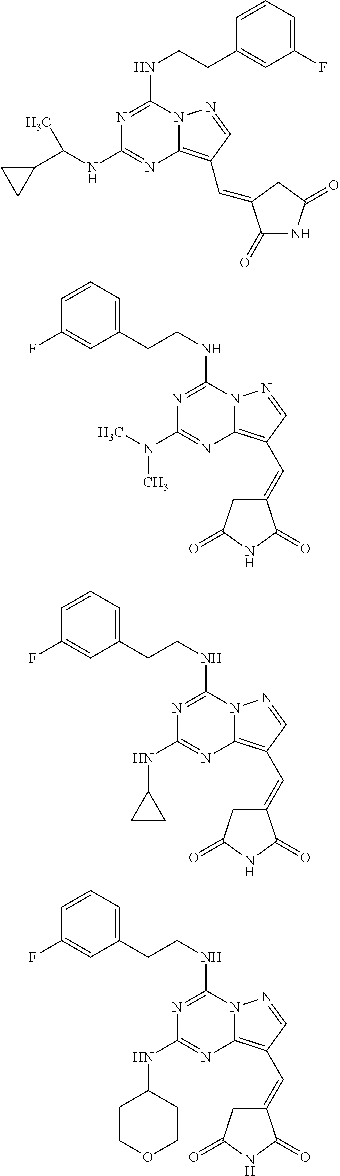

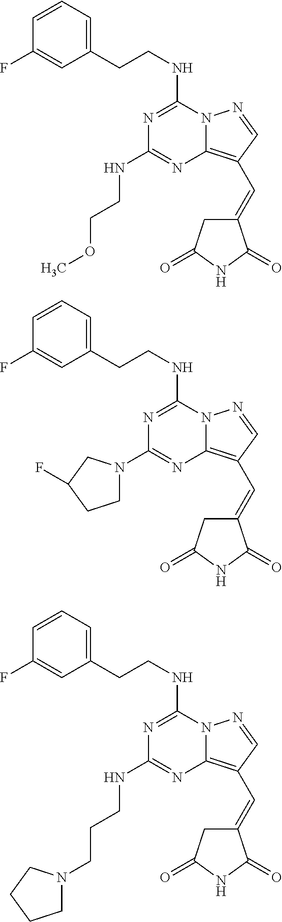

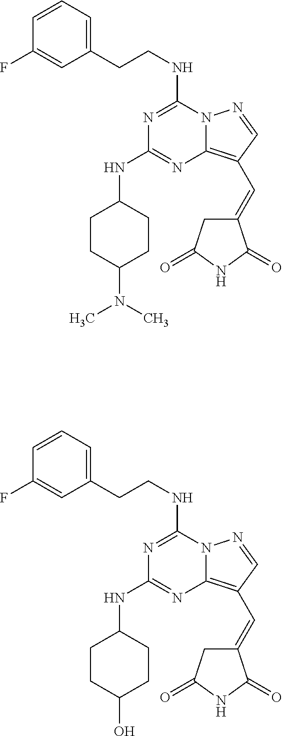

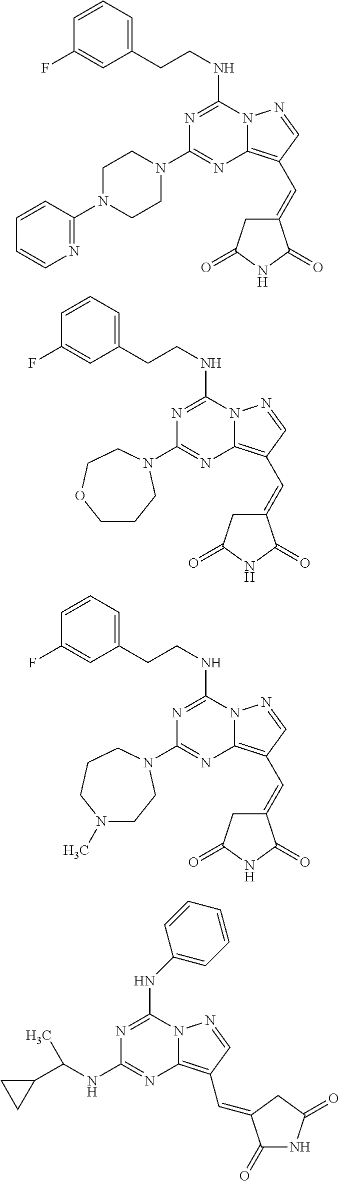

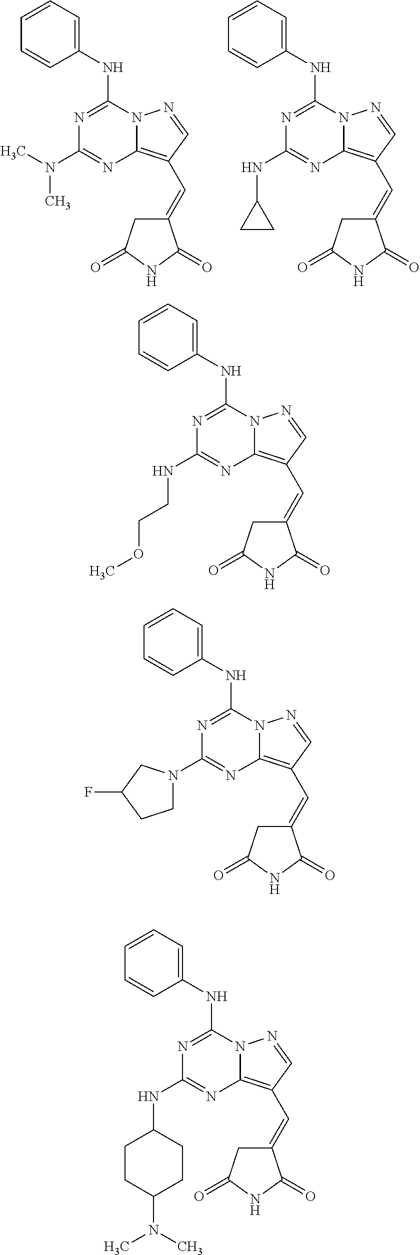

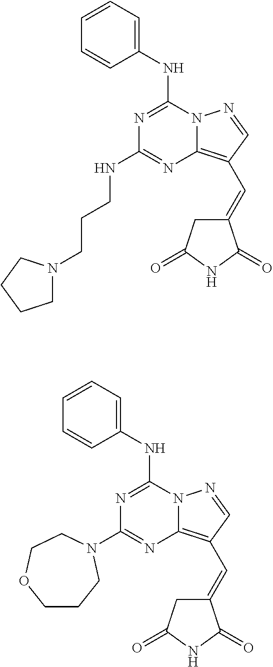

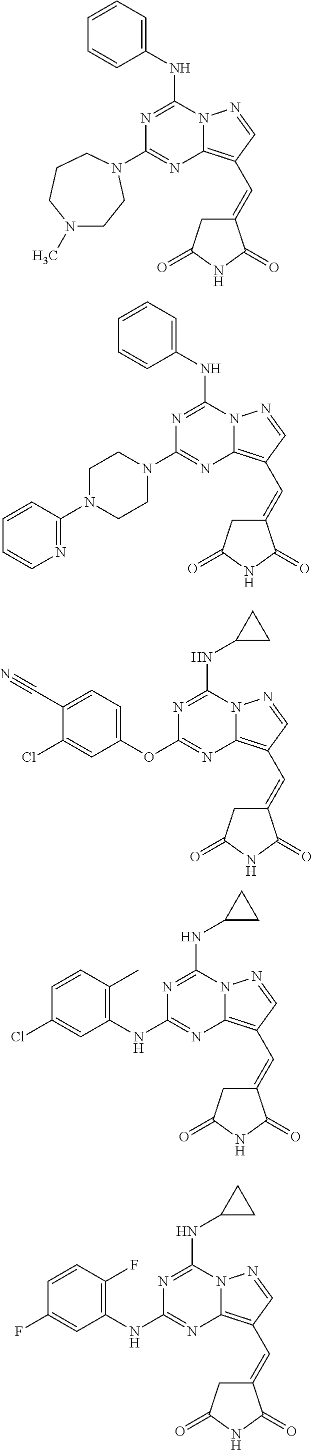

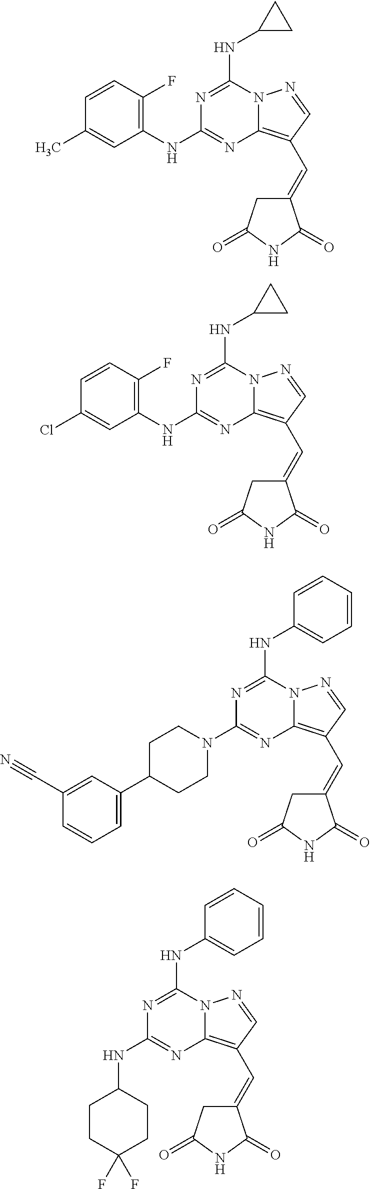

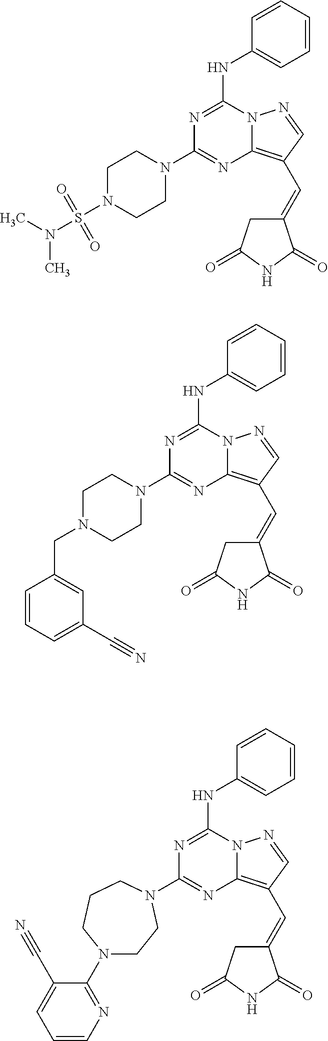

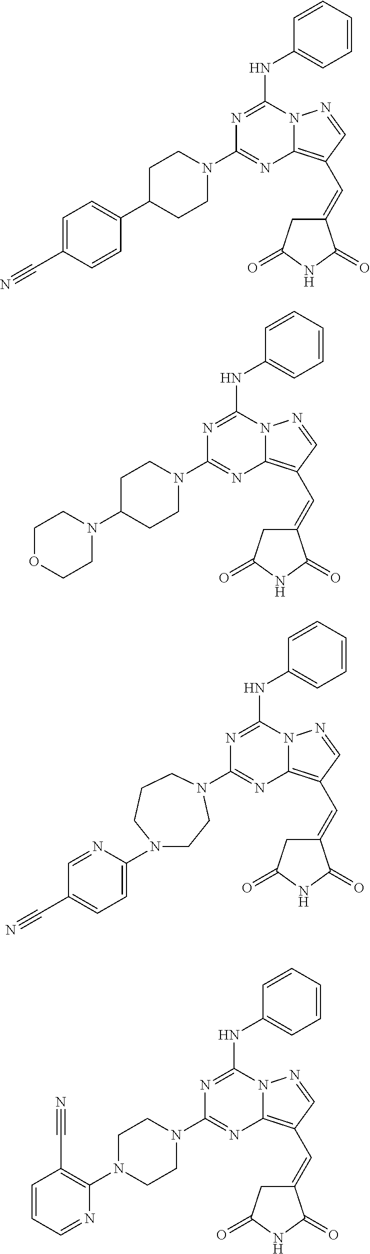

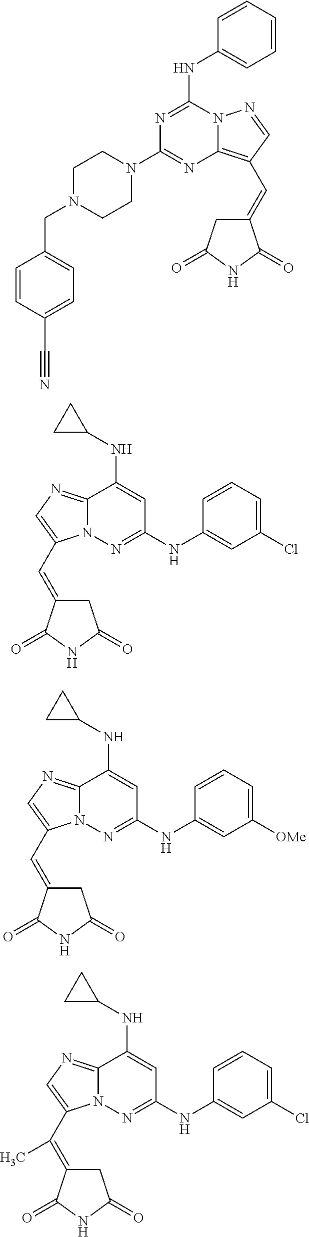

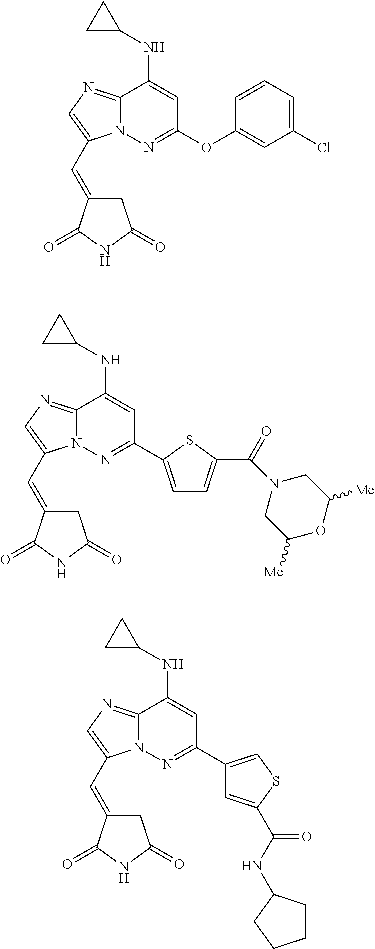

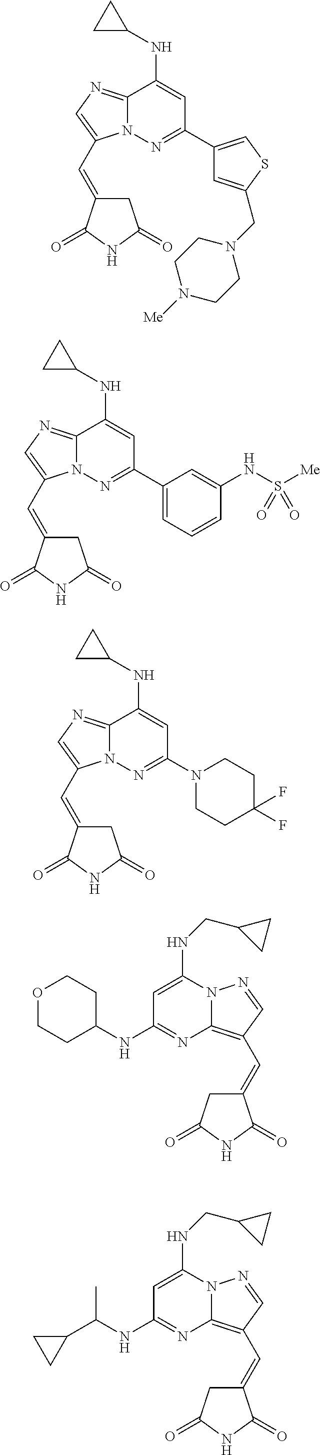

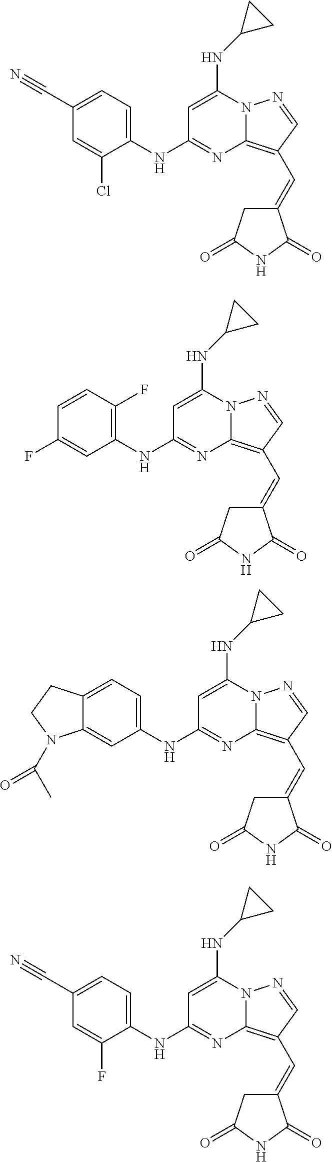

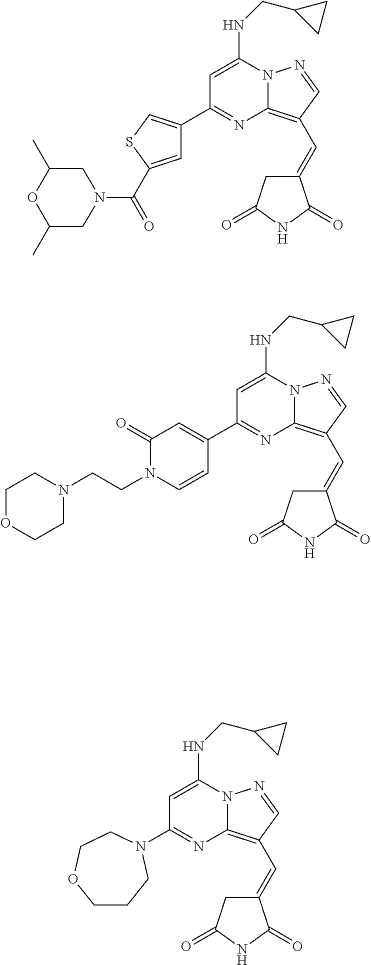

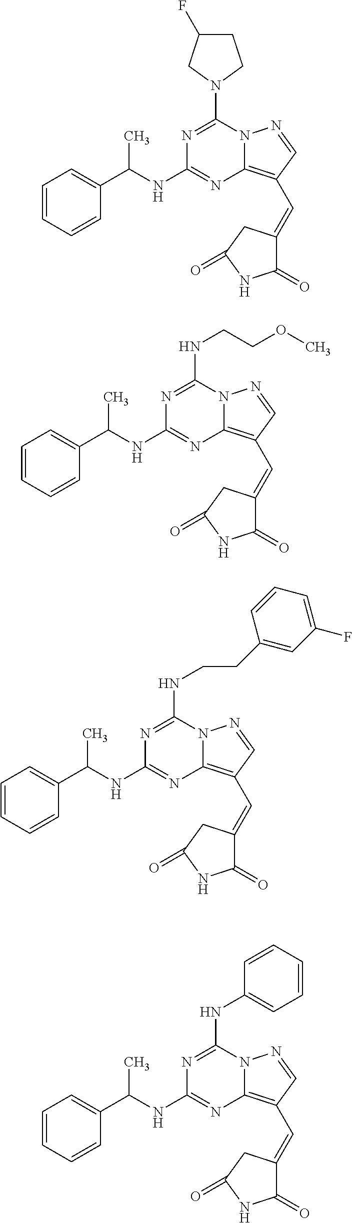

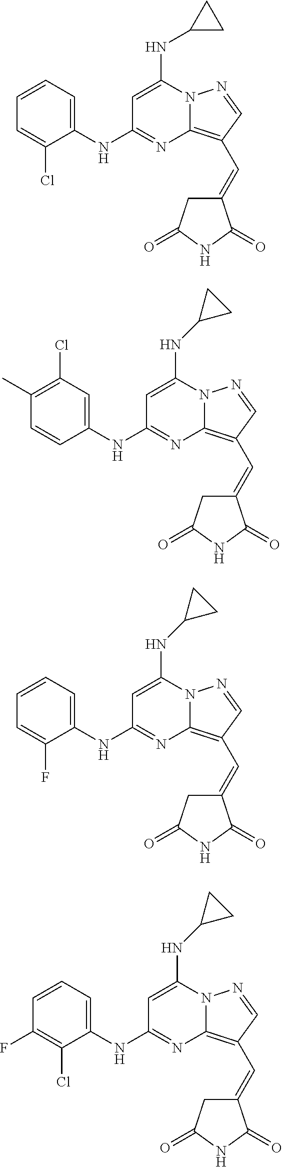

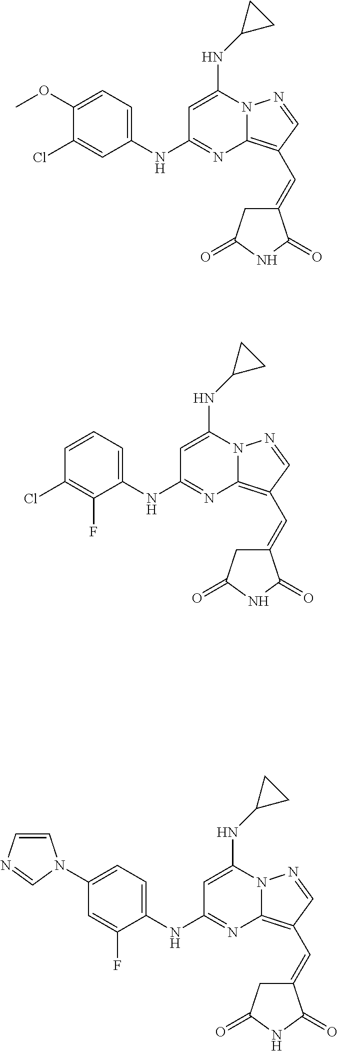

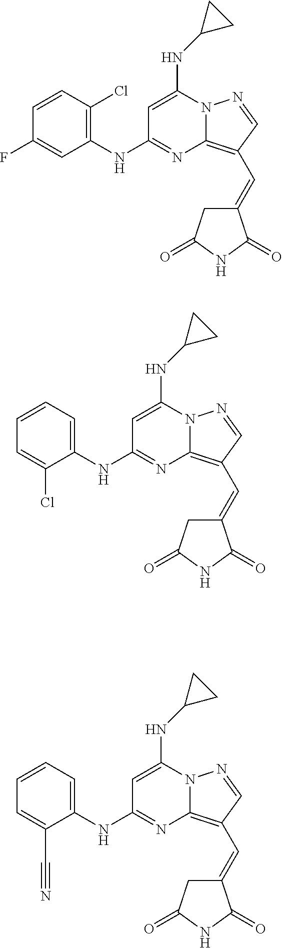

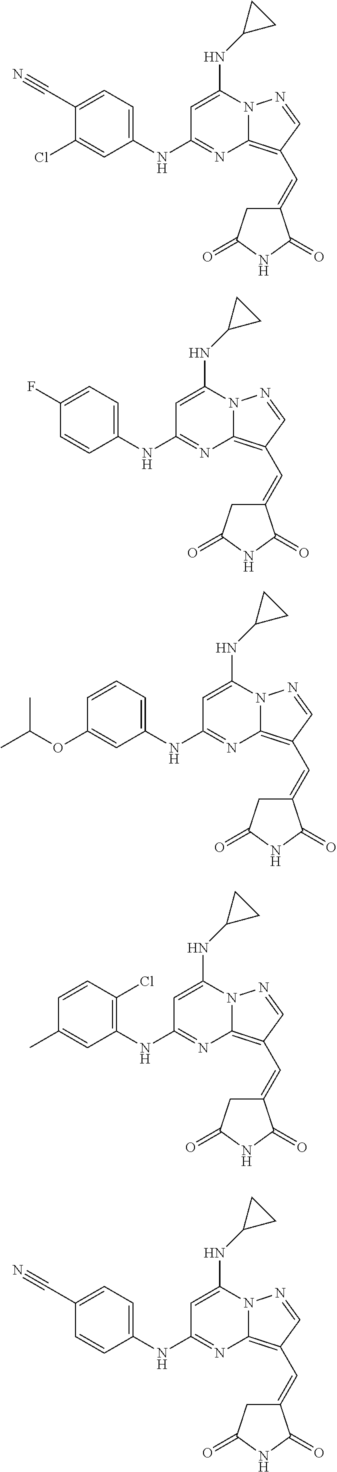

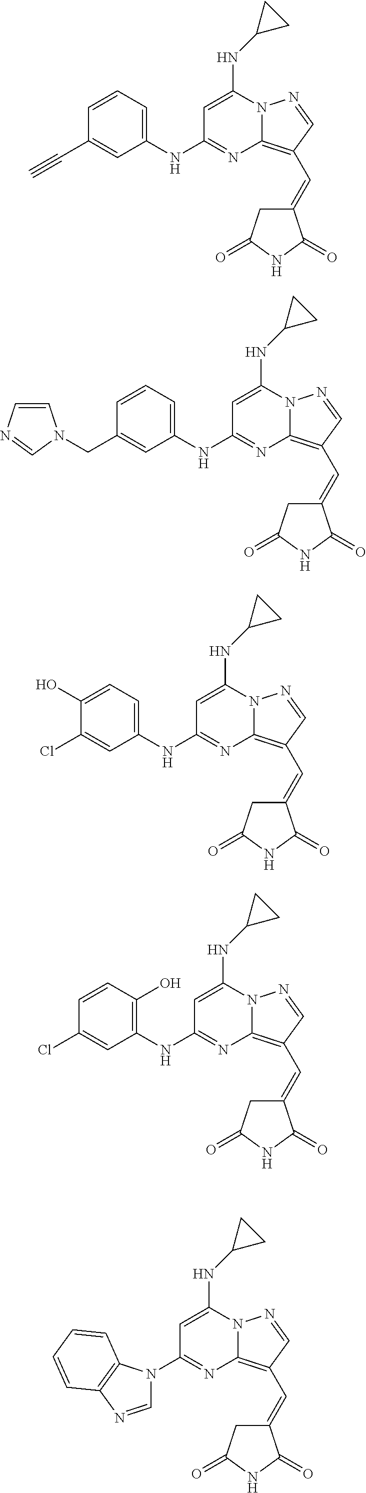

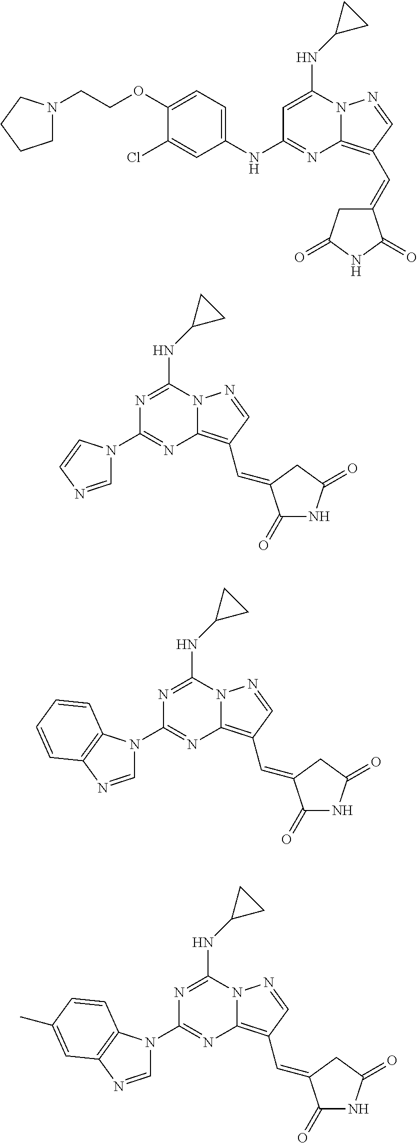

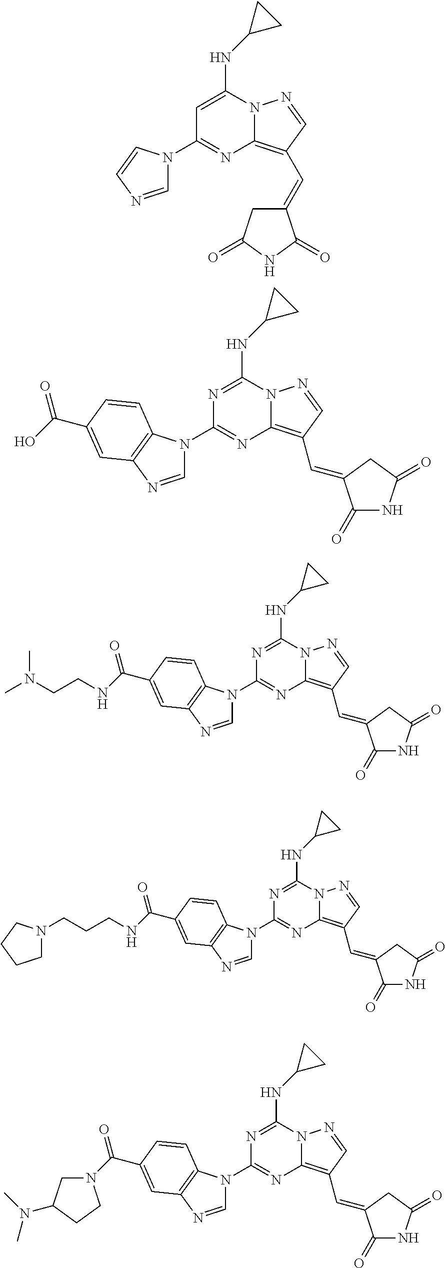

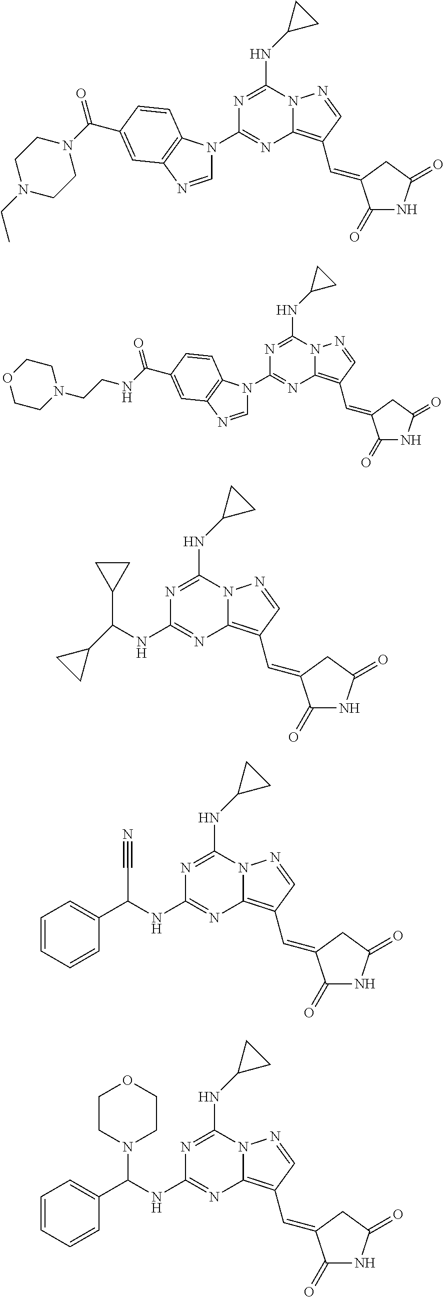

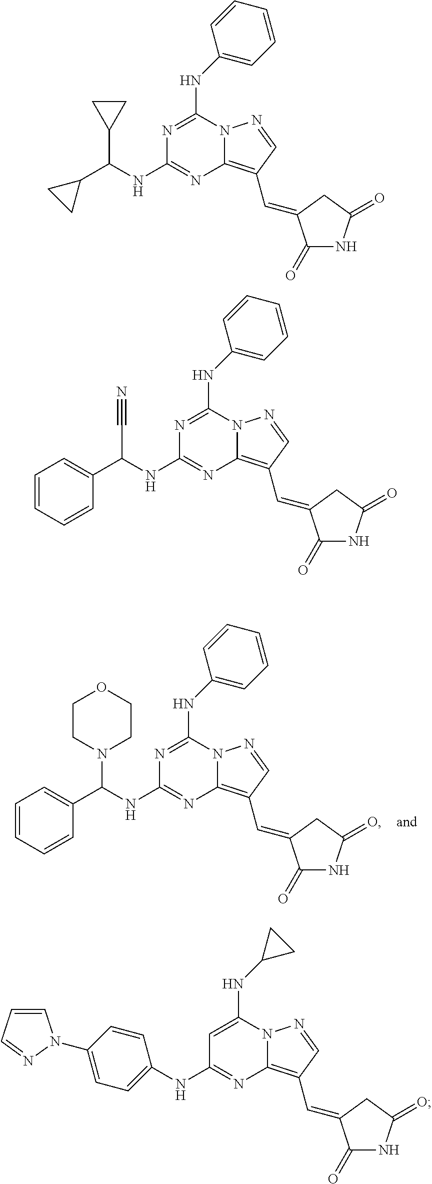

or a pharmaceutically acceptable salt, solvate, and/or prodrug thereof.

In certain embodiments, the present compounds may be in a prodrug form, such as compounds represented by Formula (Ie):

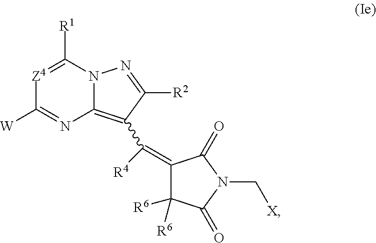

or a pharmaceutically acceptable salt and/or solvate thereof;
wherein,

Z⁴ are independently CR^{1a} or N, R¹ and R^{1a} are independently H, halo, CN, optionally substituted C1-C4 alkyl, optionally substituted C2-C4 alkenyl, optionally substituted C2-C4 alkynyl, optionally substituted C1-C4 alkoxy, or —NR⁷R⁸;

R² is H, halo, CN, or an optionally substituted group selected from C1-C4 alkyl, C2-C4 alkenyl, and C2-C4 alkynyl;

R⁴ is H or optionally substituted C1-C10 alkyl;

each R⁶ is independently H or optionally substituted C1-C10 alkyl

W is halo, —OR⁷, —NR⁷R⁸, —S(O)ₙR⁷, —C(O)OR⁷, optionally substituted aryl, optionally substituted heteroaryl, optionally substituted heterocyclyl, optionally substituted C3-C8 cycloalkyl, or CR⁷R⁸R⁹,

wherein n is 0, 1 or 2,

each R⁷, R⁸, and R⁹ is independently selected from H, optionally substituted C1-C10 alkyl, optionally substituted aryl, optionally substituted arylalkyl, optionally substituted heteroaryl, optionally substituted heteroarylalkyl, and optionally substituted heterocyclyl; or alternatively, R⁷ and R⁸ in NR⁷R⁸, taken together with the nitrogen atom to which they are attached, form a 5 to 8 membered ring that is optionally substituted and optionally contain an additional heteroatom selected from N, O and S as a ring member;

X is hydroxyl or a group having structural formula (II), (III), (IV), or (V):

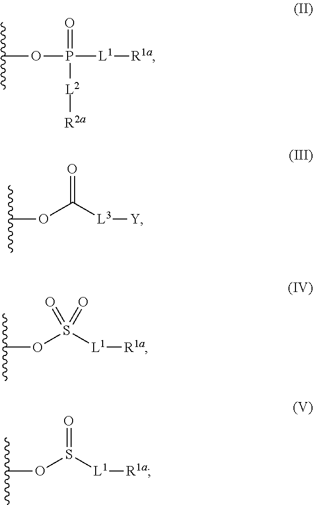

L¹ and L² are each independently a covalent bond, —O—, or —NR^{3a}—;

R^{1a} and R^{2a} are each independently hydrogen, alkyl, heteroalkyl, heteroaryl, heterocyclyl, alkenyl, alkynyl, arylalkyl, heteroarylalkyl, heterocyclylalkyl, -alkylene-C(O)—O—R^{4a}, or -alkylene-O—C(O)—O—R^{4a}; and

R^{3a} and R^{4a} are each independently hydrogen, alkyl, heteroalkyl, cyclylalkyl, heterocyclyl, aryl, heteroaryl, alkenyl, alkynyl, arylalkyl, heterocyclylalkyl, or heteroarylalkyl;

L³ is a covalent bond or alkylene;

Y is OR^{5a}, NR^{5a}R^{6a}, or C(O)OR^{7a}, provided that when Y is C(O)OR^{7a}, then L³ is not a covalent bond; and

R^{5a}, R^{6a}, and R^{7a} are each independently hydrogen, alkyl, arylalkyl, aryl, heteroalkyl, alkylheteroaryl, heterocyclyl, or heteroaryl; or alternatively, R^{5a} and R^{6a}, taken together with the nitrogen atom to which they are attached, form a heterocyclyl ring optionally containing one or more additional heteroatom such as N, O, or S.

It should be understood that when alkylene is substituted as described herein, for example, by —C(O)—O—R^{4a}, —O—C(O)—O—R^{4a}, —OR″, —NR^{5a}R^{6a}, or —C(O)OR^{7a}, the substituent can be attached to any of the carbon atom(s) of the alkylene.

In certain embodiments of Formula (Ie) described above, R² is H.

In certain embodiments of Formula (Ie) described above, R⁴ is H.

In certain embodiments of Formula (Ie) described above, R¹ is —NR⁷R⁸.

In certain embodiments of Formula (Ie) described above, W is —OR⁷ or —NR⁷R⁸.

In certain embodiments of Formula (Ie) described above, R⁷ is optionally substituted aryl or optionally substituted heteroaryl; and R⁸ is H.

In certain embodiments of Formula (Ie) described above, R⁸ is optionally substituted phenyl.

In certain embodiments of Formula (Ie) described above, L¹ and L² are —O—; and R^{1a} and R^{2a} are each independently hydrogen or alkyl.

In certain embodiments of Formula (Ie) described above, L³ is alkylene; and Y is C(O)OR^{7a} or NR^{5a}R^{6a}.

In certain embodiments of Formula (Ie) described above, L³ is a covalent bond; and Y is OR^{5a} or NR^{5a}R^{6a}.

In certain specific embodiments, the present invention provides compounds selected from the group consisting of

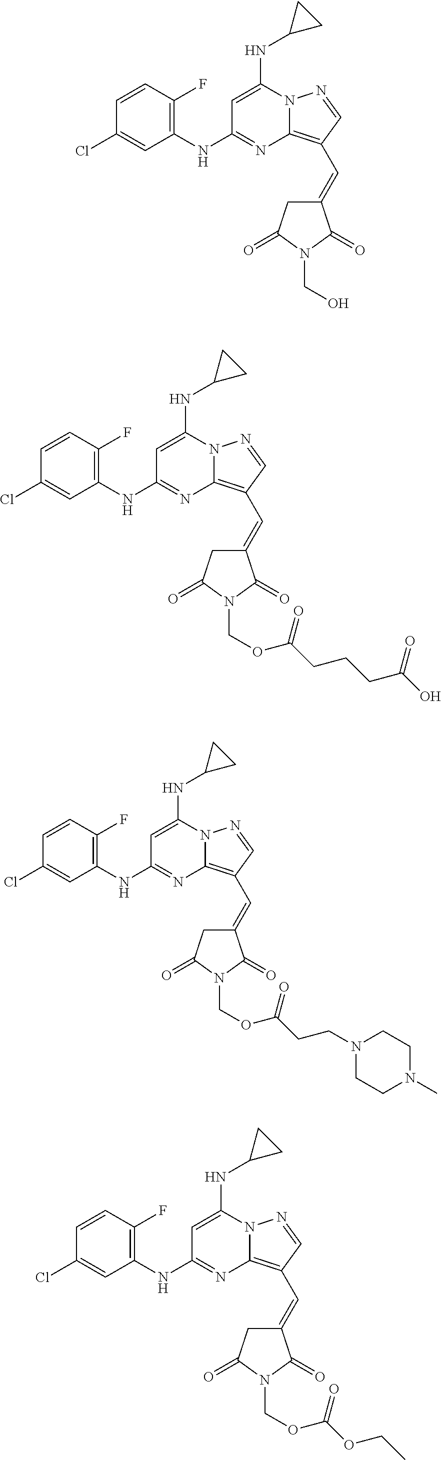

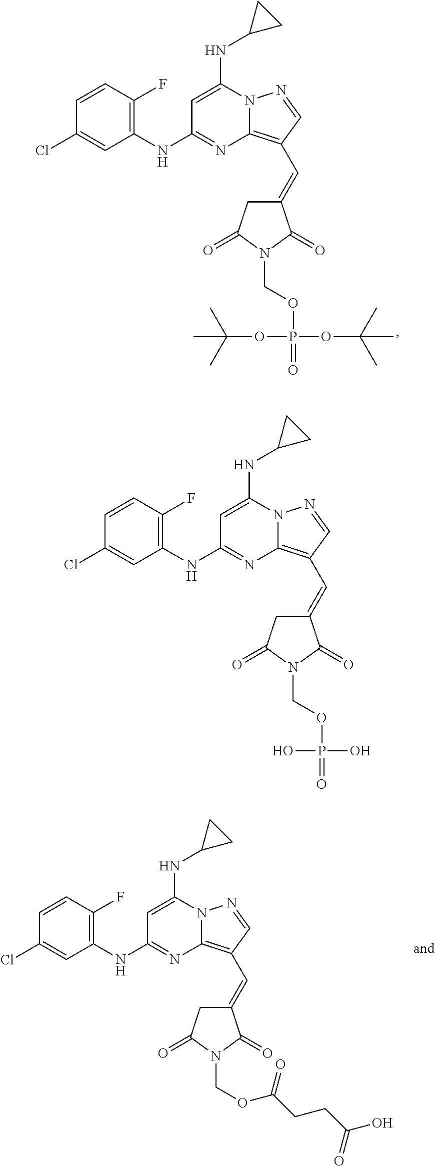

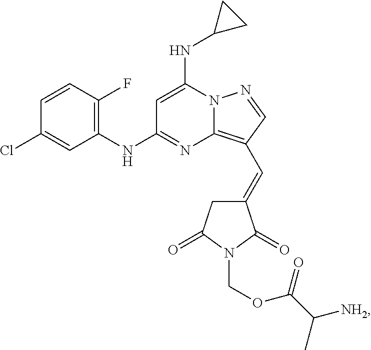

or a pharmaceutically acceptable salt, solvate, and/or prodrug thereof.

### Utilities of the Compounds:

In another aspect, the invention provides a method to treat cancer, a vascular disorder, inflammation, or a pathogenic infection, comprising administering to a subject in need of such treatment, an effective amount of any of the above-described compounds.

The compounds of the invention are useful as medicaments, and are useful for the manufacture of medicaments, including medicaments to treat conditions disclosed herein, such as cancers, inflammatory conditions, infections, pain, and immunological disorders.

The compounds of Formula (I) are active as inhibitors of CK2 and/or Pim kinases, and are thus useful to treat infections by certain pathogens, including protozoans and viruses. The invention thus provides methods for treating protozoal disorders such as protozoan parasitosis, including infection by parasitic protozoa responsible for neurological disorders such as schizophrenia, paranoia, and encephalitis in immunocompromised patients, as well as Chagas' disease. It also provides methods to treat various viral diseases, including human immunodeficiency virus type 1 (HIV-1), human papilloma viruses (HPVs), herpes simplex virus (HSV), Epstein-Barr virus (EBV), human cytomegalovirus, hepatitis C and B viruses, influenza virus, Boma disease virus, adenovirus, coxsackievirus, coronavirus and varicella zoster virus. The methods for treating these disorders comprise administering to a subject in need thereof an effective amount of a compound of Formula (I).

Furthermore, the invention in part provides methods for identifying a candidate molecule that interacts with a CK2 and/or Pim, which comprises contacting a composition containing a CK2 or Pim protein and a molecule described herein with a candidate molecule and determining whether the amount of the molecule described herein that interacts with the protein is modulated, whereby a candidate molecule that modulates the amount of the molecule described herein that interacts with the protein is identified as a candidate molecule that interacts with the protein.

Also provided by the invention are methods for modulating certain protein kinase activities. Protein kinases catalyze the transfer of a gamma phosphate from adenosine triphosphate to a serine or threonine amino acid (serine/thteonine protein kinase), tyrosine amino acid (tyrosine protein kinase), tyrosine, serine or threonine (dual specificity protein kinase) or histidine amino acid (histidine protein kinase) in a peptide or protein substrate.

Thus, included herein are methods which comprise contacting a system comprising a protein kinase protein with a compound described herein in an amount effective for modulating (e.g., inhibiting) the activity of the protein kinase. In some embodiments, the activity of the protein kinase is the catalytic activity of the protein (e.g., catalyzing the transfer of a gamma phosphate from adenosine triphosphate to a peptide or protein substrate). In certain embodiments, provided are methods for identifying a candidate molecule that interacts with a protein kinase, which comprise: contacting a composition containing a protein kinase and a compound described herein with a candidate molecule under conditions in which the compound and the protein kinase interact, and determining whether the amount of the compound that interacts with the protein kinase is modulated relative to a control interaction between the compound and the protein kinase without the candidate molecule, whereby a candidate molecule that modulates the amount of the compound interacting with the protein kinase relative to the control interaction is identified as a candidate molecule that interacts with the protein kinase. Systems in such embodiments can be a cell-free system or a system comprising cells (e.g., in vitro).

The protein kinase, the compound or the molecule in some embodiments is in association with a solid phase. In certain embodiments, the interaction between the compound and the protein kinase is detected via a detectable label, where in some embodiments the protein kinase comprises a detectable label and in certain embodiments the compound comprises a detectable label. The interaction between the compound and the protein kinase sometimes is detected without a detectable label.

Provided also are compositions of matter comprising a protein kinase and a compound described herein. In some embodiments, the protein kinase in the composition is a serine-threonine protein kinase. In some embodiments, the protein kinase in the composition is, or contains a subunit (e.g., catalytic subunit, SH2 domain, SH3 domain) of, CK2 or a Pim subfamily protein kinase (e.g., PIM1, PIM2, PIM3). In certain embodiments the composition is cell free and sometimes the protein kinase is a recombinant protein.

The protein kinase can be from any source, such as cells from a mammal, ape or human, for example. Examples of serine-threonine protein kinases that can be inhibited, or may potentially be inhibited, by compounds disclosed herein include without limitation human versions of CK2, CK2α2, and Pim subfamily kinases (e.g., PIM1, PIM2, PIM3). A serine-threonine protein kinase sometimes is a member of a sub-family containing one or more of the following amino acids at positions corresponding to those listed in human CK2: leucine at position 45, methionine at position 163 and isoleucine at position 174. Examples of such protein kinases include without limitation human versions of CK2, STK10, HIPK2, HIPK3, DAPK3, DYK2 and Pim-1. Nucleotide and amino acid sequences for protein kinases and reagents are publicly available (e.g., World Wide Web URLs ncbi.nlm.nih.gov/sites/entrez/and Invitrogen.com). For example, various nucleotide sequences can be accessed using the following accession numbers: NM_{—}002648.2 and NP_{—}002639.1 for PIM I; NM_{—}006875.2 and NP_{—}006866.2 for PIM2; XM_{—}938171.2 and XP_{—}943264.2 for PIM3.

The invention also in part provides methods for treating a condition related to aberrant cell proliferation. For example, provided are methods of treating a cell proliferative condition in a subject, which comprises administering a compound described herein to a subject in need thereof in an amount effective to treat the cell proliferative condition. The subject may be a research animal (e.g., rodent, dog, cat, monkey), optionally containing a tumor such as a xenograft tumor (e.g., human tumor), for example, or may be a human. A cell proliferative condition sometimes is a tumor or non-tumor cancer, including but not limited to, cancers of the colorectum, breast, lung, liver, pancreas, lymph node, colon, prostate, brain, head and neck, skin, liver, kidney, blood and heart (e.g., leukemia, lymphoma, carcinoma).

Also provided are methods for treating a condition related to inflammation or pain. For example, methods are provided for treating pain in a subject, which comprise administering a compound described herein to a subject in need thereof in an amount effective to treat the pain. Provided also are methods of treating inflammation in a subject, which comprise administering a compound described herein to a subject in need thereof in an amount effective to treat the inflammation. The subject may be a research animal (e.g., rodent, dog, cat; monkey), for example, or may be a human. Conditions associated with inflammation and pain include without limitation acid reflux, heartburn, acne, allergies and allergen sensitivities, Alzheimer's disease, asthma, atherosclerosis, bronchitis, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, heart disease, hepatitis, high blood pressure, insulin resistance, interstitial cystitis, joint pain/arthritis/rheumatoid arthritis, metabolic syndrome (syndrome X), myositis, nephritis, obesity, osteopenia, glomerulonephritis (GN), juvenile cystic kidney disease, and type I nephronophthisis (NPHP), osteoporosis, Parkinson's disease, Guam-Parkinson dementia, supranuclear palsy, Kuf's disease, and Pick's disease, as well as memory impairment, brain ischemia, and schizophrenia, periodontal disease, polyarteritis, polychondritis, psoriasis, scleroderma, sinusitis, Sjögren's syndrome, spastic colon, systemic candidiasis, tendonitis, urinary track infections, vaginitis, inflammatory cancer (e.g., inflammatory breast cancer) and the like.

Methods for determining and monitoring effects of compounds herein on pain or inflammation are known. For example, formalin-stimulated pain behaviors in research animals can be monitored after administration of a compound described herein to assess treatment of pain (e.g., Li et al., Pain 115(1-2): 182-90 (2005)). Also, modulation of pro-inflammatory molecules (e.g., IL-8, GRO-alpha, MCP-1, TNFalpha and iNOS) can be monitored after administration of a compound described herein to assess treatment of inflammation (e.g., Parhar et al., Int J Colorectal Dis. 22(6): 601-9 (2006)), for example. Thus, also provided are methods for determining whether a compound herein reduces inflammation or pain, which comprise contacting a system with a compound described herein in an amount effective for modulating (e.g., inhibiting) the activity of a pain signal or inflammation signal.

Provided also are methods for identifying a compound that reduces inflammation or pain, which comprise: contacting a system with a compound of Formula (I); and detecting a pain signal or inflammation signal, whereby a compound that modulates the pain signal relative to a control molecule is identified as a compound that reduces inflammation of pain. Non-limiting examples of pain signals are formalin-stimulated pain behaviors and examples of inflammation signals include without limitation a level of a pro-inflammatory molecule. The invention thus in part pertains to methods for modulating angiogenesis in a subject, and methods for treating a condition associated with aberrant angiogenesis in a subject. proliferative diabetic retinopathy.

CK2 has also been shown to play a role in the pathogenesis of atherosclerosis, and may prevent atherogenesis by maintaining laminar shear stress flow. CK2 plays a role in vascularization, and has been shown to mediate the hypoxia-induced activation of histone deacetylases (HDACs). CK2 is also involved in diseases relating to skeletal muscle and bone tissue, including, e.g., cardiomyocyte hypertrophy, heart failure, impaired insulin signaling and insulin resistance, hypophosphatemia and inadequate bone matrix mineralization.

Thus in one aspect, the invention provides methods to treat each of these conditions, comprising administering to a subject in need of such treatment an effect amount of a CK2 inhibitor, such as a compound of Formula (I) as described herein.

The invention also in part pertains to methods for modulating an immune response in a subject, and methods for treating a condition associated with an aberrant immune response in a subject. Thus, provided are methods for determining whether a compound herein modulates an immune response, which comprise contacting a system with a compound described herein in an amount effective for modulating (e.g., inhibiting) an immune response or a signal associated with an immune response. Signals associated with immunomodulatory activity include, e.g., stimulation of T-cell proliferation, suppression or induction of cytokines, including, e.g., interleukins, interferon-γ and TNF. Methods of assessing immunomodulatory activity are known in the art.

Also provided are methods for treating a condition associated with an aberrant immune response in a subject, which comprise administering a compound described herein to a subject in need thereof in an amount effective to treat the condition. Conditions characterized by an aberrant immune response include without limitation, organ transplant rejection, asthma, autoimmune disorders, including rheumatoid arthritis, multiple sclerosis, myasthenia gravis, systemic lupus erythematosus, scleroderma, polymyositis, mixed connective tissue disease (MCTD), Crohn's disease, and ulcerative colitis. In certain embodiments, an immune response may be modulated by administering a compound herein in combination with a molecule that modulates (e.g., inhibits) the biological activity of an mTOR pathway member or member of a related pathway (e.g., mTOR, PI3 kinase, AKT). In certain embodiments the molecule that modulates the biological activity of an mTOR pathway member or member of a related pathway is rapamycin. In certain embodiments, provided herein is a composition comprising a compound described herein in combination with a molecule that modulates the biological activity of an mTOR pathway member or member of a related pathway, such as rapamycin, for example.

### Compositions and Routes of Administration:

In another aspect, the invention provides pharmaceutical compositions (i.e., formulations). The pharmaceutical compositions can comprise a compound of any of Formulae I, (Ia), (Ib), (Ic), and (Id) as described herein which is admixed with at least one pharmaceutically acceptable excipient or carrier. Frequently, the composition comprises at least two pharmaceutically acceptable excipients or carriers.

Any suitable formulation of a compound described above can be prepared for administration by methods known in the art. Selection of useful excipients or carriers can be achieved without undue experimentation, based on the desired route of administration and the physical properties of the compound to be administered.

Any suitable route of administration may be used, as determined by a treating physician, including, but not limited to, oral, parenteral, intravenous, intramuscular, transdermal, topical and subcutaneous routes. Depending on the subject to be treated, the mode of administration, and the type of treatment desired—e.g., prevention, prophylaxis, therapy; the compounds are formulated in ways consonant with these parameters. Preparation of suitable formulations for each route of administration are known in the art. A summary of such formulation methods and techniques is found in Remington's Pharmaceutical Sciences, latest edition, Mack Publishing Co., Easton, Pa. The formulation of each substance or of the combination of two substances will frequently include a diluent as well as, in some cases, adjuvants, buffers, preservatives and the like. The substances to be administered can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the appropriate dosage of a compound described above often is 0.01-15 mg/kg, and sometimes 0.1-10 mg/kg. In some embodiments, a suitable dosage of the compound of the invention for an adult patient will be between 1 and 1000 mg per'dose, frequently between 10 and 300 mg, and the dosage may be administered 1-4 times per day. Dosage levels are dependent on the nature of the condition, drug efficacy, the condition of the patient, the judgment of the practitioner, and the frequency and mode of administration; however, optimization of such parameters is within the ordinary level of skill in the art.

### Therapeutic Combinations:

Compounds of the invention may be used alone or in combination with another therapeutic agent. The invention provides methods to treat conditions such as cancer, inflammation and immune disorders by administering to a subject in need of such treatment a therapeutically effective amount of a therapeutic agent useful for treating said disorder and administering to the same subject a therapeutically effective amount of a modulator of the present invention, i.e., a compound of the invention. The therapeutic agent and the modulator may be “co-administered”, i.e, administered together, either as separate pharmaceutical compositions or admixed in a single pharmaceutical composition. By “administered together”, the therapeutic agent and the modulator may also be administered separately, including at different times and with different frequencies. The modulator may be administered by any known route, such as orally, intravenously, intramuscularly, nasally, and the like; and the therapeutic agent may also be administered by any conventional route. In many embodiments, at least one and optionally both of the modulator and the therapeutic agent may be administered orally. Preferably, the modulator is an inhibitor, and it may inhibit either one of CK2 and Pim, or both of them to provide the treatment effects described herein.

In certain embodiments, a “modulator” as described above may be used in combination with a therapeutic agent that can act by binding to regions of DNA that can form certain quadruplex structures. In such embodiments, the therapeutic agents have anticancer activity on their own, but their activity is enhanced when they are used in combination with a modulator. This synergistic effect allows the therapeutic agent to be administered in a lower dosage while achieving equivalent or higher levels of at least one desired effect.

A modulator may be separately active for treating a cancer. For combination therapies described above, when used in combination with a therapeutic agent, the dosage of a modulator will frequently be two-fold to ten-fold lower than the dosage required when the modulator is used alone to treat the same condition or subject. Determination of a suitable amount of the modulator for use in combination with a therapeutic agent is readily determined by methods known in the art.

Compounds and compositions of the invention may be used in combination with anticancer or other agents, such as palliative agents, that are typically administered to a patient being treated for cancer. Such “anticancer agents” include, e.g., classic chemotherapeutic agents, as well as molecular targeted therapeutic agents, biologic therapy agents, and radiotherapeutic agents.

When a compound or composition of the invention is used in combination with an anticancer agent to another agent, the present invention provides, for example, simultaneous, staggered, or alternating treatment. Thus, the compound of the invention may be administered at the same time as an anticancer agent, in the same pharmaceutical composition; the compound of the invention may be administered at the same time as the anticancer agent, in separate pharmaceutical compositions; the compound of the invention may be administered before the anticancer agent, or the anticancer agent may be administered before the compound of the invention, for example, with a time difference of seconds, minutes, hours, days, or weeks.

In examples of a staggered treatment, a course of therapy with the compound of the invention may be administered, followed by a course of therapy with the anticancer agent, or the reverse order of treatment may be used, and more than one series of treatments with each component may also be used. In certain examples of the present invention, one component, for example, the compound of the invention or the anticancer agent, is administered to a mammal while the other component, or its derivative products, remains in the bloodstream of the mammal. For example, a compound for formulae (I)-(IV) may be administered while the anticancer agent or its derivative products remains in the bloodstream, or the anticancer agent may be administered while the compound of formulae (I)-(IV) or its derivatives remains in the bloodstream. In other examples, the second component is administered after all, or most of the first component, or its derivatives, have left the bloodstream of the mammal.

The compound of the invention and the anticancer agent may be administered in the same dosage form, e.g., both administered as intravenous solutions, or they may be administered in different dosage forms, e.g., one compound may be administered topically and the other orally. A person of ordinary skill in the art would be able to discern which combinations of agents would be useful based on the particular characteristics of the drugs and the cancer involved.

Anticancer agents useful in combination with the compounds of the present invention may include agents selected from any of the classes known to those of ordinary skill in the art, including, but not limited to, antimicrotubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; nonreceptor tyrosine kinase angiogenesis inhibitors; immunotherapeutic agents; pro-apoptotic agents; and cell cycle signaling inhibitors; and other agents described below.

Anti-microtubule or anti-mitotic agents are phase specific agents that are typically active against the microtubules of tumor cells during M or the mitosis phase of the cell cycle. Examples of anti-microtubule agents include, but are not limited to, diterpenoids and vinca alkaloids.

Plant alkaloid and terpenoid derived agents include mitotic inhibitors such as the vinca alkaloids vinblastine, vincristine, vindesine, and vinorelbine; and microtubule polymer stabilizers such as the taxanes, including, but not limited to paclitaxel, docetaxel, larotaxel, ortataxel, and tesetaxel.

Diterpenoids, which are derived from natural sources, are phase specific anti-cancer agents that are believed to operate at the G2/M phases of the cell cycle. It is believed that the diterpenoids stabilize the p-tubulin subunit of the microtubules, by binding with this protein. Disassembly of the protein appears then to be inhibited with mitosis being arrested and cell death following.

Examples of diterpenoids include, but are not limited to, taxanes such as paclitaxel, docetaxel, larotaxel, ortataxel, and tesetaxel. Paclitaxel is a natural diterpene product isolated from the Pacific yew tree Taxus brevifolia and is commercially available as an injectable solution TAXOL®. Docetaxel is a semisynthetic derivative of paclitaxel q. v., prepared using a natural precursor, 10-deacetyl-baccatin III, extracted from the needle of the European Yew tree. Docetaxel is commercially available as an injectable solution as TAXOTERE®.

Vinca alkaloids are phase specific anti-neoplastic agents derived from the periwinkle plant. Vinca alkaloids that are believed to act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, vindesine, and vinorelbine. Vinblastine, vincaleukoblastine sulfate, is commercially available as VELBAN® as an injectable solution. Vincristine, vincaleukoblastine 22-oxo-sulfate, is commercially available as ONCOVIN® as an injectable solution. Vinorelbine, is commercially available as an injectable solution of vinorelbine tartrate (NAVELBINE®), and is a semisynthetic vinca alkaloid derivative.

Platinum coordination complexes are non-phase specific anti-cancer agents, which are interactive with DNA. The platinum complexes are believed to enter tumor cells, undergo, aquation and form intra- and interstrand crosslinks with DNA causing adverse biological effects to the tumor. Platinum-based coordination complexes include, but are not limited to cisplatin, carboplatin, nedaplatin, oxaliplatin, satraplatin, and (SP-4-3)-(cis)-amminedichloro-[2-methylpyridine]platinum(II). Cisplatin, cis-diamminedichloroplatinum, is commercially available as PLATINOL® as an injectable solution. Carboplatin, platinum, diammine [1,1-cyclobutane-dicarboxylate(2-)-0,0′], is commercially available as PARAPLATIN® as an injectable solution.

Alkylating agents are generally non-phase specific agents and typically are strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, alkyl sulfonates such as busulfan; ethyleneimine and methylmelamine derivatives such as altretamine and thiotepa; nitrogen mustards such as chlorambucil, cyclophosphamide, estramustine, ifosfamide, mechlorethamine, melphalan, and uramustine; nitrosoureas such as carmustine, lomustine, and streptozocin; triazenes and imidazotetrazines such as dacarbazine, procarbazine, temozolamide, and temozolomide. Cyclophosphamide, 2-[bis(2-chloroethyl)-amino]tetrahydro-2H-1,3,2-oxazaphosphorine 2-oxide monohydrate, is commercially available as an injectable solution or tablets as CYTOXAN®. Melphalan, 4-[bis(2-chloroethyl)amino]-L-phenylalanine, is commercially available as an injectable solution or tablets as ALKERAN®. Chlorambucil, 4-[bis(2-chloroethyl)amino]-benzenebutanoic acid, is commercially available as LEUKERAN® tablets. Busulfan, 1,4-butanediol dimethanesulfonate, is commercially available as MYLERAN® TABLETS. Carmustine, 1,3-[bis(2-chloroethyl)-1-nitrosourea, is commercially available as single vials of lyophilized material as BiCNU®, 5-(3,3-dimethyl-1-triazeno)-imidazole-4-carboxamide, is commercially available as single vials of material as DTIC-Dome®. Furthermore, alkylating agents include (a) alkylating-like platinum-based chemotherapeutic agents such as cisplatin, carboplatin, nedaplatin, oxaliplatin, satraplatin, and (SP-4-3)-(cis)-amminedichloro-[2-methylpyridine]platinum(II); (b) alkyl sulfonates such as busulfan; (c) ethyleneimine and methylmelamine derivatives such as altretamine and thiotepa; (d) nitrogen mustards such as chlorambucil, cyclophosphamide, estramustine, ifosfamide, mechlorethamine, trofosamide, prednimustine, melphalan, and uramustine; (e) nitrosoureas such as carmustine, lomustine, fotemustine, nimustine, ranimustine and streptozocin; (f) triazenes and imidazotetrazines such as dacarbazine, procarbazine, temozolamide, and temozolomide.

Anti-tumor antibiotics are non-phase specific agents which are believed to bind or intercalate with DNA. This may result in stable DNA complexes or strand breakage, which disrupts ordinary function of the nucleic acids, leading to cell death. Examples of anti-tumor antibiotic agents include, but are not limited to, anthracyclines such as daunorubicin (including liposomal daunorubicin), doxorubicin (including liposomal doxorubicin), epirubicin, idarubicin, and valrubicin; streptomyces-related agents such as bleomycin, actinomycin, mithramycin, mitomycin, porfiromycin; and mitoxantrone. Dactinomycin, also know as Actinomycin D, is commercially available in injectable form as COSMEGEN®. Daunorubicin, (8S-cis-)-8-acetyl-10-[(3-amino-2,3,6-trideoxy-a-L-lyxohexopyranosyl)oxy]-7,8,9,10-tetrahydro-6,8,11-trihydroxy-1-methoxy-5,12-naphthacenedione hydrochloride, is commercially available as a liposomal injectable form as DAUNOXOME® or as an injectable as CERUBIDINE®. Doxorubicin, (8S, 10S)-10-[(3-amino-2,3,6-trideoxy-α-L-lyxohexopyranosyl)oxy]-8-glycoloyl, 7,8,9,10-tetrahydro-6,8,11-trihydroxy-1-methoxy-5,12-naphthacenedione hydrochloride, is commercially available in an injectable form as RUBEX® or ADRIAMYCIN RDF®. Bleomycin, a mixture of cytotoxic glycopeptide antibiotics isolated from a strain of Streptomyces verticil/us, is commercially available as BLENOXANE®.

Topoisomerase inhibitors include topoisomerase I inhibitors such as camptothecin, topotecan, irinotecan, rubitecan, and belotecan; and topoisomerase II inhibitors such as etoposide, teniposide, and amsacrine.

Topoisomerase II inhibitors include, but are not limited to, epipodophyllotoxins, which are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide, teniposide, and amsacrine. Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Teniposide, 4′-demethyl-epipodophyllotoxin 9[4,6-0-(R)-thenylidene-β-D-glucopyranoside], is commercially available as an injectable solution as VUMON® and is commonly known as VM-26.

Topoisomerase I inhibitors including, camptothecin and camptothecin derivatives. Examples of topoisomerase I inhibitors include, but are not limited to camptothecin, topotecan, irinotecan, rubitecan, belotecan and the various optical forms (i.e., (R), (S) or (R,S)) of 7-(4-methylpiperazino-methylene)-10,11-ethylenedioxy-camptothecin, as described in U.S. Pat. Nos. 6,063,923; 5,342,947; 5,559,235; 5,491,237 and pending U.S. patent application Ser. No. 08/977,217 filed Nov. 24, 1997. Irinotecan HCl, (4S)-4,11-diethyl-4-hydroxy-9-[(4-piperidinopiperidino)-carbonyloxy]-1H-yrano[3′,4′,6,7]indolizino[1,2-b]quinoline-3, 14(4H, 12H)-dione hydrochloride, is commercially available as the injectable solution CAMPTOSAR®. Irinotecan is a derivative of camptothecin which binds, along with its active metabolite 8N-38, to the topoisomerase I-DNA complex. Topotecan HCl, (S)-10[(dimethylamino)methyl]-4-ethyl-4,9-dihydroxy-1H-pyrano[3′,4′,6,7]indolizino[1,2-b]quinoline-3,14-(4H, 12H)-dione monohydrochloride, is commercially available as the injectable solution HYCAMTIN®.

Anti-metabolites include (a) purine analogs such as fludarabine, cladribine, chlorodeoxyadenosine, clofarabine, mercaptopurine, pentostatin, and thioguanine; (b) pyrimidine analogs such as fluorouracil, gemcitabine, capecitabine, cytarabine, azacitidine, edatrexate, floxuridine, and troxacitabine; (c) antifolates, such as methotrexate, pemetrexed, raltitrexed, and trimetrexate. Anti-metabolites also include thymidylate synthase inhibitors, such as fluorouracil, raltitrexed, capecitabine, floxuridine and pemetrexed; and ribonucleotide reductase inhibitors such as claribine, clofarabine and fludarabine. Antimetabolite neoplastic agents are phase specific anti-neoplastic agents that typically act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Anti-metabolites, include purine analogs, such as fludarabine, cladribine, chlorodeoxyadenosine, clofarabine, mercaptopurine, pentostatin, erythrohydroxynonyladenine, fludarabine phosphate and thioguanine; pyrimidine analogs such as fluorouracil, gemcitabine, capecitabine, cytarabine, azacitidine, edatrexate, floxuridine, and troxacitabine; antifolates, such as methotrexate, pemetrexed, raltitrexed, and trimetrexate. Cytarabine, 4-amino-1-p-D-arabinofuranosyl-2 (1H)-pyrimidinone, is commercially available as CYTOSAR-U® and is commonly known as Ara-C. Mercaptopurine, 1,7-dihydro-6H-purine-6-thione monohydrate, is commercially available as PURINETHOL®. Thioguanine, 2-amino-1,7-dihydro-6H-purine-6-thione, is commercially available as TABLOID®. Gemcitabine, 2′-deoxy-2′,2′-difluorocytidine monohydrochloride (p-isomer), is commercially available as GEMZAR®.

Hormonal therapies include (a) androgens such as fluoxymesterone and testolactone; (b) antiandrogens such as bicalutamide, cyproterone, flutamide, and nilutamide; (c) aromatase inhibitors such as aminoglutethimide, anastrozole, exemestane, formestane, and letrozole; (d) corticosteroids such as dexamethasone and prednisone; (e) estrogens such as diethylstilbestrol; (f) antiestrogens such as fulvestrant, raloxifene, tamoxifen, and toremifine; (g) LHRH agonists and antagonists such as buserelin, goserelin, leuprolide, and triptorelin; (h) progestins such as medroxyprogesterone acetate and megestrol acetate; and (i) thyroid hormones such as levothyroxine and liothyronine. Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, androgens such as fluoxymesterone and testolactone; antiandrogens such as bicalutamide, cyproterone, flutamide, and nilutamide; aromatase inhibitors such as aminoglutethimide, anastrozole, exemestane, formestane, vorazole, and letrozole; corticosteroids such as dexamethasone, prednisone and prednisolone; estrogens such as diethylstilbestrol; antiestrogens such as fulvestrant, raloxifene, tamoxifen, toremifine, droloxifene, and iodoxyfene, as well as selective estrogen receptor modulators (SERMS) such those described in U.S. Pat. Nos. 5,681,835, 5,877,219, and 6,207,716; 5α-reductases such as finasteride and dutasteride; gonadotropin-releasing hormone (GnRH) and analogues thereof which stimulate the release of leutinizing hormone (LH) and/or follicle stimulating hormone (FSH), for example LHRH agonists and antagonists such as buserelin, goserelin, leuprolide, and triptorelin; progestins such as medroxyprogesterone acetate and megestrol acetate; and thyroid hormones such as levothyroxine and liothyronine.

Signal transduction pathway inhibitors are those inhibitors, which block or inhibit a chemical process which evokes an intracellular change, such as cell proliferation or differentiation. Signal tranduction inhibitors useful in the present invention include, e.g., inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Molecular targeted agents include (a) receptor tyrosine kinase (‘RTK’) inhibitors, such as inhibitors of EGFR, including erlotinib, gefitinib, and neratinib; inhibitors of VEGFR including vandetanib, semaxinib, and cediranib; and inhibitors of PDGFR; further included are RTK inhibitors that act at multiple receptor sites such as lapatinib, which inhibits both EGFR and HER2, as well as those inhibitors that act at each of C-kit, PDGFR and VEGFR, including but not limited to axitinib, sunitinib, sorafenib and toceranib; also included are inhibitors of BCR-ABL, c-kit and PDGFR, such as imatinib; (b) FKBP binding agents, such as an immunosuppressive macrolide antibiotic, including bafilomycin, rapamycin (sirolimus) and everolimus; (c) gene therapy agents, antisense therapy agents, and gene expression modulators such as the retinoids and rexinoids, e.g. adapalene, bexarotene, trans-retinoic acid, 9-cis-retinoic acid, and N-(4-hydroxyphenyl)retinamide; (d) phenotype-directed therapy agents, including monoclonal antibodies such as alemtuzumab, bevacizumab, cetuximab, ibritumomab tiuxetan, rituximab, and trastuzumab; (e) immunotoxins such as gemtuzumab ozogamicin; (f) radioimmunoconjugates such as 131I-tositumomab; and (g) cancer vaccines.

Several protein tyrosine kinases catalyse the phosphorylation of specific tyrosyl residues in various proteins involved in the regulation of cell growth. Such protein tyrosine kinases can be broadly classified as receptor or non-receptor kinases. Receptor tyrosine kinases are transmembrane proteins having an extracellular ligand binding domain, a transmembrane domain, and a tyrosine kinase domain. Receptor tyrosine kinases are involved in the regulation of cell growth and are sometimes termed growth factor receptors.

Inappropriate or uncontrolled activation of many of these kinases, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGF1) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene.

Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al., Drug Discov. Today (1997), 2(2):50-63; and Lofts, F. J. et al., “Growth factor receptors as targets”, New Molecular Targets for Cancer Chemotherapy, ed. Workman, Paul and Kerr, David, CRC press 1994, London. Specific examples of receptor tyrosine kinase inhibitors include, but are not limited to, sunitinib, erlotinib, gefitinib, and imatinib.

Tyrosine kinases which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non: receptor kinases and agents which inhibit non-receptor tyrosine kinase function are described in Sinh, S, and Corey, S. J., J. Hematotherapy & Stem Cell Res. (1999) 8(5): 465-80; and Bolen, J. B., Brugge, J. S., Annual Review of Immunology. (1997) 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E., J. Pharmacol. Toxicol. Methods. (1995), 34-(3): 125-32. Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., J. Biochemistry. (1999) 126 (5): 799-803; Brodt, P, Samani, A, & Navab, R, Biochem. Pharmacol. (2000) 60:1101-1107; Massague, J., Weis-Garcia, F., Cancer Surv. (1996) 27:41-64; Philip, P. A, and Harris, A L, Cancer Treat. Res. (1995) 78: 3-27; Lackey, K. et al. Bioorg. Med. Chem. Letters, (2000) 10(3): 223-226; U.S. Pat. No. 6,268,391; and Martinez-Lacaci, I., et al., Int. J. Cancer (2000), 88(1): 44-52. Inhibitors of Phosphotidyl inositol-3 Kinase family members including blockers of PI3-kinase, ATM, DNA-PK, and Ku are also useful in the present invention. Such kinases are discussed in Abraham, R T. Current Opin. Immunol. (1996), 8(3): 412-8; Canman, C. E., Lim, D. S., Oncogene (1998) 17(25): 3301-8; Jackson, S. P., Int. J. Biochem. Cell Biol. (1997) 29(7):935-8; and Zhong, H. et al., Cancer Res. (2000) 60(6):1541-5. Also useful in the present invention are Myo-inositol signaling inhibitors such as phospholipase C blockers and Myoinositol analogues. Such signal inhibitors are described in Powis, G., and Kozikowski A, (1994) New Molecular Targets for Cancer Chemotherapy, ed., Paul Workman and David Kerr, CRC Press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R, Gervasoni, S I, Matar, P., J. Biomed. Sci. (2000) 7(4): 292-8; Ashby, M. N., Curr. Opin. Lipidol. (1998) 9(2): 99-102; and Oliff, A., Biochim. Biophys. Acta, (1999) 1423(3):C19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al., Cancer Treat. Rev., (2000) 26(4): 269-286); Herceptin® erbB2 antibody (see Stern, D F, Breast Cancer Res. (2000) 2(3):176-183); and 2CB VEGFR2 specific antibody (see Brekken, R. A. et al., Cancer Res. (2000) 60(18):5117-24).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphav beta3) that will inhibit angiogenesis; endostatin and angiostatin (non-RTK) may also prove useful in combination with the disclosed erb family inhibitors. (See Bruns, C J et al., Cancer Res. (2000), 60(11): 2926-2935; Schreiber A B, Winkler M E, & Derynck R., Science (1986) 232(4755):1250-53; Yen L. et al., Oncogene (2000) 19(31): 3460-9).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T, et al., Cancer Res. (2000) 60(13)3569-76; and Chen Y, et al., Cancer Res. (1998) 58(9):1965-71.

Agents used in pro-apoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family. Therefore, strategies designed to downregulate the expression of bcl-2 in tumors have demonstrated clinical benefit and are now in Phase II/III trials, namely Genta's G3139 bcl-2 antisense oligonucleotide. Such pro-apoptotic strategies using the antisense oligonucleotide strategy for bcl-2 are discussed in Waters J S, et al., J. Clin. Oncol. (2000) 18(9): 1812-23; and Kitada S, et al. Antisense Res. Dev. (1994) 4(2): 71-9. Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania G R & Chang Y-T., Exp. Opin. Ther. Patents (2000) 10(2):215-30.

Other molecular targeted agents include FKBP binding agents, such as the immunosuppressive macrolide antibiotic, rapamycin; gene therapy agents, antisense therapy agents, and gene expression modulators such as the retinoids and rexinoids, e.g. adapalene, bexarotene, trans-retinoic acid, 9-cisretinoic acid, and N-(4 hydroxyphenyl)retinamide; phenotype-directed therapy agents, including: monoclonal antibodies such as alemtuzumab, bevacizumab, cetuximab, ibritumomab tiuxetan, rituximab, and trastuzumab; immunotoxins such as gemtuzumab ozogamicin, radioimmunoconjugates such as 131-tositumomab; and cancer vaccines.

Anti-tumor antibiotics include (a) anthracyclines such as daunorubicin (including liposomal daunorubicin), doxorubicin (including liposomal doxorubicin), epirubicin, idarubicin, and valrubicin; (b) streptomyces-related agents such as bleomycin, actinomycin, mithramycin, mitomycin, porfiromycin; and (c) anthracenediones, such as mitoxantrone and pixantrone. Anthracyclines have three mechanisms of action: intercalating between base pairs of the DNA/RNA strand; inhibiting topoiosomerase II enzyme; and creating iron-mediated free oxygen radicals that damage the DNA and cell membranes. Anthracyclines are generally characterized as topoisomerase II inhibitors.

Monoclonal antibodies include, but are not limited to, murine, chimeric, or partial or fully humanized monoclonal antibodies. Such therapeutic antibodies include, but are not limited to antibodies directed to tumor or cancer antigens either on the cell surface or inside the cell. Such therapeutic antibodies also include, but are not limited to antibodies directed to targets or pathways directly or indirectly associated with CK2. Therapeutic antibodies may further include, but are not limited to antibodies directed to targets or pathways that directly interact with targets or pathways associated with the compounds of the present invention. In one variation, therapeutic antibodies include, but are not limited to anticancer agents such as Abagovomab, Adecatumumab, Afutuzumab, Alacizumab pegol, Alemtuzumab, Altumomab pentetate, Anatumomab mafenatox, Apolizumab, Bavituximab, Belimumab, Bevacizumab, Bivatuzumab mertansine, Blinatumomab, Brentuximab vedotin, Cantuzumab mertansine, Catumaxomab, Cetuximab, Citatuzumab bogatox, Cixutumumab, Clivatuzumab tetraxetan, Conatumumab, Dacetuzumab, Detumomab, Ecromeximab, Edrecolomab, Elotuzumab, Epratuzumab, Ertumaxomab, Etaracizumab, Farletuzumab, Figitumumab, Fresolimumab, Galiximab, Glembatumumab vedotin, Ibritumomab tiuxetan, Intetumumab, Inotuzumab ozogamicin, Ipilimumab, Iratumumab, Labetuzumab, Lexatumumab, Lintuzumab, Lucatumumab, Lumiliximab, Mapatumumab, Matuzumab, Milatuzumab, Mitumomab, Nacolomab tafenatox, Naptumomab estafenatox, Necitumumab, Nimotuzumab, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Sibrotuzumab, Tacatuzumab tetraxetan, Taplitumomab paptox, Tenatumomab, Ticilimumab, Tigatuzumab, Tositumomab, Trastuzumab, Tremelimumab, Tucotuzumab celmoleukin, Veltuzumab, Volociximab, Votumumab, Zalutumumab, and Zanolimumab. In some embodiments, such therapeutic antibodies include, alemtuzumab, bevacizumab, cetuximab, daclizumab, gemtuzumab, ibritumomab tiuxetan, pantitumumab, rituximab, tositumomab, and trastuzumab; in other embodiments, such monoclonal antibodies include alemtuzumab, bevacizumab, cetuximab, ibritumomab tiuxetan, rituximab, and trastuzumab; alternately, such antibodies include daclizumab, gemtuzumab, and pantitumumab. In yet another embodiment, therapeutic antibodies useful in the treatment of infections include but are not limited to Afelimomab, Efungumab, Exbivirumab, Felvizumab, Foravirumab, Ibalizumab, Libivirumab, Motavizumab, Nebacumab, Pagibaximab, Palivizumab, Panobacumab, Rafivirumab, Raxibacumab, Regavirumab, Sevirumab, Tefibazumab, Tuvirumab, and Urtoxazumab. In a further embodiment, therapeutic antibodies can be useful in the treatment of inflammation and/or autoimmune disorders, including, but are not limited to, Adalimumab, Atlizumab, Atorolimumab, Aselizumab, Bapineuzumab, Basiliximab, Benralizumab, Bertilimumab, Besilesomab, Briakinumab, Canakinumab, Cedelizumab, Certolizumab pegol, Clenoliximab, Daclizumab, Denosumab, Eculizumab, Edobacomab, Efalizumab, Erlizumab, Fezakinumab, Fontolizumab, Fresolimumab, Gantenerumab, Gavilimomab, Golimumab, Gomiliximab, Infliximab, Inolimomab, Keliximab, Lebrikizumab, Lerdelimumab, Mepolizumab, Metelimumab, Muromonab-CD3, Natalizumab, Ocrelizumab, Odulimomab, Omalizumab, Otelixizumab, Pascolizumab, Priliximab, Reslizumab, Rituximab, Rontalizumab, Rovelizumab, Ruplizumab, Sifalimumab, Siplizumab, Solanezumab, Stamulumab, Talizumab, Tanezumab, Teplizumab, Tocilizumab, Toralizumab, Ustekinumab, Vedolizumab, Vepalimomab, Visilizumab, Zanolimumab, and Zolimomab aritox. In yet another embodiment, such therapeutic antibodies include, but are not limited to adalimumab, basiliximab, certolizumab pegol, eculizumab, efalizumab, infliximab, muromonab-CD3, natalizumab, and omalizumab. Alternately the therapeutic antibody can include abciximab or ranibizumab. Generally a therapeutic antibody is non-conjugated, or is conjugated with a radionuclide, cytokine, toxin, drug-activating enzyme or a drug-filled liposome.

Akt inhibitors include 1L6-Hydroxymethyl-chiro-inositol-2-(R)-2-O-methyl-3-O-octadecyl-sn-glycerocarbonate, SH-5 (Calbiochem Cat. No. 124008), SH-6 (Calbiochem Cat. No. Cat. No. 124009), Calbiochem Cat. No. 124011, Triciribine (NSC 154020, Calbiochem Cat. No. 124012), 10-(4′-(N-diethylamino)butyl)-2-chlorophenoxazine, Cu(II)Cl₂(3-Formylchromone thiosemicarbazone), 1,3-dihydro-1-((4-(6-phenyl-1H-imidazo[4,5-g]quinoxalin-7-yl)phenyl)methyl)-4-piperidinyl)-2H-benzimidazol-2-one, GSK690693 (4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol), SR13668 ((2,10-dicarbethoxy-6-methoxy-5,7-dihydro-indolo[2,3-b]carbazole), GSK2141795, Perifosine, GSK21110183, XL418, XL147, PF-04691502, BEZ-235 [2-Methyl-2-[4-(3-methyl-2-oxo-8-quinolin-3-yl-2,3-dihydro-imidazo[4,5-c]quinolin-1-yl)-phenyl]-propionitrile], PX-866 ((acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl ester)), D-106669, CAL-101, GDC0941 (2-(1H-indazol-4-yl)-6-(4-methanesulfonyl-piperazin-1-ylmethyl)-4-morpholin-4-yl-thieno[3,2-d]pyrimidine), SF1126, SF1188, SF2523, TG100-115 [3-[2,4-diamino-6-(3-hydroxyphenyl)pteridin-7-yl]phenol]. A number of these inhibitors, such as, for example, BEZ-235, PX-866, D 106669, CAL-101, GDC0941, SF1126, SF2523 are also identified in the art as PI3K/mTOR inhibitors; additional examples, such as PI-103 [3-[4-(4-morpholinylpyrido[3′,2′:4,5]furo[3,2-d]pyrimidin-2-yl]phenol hydrochloride] are well-known to those of skill in the art. Additional well-known PI3K inhibitors include LY294002 [2-(4-morpholinyl)-8-phenyl-4H-1-benzopyran-4-one] and wortmannin. mTOR inhibitors known to those of skill in the art include temsirolimus, deforolimus, sirolimus, everolimus, zotarolimus, and biolimus A9. A representative subset of such inhibitors includes temsirolimus, deforolimus, zotarolimus, and biolimus A9.

HDAC inhibitors include (i) hydroxamic acids such as Trichostatin A, vorinostat (suberoylanilide hydroxamic acid (SAHA)), panobinostat (LBH589) and belinostat (PXD101) (ii) cyclic peptides, such as trapoxin B, and depsipeptides, such as romidepsin (NSC 630176), (iii) benzamides, such as MS-275 (3-pyridylmethyl-N-{4-[(2-aminophenyl)-carbamoyl]-benzyl}-carbamate), C1994 (4-acetylamino-N-(2-aminophenyl)-benzamide) and MGCD0103 (N-(2-aminophenyl)-4-((4-(pyridin-3-yl)pyrimidin-2-ylamino)methyl)benzamide), (iv) electrophilic ketones, (v) the aliphatic acid compounds such as phenylbutyrate and valproic acid.

Hsp90 inhibitors include benzoquinone ansamycins such as geldanamycin, 17-DMAG (17-Dimethylamino-ethylamino-17-demethoxygeldanamycin), tanespimycin (17-AAG, 17-allylamino-17-demethoxygeldanamycin), ECS, retaspimycin (IPI-504, 18,21-didehydro-17-demethoxy-18,21-dideoxo-18,21-dihydroxy-17-(2-propenylamino)-geldanamycin), and herbimycin; pyrazoles such as CCT 018159 (4-[4-(2,3-dihydro-1,4-benzodioxin-6-yl)-5-methyl-1H-pyrazol-3-yl]-6-ethyl-1,3-benzenediol); macrolides, such as radicocol; as well as BIIB021 (CNF2024), SNX-5422, STA-9090, and AUY922.

Miscellaneous agents include altretamine, arsenic trioxide, gallium nitrate, hydroxyurea, levamisole, mitotane, octreotide, procarbazine, suramin, thalidomide, lenalidomide, photodynamic compounds such as methoxsalen and sodium porfimer, and proteasome inhibitors such as bortezomib.

Biologic therapy agents include: interferons such as interferon-α2a and interferon-α2b, and interleukins such as aldesleukin, denileukin diftitox, and oprelvekin.

In addition to these anticancer agents intended to act against cancer cells, combination therapies including the use of protective or adjunctive agents, including: cytoprotective agents such as armifostine, dexrazonxane, and mesna, phosphonates such as pammidronate and zoledronic acid, and stimulating factors such as epoetin, darbepoetin, filgrastim, PEG-filgrastim, and sargramostim, are also envisioned.

### EXAMPLES

In general, the compounds of the invention can be synthesized according to the methods known to one skilled in the art and/or the following exemplary procedures and schemes. The following examples illustrate and do not limit the invention.

### Example 1

### Synthesis of 3-((5-(3-chlorophenylamino)pyrazolo[1,5-a]pyrimidin-3-yl)methylene)-5-fluoroindolin-2-one

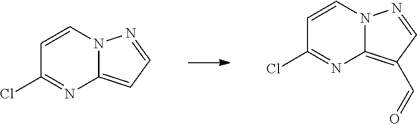

To 5-chloropyrazolo[1,5-a]pyrimidine (200 mg, 1.31 mmol) in 1.5 ml DMF was added POCl₃ (358 μL, 3.92 mmol). The reaction was stirred at room temperature overnight. The mixture was cooled to 0° C. in ice bath and the then neutralized with 6M NaOH. The solid formed was isolated by filtration and air dried to give 165 mg of 5-chloropyrazolo[1,5-a]pyrimidine-3-carbaldehyde as yellow solid (70% yield). LCMS (M+1=182)

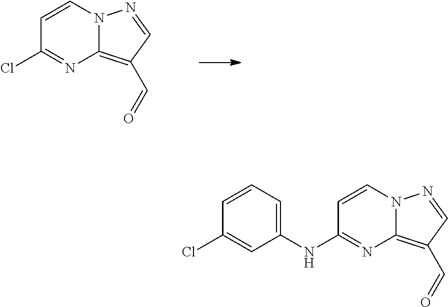

To 5-chloropyrazolo[1,5-a]pyrimidine-3-carbaldehyde (120 mg, 0.66 mmol) in 1.5 ml dioxane was added 3-chloroaniline (35 μL, 3.31 mmol). The mixture was heated in microwave 10 minutes at 120° C. The solid formed was isolated by filtration and air dried to give 5-(3-chlorophenylamino)pyrazolo[1,5-a]pyrimidine-3-carbaldehyde as orange solid. LCMS (M+1=273)

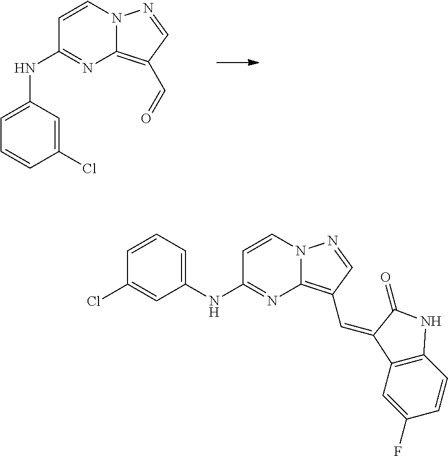

To 5-(3-chlorophenylamino)pyrazolo[1,5-a]pyrimidine-3-carbaldehyde (50 mg, 0.184 mmol) in 1 mL EtOH was added 5-fluorooxindole (28 mg, 0.184 mmol) and piperidine (18 μL, 0.184 mmol). The mixture was stirred at room temperature overnight. The solvent was removed under reduced pressure and the resulting was prepared by HPLC to give 3-((5-(3-chlorophenylamino)pyrazolo[1,5-a]pyrimidin-3-yl(methylene)-5-fluoroindolin-2-one. LCMS (M+1=406)

### Example 2

### Synthesis of 4-((5-(3-chlorophenylamino)pyrazolo[1,5-a]pyrimidin-3-yl)methylene)-3-methyl-1H-pyrazol-5(4H)-one

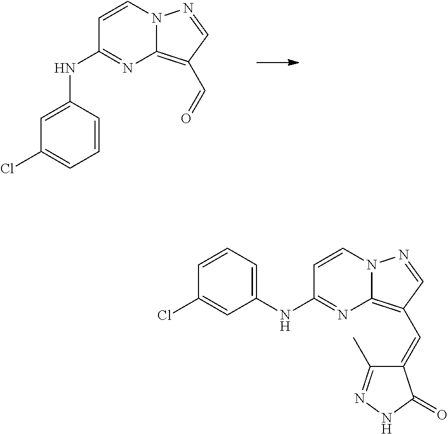

To 5-(3-chlorophenylamino)pyrazolo[1,5-a]pyrimidine-3-carbaldehyde (80 mg, 0.294 mmol) in EtOH was added 3-methyl-1H-pyrazol-5(4H)-one (29 mg, 0.294 mmol) and piperidine (30 μL, 0.294 mmol). The mixture was heated at 70° C. overnight. The solid formed was isolated by filtration to yield 4-((5-(3-chlorophenylamino)pyrazolo[1,5-a]pyrimidin-3-yl)methylene)-3-methyl-1H-pyrazol-5(4H)-one. LCMS (M+1=353)

### Example 3

### Synthesis of 3-((5-(3-chlorophenylamino)pyrazolo[1,5-a]pyrimidin-3-yl)methylene)piperidine-2,6-dione

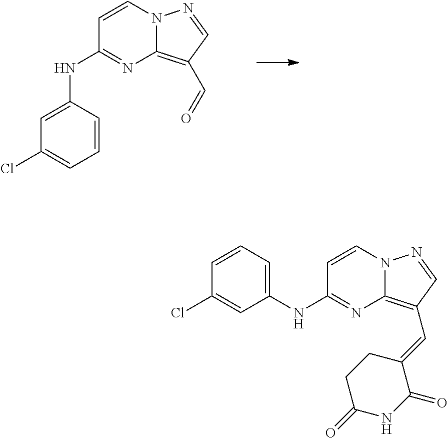

To 5-(3-chlorophenylamino)pyrazolo[1,5-a]pyrimidine-3-carbaldehyde (80 mg, 0.294 mmol) in Toluene was added piperidine-2,6-dione (99 mg, 0.882 mmol), piperidine (60 μL, 0.588 mmol), and molecular sieve. The mixture was heated at 105° C. overnight. The solid formed was filtered off and the filtrate was purified by HPLC to yield 3-((5-(3-chlorophenylamino)pyrazolo[1,5-a]pyrimidin-3-yl)methylene)piperidine-2,6-dione. LCMS (M+1=368)

### Example 4

### Synthesis of 4-((5-(3-chlorophenylamino)pyrazolo[1,5-a]pyrimidin-3-yl)methylene)-3-(trifluoromethyl)-1H-pyrazol-5(4H)-one

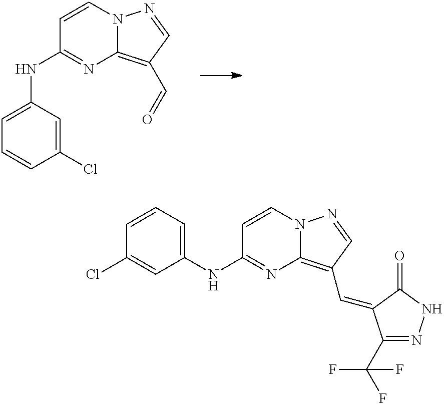

To 5-(3-chlorophenylamino)pyrazolo[1,5-a]pyrimidine-3-carbaldehyde (76 mg, 0.279 mmol) in EtOH was added 3-(trifluoromethyl)-1H-pyrazol-5(4H)-one (42 mg, 0.279 mmol) and piperidine (28 μL, 0.279 mmol). The mixture was heated at 70° C. overnight two times. The solid formed was isolated by filtration and air dried to yield 4((5-(3-chlorophenylamino)pyrazolo[1,5-a]pyrimidin-3-yl)methylene)-3-(trifluoromethyl)-1H-pyrazol-5(4H)-one. LCMS (M+1=407)

### Example 5

### Synthesis of Additional Aldehydes for Use in Making Related Compounds

The methods illustrated above can be adapted to the synthesis of a variety of additional compounds of Formula (I); syntheses of a number of exemplary aldehydes for use in such methods are provided below.

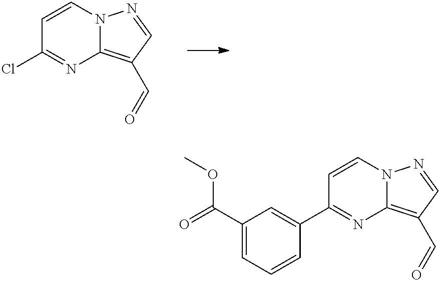

To 5-chloropyrazolo[1,5-a]pyrimidine-3-carbaldehyde (115 mg, 0.64 mmol) in dioxane/water (2850 μL/150 μL) was added 3-(methoxycarbonyl)phenylboronic acid (171 mg, 0.95 mmol), and cesium carbonate (623 mg, 1.91 mmol). The mixture was degassed under nitrogen for 10 minutes and then PdCl₂dppf (23 mg, 0.03 mmol) was added. The mixture was heated at 105° C. overnight. Water was added and the resulting solid was isolated by filtration. The solid was then dissolved in dichloromethane and washed with water, dried over Na₂SO₄ and passed through a plug of silica. The resulting solution was concentrated under vacuum to yield 125 mg of 3-(3-formylpyrazolo[1,5-a]pyrimidin-5-yl)benzoate as a yellow solid (70% yield). LCMS (M+1=282)

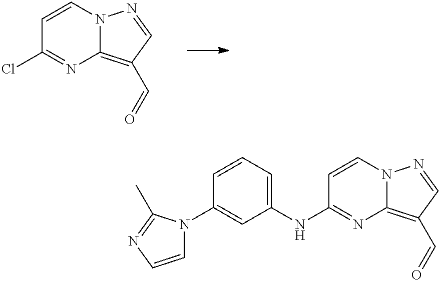

To 5-chloropyrazolo[1,5-a]pyrimidine-3-carbaldehyde (39 mg, 0.215 mmol) in dioxane was added 3-(2-methyl-1H-imidazol-1-yl)aniline (90 mg, 0.520 mmol). The mixture was heated in microwave (200 W) for 50 minutes at 120° C. The solid formed was isolated by filtration and air dried to yield 48 mg 5-(3-(2-methyl-1H-imidazol-1-yl)phenylamino)pyrazolo[1,5-a]pyrimidine-3-carbaldehyde (70% yield). LCMS (M+1=319)

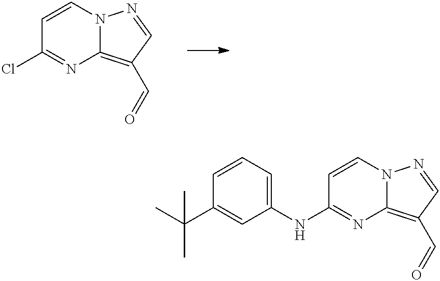

To 5-chloropyrazolo[1,5-a]pyrimidine-3-carbaldehyde (50 mg, 0.276 mmol) in dioxane was added 3-tert-butylaniline (206 mg, 1.381 mmol). The mixture was heated in microwave for 10 minutes at 120° C. The solid formed was isolated by filtration and air dried to yield 78 mg 5-(3-tert-butylphenylamino)pyrazolo[1,5-a]pyrimidine-3-carbaldehyde (96% yield). LCMS (M+1=295)

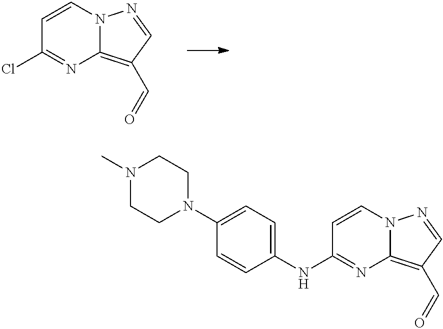

To 5-chloropyrazolo[1,5-a]pyrimidine-3-carbaldehyde (50 mg, 0.276 mmol) in dioxane was added 4-(4-methylpiperazin-1-yl)aniline (264 mg, 1.381 mmol). The mixture was heated in microwave for 20 minutes at 120° C. The solid formed was isolated by filtration to yield 5-(4-(4-methylpiperazin-1-yl)phenylamino)pyrazolo[1,5-a]pyrimidine-3-carbaldehyde. The residue was used in the next step without further purification. LCMS (M+1=337).

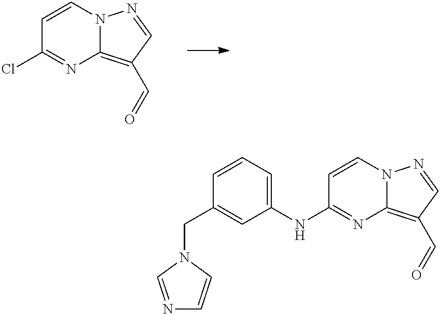

To 5-chloropyrazolo[1,5-a]pyrimidine-3-carbaldehyde (40 mg, 0.221 mmol) in dioxane was added 3-((1H-imidazol-1-yl)methyl)aniline (115 mg, 0.663 mmol). The mixture was heated in microwave for 120 minutes at 120° C. EtOAc was added to the mixture, and washed with water. The organic layer was then dried over Na₂SO₄ and solvent was removed under reduced pressure to yield 5-(3-((1H-imidazol-1-yl)methyl)phenylamino)pyrazolo[1,5-a]pyrimidine-3-carbaldchyde. The resulting solid was used in the next step without further purification. LCMS (M+1=319)

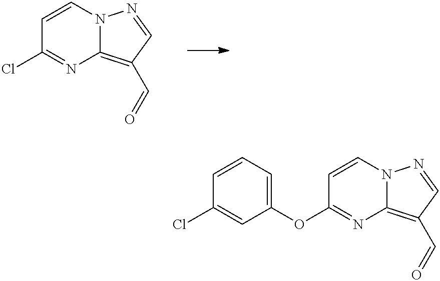

To 5-chloropyrazolo[1,5-a]pyrimidine-3-carbaldehyde (50 mg, 0.276 mmol) in DMF was added 3-chlorophenol (42 mg, 0.331 mmol) and K₂CO₃ (190 mg, 1.380 mmol). The mixture was heated at 70° C. for several hours. Water was added and the solid formed was isolated by filtration and air dried to yield 70 mg 5-(3-chlorophenoxy)pyrazolo[1,5-a]pyrimidine-3-carbaldehyde as an orange solid (93% yield). LCMS (M+1=274)

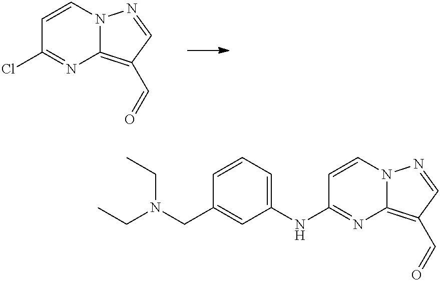

To 5-chloropyrazolo[1,5-a]pyrimidine-3-carbaldehyde (50 mg, 0.276 mmol) in dioxane was added 3-((diethylamino)methyl)aniline (148 mg, 0.829 mmol). The mixture was heated in microwave for 140 minutes at 120° C. Dichloromethane was added, and washed with water. The organic layer was dried over Na₂SO₄ and concentrated under reduced pressure. The resulting solution was prepared by TLC (10% MeOH/DCM) to yield 10 mg 5-(3-((diethylamino)methyl)phenylamino)pyrazolo[1,5-a]pyrimidine-3-carbaldehyde (11% yield). LCMS (M+1=324)

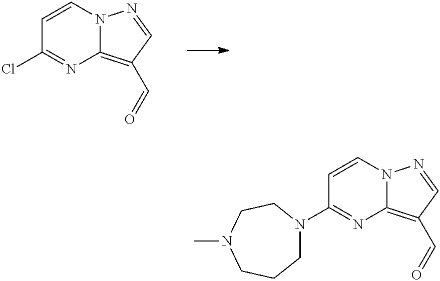

To 5-chloropyrazolo[1,5-a]pyrimidine-3-carbaldehyde (50 mg, 0.276 mmol) in NMP was added 1-methylhomopiperazine (103 μL, 0.829 mmol). The mixture was heated in microwave for 10 minutes at 140° C. Dichloromethane and water were added, and the product extracted in dichloromethane. The organic layer was then washed with water and dried over Na₂SO₄ and concentrated under reduced pressure to yield 5-(4-methyl-1,4-diazepan-1-yl)pyrazolo[1,5-a]pyrimidine-3-carbaldehyde. LCMS (M+1=260)

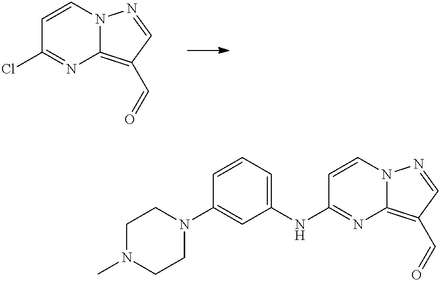

To 5-chloropyrazolo[1,5-a]pyrimidine-3-carbaldehyde (40 mg, 0.221 mmol) in dioxane was added 3-(4-methylpiperazin-1-yl)aniline (127 mg, 0.663 mmol). The mixture was heated in microwave at 120° C. Dichloromethane and water were added, and the product extracted in dichloromethane. The organic layer was then dried over Na₂SO₄ and concentrated under reduced pressure to yield 5-(3-(4-methylpiperazin-1-yl)phenylamino)pyrazolo[1,5-a]pyrimidine-3-carbaldehyde. LCMS (M+1=337)

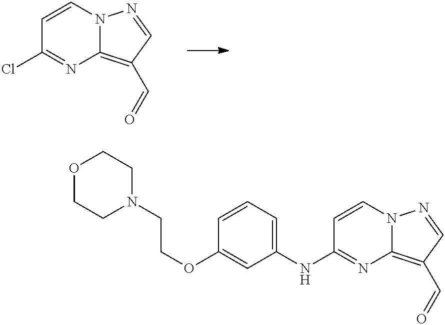

To 5-chloropyrazolo[1,5-a]pyrimidine-3-carbaldehyde (40 mg, 0.221 mmol) in dioxane was added 3-(2-morpholinoethoxy)aniline (147 mg, 0.663 mmol). The mixture was heated in microwave at 120° C. Dichloromethane was added, and washed with water. The organic layer was dried over Na₂SO₄ and concentrated under reduced pressure to yield 543-(2-morpholinoethoxy)phenylamino)pyrazolo[1,5-a]pyrimidine-3-carbaldehyde. The solid was used in the next step without further purification. LCMS (M+1=368)

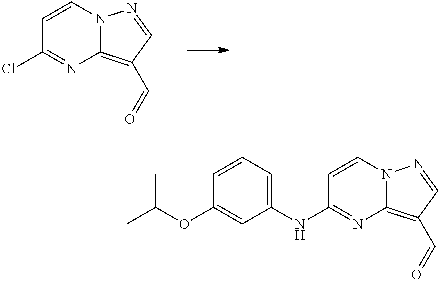

To 5-chloropyrazolo[1,5-a]pyrimidine-3-carbaldehyde (50 mg, 0.276 mmol) in dioxane was added 3-isopropoxyaniline (125 mg, 0.829 mmol). The mixture was heated in microwave for 20 minutes at 120° C. The solid produced was isolated by filtration and then purified by preparative TLC (2% MeOH/DCM) to yield 5-(3-isopropoxyphenylamino)pyrazolo[1,5-a]pyrimidine-3-carbaldehyde. LCMS (M+1=297)

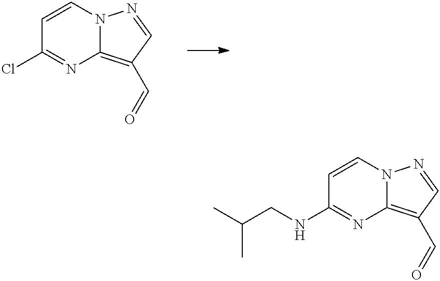

To 5-chloropyrazolo[1,5-a]pyrimidine-3-carbaldehyde (20 mg, 0.110 mmol) in acetonitrile was added 2-methylpropan-1-amine (22 μL, 0.221 mmol). The mixture was heated at 70° C. and produced the desired product, 5-(isobutylamino)pyrazolo[1,5-a]pyrimidine-3-carbaldehyde. LCMS (M+1=219)

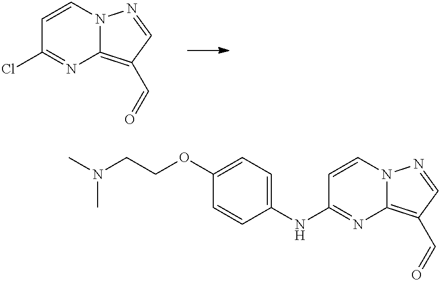

To 5-chloropyrazolo[1,5-a]pyrimidine-3-carbaldehyde (50 mg, 0.276 mmol) in dioxane was added 4-(2-(dimethylamino)ethoxy)aniline (149 mg, 0.829 mmol). The mixture was heated in microwave 100 minutes at 120° C. Water and dichloromethane were added, and the product was extracted into dichloromethane. The organic layer was dried over Na₂SO₄ and concentrated under reduced pressured to yield 5-(4-(2-(dimethylamino)ethoxy)phenylamino)pyrazolo[1,5-a]pyrimidine-3-carbaldehyde. LCMS (M+1=408)

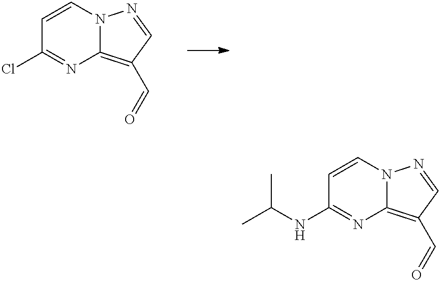

To 5-chloropyrazolo[1,5-a]pyrimidine-3-carbaldehyde (20 mg, 0.11 mmol) in acetonitrile was added isopropylamine (19 μL, 0.22 mmol). The mixture was heated at 70° C. The desired product, 5-(isopropylamino)pyrazolo[1,5-a]pyrimidine-3-carbaldehyde, formed in solution. LCMS (M+1=205)

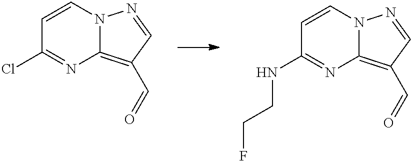

To 5-chloropyrazolo[1,5-a]pyrimidine-3-carbaldehyde (20 mg, 0.11 mmol) in ACN was added 2-fluoroethanamine hydrochloride (22 mg, 0.22 mmol). The mixture was heated at 70° C. The desired product, 5-(2-fluoroethylamino)pyrazolo[1,5-a]pyrimidine-3-carbaldehyde, formed in solution. LCMS (M+1=209)

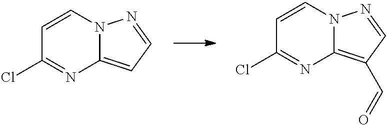

To 5-chloropyrazolo[1,5-a]pyrimidine (200 mg, 1.31 mmol) in 1.5 mL DMF was added POCl₃ (358 μL, 3.92 mmol). The reaction was stirred at room temperature overnight. The mixture was cooled to 0° C. in ice bath and then neutralized with 6M NaOH. The solid formed was isolated by filtration and air dried to give 165 mg of 5-chloropyrazolo[1,5-a]pyrimidine-3-carbaldehyde as yellow solid (70% yield). LCMS (M+1=182)

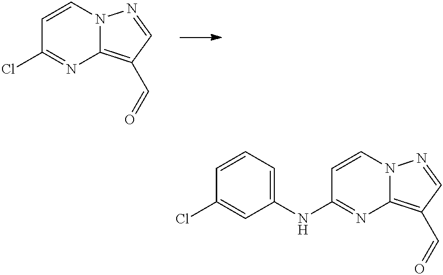

To 5-chloropyrazolo[1,5-a]pyrimidine-3-carbaldehyde (120 mg, 0.66 mmol) in 1.5 mL dioxane was added 3-chloroaniline (351 μL, 3.31 mmol). The mixture was heated in microwave 10 minutes at 120° C. The solid formed was isolated by filtration and air dried to give 5-(3-chlorophenylamino)pyrazolo[1,5-a]pyrimidine-3-carbaldehyde as orange solid. LCMS (M+1=273)

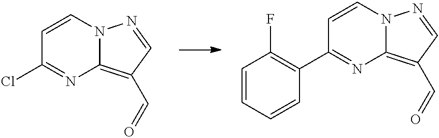

To 5-chloropyrazolo[1,5-a]pyrimidine-3-carbaldehyde (150 mg, 0.83 mmol) in 4 mL DMF/water (0.05%) was added 2-fluorophenylboronic acid (174 mg, 1.245 mmol) and cesium carbonate (812 mg, 2.49 mmol). The mixture was degassed under nitrogen during 10 minutes. PdCl2(dppf)₂ (30.3 mg, 0.041 mmol) was then added. The mixture was heated in the microwave at 100° C. for 10 minutes. Water was added, the precipitate was isolated by filtration and air dried to give 5-(2-fluorophenyl)pyrazolo[1,5-a]pyrimidine-3-carbaldehyde. LCMS (M+1)=241

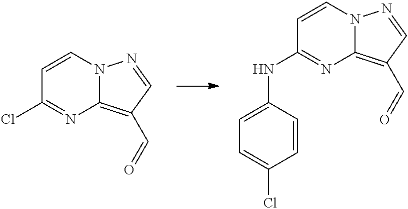

To 5-chloropyrazolo[1,5-a]pyrimidine-3-carbaldehyde (120 mg, 0.633 mmol) in dioxane was added 3-chloroaniline (421 mg, 3.315 mmol). The mixture was heated in microwave for 20 minutes at 120° C. The solid formed was isolated by filtration and air dried to yield 5-(4-chlorophenylamino)pyrazolo[1,5-a]pyrimidine-3-carbaldehyde. LCMS (M+1=273)

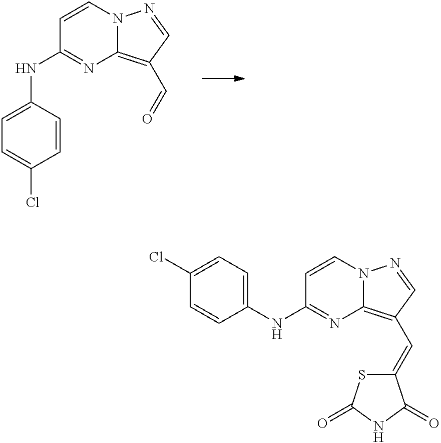

To 5-(4-chlorophenylamino)pyrazolo[1,5-a]pyrimidine-3-carbaldehyde (117 mg, 0.430 mmol) in EtOH was added thiazolidine-2,4-dione (50 mg, 0.430 mmol) and piperidine (43 μl, 0.430 mmol). The mixture was heated at 70° C. and the product formed quickly. The solid formed was isolated by filtration and air dried to yield 5-((5-(4-chlorophenylamino)pyrazolo[1,5-a]pyrimidin-3-yl)methylene)thiazolidine-2,4-dione. LCMS (M+1=372)

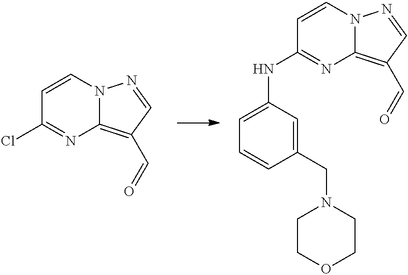

To 5-chloropyrazolo[1,5-a]pyrimidine-3-carbaldehyde (30 mg, 0.166 mmol) in DMF was added 3-(morpholinomethyl)aniline (233 mg, 1.213 mmol). The mixture was heated in microwave for 40 minutes at 140° C. Water was added and the solid formed was isolated by filtration to yield 5-(3-(morpholinomethyl)phenylamino)pyrazolo[1,5-a]pyrimidine-3-carbaldehyde. LCMS (M+1=338)

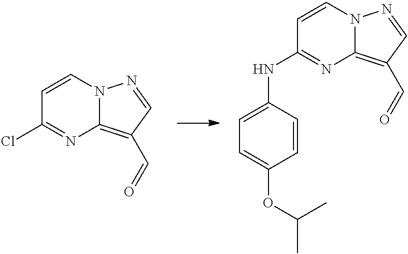

To 5-chloropyrazolo[1,5-a]pyrimidine-3-carbaldehyde (30 mg, 0.166 mmol) in dioxane was added 4-isopropoxyaniline (125 mg, 0.829 mmol). The mixture was heated in microwave for 20 minutes at 120° C. The solid formed was isolated by filtration and air dried to yield 5-(4-isopropoxyphenylamino)pyrazolo[1,5-a]pyrimidine-3-carbaldehyde with impurities that will be removed in the final step. LCMS (M+1=297)

### Example 6

### Synthesis of Triphenylphosphoranylidene Succinimide

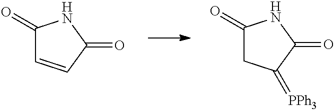

To Maleimide (1.0 g, 10.3 mmol in acetone (11 mL) was added Triphenylphosphine (2.7 g, 10.3 mmol). The reaction mixture was stirred at reflux for 1 hour. The reaction mixture was cooled to room temperature and the resulting precipitate was filtered off and rinsed with 50 mL of acetone. Dried under vacuum to provide 3.30 g of Triphenylphosphoranylidene succinimide. LCMS (M+1=360.3)

### Example 7

### Synthesis of 5-chloro-N-cyclopropylpyrazolo[1,5-a]pyrimidin-7-amine

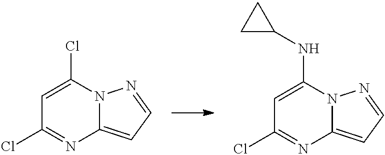

To 5,7-dichloropyrazolo[1,5-a]pyrimidine (200 mg, 1.06 mmol) in ACN was added Et₃N (148 μL, 1.06 mmol) and cyclopropanamine (75 μl, 1.06 mmol). The reaction was refluxed at 80° C. overnight. The mixture was concentrated under reduced pressure, dissolved me DCM, and washed with water. The resulting organic layer was dried over Na₂SO₄ and concentrated under reduced pressure to afford 156 mg of 5-chloro-N-cyclopropylpyrazolo[1,5-a]pyrimidin-7-amine (70% yield). LCMS (M+1=209)

### Example 8

### Synthesis of 5-chloro-7-(cyclopropylamino)pyrazolo[1,5-a]pyrimidine-3-carbaldehyde

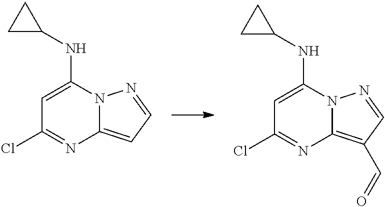

To 5-chloro-N-cyclopropylpyrazolo[1,5-a]pyrimidin-7-amine (156 mg, 0.75 mmol) in DMF was added POCl₃ (205 μl, 2.25 mmol). The mixture was stirred at room temperature for 3 hours. Ice was added to quench POCl₃, then the mixture was neutralized with 1M NaOH. DCM was added and the product was extracted three times. The organic layer was dried over Na₂SO₄ and concentrated under reduced pressure to yield 5-chloro-7-(cyclopropylamino)pyrazolo[1,5-a]pyrimidine-3-carbaldehyde. Some residual DMF could not be removed. LCMS (M+1=237)

### Example 9

### Synthesis of tert-butyl 5-chloro-3-formylpyrazolo[1,5-a]pyrimidin-7-yl(cyclopropyl)carbamate

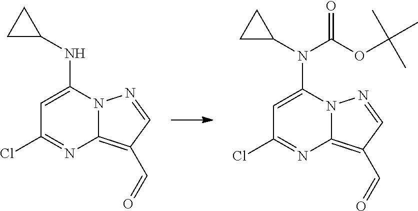

To 5-chloro-7-(cyclopropylamino)pyrazolo[1,5-a]pyrimidine-3-carbaldehyde (4.52 g, 19.15 mmol) in methylene chloride (80 mL) was added triethylamine (3.2 mL, 23 mmol), dimethylaminopyridine (350 mg, 2.87 mmol), and di-t-butyldicarbonate (12.53 g, 57.44 mmol) The mixture was stirred at room temperature for 60 minutes. The reaction mixture was transferred to a separatory funnel and washed 1× with H₂O, 2× with brine. Dried over MgSO₄, filtered and removed solvent to provide an oily residue which was purified by silica gel chromatography (0%-20% ethyl acetate/hexanes) to yield 5.68 g (88% yield) of tert-butyl 5-chloro-3-formylpyrazolo[1,5-a]pyrimidin-7-yl(cyclopropyl)carbamate. LCMS (M+1=337)

### Example 10

### Synthesis of tert-butyl 5-chloro-3-((2,5-dioxopyrrolidin-3-ylidene)methyl)pyrazolo[1,5-a]pyrimidin-7-yl(cyclopropyl)carbamate

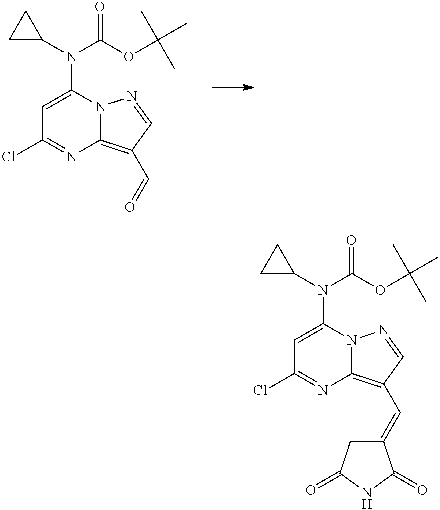

To 7 tert-butyl 5-chloro-3-formylpyrazolo[1,5-a]pyrimidin-7-yl(cyclopropyl)carbamate (1.87 g, 5.56 mmol) in methanol (55 mL) was added triphenylphosphoranylidene succinimide (2.0 g, 5.56 mmol). The reaction mixture was stirred at reflux for 2 hours. The reaction mixture was cooled to 0° and the resulting precipitate was filtered off and rinsed with cold methanol. Dried under vacuum to provide tert-butyl 5-chloro-3-((2,5-dioxopyrrolidin-3-ylidene)methyl)pyrazolo[1,5-a]pyrimidin-7-yl(cyclopropyl)carbamate. LCMS (M+1=318.3)

### Example 11

### Synthesis of tert-butyl cyclopropyl(3-((2,5-dioxopyrrolidin-3-ylidene)methyl)-5-(4-(pyridin-2-yl)piperazin-1-yl)pyrazolo[1,5-a]pyrimidin-7-yl)carbamate

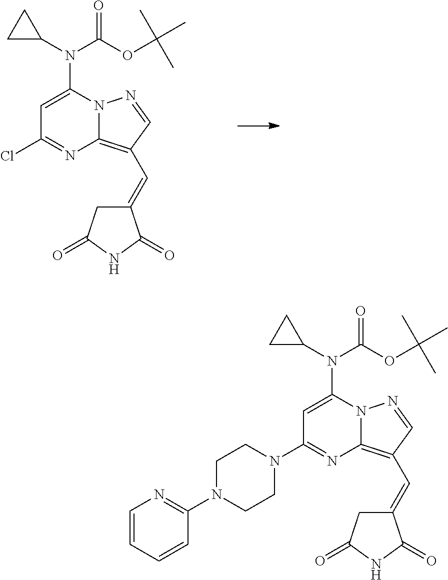

To tert-butyl 5-chloro-3-((2,5-dioxopyrrolidin-3-ylidene)methyl)pyrazolo[1,5-a]pyrimidin-7-yl(cyclopropyl)carbamate (100 mg, 0.239 mmol) in DMF (3 mL) was added K₂CO₃ (50 mg, 0.358 mmol) and 1-(pyridin-2-yl)piperazine (58 mg, 0.358 mmol). The reaction mixture was stirred at 80° for 30 minutes. The reaction mixture was partitioned between EtOAc and H₂O and the layers were separated. Organic layer was washed 2× with brine, dried with MgSO₄, filtered and removed solvent. The residue was purified by flash chromatography eluting with 1:1 EtOAc/Hexane to provide 58 mg of tert-butyl cyclopropyl(3-((2,5-dioxopyrrolidin-3-ylidene)methyl)-5-(4-(pyridin-2-yl)piperazin-1-yl)pyrazolo[1,5-a]pyrimidin-7-yl)carbamate. (45%) LCMS (M+1=545)

### Example 12

### Synthesis of 3-((7-(cyclopropylamino)-5-(4-(pyridin-2-yl)piperazin-1-yl)pyrazolo[1,5-a]pyrimidin-3-yl)methylene)pyrrolidine-2,5-dione

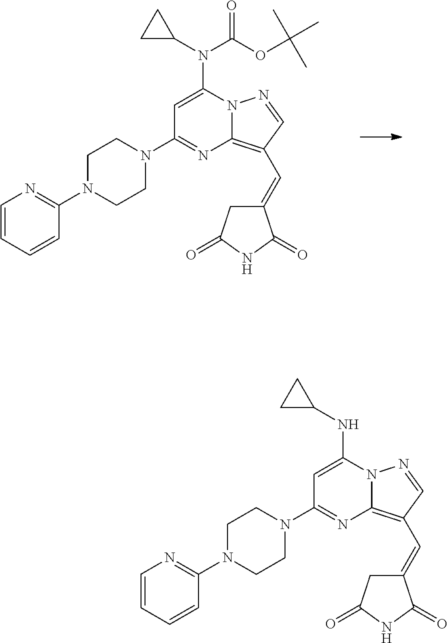

To tert-butyl cyclopropyl(3-((2,5-dioxopyrrolidin-3-ylidene)methyl)-5-(4-(pyridin-2-yl)piperazin-1-yl)pyrazolo[1,5-a]pyrimidin-7-yl)carbamate (58 mg, 0.106 mmol) was added 4 mL of a 1:1 mixture of TFA/methylene chloride. The reaction mixture was stirred at rt for 1 h. Removed solvent to provide 3-((7-(cyclopropylamino)-5-(4-(pyridin-2-yl)piperazin-1-yl)pyrazolo[1,5-a]pyrimidin-3-yl)methylene)pyrrolidine-2,5-dione as the TFA salt. LCMS (M+1=445)

### Example 13

### Synthesis of tert-butyl 4-(7-(tert-butoxycarbonyl(cyclopropyl)amino)-3-((2,5-dioxopyrrolidin-3-ylidene)methyl)pyrazolo[1,5-a]pyrimidin-5-yl)piperazine-1-carboxylate

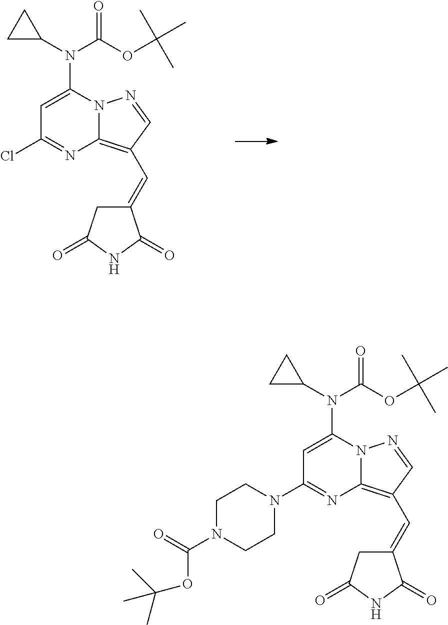

Same procedure as [synthesis f]. LCMS (M+1=568)

### Example 14

### Synthesis of 3-((7-(cyclopropylamino)-5-(piperazin-1-yl)pyrazolo[1,5-a]pyrimidin-3-yl)methylene)pyrrolidine-2,5-dione

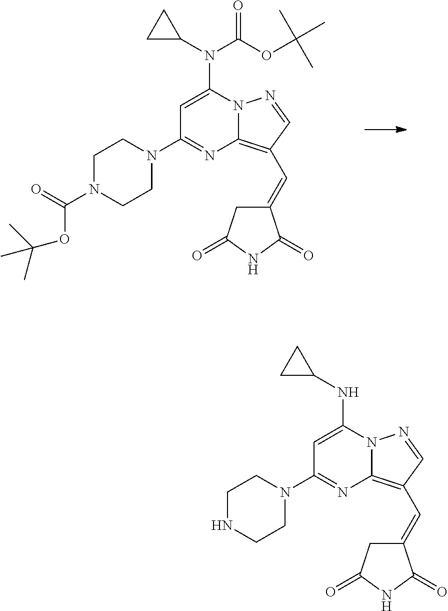

To tert-butyl 4-(7-(tert-butoxycarbonyl(cyclopropyl)amino)-3-((2,5-dioxopyrrolidin-3-ylidene)methyl)pyrazolo[1,5-a]pyrimidin-5-yl)piperazine-1-carboxylate (2.2 g, 3.87 mmol) was added 8 mL of 4M HCl/dioxane. The reaction mixture was stirred at 80° for 30 min. Cool to rt and filter off solid to provide 1.75 g of 3-((7-(cyclopropylamino)-5-(piperazin-1-yl)pyrazolo[1,5-a]pyrimidin-3-yl)methylene)pyrrolidine-2,5-dione as the HCl salt. LCMS (M+1=368)

### Example 15

### Synthesis of 3-((7-(cyclopropylamino)-5-((tetrahydrofuran-2-yl)methylamino)pyrazolo[1,5-a]pyrimidin-3-yl)methylene)pyrrolidine-2,5-dione

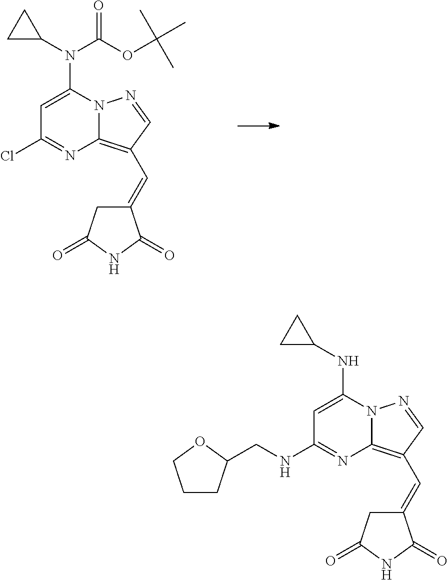

To tert-butyl 5-chloro-3-((2,5-dioxopyrrolidin-3-ylidene)methyl)pyrazolo[1,5-a]pyrimidin-7-yl(cyclopropyl)carbamate (30 mg, 0.072 mmol) in 1 mL of DMF was added K₂CO₃ (15 mg, 0.108 mmol), and (tetrahydrofuran-2-yl)methanamine (11 mg, 0.108 mmol). The reaction mixture was stirred at 95° for 30 min. Cool to rt and dilute with EtOAc. Wash organic layer 1× with brine. Organic layer dried with MgSO₄ and filtered. To the vial was added 1 mL of 4M HCl/dioxane. Stir at 75° for 45 min. Cool to rt and the resulting solid was filtered and rinsed with EtOAc to provide 3-((7-(cyclopropylamino)-5-((tetrahydrofuran-2-yl)methylamino)pyrazolo[1,5-a]pyrimidin-3-yl)methylene)pyrrolidine-2,5-dione as the HCl salt. LCMS (M+1=383)

### Examples 16 to 19 below were prepared by the procedures described above including the procedures for Example 15

### Example 16

### Synthesis of 3-((7-(cyclopropylamino)-5-(3-hydroxypiperidin-1-yl)pyrazolo[1,5-a]pyrimidin-3-yl)methylene)pyrrolidine-2,5-dione

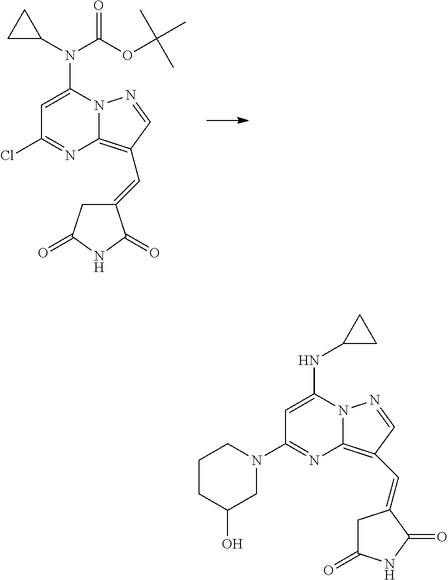

LCMS (M+1=383)

### Example 17

### Synthesis of 3-((7-(cyclopropylamino)-5-(4-methylpiperazin-1-yl)pyrazolo[1,5-a]pyrimidin-3-yl)methylene)pyrrolidine-2,5-dione

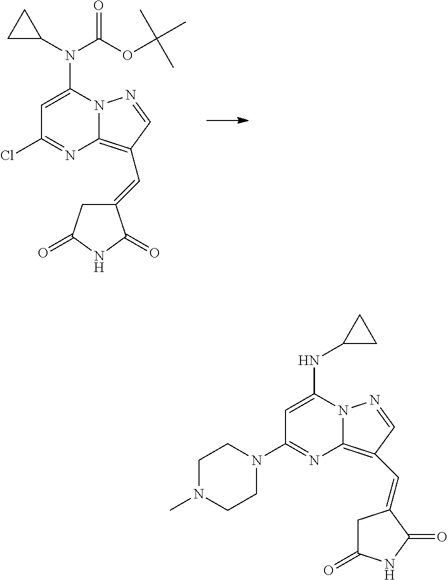

LCMS (M+1=382)

### Example 18

### Synthesis of 3-((7-(cyclopropylamino)-5-(4-methyl-1,4-diazepan-1-yl)pyrazolo[1,5-a]pyrimidin-3-yl)methylene)pyrrolidine-2,5-dione

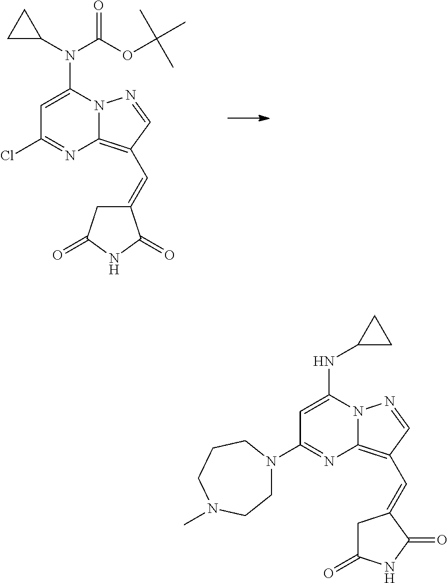

LCMS (M+1=396)

### Example 19

### Synthesis of 3-((7-(cyclopropylamino)-5-(3-(pyrrolidin-1-yl)propylamino)pyrazolo[1,5-a]pyrimidin-3-yl)methylene)pyrrolidine-2,5-dione

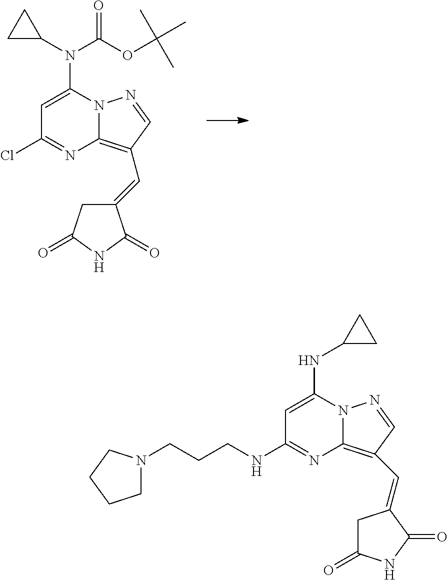

LCMS (M+1=410)

### Example 20

### Synthesis of 3-((7-(cyclopropylamino)-5-(pyrrolidin-1-yl)pyrazolo[1,5-a]pyrimidin-3-yl)methylene)pyrrolidine-2,5-dione

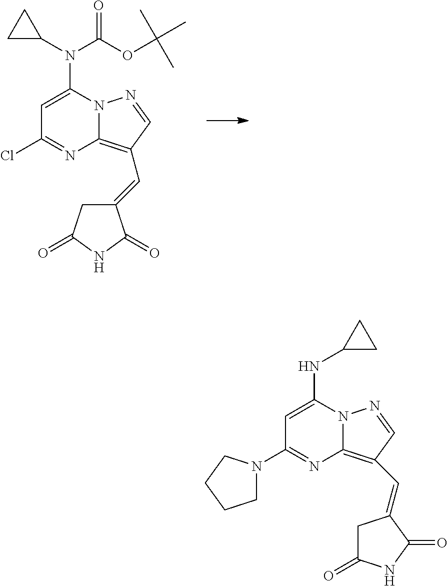

To tert-butyl 5-chloro-3-((2,5-dioxopyrrolidin-3-ylidene)methyl)pyrazolo[1,5-a]pyrimidin-7-yl(cyclopropyl)carbamate (30 mg, 0.072 mmol) in 1 mL of DMF was added K₂CO₃ (15 mg, 0.108 mmol), and pyrrolidine (8 mg, 0.108 mmol). The reaction mixture was stirred at 70° for 2 h. Cool to rt and dilute with EtOAc. Wash organic layer 1× with brine. Organic layer dried with MgSO₄ and filtered. To the vial was added 1 mL of 4M HCl/dioxane. Stir at 75° for 1 h. Cool to rt and the solvent was decanted off. EtOAc was added to the solid and again decanted off to provide 3-((7-(cyclopropylamino)-5-(pyrrolidin-1-yl)pyrazolo[1,5-a]pyrimidin-3-yl)methylene)pyrrolidine-2,5-dione as the HCl salt. LCMS (M+1=353)

### Example 21

### Synthesis of 3-((7-(cyclopropylamino)-5-morpholinopyrazolo[1,5-a]pyrimidin-3-yl)methylene)pyrrolidine-2,5-dione

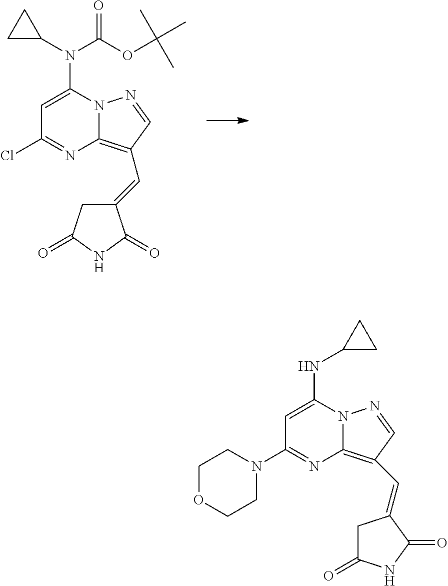

Example 21 was prepared by the procedures described above including the procedures for Example 20. LCMS (M+1=367)

### Example 22

### Synthesis of 3-((7-(cyclopropylamino)-5-(4-ethylpiperazin-1-yl)pyrazolo[1,5-a]pyrimidin-3-yl)methylene)pyrrolidine-2,5-dione

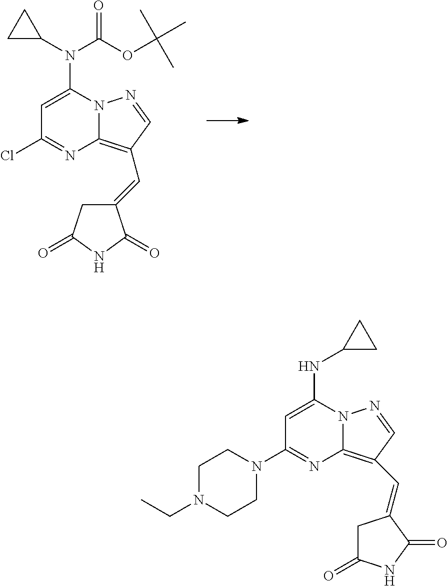

To tert-butyl 5-chloro-3-((2,5-dioxopyrrolidin-3-ylidene)methyl)pyrazolo[1,5-a]pyrimidin-7-yl(cyclopropyl)carbamate (30 mg, 0.072 mmol) in 1 mL of DMF was added K₂CO₃ (15 mg, 0.108 mmol), and pyrrolidine (8 mg, 0.108 mmol). The reaction mixture was stirred at 70° for 2 h. Cool to rt and dilute with EtOAc. Wash organic layer 1× with brine. Organic layer dried with MgSO₄ and filtered. To the vial was added 1 mL of 4M HCl/dioxane. Stir at 75° for 1 h. Cool to rt and the solvent was decanted off. EtOAc was added to the solid and again decanted off. This solid was further purified by mass-directed prep LC/MS to provide 3-((7-(cyclopropylamino)-5-(4-ethylpiperazin-1-yl)pyrazolo[1,5-a]pyrimidin-3-yl)methylene)pyrrolidine-2,5-dione. LCMS (M+1=396)

### Example 23

### Synthesis of 3-(7-(cyclopropylamino)-5-(methyl(1-methylpyrrolidin-3-yl)amino)pyrazolo[1,5-a]pyrimidin-3-yl)methylene)pyrrolidine-2,5-dione

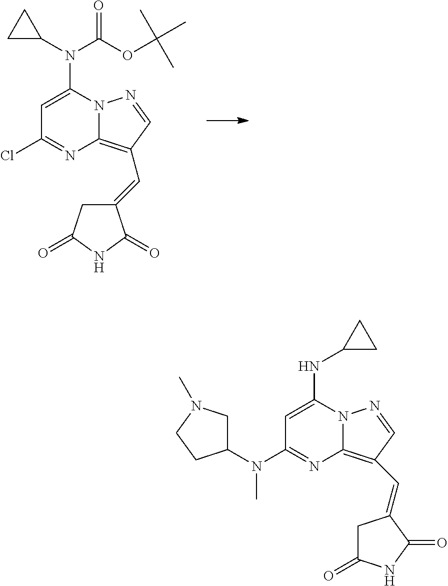

Example 23 was prepared by the procedures described above including the procedures for Example 22. LCMS (M+1=396)

### Example 24

### Synthesis of 3-((7-(cyclopropylamino)-5-(4-hydroxycyclohexylamino)pyrazolo[1,5-a]pyrimidin-3-yl)methylene)pyrrolidine-2,5-dione

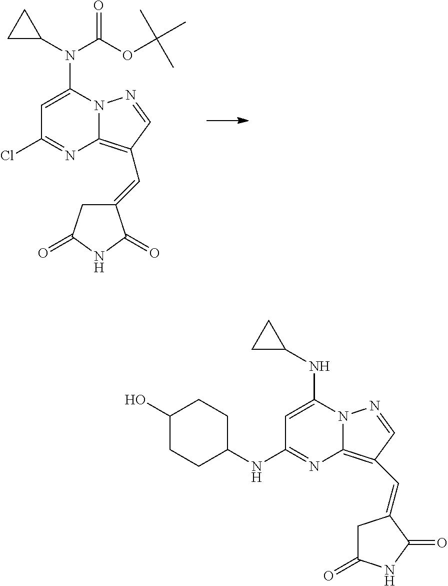

To tert-butyl 5-chloro-3-((2,5-dioxopyrrolidin-3-ylidene)methyl)pyrazolo[1,5-a]pyrimidin-7-yl(cyclopropyl)carbamate (15 mg, 0.03 mmol) in 1 mL of DMF was added K₂CO₃ (6 mg, 0.05 mmol), and trans-4-aminocyclohexanol (7 mg, 0.06 mmol). The reaction mixture was stirred at rt for 16 h. Dilute with EtOAc and wash 1× with 0.5M HCl. Organic layer dried with MgSO₄, filtered, and removed the solvent. To the residue was added 1 mL of 4M HCl/dioxane. Stir at 50° for 45 min. Remove excess HCl/dioxane on rotavap, add 1 mL of DMSO and purify by mass-directed prep LC/MS to provide 3-((7-(cyclopropylamino)-5-(4-hydroxycyclohexylamino)pyrazolo[1,5-a]pyrimidin-3-yl)methylene)pyrrolidine-2,5-dionc. LCMS (M+1=397)

### Example 25

### Synthesis of (S)-3-((7-(cyclopropylamino)-5-(1-phenylethylamino)pyrazolo[1,5-a]pyrimidin-3-yl)methylene)pyrrolidine-2,5-dione

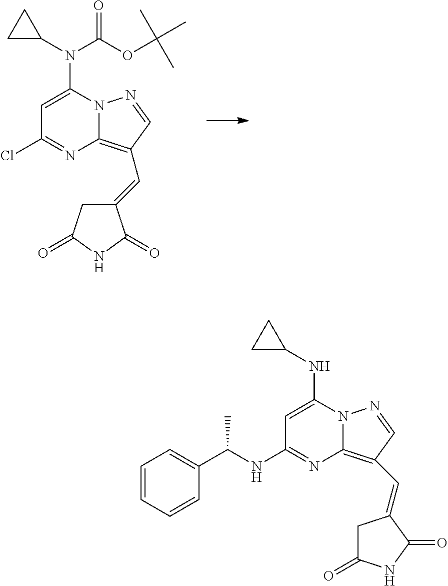

Example 25 was prepared by the procedures described above including the procedures for Example 24. LCMS (M+1=403)

The enantiomer of Example 25, the structure of which is shown below, can be prepared by procedures similar to Example 25.

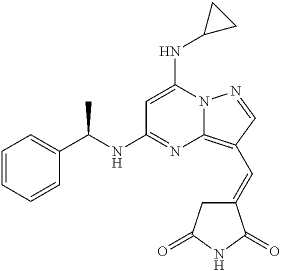

### Example 26

### Synthesis of 3-((5-((1r,4r)-4-aminocyclohexylamino)-7-(cyclopropylamino)pyrazolo[1,5-a]pyrimidin-3-yl)methylene)pyrrolidine-2,5-dione

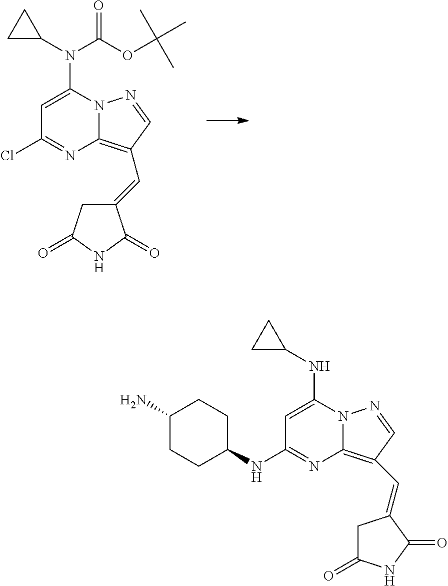

Example 26 was prepared by the procedures described above including the procedures for Example 24. LCMS (M+=396)

### Example 27

### Synthesis of 3-((7-(cyclopropylamino)-5-(pyridin-3-ylmethylamino)pyrazolo[1,5-a]pyrimidin-3-yl)methylene)pyrrolidine-2,5-dione

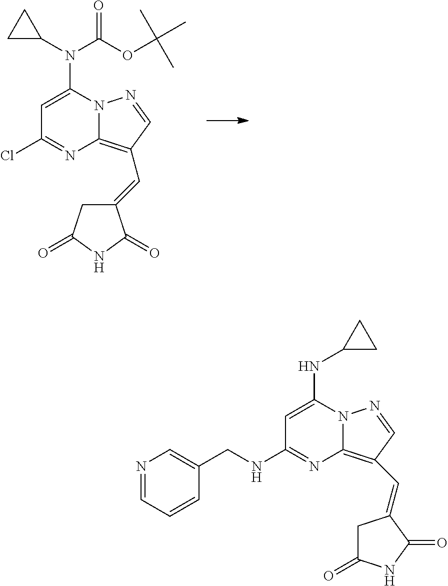

To tert-butyl 5-chloro-3-((2,5-dioxopyrrolidin-3-ylidene)methyl)pyrazolo[1,5-a]pyrimidin-7-yl(cyclopropyl)carbamate (15 mg, 0.036 mmol) in 1 mL of DMF was added K₂CO₃ (7 mg, 0.072 mmol), and pyridin-3-ylmethanamine (8 mg, 0.072 mmol). The reaction mixture was stirred at 60° for 2 h. Dilute with CH₂Cl₂ and wash 1× with 1M NH₄Cl. Organic layer dried with MgSO₄, filtered, and removed the solvent. To the residue was added 0.6 mL of 4M HCl/dioxane. Stir at 60° for 1 h. Add 0.5 mL of DMSO and purify by mass-directed prep LC/MS to provide 3-((7-(cyclopropylamino)-5-(pyridin-3-ylmethylamino)pyrazolo[1,5-a]pyrimidin-3-yl)methylene)pyrrolidine-2,5-dione. LCMS (M+1=390)

Examples 28 to 35 below were prepared by the procedures described above including the procedures for Example 27.

### Example 28

### Synthesis of 3-((7-(cyclopropylamino)-5-(pyridin-4-ylmethylamino)pyrazolo[1,5-a]pyrimidin-3-yl)methylene)pyrrolidine-2,5-dione

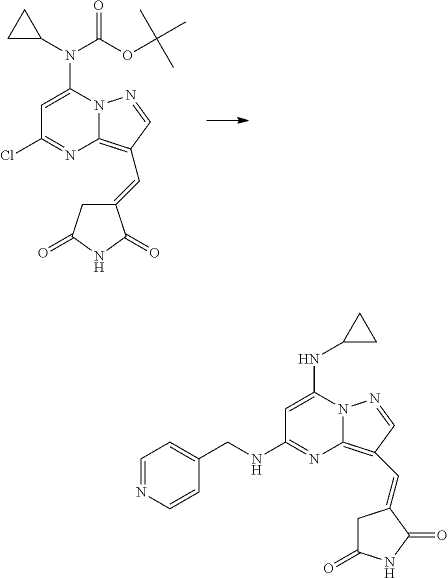

LCMS (M+1=390)

### Example 29

### Synthesis of 3-((7-cyclopropylamino)-5-(2-(pyridin-2-yl)ethylamino)pyrazolo[1,5-a]pyrimidin-3-yl)methylene)pyrrolidine-2,5-dione

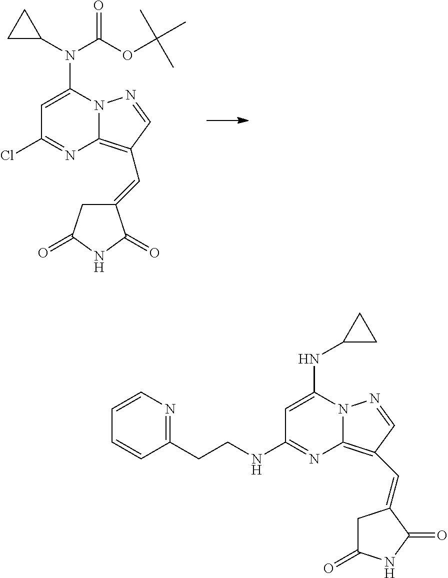

LCMS (M+1=404)

### Example 30

### Synthesis of 3-((7-(cyclopropylamino)-5-((5-methylpyrazin-2-yl)methylamino)pyrazolo[1,5-a]pyrimidin-3-yl)methylene)pyrrolidine-2,5-dione

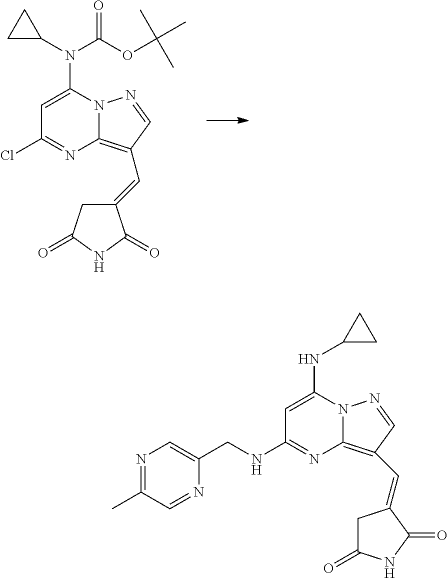

LCMS (M+1=405)

### Example 31

### Synthesis of 3-((7-(cyclopropylamino)-5-(6-methylpyridin-2-yl)methylamino)pyrazolo[1,5-a]pyrimidin-3-yl)methylene)pyrrolidine-2,5-dione

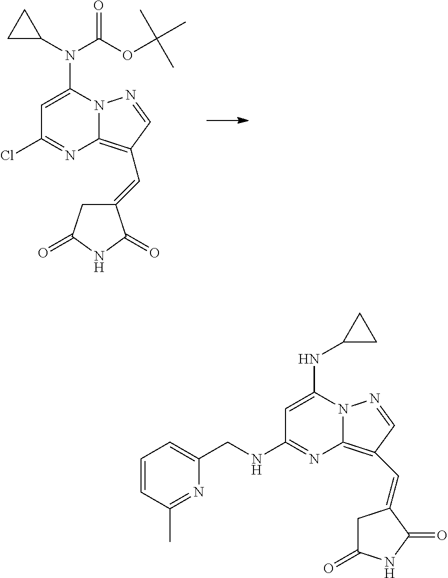

LCMS (M+1=404)

### Example 32

### Synthesis of 3-((7-(cyclopropylamino)-5-(imidazo[1,2-a]pyridin-2-ylmethylamino)pyrazolo[1,5-a]pyrimidin-3-yl)methylene)pyrrolidine-2,5-dione

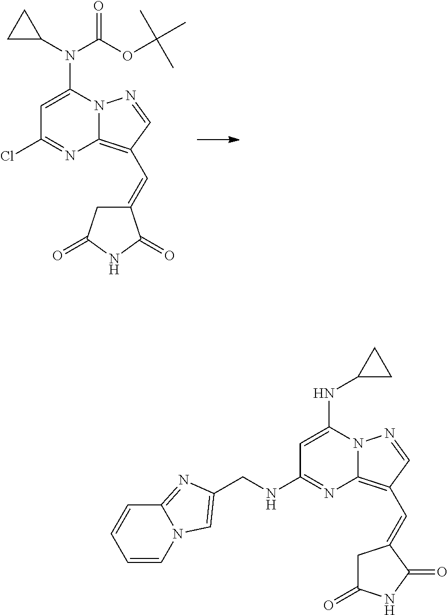

LCMS (M+1=429)

### Example 33

### Synthesis of 3-((5-(2-chlorobenzylamino)-7-(cyclopropylamino)pyrazolo[1,5-a]pyrimidin-3-yl)methylene)pyrrolidine-2,5-dione

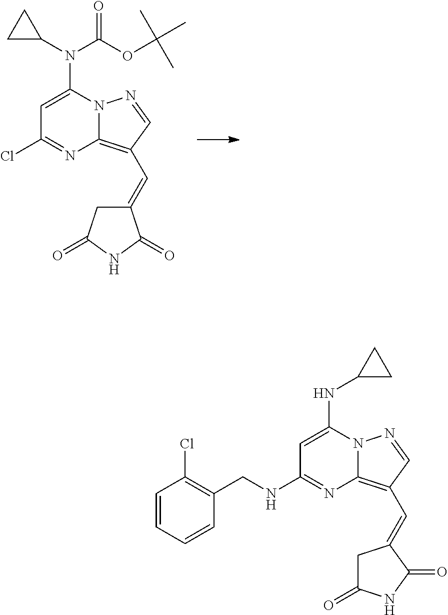

LCMS (M+1=423)

### Example 34

### Synthesis of 3-((5-(3-chlorobenzylamino)-7-(cyclopropylamino)pyrazolo[1,5-a]pyrimidin-3-yl)methylene)pyrrolidine-2,5-dione

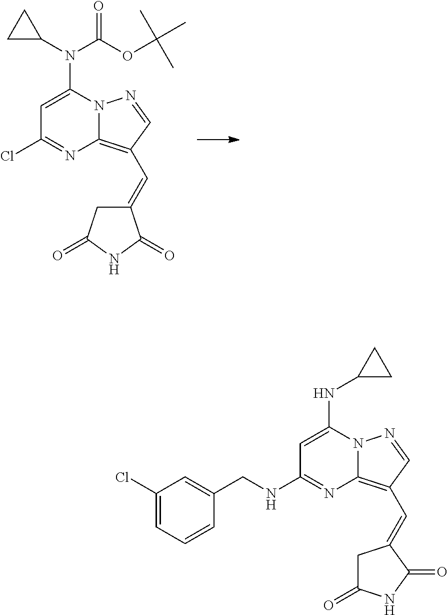

LCMS (M+1=423)

### Example 35

### Synthesis of 3-((5-(4-chlorobenzylamino)-7-(cyclopropylamino)pyrazolo[1,5-a]pyrimidin-3-yl)methylene)pyrrolidine-2,5-dione

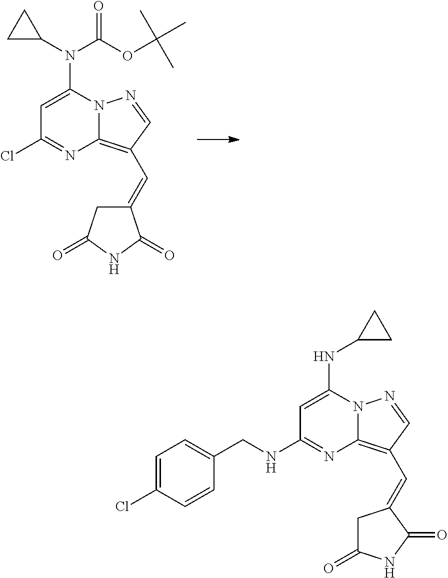

LCMS (M+1=423)

### Example 36

### Synthesis of 3-((7-(cyclopropylamino)-5-(3,5-dimethoxybenzylamino)pyrazolo[1,5-a]pyrimidin-3-yl)methylene)pyrrolidine-2,5-dione

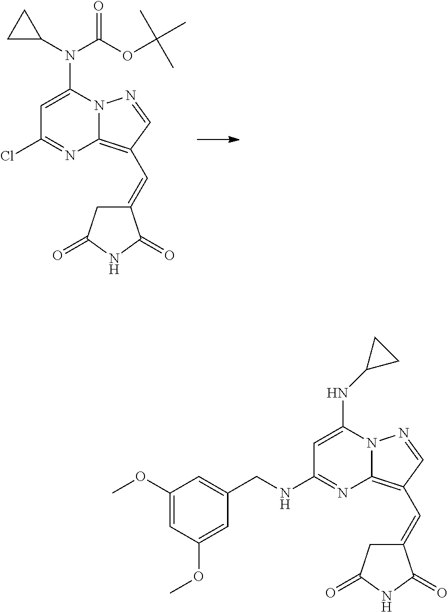

To tert-butyl 5-chloro-3-((2,5-dioxopyrrolidin-3-ylidene)methyl)pyrazolo[1,5-a]pyrimidin-7-yl(cyclopropyl)carbamate (10 mg, 0.024 mmol) in 0.5 mL of NMP was added K₂CO₃ (7 mg, 0.048 mmol), and (3,5-dimethoxyphenyl)methanamine (240 μL of a 0.2M solution in NMP). The reaction mixture was stirred at rt^{o} for 16 h. To the vial was added 0.3 mL of 4M HCl/dioxane. Stir at 80° for 2 h. Filter through a PTFE filter and purify by mass-directed prep LC/MS to provide 3-((7-(cyclopropylamino)-5-(3,5-dimethoxybenzyl amino)pyrazolo[1,5-a]pyrimidin-3-yl)methylene)pyrrolidine-2,5-dione. LCMS (M+1=449)

Examples 37 to 55 below were prepared by the procedures described above including the procedures for Example 36.

### Example 37

### Synthesis of 3-((5-(2-chloro-4-fluorobenzylamino)-7-(cyclopropylamino)pyrazolo[1,5-a]pyrimidin-3-yl)methylene)pyrolidine-2,5-dione

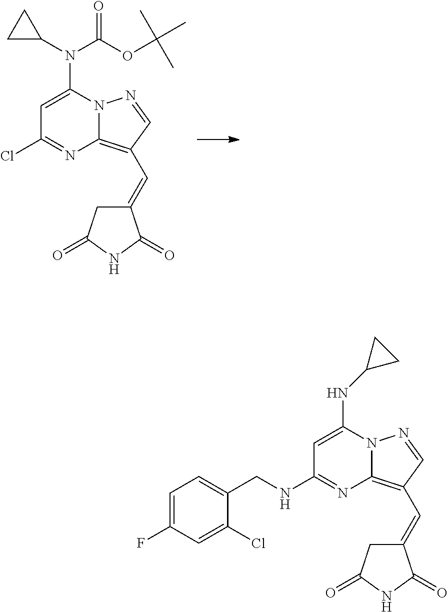

LCMS (M+1=441)

### Example 38

### Synthesis of 3-((7-(cyclopropylamino)-5-(4-methylthiophen-2-yl)methylamino)pyrazolo[1,5-a]pyrimidin-3-yl)methylene)pyrrolidine-2,5-dione

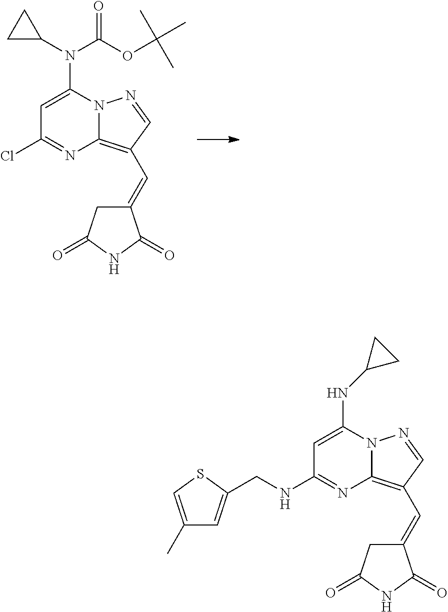

LCMS (M+1=409)

### Example 39

### Synthesis of 3-((7-(cyclopropylamino)-5-(thiophen-3-ylmethylamino)pyrazolo[1,5-a]pyrimidin-3-yl)methylene)pyrrolidine-2,5-dione

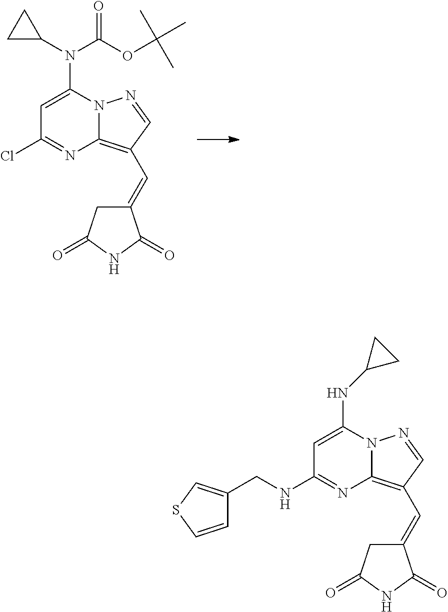

LCMS (M+1=395)

### Example 40

### Synthesis of 3-((7-(cyclopropylamino)-5-(1,2,3,4-tetrahydronaphthalen-1-ylamino)pyrazolo[1,5-a]pyrimidin-3-yl)methylene)pyrrolidine-2,5-dione

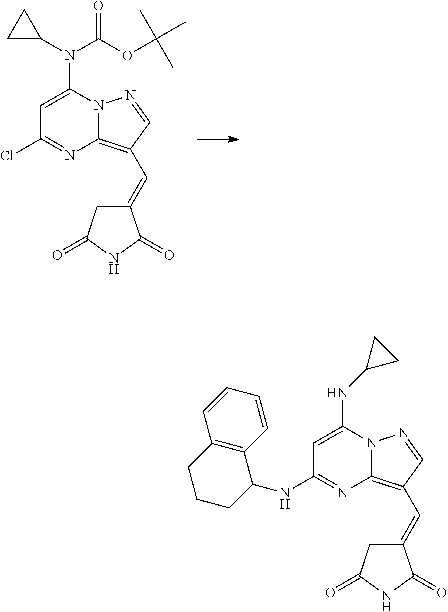

LCMS (M+1=429)

### Example 41

### Synthesis of (S)-3-((7-(cyclopropylamino)-5-(1-phenylpropylamino)pyrazolo[1,5-a]pyrimidin-3-yl)methylene)pyrrolidine-2,5-dione

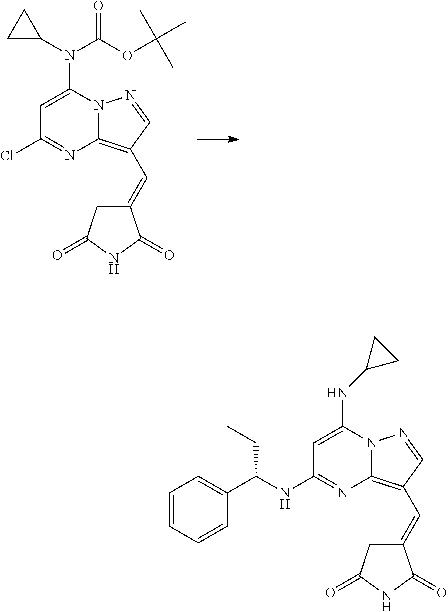

LCMS (M+1=417)

### Example 42

### Synthesis of 3-((7-(cyclopropylamino)-5-(2,6-difluorobenzylamino)pyrazolo[1,5-a]pyrimidin-3-yl)methylene)pyrrolidine-2,5-dione

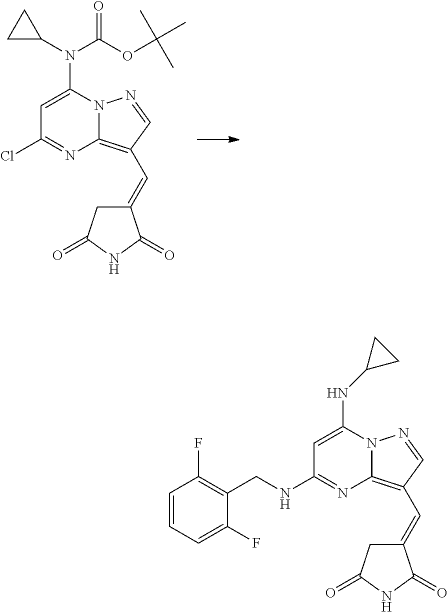

### Example 43

### Synthesis of 3-((7-(cyclopropylamino)-5-(3-methylbenzylamino)pyrazolo[1,5-a]pyrimidin-3-yl)methylene)pyrrolidine-2,5-dione

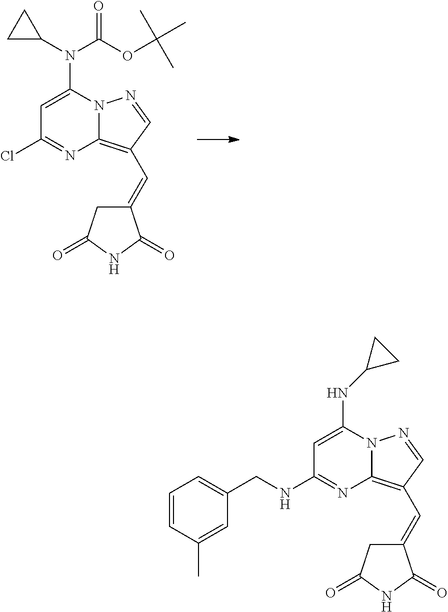

LCMS (M+1=403)

### Example 44

### Synthesis of 3-((7-(cyclopropylamino)-5-(thiophen-2-ylmethylamino)pyrazolo[1,5-a]pyrimidin-3-yl)methylene)pyrrolidine-2,5-dione

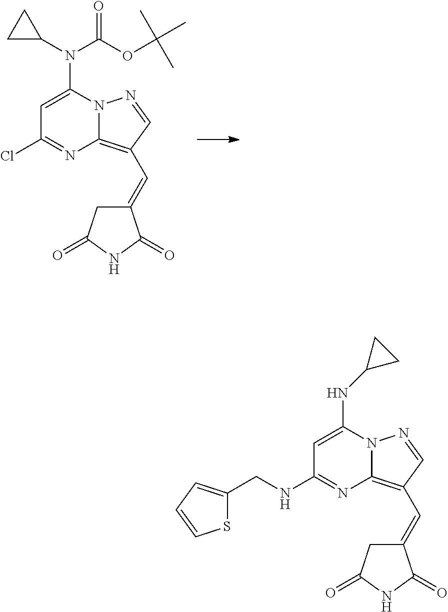

### Example 45

### Synthesis of 3-((7-(cyclopropylamino)-5-(2,3-difluorobenzylamino)pyrazolo[5-a]pyrimidin-3-yl)methylene)pyrrolidine-2,5-dione

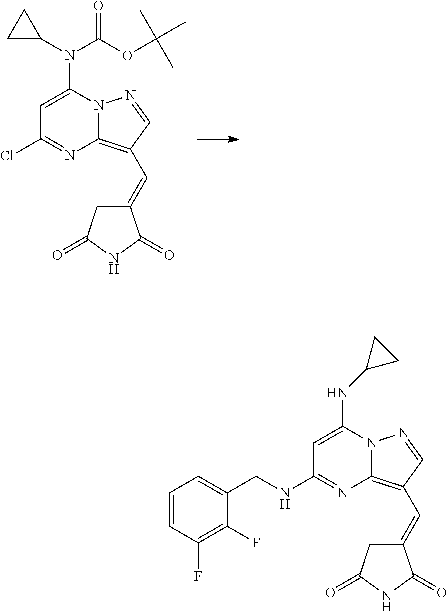

LCMS (M+1=425)

### Example 46

### Synthesis of 3-((7-(cyclopropylamino)-5-(2,4-difluorobenzylamino)pyrazolo[1,5-a]pyrimidin-3-yl)methylene)pyrrolidine-2,5-dione

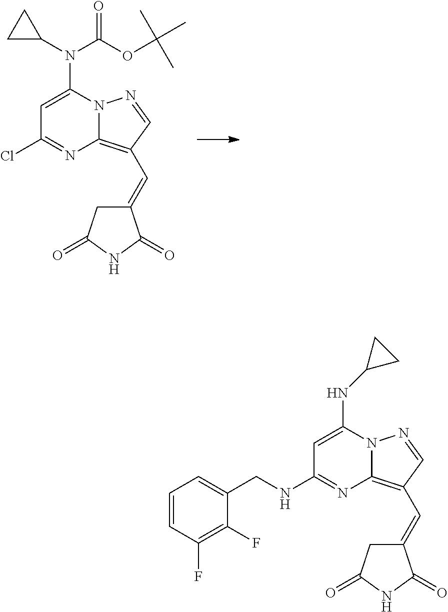

### Example 47

### Synthesis of 3-((7-(cyclopropylamino)-5-(3,5-difluorobenzylamino)pyrazolo[1,5-a]pyrimidin-3-yl)methylene)pyrrolidine-2,5-dione

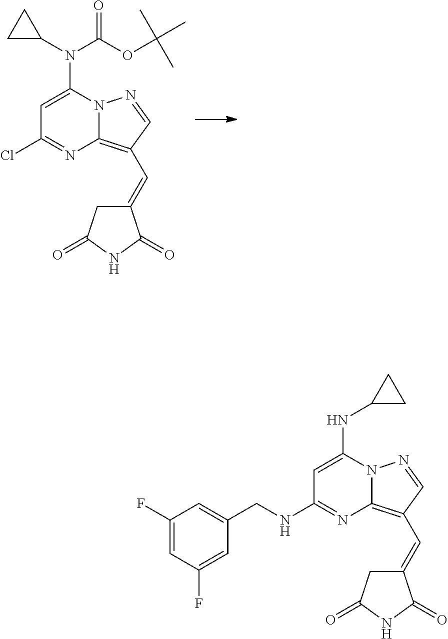

LCMS (M+1=425)

### Example 48

### Synthesis of 3-((7-(cyclopropylamino)-5-(2,3-dihydro-1H-inden-1-ylamino)pyrazolo[1,5-a]pyrimidin-3-yl)methylene)pyrrolidine-2,5-dione

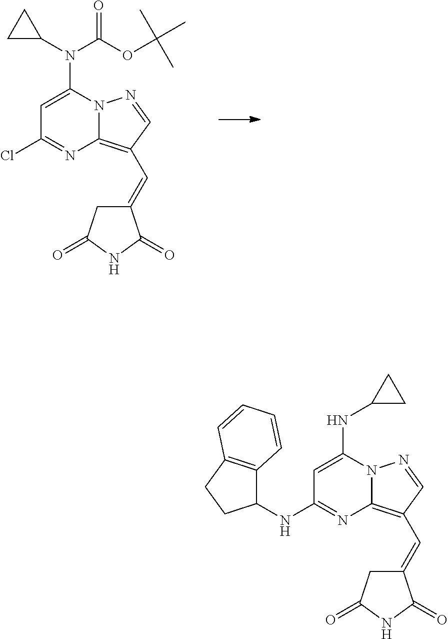

LCMS (M+1=415)

### Example 49

### Synthesis of (R)-3-((7-(cyclopropylamino)-5-(1-(4-fluorophenyl)ethylamino)pyrazolo[1,5-a]pyrimidin-3-yl)methylene)pyrrolidine-2,5-dione

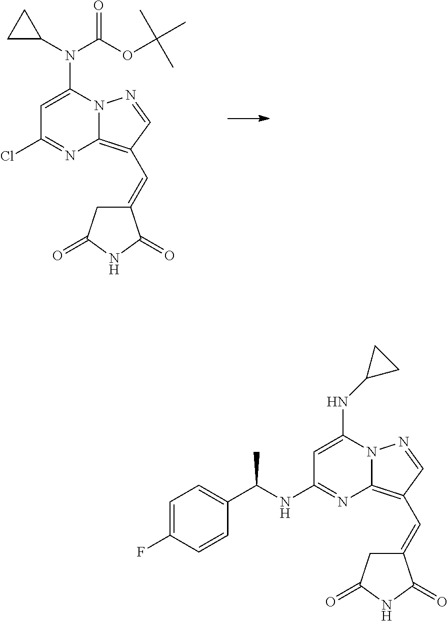

LCMS (M+1=421)

### Example 50

### Synthesis of (R)-3-((7-(cyclopropylamino)-5-(1-phenylpropylamino)pyrazolo[1,5-a]pyrimidin-3-yl)methylene)pyrrolidine-2,5-dione

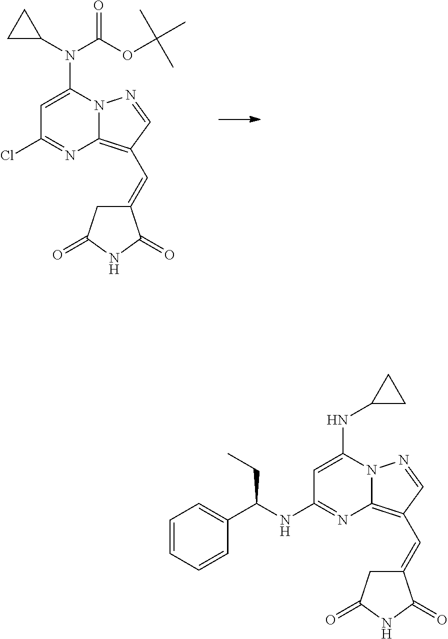

LCMS (M+1=417)

### Example 51

### Synthesis of (S)-3-((7-(cyclopropylamino)-5-(1-(4-fluorophenyl)ethylamino)pyrazolo[1,5-a]pyrimidin-3-yl)methylene)pyrrolidine-2,5-dione

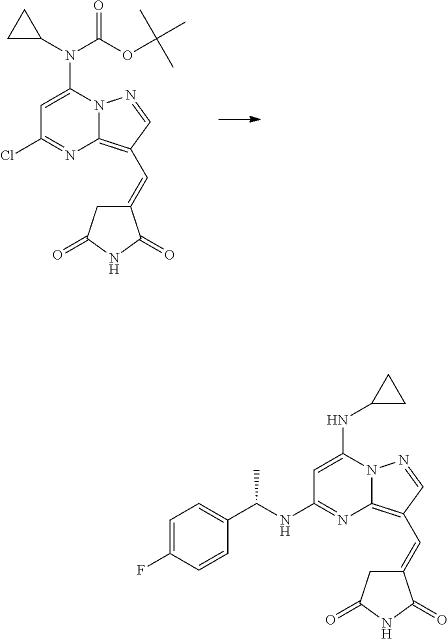

LCMS (M+1=421)

### Example 52

### Synthesis of (R)-3-((7-cyclopropylamino-5-(1-phenylethylamino)pyrazolo[1,5-a]pyrimidin-3-yl)methylene)pyrrolidine-2,5-dione

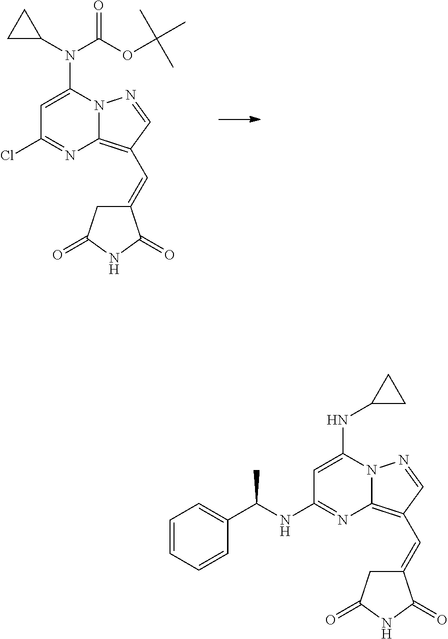

LCMS (M+1=403)

### Example 53

### Synthesis of 3-((7-(cyclopropylamino)-5-(2-morpholino-1-phenylethylamino)pyrazolo[1,5-a]pyrimidin-3-yl)methylene)pyrrolidine-2,5-dione

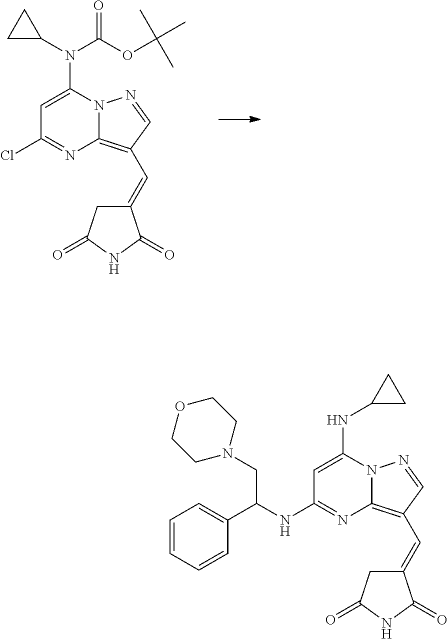

LCMS (M+1=488)

### Example 54

### Synthesis of 3-((7-(cyclopropylamino)-5-(methylamino)pyrazolo[1,5-a]pyrimidin-3-yl)methylene)pyrrolidine-2,5-dione

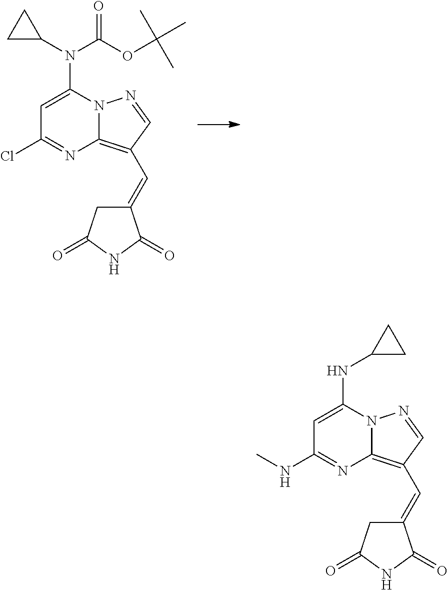

LCMS (M+1=313)

### Example 55

### Synthesis of 3-((7-(cyclopropylamino)-5-(dimethylamino)pyrazolo[1,5-a]pyrimidin-3-yl)methylene)pyrrolidine-2,5-dione

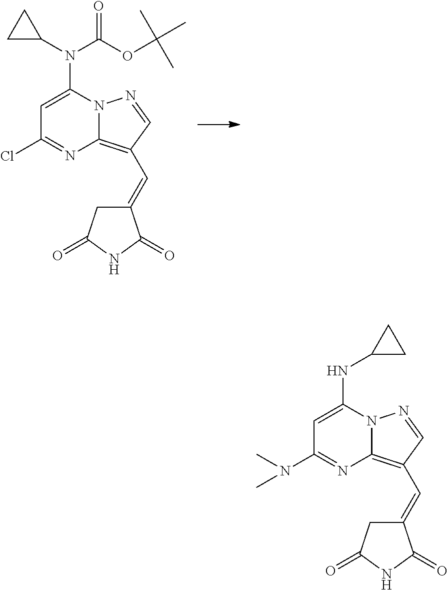

LCMS (M+1=327)

### Example 56

### Synthesis of 3-((5-(3-chlorophenylamino)-7-(cyclopropylamino)pyrazolo[1,5-a]pyrimidin-3-yl)methylene)pyrrolidine-2,5-dione

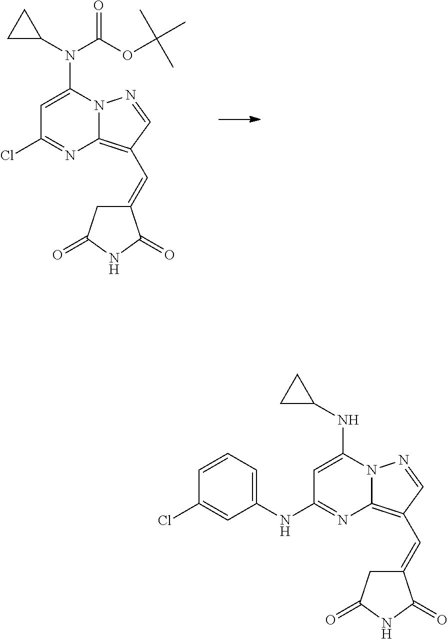

To tert-butyl 5-chloro-3-((2,5-dioxopyrrolidin-3-ylidene)methyl)pyrazolo[1,5-a]pyrimidin-7-yl(cyclopropyl)carbamatecarbamate (80 mg, 0.191 mmol) in 1,4-dioxane (3 mL) was added PTSA (7 mg, 0.038 mmol), and 3-chloroaniline (200 μL, 1.91 mmol). The reaction mixture was stirred at reflux temperature overnight. Partitioned between methylene chloride and H₂O, Separated layers. Organic layer was dried with MgSO₄, filtered, and removed solvent. The resulting residue was purified by flash chromatography (40%-60% EtOAc/hexane). Pure fractions were combined to provide 3-((5-(3-chlorophenylamino)-7-(cyclopropylamino)pyrazolo[1,5-a]pyrimidin-3-yl)methylene)pyrrolidine-2,5-dione. LCMS (M+1=409)

### Example 57

### Synthesis of 3-((5-(4-chlorophenylamino)-7-(cyclopropylamino)pyrazolo[1,5-a]pyrimidin-3-yl)methylene)pyrrolidine-2,5-dione

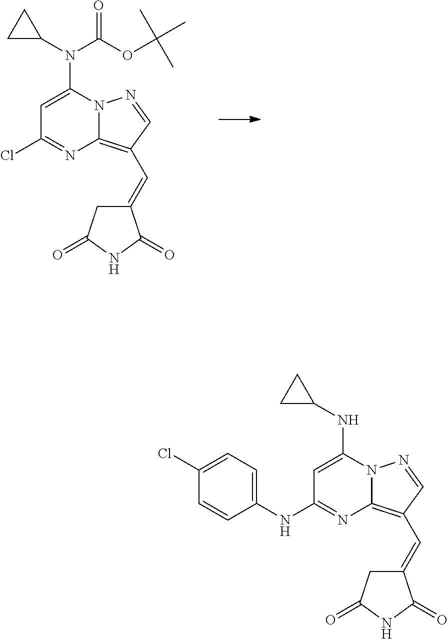

To tert-butyl 5-chloro-3-((2,5-dioxopyrrolidin-3-ylidene)methyl)pyrazolo[1,5-a]pyrimidin-7-yl(cyclopropyl)carbamate (75 mg, 0.179 mmol) in 1,4-dioxane (2 mL) was added cesium carbonate (82 mg, mg, 0.358 mmol), 4-chloroaniline (34 mg, 0.197 mmol); Pd(OAc)₂ (2 mg, 0.007 mmol), and racemic BINAP (7 mg, 0.011 mmol). The reaction mixture was stirred under microwave heating at 150° C. for 20 minutes. Dilute with CH₂Cl₂ and wash 1× with 0.5M HCl. Dry organic layer with MgSO₄, filter, and remove solvent to provide residue which was treated with 1 mL of 4M HCl in dioxane. Stir at 50° C. for 1 h. Cool to room temperature and the excess HCl/dioxane was removed on rotavap. Add 4 mL of saturated NaHCO₃. The resulting precipitate was filtered off and rinsed with H₂O followed by methanol. Dry under vacuum to provide 20 mg of 3-((5-(4-chlorophenylamino)-7-(cyclopropylamino)pyrazolo[1,5-a]pyrimidin-3-yl)methylene)pyrrolidine-2,5-dione.

LCMS (M+1=409)

Examples 58 to 90 were prepared by the procedures described above including the procedures for Examples 56 and 57.

### Example 58

### Synthesis of 3-((7-(cyclopropylamino)-5-(3-(trifluoromethyl)phenylamino)pyrazolo[1,5-a]pyrimidin-3-yl)methylene)pyrrolidine-2,5-dione

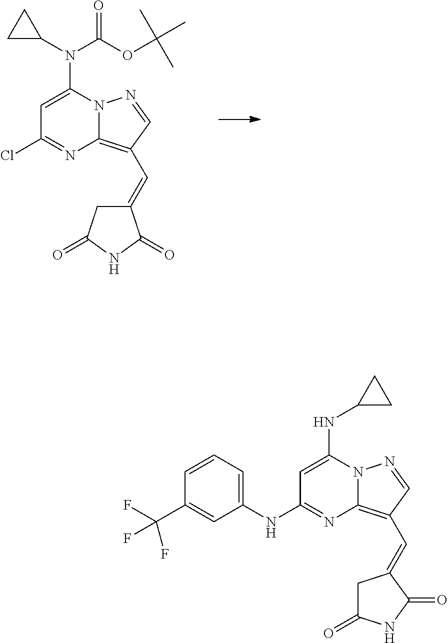

LCMS (M+1=443)

### Example 59

### Synthesis of 3-((7-(cyclopropylamino)-5-(3-methoxyphenylamino)pyrazolo[1,5-a]pyrimidin-3-yl)methylene)pyrrolidine-2,5-dione

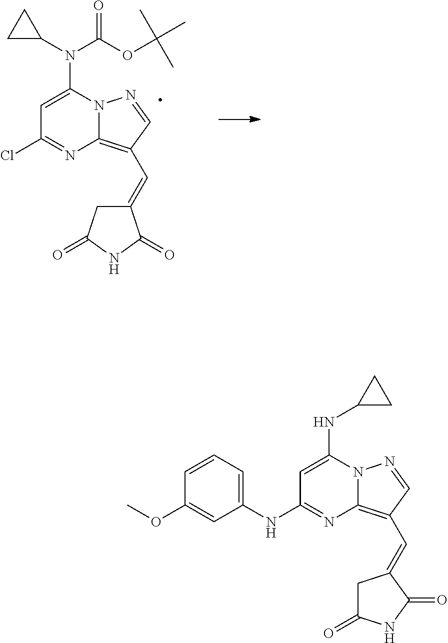

LCMS (M+1=405)

### Example 60

### Synthesis of 3-((7-(cyclopropylamino)-5-(3-(trifluoromethoxy)phenylamino)pyrazolo[1,5-a]pyrimidin-3-yl)methylene)pyrrolidine-2,5-dione

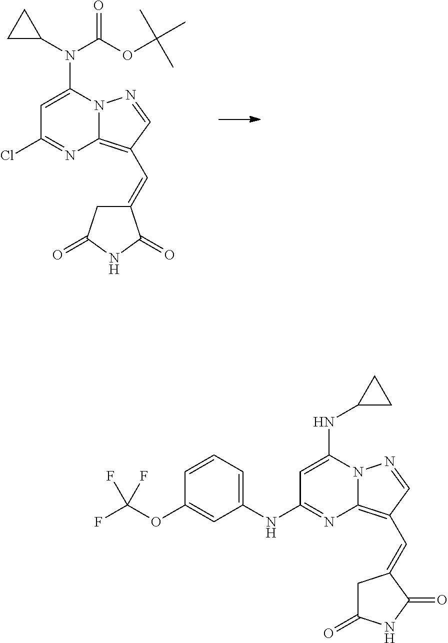

### Example 61

### Synthesis of 3-((7-(cyclopropylamino)-5-(3-fluorophenylamino)pyrazolo[1,5-a]pyrimidin-3-yl)methylene)pyrrolidine-2,5-dione

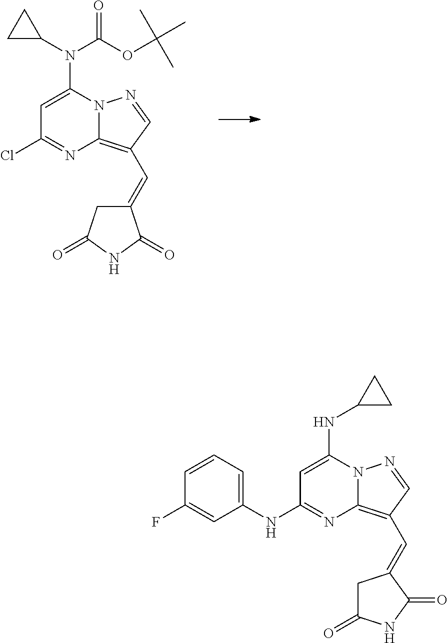

LCMS (M+1=393)

### Example 62

### Synthesis of 3-((7-(cyclopropylamino)-5-(m-tolylamino)pyrazolo[1,5-a]pyrimidin-3-yl)methylene)pyrrolidine-2,5-dione

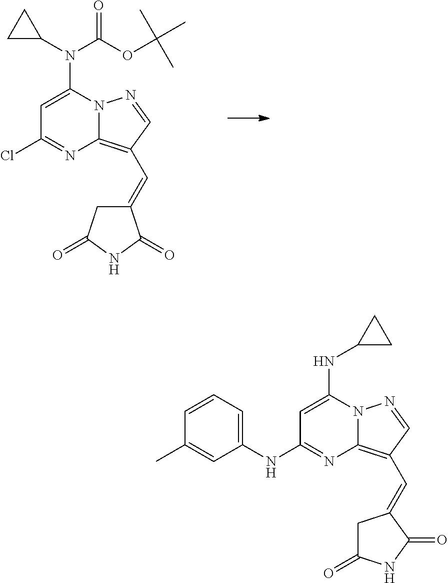

LCMS (M+1=389)

### Example 63

### Synthesis of 3-((7-(cyclopropylamino)-5-(3,5-difluorophenylamino)pyrazolo[1,5-a]pyrimidin-3-yl)methylene)pyrrolidine-2,5-dione

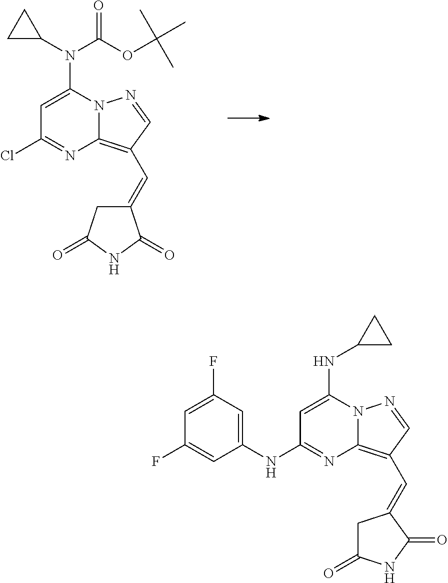

LCMS (M+1=411)

### Example 64

### Synthesis of 3-((7-(cyclopropylamino)-5-(3-(morpholinomethyl)phenylamino)pyrazolo[1,5-a]pyrimidin-3-yl)methylene)pyrrolidine-2,5-dione

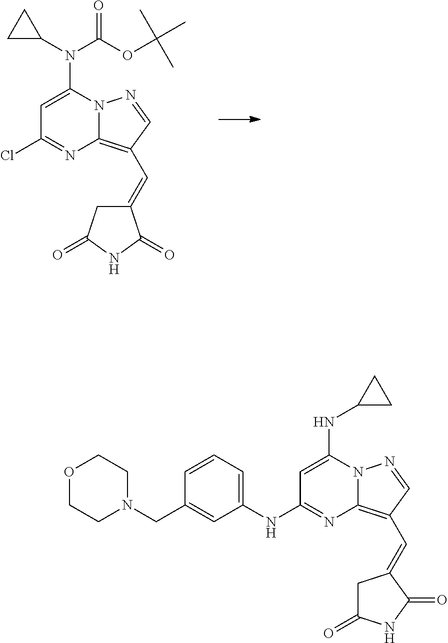

LCMS (M+1=474)

### Example 65

### Synthesis of 3-((7-(cyclopropylamino)-5-(4-(4-methylpiperazin-1-yl)phenylamino)pyrazolo[1,5-a]pyrimidin-3-yl)methylene)pyrrolidine-2,5-dione

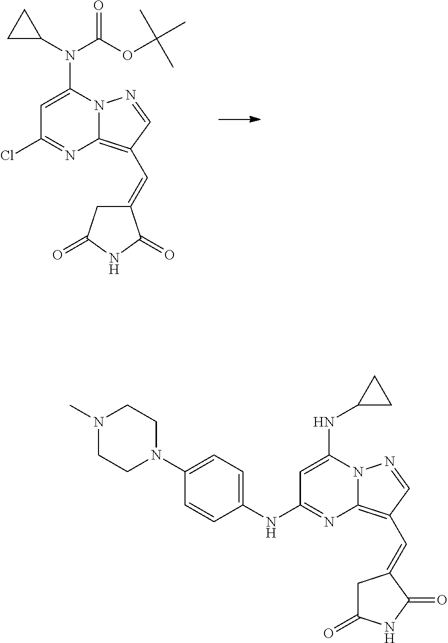

### Example 66

### Synthesis of 3-((5-(3-chloro-4-fluorophenylamino)-7-(cyclopropylamino)pyrazolo[1,5-a]pyrimidin-3-yl)methylene)pyrrolidine-2,5-dione

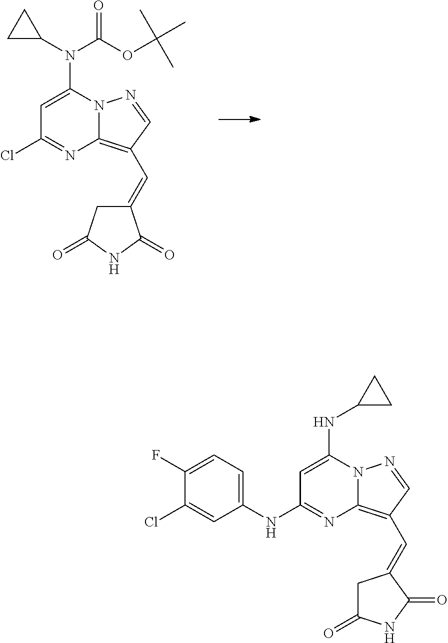

LCMS (M+1-427)

### Example 67

### Synthesis of 3-((5-(2-chloro-4-fluorophenylamino)-7-(cyclopropylamino)pyrazolo[1,5-a]pyrimidin-3-yl)methylene)pyrrolidine-2,5-dione

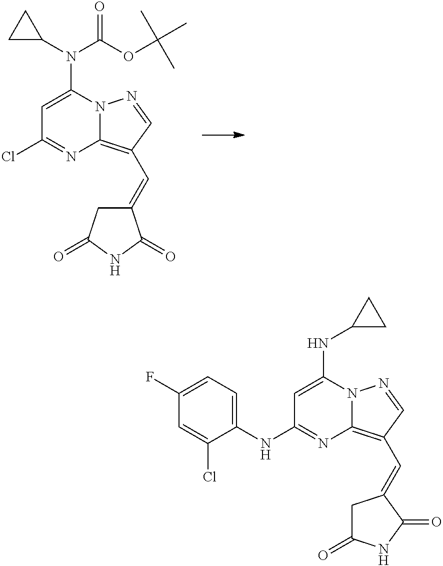

LCMS (M+1=427)

### Example 68

### Synthesis of 3-((5-(5-chloro-2-fluorophenylamino)-7-(cyclopropylamino)pyrazolo[1,5-a]pyrimidin-3-yl)methylene)pyrrolidine-2,5-dione

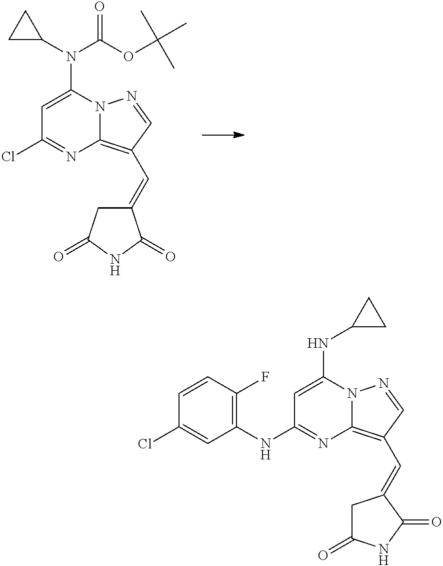

### Example 69

### Synthesis of 3-((7-(cyclopropylamino)-5-(2,4-difluorophenylamino)pyrazolo[1,5-a]pyrimidin-3-yl)methylene)pyrrolidine-2,5-dione

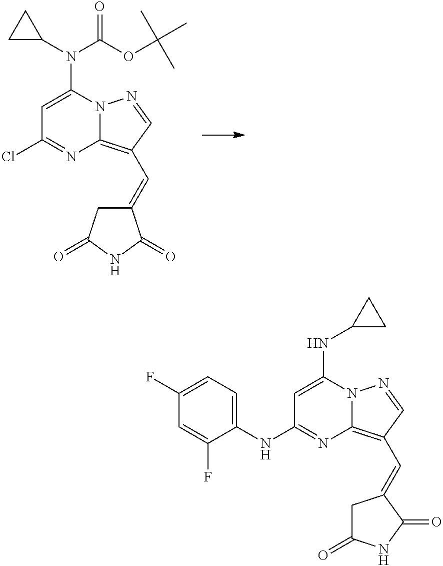

LCMS (M+1=411)

### Example 70

### Synthesis of 3-((7-(cyclopropylamino)-5-(3,4-difluorophenylamino)pyrazolo[1,5-a]pyrimidin-3-yl)methylene)pyrrolidine-2,5-dione

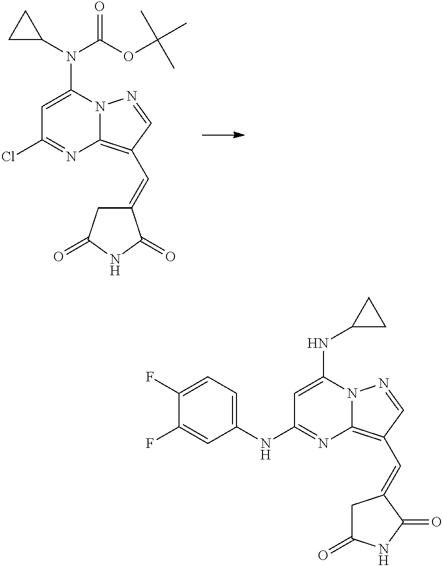

LCMS (M+1=411)

### Example 71

### Synthesis of 3-((7-(cyclopropylamino)-5-(2-(trifluoromethyl)phenylamino)pyrazolo[1,5-a]pyrimidin-3-yl)methylene)pyrrolidine-2,5-dione

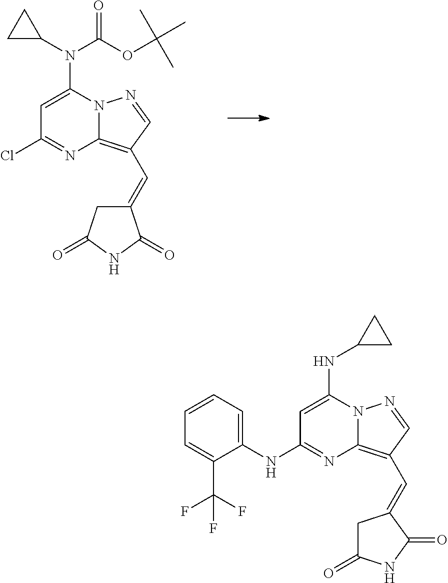

LCMS (M+1=443)

### Example 72

### Synthesis of 3-((5-(benzo[d][1,3]dioxol-5-ylamino)-7-(cyclopropylamino)pyrazolo[1,5-a]pyrimidin-3-yl)methylene)pyrrolidine-2,5-dione

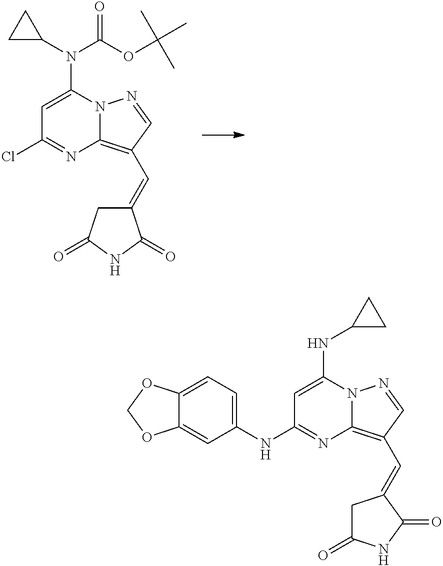

LCMS (M+1=419)

### Example 73

### Synthesis of 3-((7-(cyclopropylamino)-5-(methyl(phenyl)amino)pyrazolo[1,5-a]pyrimidin-3-yl)methylene)pyrrolidine-2,5-dione

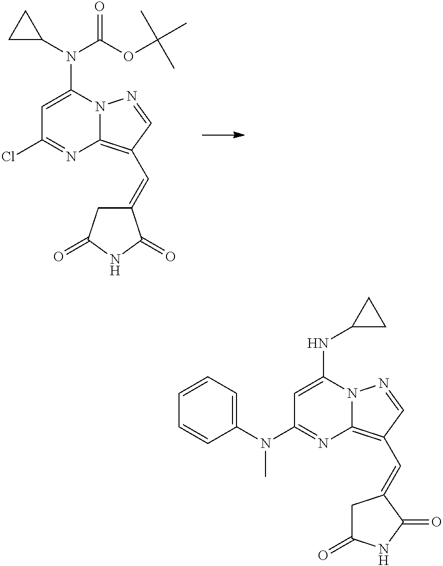

### Example 74

### Synthesis of 3-((7-(cyclopropylamino)-5-(4-isopropoxyphenylamino)pyrazolo[1,5-a]pyrimidin-3-yl)methylene)pyrrolidine-2,5-dione

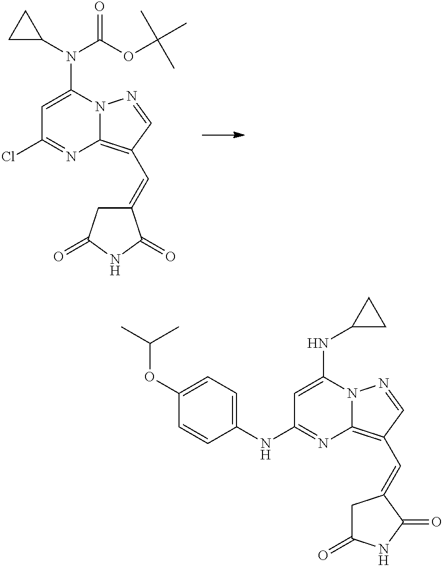

LCMS (M+1=433)

### Example 75

### Synthesis of 3-((7-(cyclopropylamino)-5-(3-isopropylphenylamino)pyrazolo[1,5-a]pyrimidin-3-yl)methylene)pyrrolidine-2,5-dione

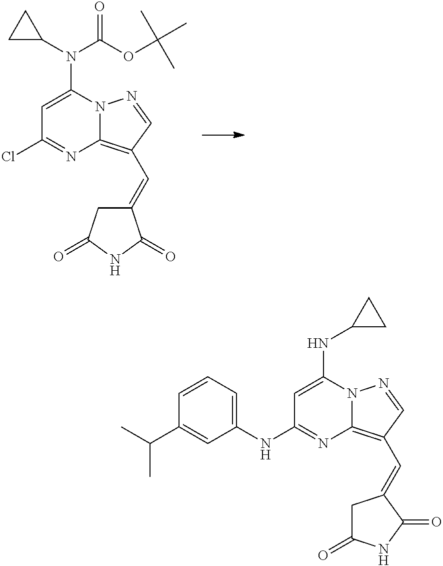

LCMS (M+1=417)

### Example 76

### Synthesis of 3-((5-(2-chloro-3-methoxyphenylamino)-7-(cyclopropylamino)pyrazolo[1,5-a]pyrimidin-3-yl)methylene)pyrrolidine-2,5-dione

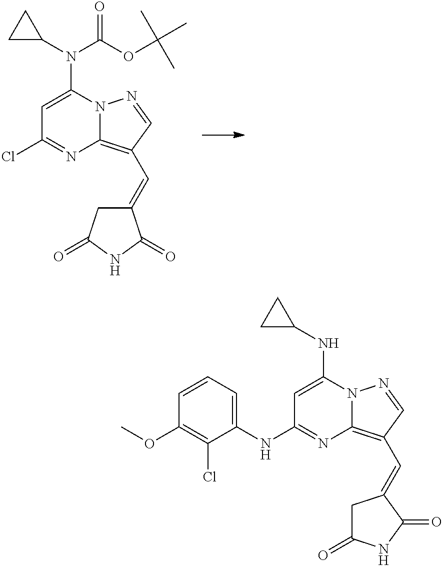

### Example 77

### Synthesis of 3-((7-(cyclopropylamino)-5-(4-methoxyphenylamino)pyrazolo[1,5-a]pyrimidin-3-yl)methylene)pyrrolidine-2,5-dione

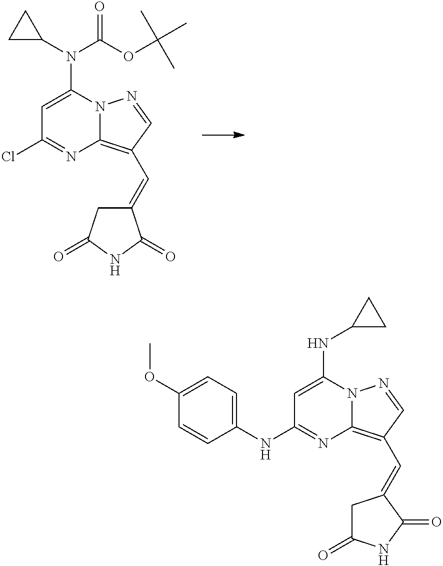

LCMS (M+1=405)

### Example 78

### Synthesis of 3-((5-(3-acetylphenylamino)-7-(cyclopropylamino)pyrazolo[1,5-a]pyrimidin-3-yl)methylene)pyrrolidine-2,5-dione

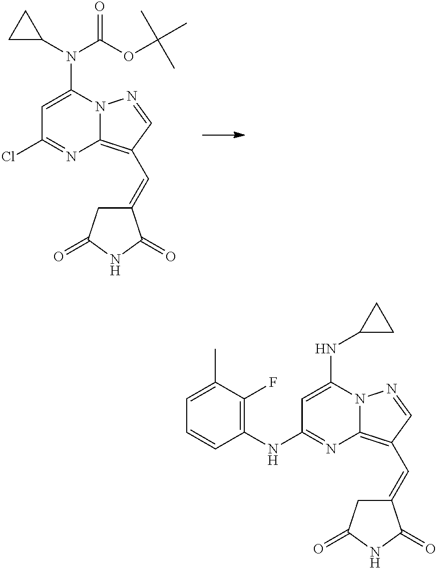

LCMS (M+1=417)

### Example 79

### Synthesis of 3-((7-(cyclopropylamino)-5-(2-fluoro-3-methylphenylamino)pyrazolo[1,5-a]pyrimidin-3-yl)methylene)pyrrolidine-2,5-dione

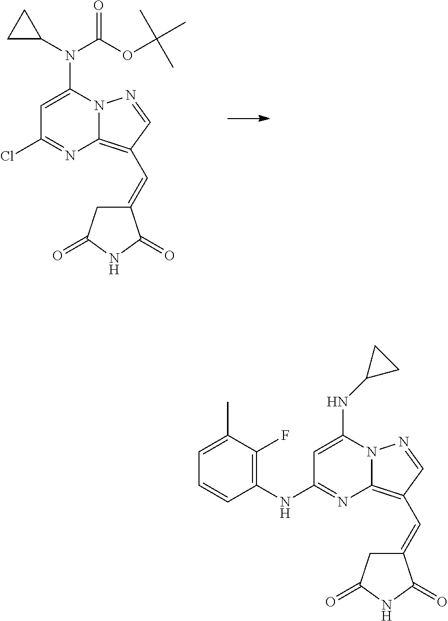

LCMS (M+1=407)

### Example 80

### Synthesis of 3-((5-(2-chloro-4-fluoro-5-methylphenylamino)-7-(cyclopropylamino)pyrazolo[1,5-a]pyrimidin-3-yl)methylene)pyrrolidine-2,5-dione

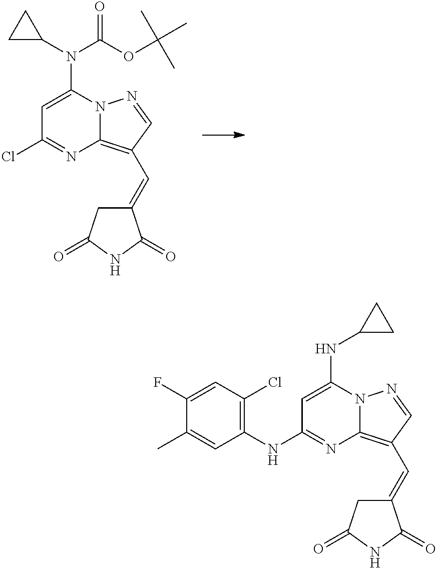

LCMS (M+1=441)

### Example 81

### Synthesis of 3-((7-(cyclopropylamino)-5-(4-fluoro-3-methylphenylamino)pyrazolo[1,5-a]pyrimidin-3-yl)methylene)pyrrolidine-2,5-dione

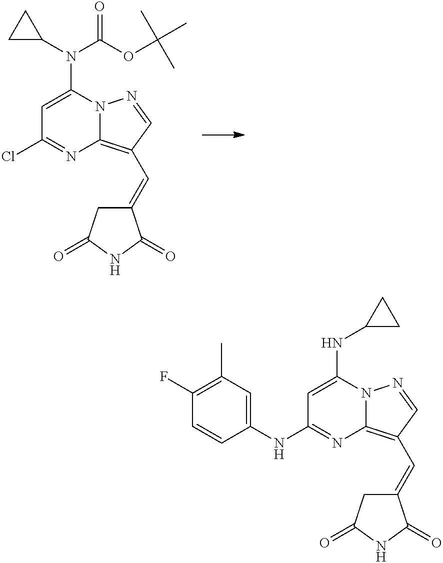

### Example 82

### Synthesis of 3-((7-(cyclopropylamino)-5-(2-fluoro-5-methylphenylamino)pyrazolo-[1,5-a]pyrimidin-3-yl)methylene)pyrrolidine-2,5-dione

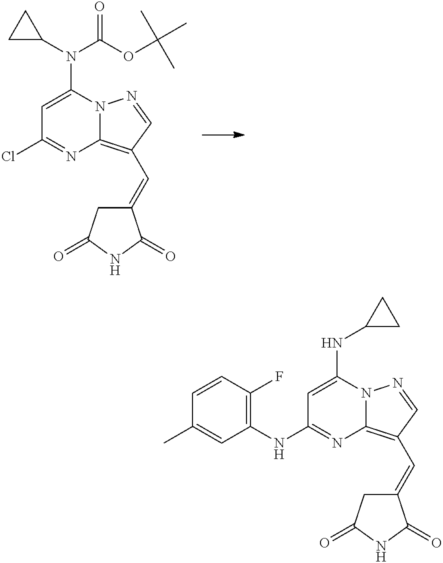

LCMS (M+1=407)

### Example 83

### Synthesis of 4-chloro-3-(7-(cyclopropylamino)-3-((2,5-dioxopyrrolidin-3-ylidene)methyl)pyrazolo[1,5-a]pyrimidin-5-ylamino)benzonitrile

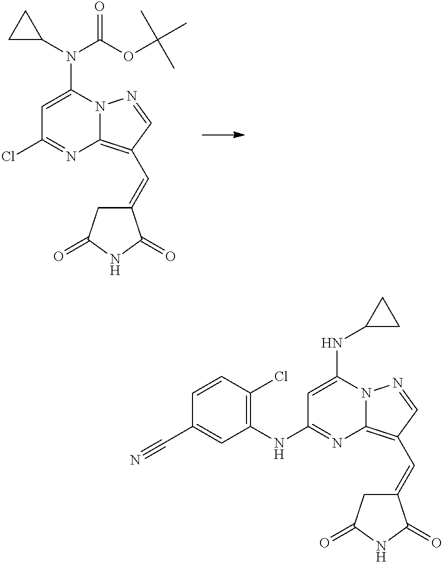

LCMS (M+1=434)

### Example 84

### Synthesis of 3-((5-(4-(1H-pyrazol-1-yl)phenylamino)-7-(cyclopropylamino)pyrazolo[1,5-a]pyrimidin-3-yl)methylene)pyrrolidine-2,5-dione

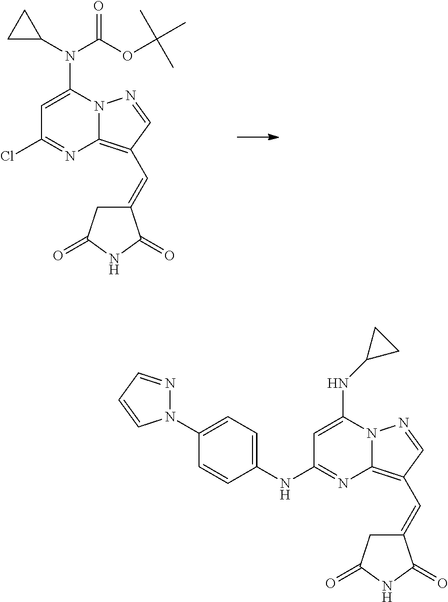

### Example 85

### Synthesis of 3-((5-(2-chloro-4-hydroxyphenylamino)-7-(cyclopropylamino)pyrazolo[1,5-a]pyrimidin-3-yl)methylene)pyrrolidine-2,5-dione

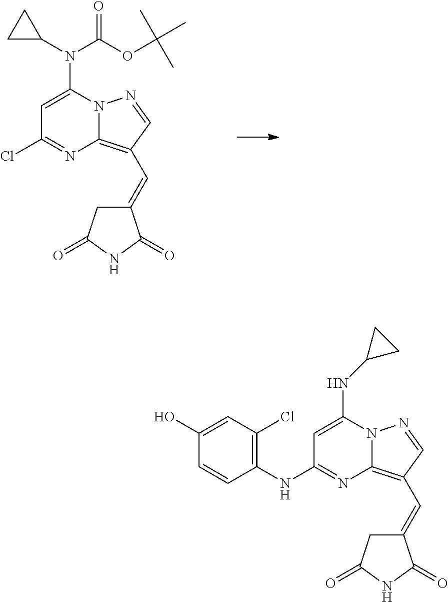

LCMS (M+1=425)

### Example 86

### Synthesis of 3-((5-(3-chloro-5-fluorophenylamino)-7-(cyclopropylamino)pyrazolo[1,5-a]pyrimidin-3-yl)methylene)pyrrolidine-2,5-dione

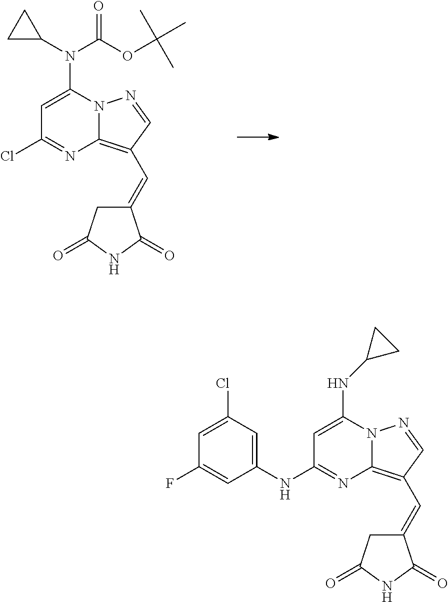

LCMS (M+1=427)

### Example 87

### Synthesis of 3-((7-(cyclopropylamino)-5-(3-fluoro-2-methylphenylamino)pyrazolo[1,5-a]pyrimidin-3-yl)methylene)pyrrolidine-2,5-dione

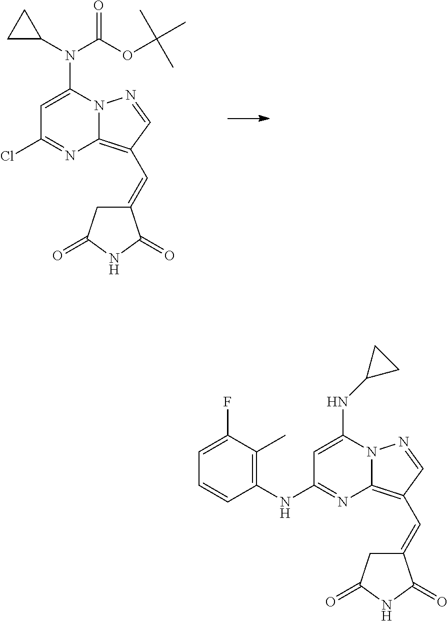

LCMS (M+1=407)

### Example 88

### Synthesis of 3-((5-(3-chloro-4-methylphenylamino)-7-(cyclopropylamino)pyrazolo[1,5-a]pyrimidin-3-yl)methylene)pyrrolidine-2,5-dione

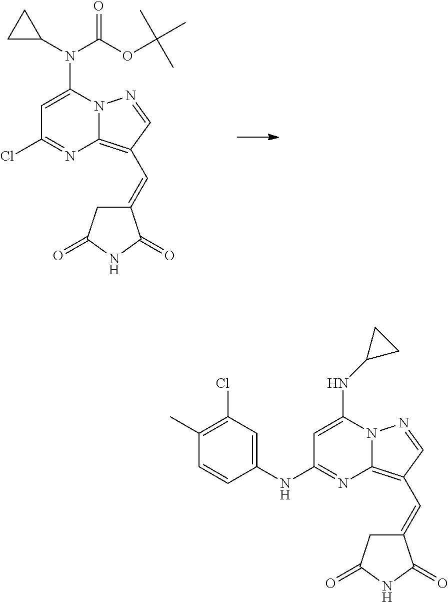

LCMS (M+1=423)

### Example 89

### Synthesis of 3-((7-(cyclopropylamino)-5-(2,3-difluorophenylamino)pyrazolo[1,5-a]pyrimidin-3-yl)methylene)pyrrolidine-2,5-dione

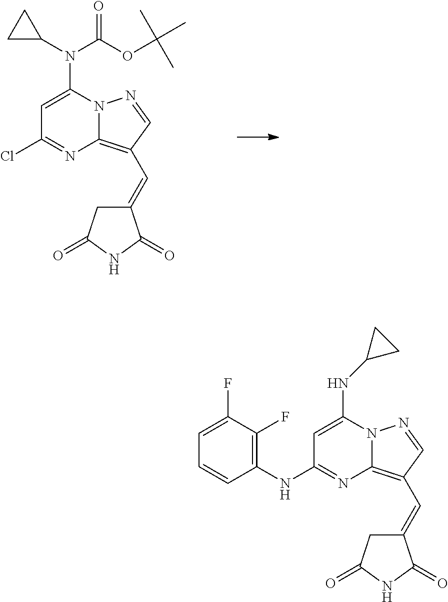

### Example 90

### Synthesis of 3-((5-(5-chloro-2-methylphenylamino)-7-(cyclopropylamino)pyrazolo[1,5-a]pyrimidin-3-yl)methylene)pyrrolidine-2,5-dione

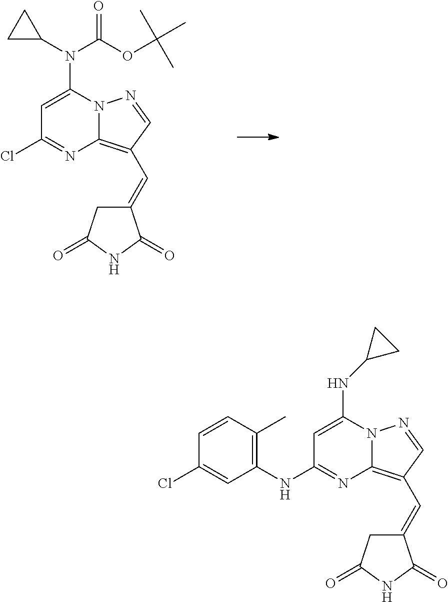

LCMS (M+1=423)

### Example 91

### Synthesis of 3-((7-(cyclopropylamino)-5-(pyridin-4-ylamino)pyrazolo[1,5-a]pyrimidin-3-yl)methylene)pyrrolidine-2,5-dione

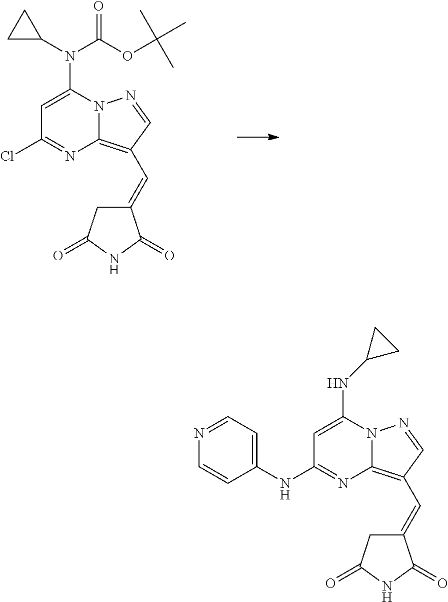

To tert-butyl 5-chloro-3-((2,5-dioxopyrrolidin-3-ylidene)methyl)pyrazolo[1,5-a]pyrimidin-7-yl(cyclopropyl)carbamatecarbamate (20 mg, 0.048 mmol) in 1,4-dioxane (1 mL) was added PTSA (2 mg, 0.01 mmol), and 4-aminopyridine (22 mg, 0.24 mmol). The reaction mixture was stirred at reflux temperature for 3 hours. Add 500 μL of 4M HCl in dioxane and 500 μL H₂O and stir at 50° overnight. The resulting yellow precipitate was filtered and rinsed with dioxane. Dried to constant weight to provide 3-((7-(cyclopropylamino)-5-(pyridin-4-ylamino)pyrazolo[1,5-a]pyrimidin-3-yl)methylene)pyrrolidine-2,5-dione. LCMS (M+1=376)

### Example 92

### Synthesis of 3-((7-(cyclopropylamino)-5-(pyridin-3-ylamino)pyrazolo[1,5-a]pyrimidin-3-yl)methylene)pyrrolidine-2,5-dione

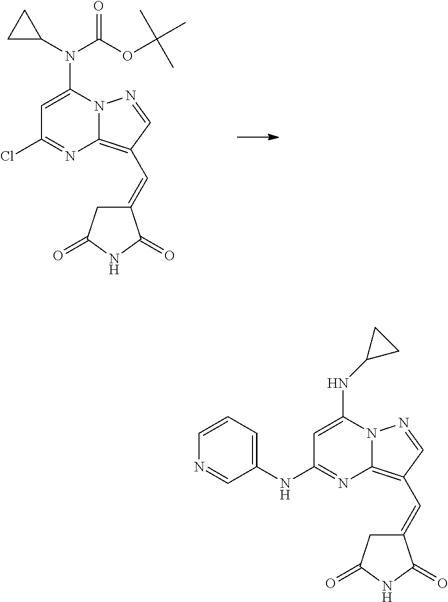

To tert-butyl 5-chloro-3-((2,5-dioxopyrrolidin-3-ylidene)methyl)pyrazolo[1,5-a]pyrimidin-7-yl(cyclopropyl)carbamatecarbamate (20 mg, 0.048 mmol) in 1,4-dioxane (1 mL) was added PTSA (2 mg, 0.01 mmol), and 3-aminopyridine (22 mg, 0.24 mmol). The reaction mixture was stirred at reflux temperature for 16 hours. Add 500 μL of 4M HCl in dioxane and 500 μL H₂O and stir at 50° for 5 h. Dilute with DMSO and purify by mass-directed prep LC/MS to provide 3-((7-(cyclopropylamino)-5-(pyridin-4-ylamino)pyrazolo[1,5-a]pyrimidin-3-yl)methylene)pyrrolidine-2,5-dione. LCMS (M+1=376)

The following four compounds were prepared by the procedures described above.

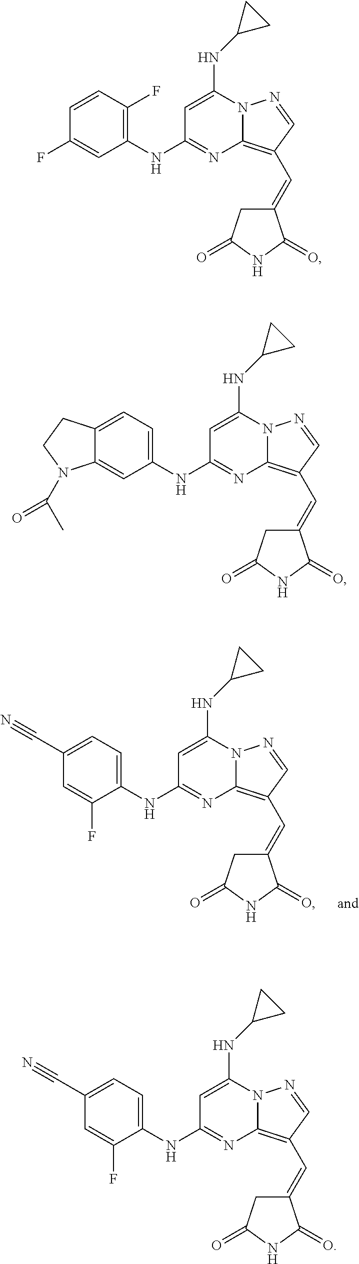

### Example 93

### Synthesis of 3-((5-chloro-7-(cyclopropylmethylamino)pyrazolo[1,5-a]pyrimidin-3-yl)methylene)pyrrolidine-2,5-dione

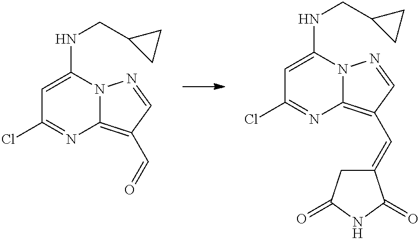

To 5-chloro-7-(cyclopropylmethylamino)pyrazolo[1,5-a]pyrimidine-3-carbaldehyde (500 mg, 1.99 mmol) in methanol (20 mL) was added triphenylphosphoranylidene succinimide (753 mg, 2.09 mmol). The reaction mixture was stirred at reflux for 4 hours. The reaction mixture was cooled to 0° and the resulting precipitate was filtered off and rinsed with cold methanol. Dried under vacuum to provide 510 mg (77%) of 3-((5-chloro-7-(cyclopropylmethylamino)pyrazolo[1,5-a]pyrimidin-3-yl)methylene)pyrrolidine-2,5-dione. LCMS (M+1=332)

### Example 94

### Synthesis of 3-((5-(3-chlorophenylamino)-7-(cyclopropylmethylamino)pyrazolo[1,5-a]pyrimidin-3-yl)methylene)pyrrolidine-2,5-dione

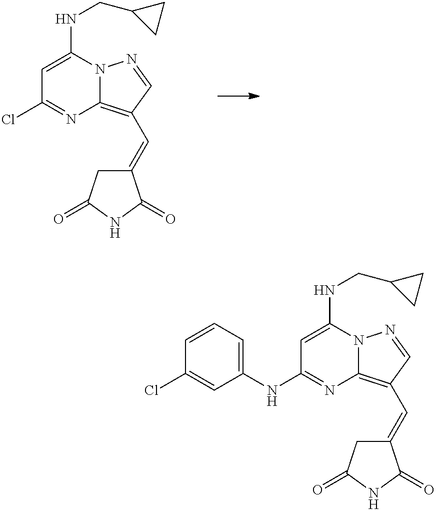

To 3-((5-chloro-7-(cyclopropylmethylamino)pyrazolo[1,5-a]pyrimidin-3-yl)methylene)pyrrolidine-2,5-dione (15 mg, 0.045 mmol) in 1,4-dioxane (1 mL) was added cesium carbonate (29 mg, mg, 0.09 mmol), 3-chloroaniline (9 mg, 0.068 mmol), Pd(OAc)₂ (1 mg, 0.002 mmol), and racemic BINAP (2 mg, 0.003 mmol). The reaction mixture was stirred under microwave heating at 180° C. for 10 minutes. Add another 0.68 mmol of aniline and stir under microwave heating at 180° C. for 20 minutes. Add 1 mL of DMSO, filter and purify by mass-directed prep LC/MS to provide 3-((5-(3-chlorophenylamino)-7-(cyclopropylmethylamino)pyrazolo[1,5-a]pyrimidin-3-yl)methylene)pyrrolidine-2,5-dione.

LCMS (M+1=423)

Examples 95 to 97 were prepared by the procedures described above including the procedures for Example 94.

### Example 95

### Synthesis of 3-((7-(cyclopropylmethylamino)-5-(m-tolylamino)pyrazolo[1,5-a]pyrimidin-3-yl)methylene)pyrrolidine-2,5-dione

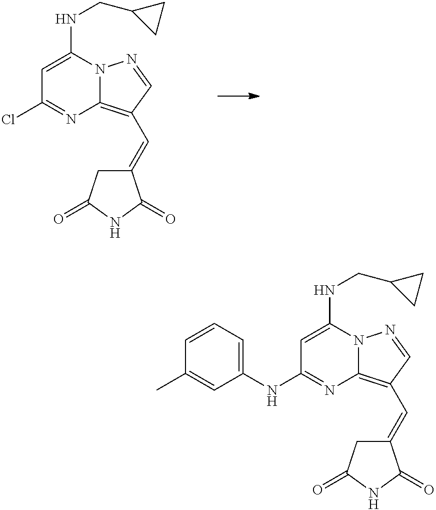

LCMS (M+1=403)

### Example 96

### Synthesis of 3-((7-(cyclopropylmethylamino)-5-(3-(trifluoromethyl)phenylamino)pyrazolo[1,5-a]pyrimidin-3-yl)methylene)pyrrolidine-2,5-dione

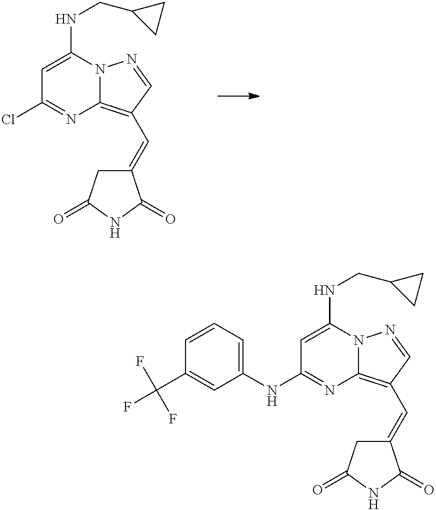

LCMS (M+1=457)

### Example 97

### Synthesis of 3-((7-(cyclopropylmethylamino)-5-(3,5-difluorophenylamino)pyrazolo[1,5-a]pyrimidin-3-yl)methylene)pyrrolidine-2,5-dione

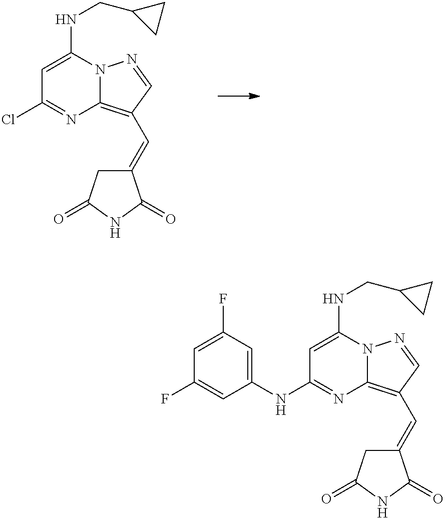

LCMS (M+1=425)

### Example 98

### Synthesis of tert-butyl 5-chloro-3-((2,5-dioxopyrrolidin-3-ylidene)methyl)pyrazolo[1,5-a]pyrimidin-7-yl(cyclopropylmethyl)carbamate

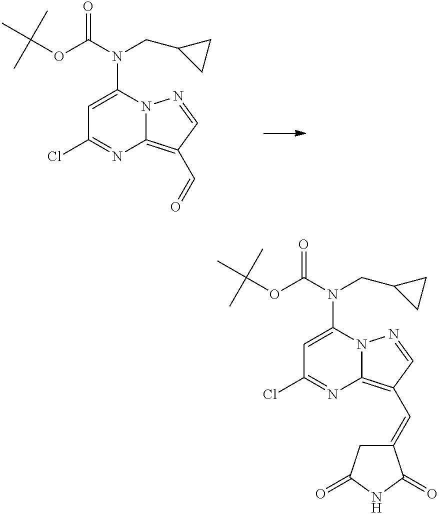

To 7 tert-butyl 5-chloro-3-formylpyrazolo[1,5-a]pyrimidin-7-yl(cyclopropyl)carbamate (1.18 g, 3.37 mmol) in methanol (34 mL) was added triphenylphosphoranylidene succinimide (1.27 g, 3.54 mmol). The reaction mixture was stirred at reflux for 4 hours. The reaction mixture was cooled to rt and the resulting precipitate was filtered off and rinsed with methanol. Dried under vacuum to provide 836 mg (58%) of tert-butyl 5-chloro-3-((2,5-dioxopyrrolidin-3-ylidene)methyl)pyrazolo[1,5-a]pyrimidin-7-yl(cyclopropylmethyl)carbamate. LCMS (M+1=432)

### Example 99

### Synthesis of 3-((5-(5-chloro-2-fluorophenylamino)-7-cyclopropylmethylamino)pyrazolo[1,5-a]pyrimidin-3-yl)methylene)pyrrolidine-2,5-dione

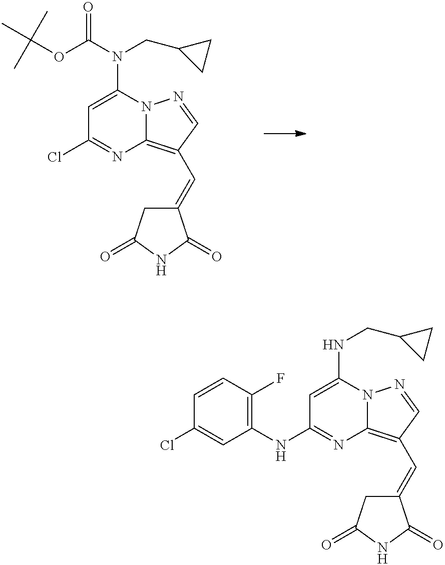

To tert-butyl 5-chloro-3-((2,5-dioxopyrrolidin-3-ylidene)methyl)pyrazolo[1,5-a]pyrimidin-7-yl(cyclopropylmethyl)carbamate (75 mg, 0.174 mmol) in 1,4-dioxane (2 mL) was added cesium carbonate (113 mg, mg, 0.348 mmol), 5-chloro-2-fluoroaniline (38 mg, 0.261 mmol), Pd(OAc)₂ (5 mg, 0.014 mmol), and racemic BINAP (7 mg, 0.011 mmol). The reaction mixture was stirred under microwave heating at 150° C. for 15 minutes. Dilute with CH₂Cl₂ and wash 1× with 0.5M HCl. Dry organic layer with MgSO₄, filter, and remove solvent to provide residue which was treated with 1 mL of 4M HCl in dioxane. Stir at 60° C. for 1 h. Cool to room temperature and the excess HCl/dioxane was removed on rotavap. Add 4 mL of saturated NaHCO₃. The resulting precipitate was filtered off and rinsed with H₂O followed by methanol. Dry under vacuum to provide 3-((5-(5-chloro-2-fluorophenylamino)-7-(cyclopropylmethylamino)pyrazolo[1,5-a]pyrimidin-3-yl)methylene)pyrrolidine-2,5-dione. LCMS (M+1=441)

### Examples 100 to 106 were prepared by the procedures described above including the procedures for Example 99

### Example 100

### Synthesis of 3-((5-(2-chloro-3-methoxyphenylamino)-7-(cyclopropylmethylamino)pyrazolo[1,5-a]pyrimidin-3-yl)methylene)pyrrolidine-2,5-dione

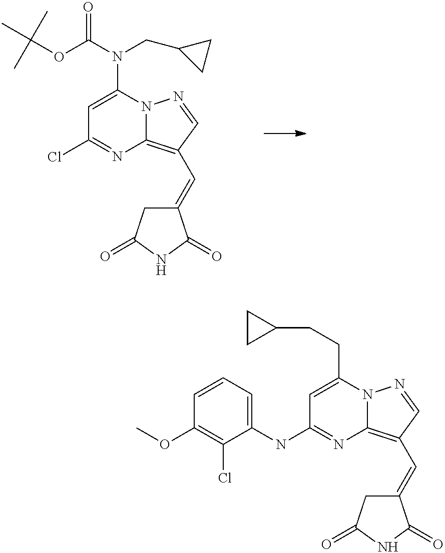

LCMS (M+1=453)

### Example 101

### Synthesis of 3-((5-(4-(1H-pyrazol-1-yl)phenylamino)-7-(cyclopropylmethylamino)pyrazolo[1,5-a]pyrimidin-3-yl)methylene)pyrrolidine-2,5-dione

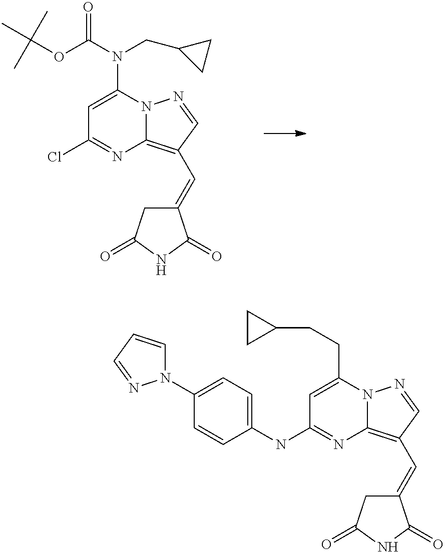

LCMS (M+1=455)

### Example 102

### Synthesis of 3-((5-(2-chloro-5-methoxyphenylamino)-7-(cyclopropylmethylamino)pyrazolo[1,5-a]pyrimidin-3-yl)methylene)pyrrolidine-2,5-dione

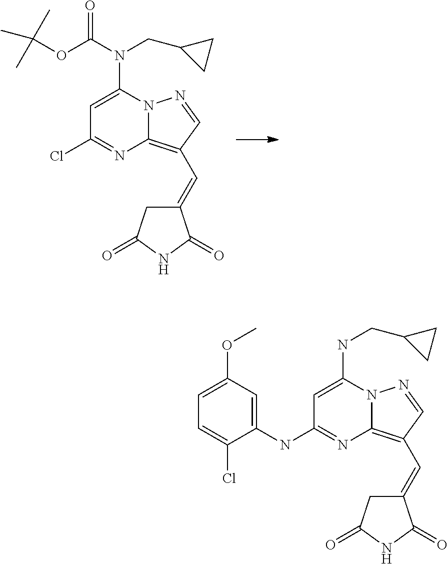

LCMS (M+1=453)

### Example 103

### Synthesis of 3-((7-(cyclopropylmethylamino)-5-(2-fluoro-5-methylphenylamino)pyrazolo[1,5-a]pyrimidin-3-yl)methylene)pyrrolidine-2,5-dione

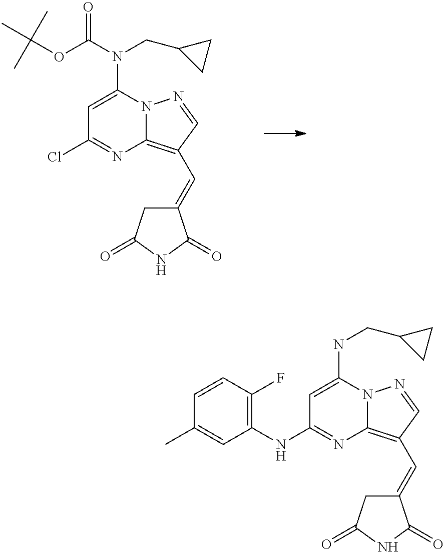

LCMS (M+1=421)

### Example 104

### Synthesis of 3-((7-(cyclopropylmethylamino)-5-(2,3-difluorophenylamino)pyrazolo[1,5-a]pyrimidin-3-yl)methylene)pyrrolidine-2,5-dione

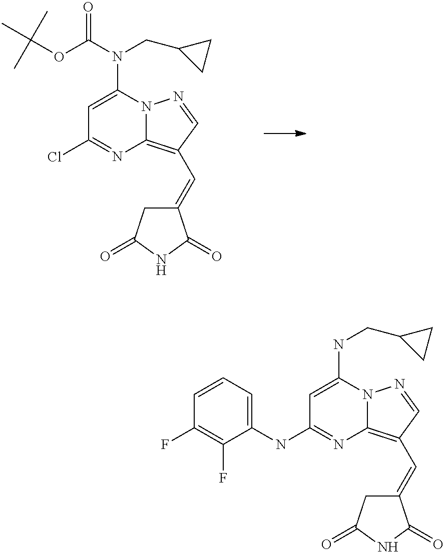

LCMS (M+1=425)

### Example 105

### Synthesis of 3-((5-(2-chloro-4-fluoro-5-methylphenylamino)-7-(cyclopropylmethylamino)pyrazolo[1,5-a]pyrimidin-3-yl)methylene)pyrrolidine-2,5-dione

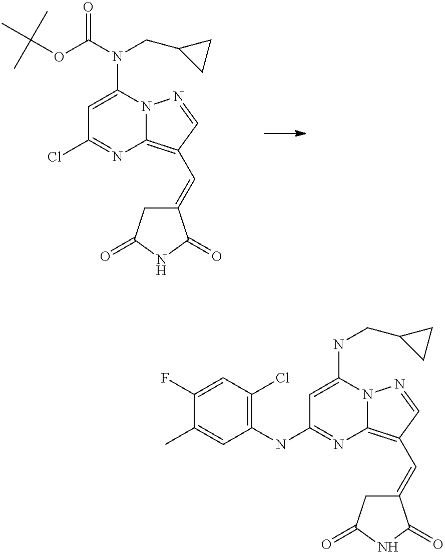

LCMS (M+1=455)

### Example 106

### Synthesis of 3-((5-(5-chloro-2-methylphenylamino)-7-(cyclopropylmethylamino)pyrazolo[1,5-a]pyrimidin-3-yl)methylene)pyrrolidine-2,5-dione

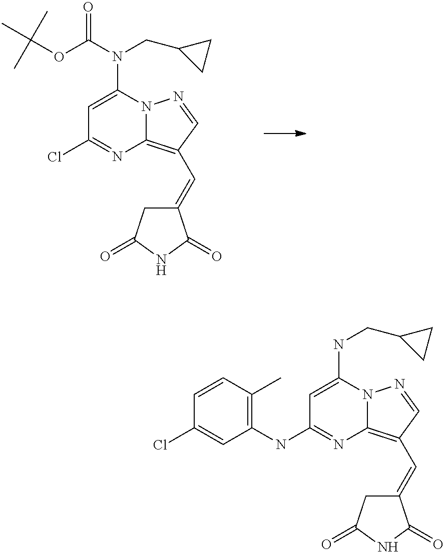

LCMS (M+1=437)

The following three compounds were prepared by the procedures described above.

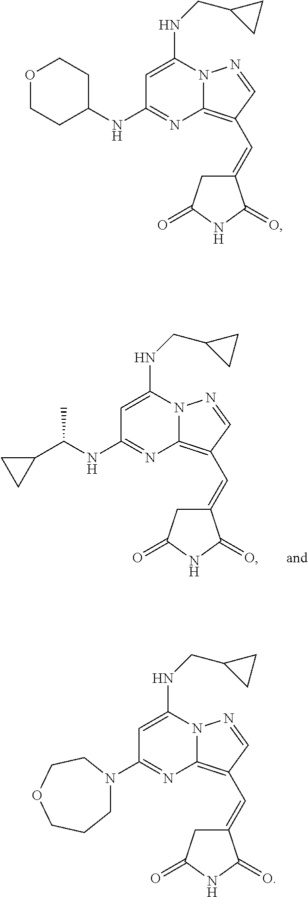

### Example 107

### Synthesis of tert-butyl cyclopropyl(3-formyl-5-(3-(hydroxymethyl)phenyl)pyrazolo[1,5-a]pyrimidin-7-yl)carbamate

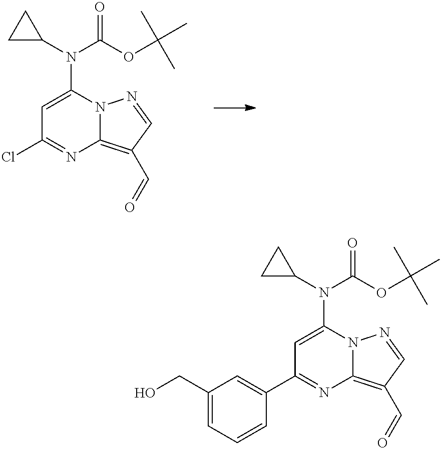

To 5 tert-butyl 5-chloro-3-formylpyrazolo[1,5-a]pyrimidin-7-yl (cyclopropyl)carbamate (200 mg, 0.594 mmol) in 6 mL of a 2:1 mixture of 1,2-Dimethoxyethane/EtOH was added 3-(hydroxymethyl)phenylboronic acid (135 mg, 0.891 mmol), tetrakis(triphenylphosphine)palladium(0) (34 mg, 0.030 mmol), and 2M aqueous solution of Na₂CO₃ (0.891 mL, 1.78 mmol). The mixture was stirred at 85° C. for 45 min. Cooled to rt and partitioned between 0.5M HCl and EtOAc. The layers were separated and the organic layer was dried with MgSO₄, filtered and the solvent removed. Purified by flash chromatography eluting with 25% EtOAc in hexane followed by 50% EtOAc in hexane to provide 275 mg of tert-butyl cyclopropyl(3-formyl-5-(3-(hydroxymethyl)phenyl)pyrazolo[1,5-a]pyrimidin-7-yl)carbamate. LCMS (M+1=409)

### Example 108

### Synthesis of 7-(cyclopropylamino)-5-(3-(hydroxymethyl)phenyl)pyrazolo[1,5-a]pyrimidine-3-carbaldehyde

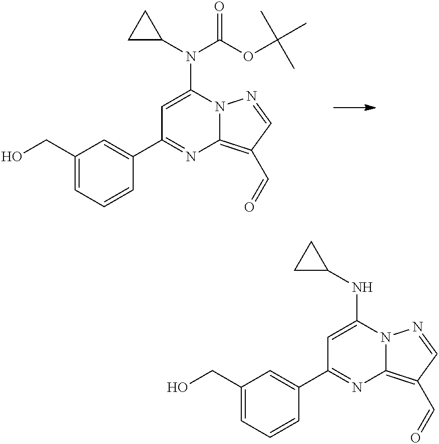

To tert-butyl cyclopropyl(3-formyl-5-(3-(hydroxymethyl)phenyl)pyrazolo[1,5-a]pyrimidin-7-yl)carbamate (275 mg, 0.674 mmol) was added 3 mL of 4M HCl in dioxane. The reaction mixture was stirred at room temperature for 2 h. Dilute with 5 mL H₂O and adjust the pH of the solution to 7-10 with 5M NaOH. Extract into methylene chloride. Dry with MgSO₄, filter and remove volatiles to provide 91 mg of 7-(cyclopropylamino)-5-(3-(hydroxymethyl)phenyl)pyrazolo[1,5-a]pyrimidine-3-carbaldehyde (44%) LCMS (M+1=309)

### Example 109

### Synthesis of 3-((7-(cyclopropylamino)-5-(3-(hydroxymethyl)phenyl)pyrazolo[1,5-a]pyrimidin-3-yl)methylene)pyrrolidine-2,5-dione

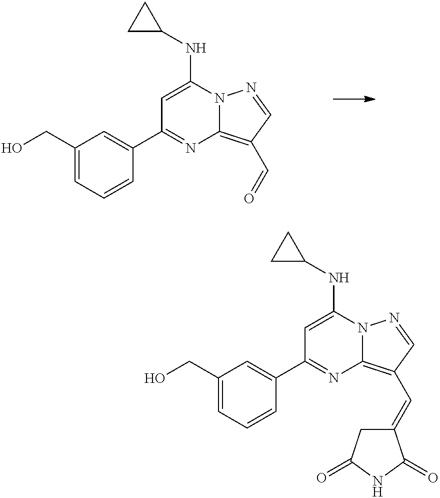

To 7-(cyclopropylamino)-5-(3-(hydroxymethyl)phenyl)pyrazolo[1,5-a]pyrimidine-3-carbaldehyde (20 mg, 0.065 mmol in ethanol (1 mL) was added triphenylphosphoranylidene succinimide (23 mg, 0.065 mmol). The reaction mixture was stirred at 90° for 3 hours. The reaction mixture was cooled to room temperature and the ethanol removed on rotavap. Add 2 mL of 1:1 ethanol/H₂O and sonicate. The resulting precipitate was filtered off and rinsed with 10 mL of ethanol. Dried under vacuum to provide 3-((7-(cyclopropylamino)-5-(3-(hydroxymethyl)phenyl)pyrazolo[1,5-a]pyrimidin-3-yl)methylene)pyrrolidine-2,5-dione as a light yellow solid. LCMS (M+1=390)

Examples 110 to 116, 118, and 120 were prepared by the procedures described above including the procedures for Examples 107 to 109.

### Example 110

### Synthesis of tert-butyl 5-(5-cyanothiophen-2-yl)-3-formylpyrazolo[1,5-a]pyrimidin-7-yl(cyclopropyl)carbamate

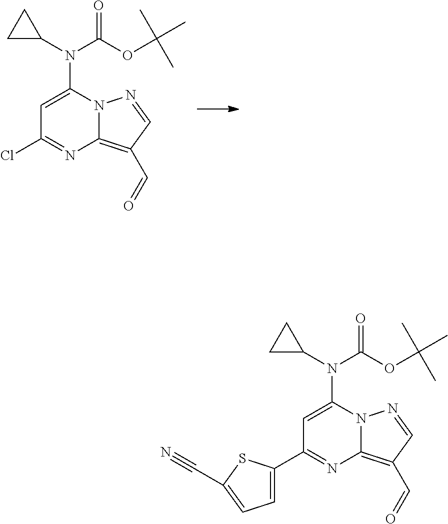

LCMS (M+1=410)

### Example 111

### Synthesis of 5-(7-(cyclopropylamino)-3-formylpyrazolo[1,5-a]pyrimidin-5-yl)thiophene-2-carbonitrile

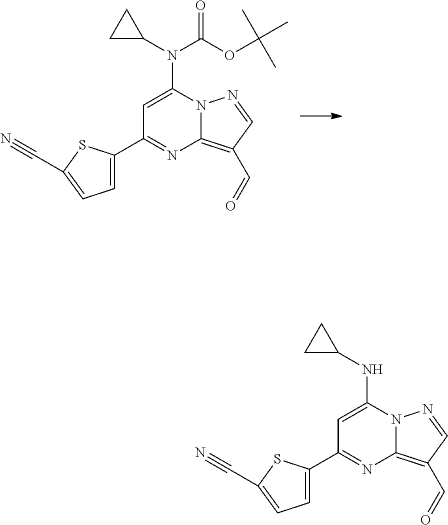

LCMS (M+1=310)

### Example 112

### Synthesis of 5-(7-(cyclopropylamino)-3-((2,5-dioxopyrrolidin-3-ylidene)methyl)pyrazolo[1,5-a]pyrimidin-5-yl)thiophene-2-carbonitrile

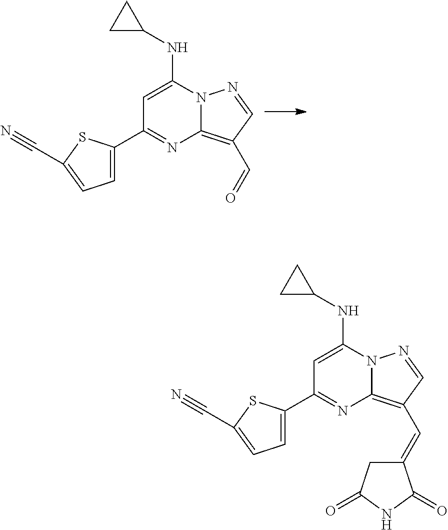

LCMS (M+1=391)

### Example 113

### Synthesis tert-butyl cyclopropyl(3-formyl-5-(3-morpholinomethyl)phenyl)pyrazolo[1,5-a]pyrimidin-7-yl)carbamate

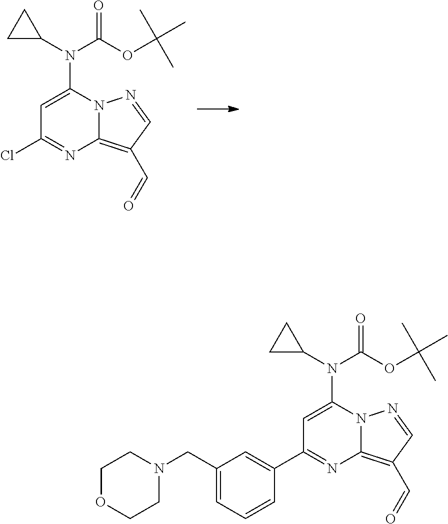

LCMS (M+1=478)

### Example 114

### Synthesis of 7-(cyclopropylamino)-5-(3-(morpholinomethyl)phenyl)pyrazolo[1,5-a]pyrimidine-3-carbaldehyde

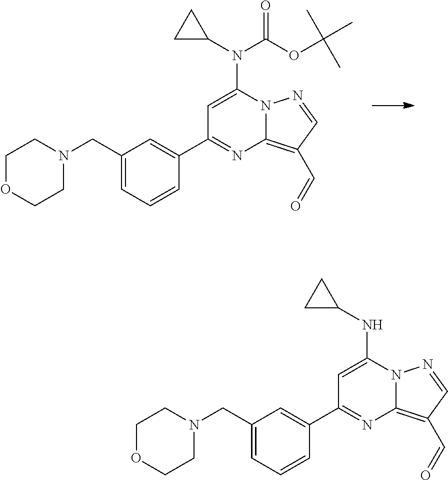

LCMS (M+1=378)

### Example 115

### Synthesis of 3-((7-(cyclopropylamino)-5-(3-(morpholinomethyl)phenyl)pyrazolo[1,5-a]pyrimidin-3-yl)methylene)pyrrolidine-2,5-dione

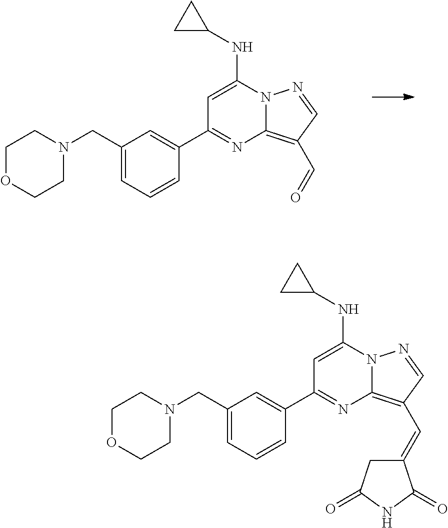

LCMS (M+1=459)

### Example 116

### Synthesis of tert-butyl cyclopropyl(3-formyl-5-(3-(methylsulfonamido)phenyl)pyrazolo[1,5-a]pyrimidin-7-yl)carbamate

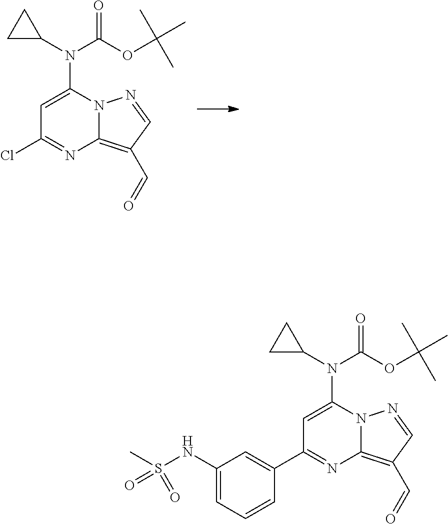

LCMS (M+1=472)

### Example 117

### Synthesis of N-(3-(7-(cyclopropylamino)-3-formylpyrazolo[1,5-a]pyrimidin-5-yl)phenyl)-N-methylmethanesulfonamide

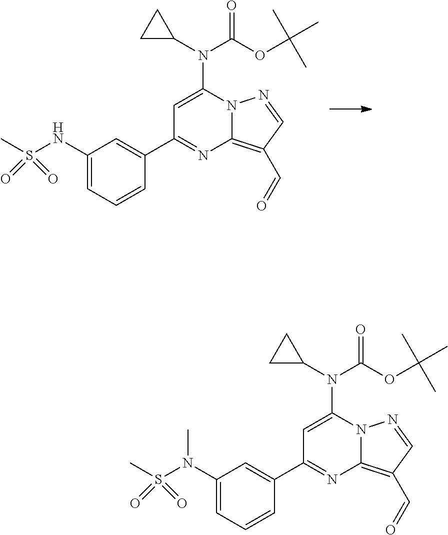

To NaH (60%) (42 mg, 1.08 mmol) in DMF (8 mL) was added tert-butyl cyclopropyl(3-formyl-5-(3-(methylsulfonamido)phenyl)pyrazolo[1,5-a]pyrimidin-7-yl)carbamate (465 mg, 0.986 mmol) followed by MeI (123 1.97 mmol) Stir at rt for 20 min. Reaction quenched with H₂O and extracted into EtOAc 2×. Combined organic layers and washed 3× with brine. Dried with MgSO₄, filtered and removed solvent to provide desired product as residue. To this was added 2 mL of 4M HCl in dioxane. Stirred at 50° for 30 min. Cool to rt, Dilute with H₂O and neutralize with 2N NaOH. Extract into CH₂Cl₂. Organic layer dried with MgSO₄, filter, and remove solvent to provide 467 mg of N-(3-(7-(cyclopropylamino)-3-formylpyrazolo[1,5-a]pyrimidin-5-yl)phenyl)-N-methylmethanesulfonamide. LCMS (M+1=386)

### Example 118

### Synthesis of N-(3-(7-(cyclopropylamino)-3-((2,5-dioxopyrrolidin-3-ylidene)methyl)pyrazolo[1,5-a]pyrimidin-5-yl)phenyl)-N-methylmethanesulfonamide

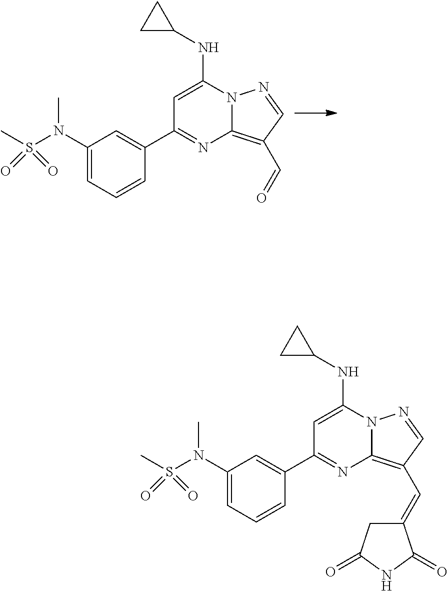

LCMS (M+1=467)

### Example 119

### Synthesis of tert-butyl cyclopropyl(3-formyl-5-(3-hydroxyphenyl)pyrazolo[1,5-a]pyrimidin-7-yl)-carbamate

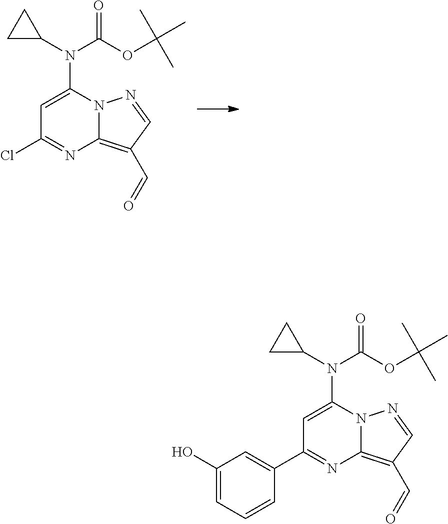

To 5 tert-butyl 5-chloro-3-formylpyrazolo[1,5-a]pyrimidin-7-yl (cyclopropyl)carbamate (650 mg, 1.93 mmol) in 14 mL of a 2:1 mixture of 1,2-Dimethoxyethane/EtOH was added 3-hydroxyphenyl boronic acid (399 mg, 2.89 mmol), tetrakis(triphenylphosphine)palladium(0) (112 mg, 0.096 mmol), and 2M aqueous solution of Na₂CO₃ (2.9 mL, 5.79 mmol). The mixture was stirred at 85° C. for 1 h. The volatiles were removed by rotary evaporation and the residue was purified by silica gel chromatography (0%-30% EtOAc/Hexanes) to provide 400 mg of tert-butyl cyclopropyl(3-formyl-5-(3-hydroxyphenyl)pyrazolo[1,5-a]pyrimidin-7-yl)carbamate. (52%) (LCMS (M+1=395)

The following two compounds can be prepared by the procedures as described above.

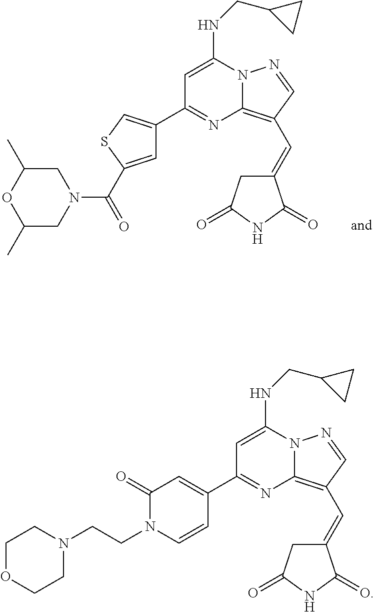

### Example 120

### Synthesis of 7-(cyclopropylamino)-5-(3-hydroxyphenyl)pyrazolo[1,5-a]pyrimidine-3-carbaldehyde

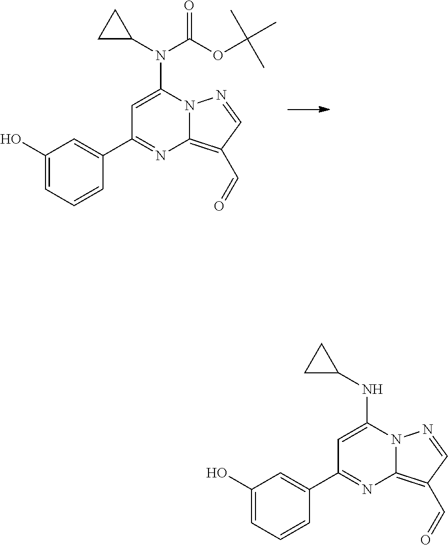

LCMS (M+1=295)

### Example 121

### Synthesis of 3-((7-(cyclopropylamino)-5-(3-hydroxyphenyl) pyrazolo[1,5-a]pyrimidin-3-yl)methylene)pyrrolidine-2,5-dione

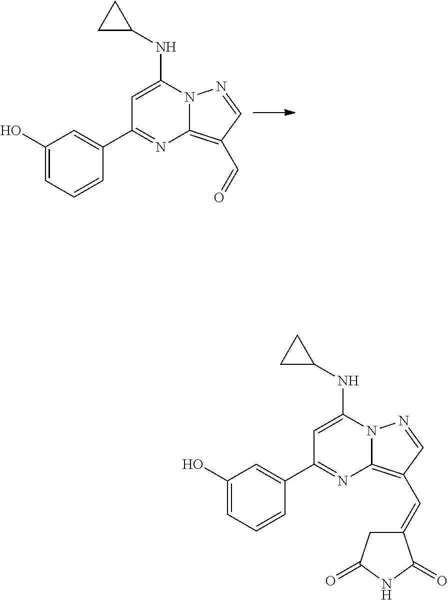

To 7-(cyclopropylamino)-5-(3-hydroxyphenyl)pyrazolo[1,5-a]pyrimidine-3-carbaldehyde (27 mg, 0.092 mmol in methanol (1 mL) was added triphenylphosphoranylidene succinimide (33 mg, 0.092 mmol). The reaction mixture was stirred at reflux for 16 hours. The reaction mixture was cooled to room temperature and the resulting precipitate was filtered off and rinsed with 10 mL of methanol. Dried under vacuum to provide 3-((7-(cyclopropylamino)-5-(3-hydroxyphenyl)pyrazolo[1,5-a]pyrimidin-3-yl)methylene)pyrrolidine-2,5-dione as a light yellow solid. LCMS (M+1=376)

### Example 122

### Synthesis of 5,7-dichloro-6-methylpyrazolo[1,5-a]pyrimidine

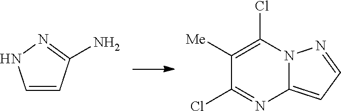

Under nitrogen gas atmosphere, sodium (3.5 g, 151 mmol) was added to ethanol (125 mL) in small portions and stirred at room temperature until all the sodium had dissolved. A solution of 3-aminopyrazole (12.5 g, 150 mmol) in ethanol (20 mL) and diethyl methylmalonate (26 mL, 153 mmol) were dropped, successively, to the above solution. The mixture was refluxed at 90° C. for 10 hours, cooled to room temperature, and filtered under vacuum. To the solid, cold 5N HCl was added and the resulting solid was collected by filtration under vacuum. The intermediate, 6-methylpyrazolo[1,5-a]pyrimidine-5,7-diol, was recovered as an off-white solid in 72% yield (17.9 g). This material was used for the next step without further purification. LCMS (M+1=166)

Under nitrogen gas atmosphere, phosphorous oxychloride (160 mL, 1.72 mol) and dimethylaniline (16 mL, 132 mmol) was added successively to the intermediate prepared above (16 g, 97 mmol). The mixture was heated at 110° C. for 4 hours then excess POCl₃ was removed under vacuum. The residue was made basic with 3N NaOH solution (pH=9-10) and extracted with ethyl acetate (3×). The combined organic layers were dried over anhydrous Na₂SO₄, filtered, and concentrated under vacuum. The residue was purified by silica gel chromatography (100% DCM) to provide 15.8 grams of the solid yellow product, 5,7-dichloro-6-methylpyrazolo[1,5-a]pyrimidine (81% yield). LCMS (M+1=203)

### Example 123

### Synthesis of 5-chloro-7-(cyclopropylamino)-6-methylpyrazolo[1,5-a]pyrimidine-3-carbaldehyde

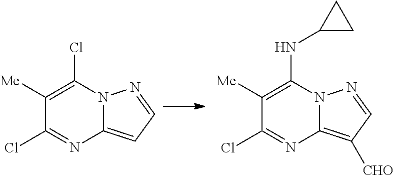

To the reaction flask, 5,7-dichloro-6-methylpyrazolo[1,5-a]pyrimidine (5 g, 25 mmol) was added along with cyclopropyl amine (1.8 mL, 25 mmol), triethylamine (3.5 mL, 25 mmol), and acetonitrile (87 mL). The reaction was stirred at room temperature for 3 hours then heated at 85° C. for an additional 6 hours. The mixture was cooled to room temperature, diluted with water, filtered and washed with water. The intermediate, 5-chloro-N-cyclopropyl-6-methylpyrazolo[1,5-a]pyrimidin-7-amine, was further purified by silica gel chromatography (10% ethyl acetate/hexanes) to provide 4.8 grams of a white solid (86% yield). LCMS (M+1=223)

To the intermediate (3.6 g, 16 mmol) isolated above in DMF (59 mL) was added phosphorous oxychloride (9 mL, 96 mmol) slowly at room temperature. The reaction mixture was allowed to stir at room temperature for 10 hours then quenched by addition to 6N NaOH solution. The pH of the mixture was adjusted with 6N HCl to pH=7-9. The solid was recovered by filtration and washed with water. The product, 5-chloro-7-(cyclopropylamino)-6-methylpyrazolo[1,5-a]pyrimidine-3-carbaldehyde, was purified by recrystallization from ethyl acetate/hexanes to yield a white solid in 73% yield (2.9 g).

LCMS (M+1=251)

### Example 124

### Synthesis of tert-butyl 5-chloro-3-formyl-6-methylpyrazolo[1,5-a]pyrimidin-7-yl(cyclopropyl)carbamate

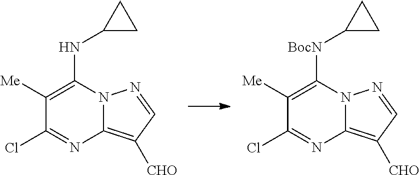

To 5-chloro-7-(cyclopropylamino)-6-methylpyrazolo[1,5-a]pyrimidine-3-carbaldehyde (2.9 g, 11.7 mmol) in methylene chloride (22 mL) was added triethylamine (2 mL, 14 mmol), dimethylaminopyridine (100 mg, 0.8 mmol), and di-t-butyldicarbonate (3.1 g, 14 mmol). The mixture was stirred at room temperature for 10 hours. The reaction mixture was transferred to a separatory funnel, washed 1× with H₂O, 2× with brine, dried over MgSO₄, filtered, and evaporated to dryness to provide an oily residue. The crude material was purified by silica gel chromatography (25% ethyl acetate/hexanes) to yield a light orange solid (3.6 g, 88% yield), tert-butyl 5-chloro-3-formyl-6-methylpyrazolo[1,5-a]pyrimidin-7-yl(cyclopropyl)carbamate. LCMS (M+1=351)

### Example 125

### Synthesis of 3-chloro-4-(7-(cyclopropylamino)-3-formyl-6-methylpyrazolo[1,5-a]pyrimidin-5-ylamino)benzonitrile

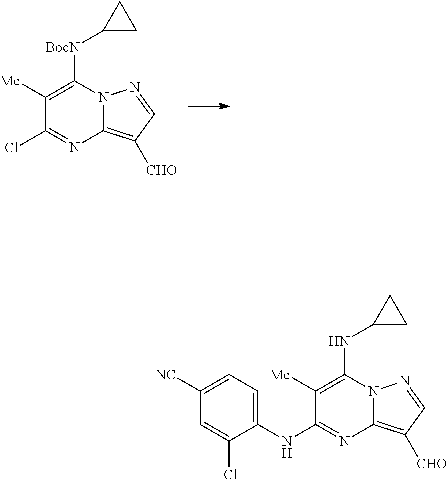

To 4-amino-3-chlorobenzonitrile (52 mg, 0.34 mmol), Cs₂CO₃ (130 mg, 0.4 mmol) were added to tert-butyl 5-chloro-3-formyl-6-methylpyrazolo[1,5-a]pyrimidin-7-yl(cyclopropyl)carbamate (100 mg, 0.29 mmol) dissolved in 1,4-dioxane (1.1 mL). Racemic BINAP (11 mg, 0.017 mmol) and palladium(II) acetate (8 mg, 0.011 mmol) were then added. The mixture was sealed and irradiated at 110° C. for 60 min in the microwave. Et₂O (3 mL) was added and the solution was filtered. The filtrate was concentrated in vacuo. The crude residue was dissolved in dichloromethane (1.5 mL) and trifluoroacetic acid (1.5 mL). After stirring at room temperature for 1 hour, the solution was concentrated under a stream of air. The crude material was purified by silica gel chromatography (3% acetone/dichloromethane) to yield the product, 3-chloro-4-(7-(cyclopropylamino)-3-formyl-6-methylpyrazolo[1,5-a]pyrimidin-5-ylamino)benzonitrile (34 mg, 33% yield). LCMS (M+1=367)

### Example 126

### Synthesis of 3-chloro-4-(7-(cyclopropylamino)-3-((2,5-dioxopyrrolidin-3-ylidene)methyl)-6-methylpyrazolo[1,5-a]pyrimidin-5-ylamino)benzonitrile

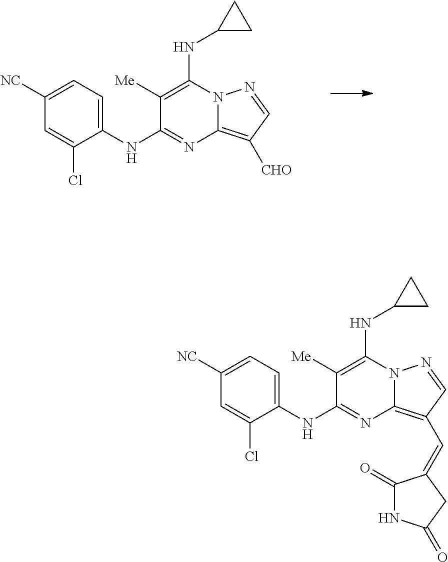

Triphenylphosphoranylidene succinimide (12 mg, 0.033 mmol) and 3-chloro-4-(7-(cyclopropylamino)-3-formyl-6-methylpyrazolo[1,5-a]pyrimidin-5-ylamino)benzonitrile (10 mg, 0.027 mmol) were dissolved in ethanol (0.4 mL). The reaction was heated at 80° C. After 10 hours, another portion of triphenylphosphoranylidene succinimide (10 mg, 0.033 mmol) was added along with DMF (0.2 mL) and the reaction was heated at 95° C. for an additional 10 hours. Then, the reaction was cooled to r.t., diluted with water, and the precipitate was collected and washed with water, 1:1 ethanol:water, then ethanol. The bright yellow solid was dried in vacuo to 3-chloro-4-(7-(cyclopropylamino)-3-((2,5-dioxoimidazolidin-4-ylidene)methyl)-6-methylpyrazolo[1,5-a]pyrimidin-5-ylamino)benzonitrile (3.1 mg, 26% yield). LCMS (M+1=448)

### Example 127

### Synthesis of 5-(4-(1H-pyrazol-1-yl)phenylamino)-7-(cyclopropylamino)-6-methylpyrazolo[1,5-a]pyrimidine-3-carbaldehyde

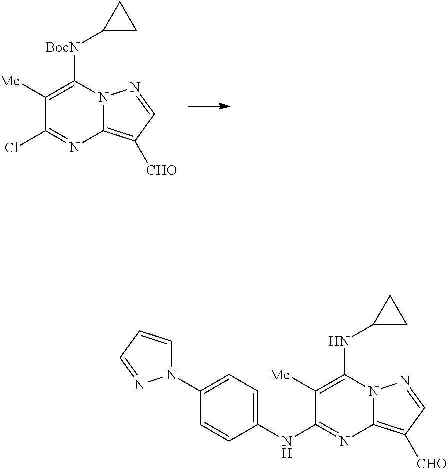

To 4-(1H-pyrazol-1-yl)aniline (54 mg, 0.34 mmol), Cs₂CO₃ (130 mg, 0.4 mmol) were added to tert-butyl 5-chloro-3-formyl-6-methylpyrazolo[1,5-a]pyrimidin-7-yl(cyclopropyl)carbamate (100 mg, 0.29 mmol) dissolved in 1,4-dioxane (1.1 mL). Racemic BINAP (11 mg, 0.017 mmol) and palladium(II) acetate (8 mg, 0.011 mmol) were then added. The mixture was sealed and irradiated at 110° C. for 60 min in the microwave. Et₂O (3 mL) was added and the solution was filtered. The filtrate was concentrated in vacuo. The crude residue was dissolved in dichloromethane (1.5 mL) and trifluoroacetic acid (1.5 mL). After stirring at room temperature for 1 hour, the solution was concentrated under a stream of air. The crude material was purified by silica gel chromatography (10% acetone/dichloromethane) to yield the product, 5-(4-(1H-pyrazol-1-yl)phenylamino)-7-(cyclopropylamino)-6-methylpyrazolo[1,5-a]pyrimidine-3-carbaldehyde (70 mg, 66% yield). LCMS (M+1=374)

### Example 128

### Synthesis of 3-((5-(4-(1H-pyrazol-1-yl)phenylamino)-7-(cyclopropylamino)-6-methylpyrazolo[1,5-a]pyrimidin-3-yl)methylene)pyrrolidine-2,5-dione

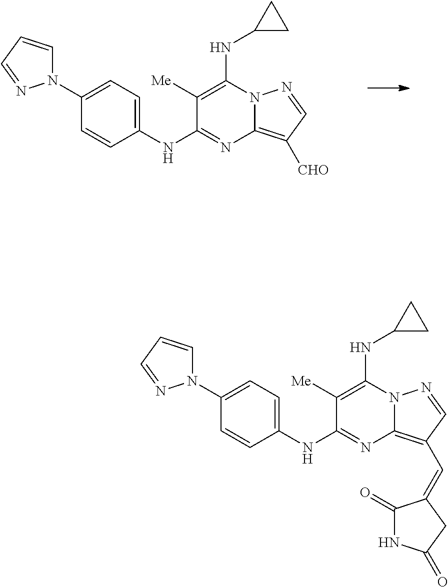

Triphenylphosphoranylidene succinimide (25 mg, 0.07 mmol) and 5-(4-(1H-pyrazol-1-yl)phenylamino)-7-(cyclopropylamino)-6-methylpyrazolo[1,5-a]pyrimidine-3-carbaldehyde (17 mg, 0.046 mmol) were dissolved in ethanol (0.4 mL) along with DMF (0.4 mL) The reaction was heated at 95° C. in the microwave for 10 hours then cooled to room temperature. The reaction mixture was diluted with water, and the precipitate was collected and washed with water, 1:1 ethanol:water, then ethanol. The bright yellow solid was dried in vacuo to give 3-((5-(4-(1H-pyrazol-1-yl)phenylamino)-7-(cyclopropylamino)-6-methylpyrazolo[1,5-a]pyrimidin-3-yl)methylene)pyrrolidine-2,5-dione (3.3 mg, 16% yield). LCMS (M+1=455)

### General Methods:

Unless otherwise specified, the various substituents of the compounds are defined in the same manner as Formula (I) of the invention.

The synthetic methods described in Scheme G1 and Scheme G2 can be used to prepare various substituted analogs of Formula (I) compound.

Substituted aminopyrazole 1 can react with isothiocyanate 2 to form intermediate 3.

Compound 3 can be cyclized to 4 in the presence of a base such as sodium hydroxide. Compound 4 can be alkylated by with R⁷—Halo (such as R⁷—Cl and R⁷—Br) in the presence of a base. Compound 5 can be converted to compound 6 using phosphorus oxychloride. Molecule 7 can be prepared by addition of amine R₇R₈NH to molecule 6 in a solvent like NMP or DMF. Compound 8 can be obtained by reacting compound 7 with DMF and Phosphorus oxychloride under Vilsmeier reaction conditions. Aldehyde 8 can be converted in two steps to substituted ketone 8b by reacting with a Grignard reagent R₄MgX, followed by reaction with an oxidant such as DCC or using Swern reaction conditions.

Compound 8 and 8a, or 8b and 8a can react upon heating in a solvent such as ethanol to form compound 9. Oxidation of 9 by an oxidant such as meta-chloroperbenzoic acid or oxone can provide compound 10, which can contain variable quantities of sulfide (n=0), sulfoxide (n=1) or sulfone (n=2).

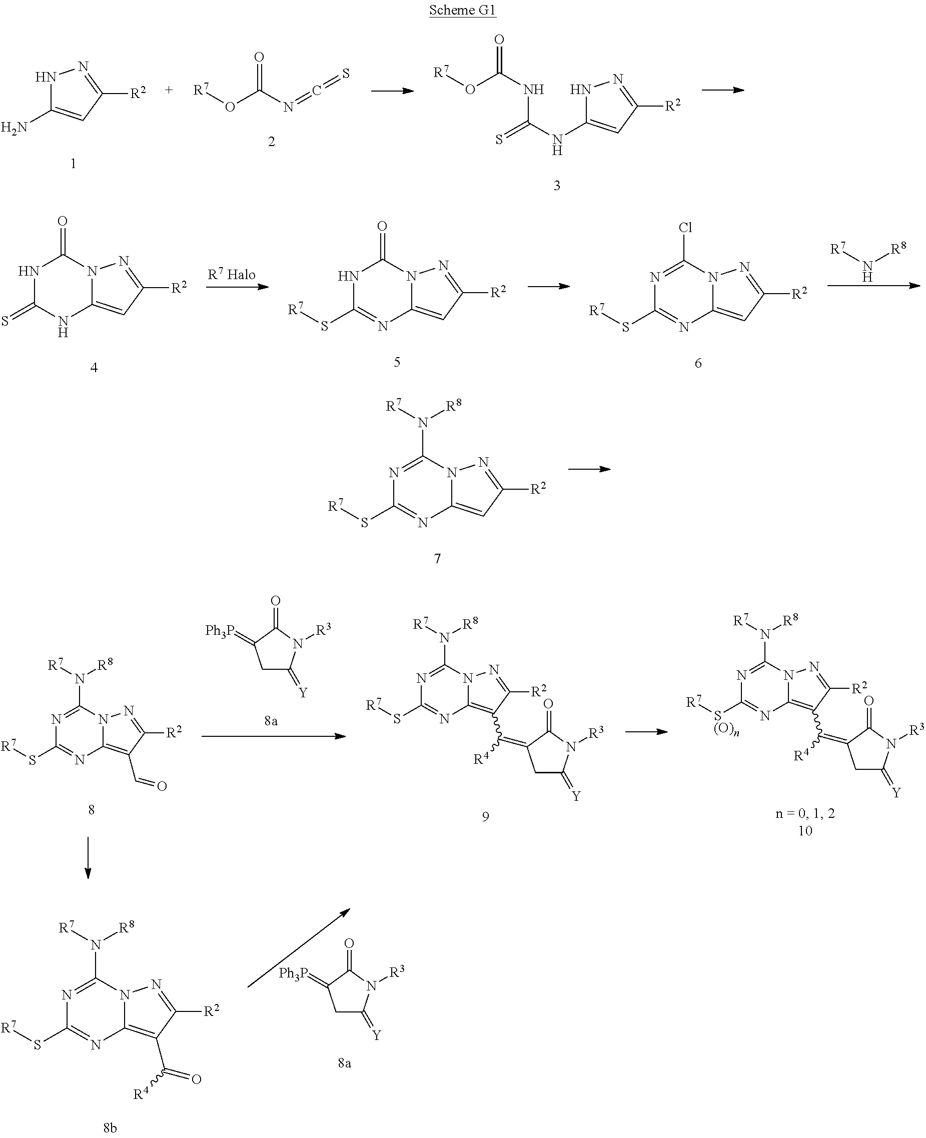

The synthetic methods depicted in Scheme G2 can be used to prepare various substituted analogs of the compounds of Formula (I).

Compound 10 can be mixed at room temperature or heated with amines R₇R₈NH to form compound 11. Compound 10 can be reacted with hydrazines R₇R₈N—NH₂ to form compound 12. Compound 10 can be reacted with alcohols or phenols R₇OH in the presence of a base such as NaH or K₂CO₃ to form compound 13. Compound 10 can be reacted with thiols or thiophenols R₇SH with or without a base to form compound 14.

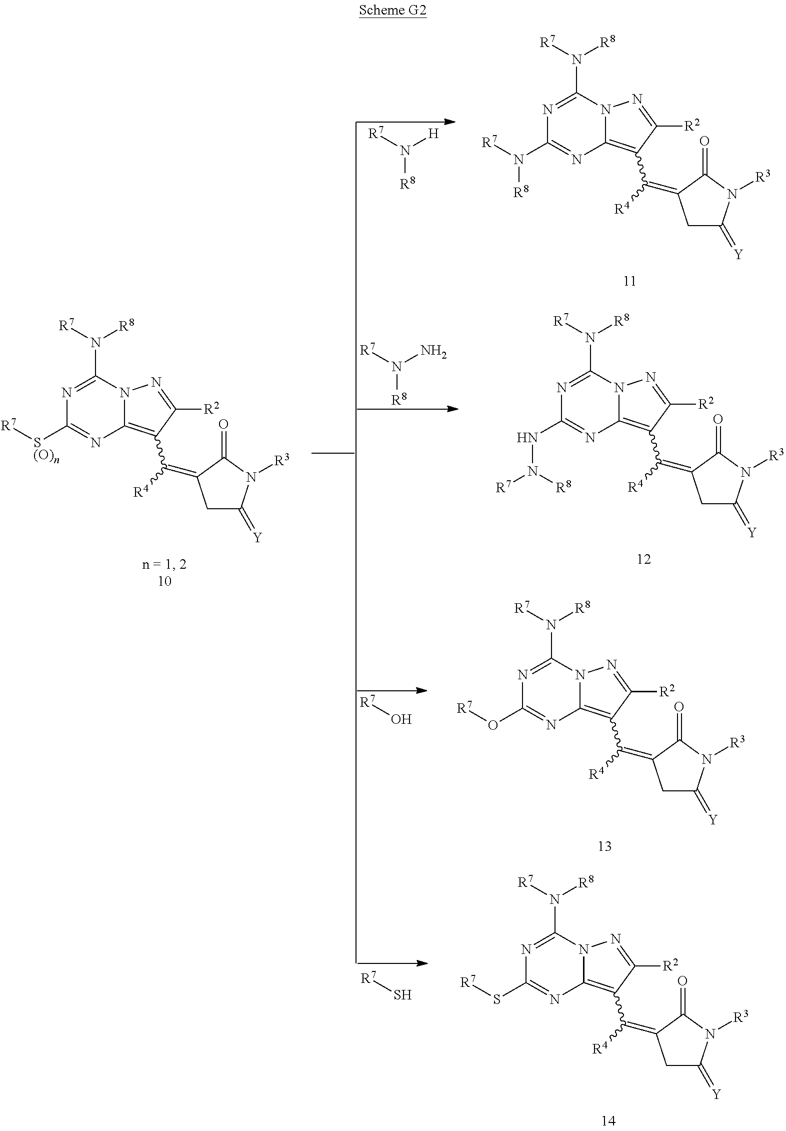

The synthetic methods described in Scheme G3 can be used to prepare analogs substituted by aryl or heteroaryls group. Compound 7 can be reacted with boronic esters or acids W—B(OR⁷)₂ or organo tin compounds W—Sn(R⁷)₃ in the presence of tri(2-furyl)phosphine, copper(I) thiophene-2-carboxylate and Pd₂ dba₃ or using conditions previously described in Organic Letters 2002, vol 4(6), pp. 979-981. Compound 15 can be converted to compound 18 using chemistries similar to the one described in Scheme G1.

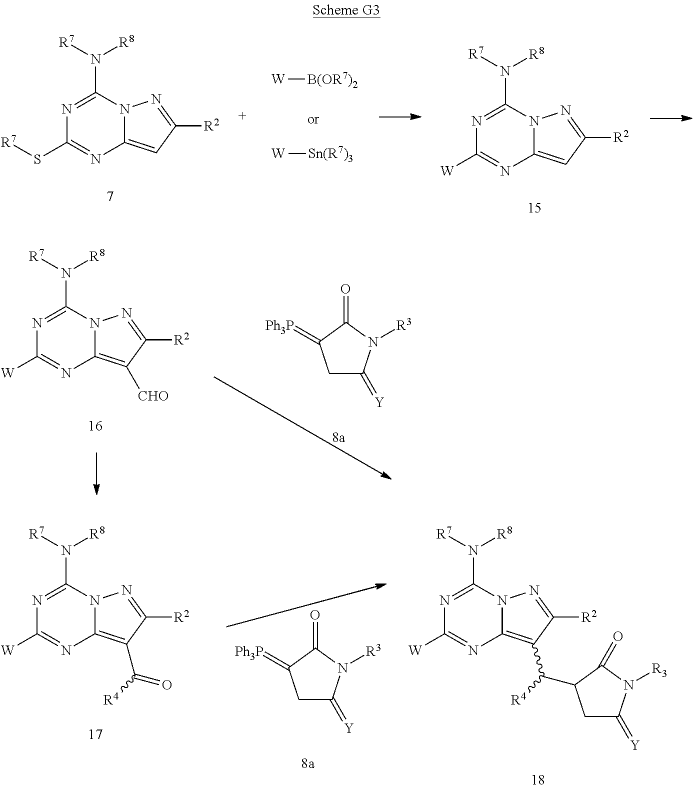

### Example 129

### Synthesis of 2-(methylthio)pyrazolo[1,5-a][1,3,5]triazin-4(3H)-one

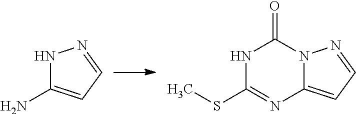

The material was prepared according to a procedure published in U.S. Pat. No. 3,846,423. Characterized by LCMS (ES): >95% pure, m/z 183 [M+H]⁺.

### Example 130

### Synthesis of 4-chloro-2-(methylthio)pyrazolo[1,5-a][1,3,5]triazine

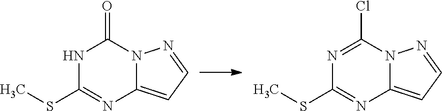

In a round bottom flask equipped with a magnetic stirbar, 2-(methylthio)pyrazolo[1,5-a][1,3,5]triazin-4(3H)-one (1.0 eq, 10.43 g, 57.24 mmol) was suspended in acetonitrile (100 ml). Phosphorus oxychloride (4.0 eq, 21 ml, 229.4 mmol) and triethylamine (1.05 eq, 8.4 ml, 60.27 mmol) were added and the mixture stirred at reflux for 3.5 hours, at which time LCMS indicated completion of the reaction. The mixture was cooled down and slowly poured into crushed ice (final total volume of about 600 ml). The solid was filtered, washed with water and dried in a vacuum oven to afford 4-chloro-2-(methylthio)pyrazolo[1,5-a][1,3,5]triazine as a tan solid (8.15 g, 71% yield). LCMS (ES): >97% pure, m/z 201 [M+H]⁺.

### Example 131

### Synthesis of N-cyclopropyl-2-(methylthio)pyrazolo[1,5-a][1,3,5]triazin-4-amine

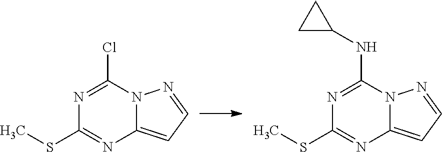

4-chloro-2-(methylthio)pyrazolo[1,5-a][1,3,5]triazine (1.0 eq, 6.26 g, 31.19 mmol) was suspended in anhydrous NMP (50 ml). Cyclopropylamine (1.5 eq, 3.2 ml, 46.26 mmol) was added through syringe dropwise. Internal temperature rose to 47° C. The mixture was stirred without any external cooling for one hour. An additional amount of cypropylamine (1 ml) was added and the mixture stirred for another 1.5 hours. The mixture was slowly poured into water (500 ml) under stirring. The resulting solid was filtered, washed with water and dried in a vacuum oven to give N-cyclopropyl-2-(methylthio)pyrazolo[1,5-a][1,3,5]triazin-4-amine as a tan solid (5.44 g, 79% yield). LCMS (ES): >95% pure, m/z 222 [M+H]⁺.

The following compounds were prepared by using procedures described above including the procedures for Example 131. Compounds were characterized by LCMS.

| | | LCMS m/z |
|---|---|---|
| Structure | MW | [M + 1]+ |
| 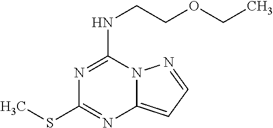 | 253.32 | 254 |
| 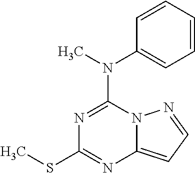 | 271.34 | 272 |
| 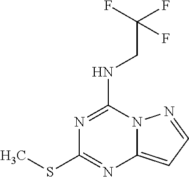 | 263.24 | 264 |
| 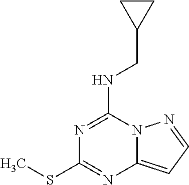 | 235.31 | 236 |
| 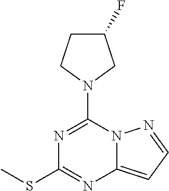 | 253.30 | 254 |
| 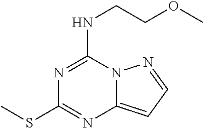 | 239.30 | 240 |
| 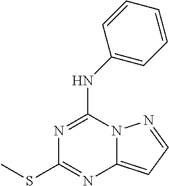 | 257.31 | 258 |
| 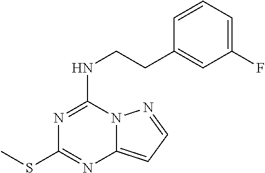 | 303.36 | 304 |

### Example 132

### Synthesis of 4-(cyclopropylamino)-2-(methylthio)pyrazolo[1,5-a][1,5]-triazine-8-carbaldehyde

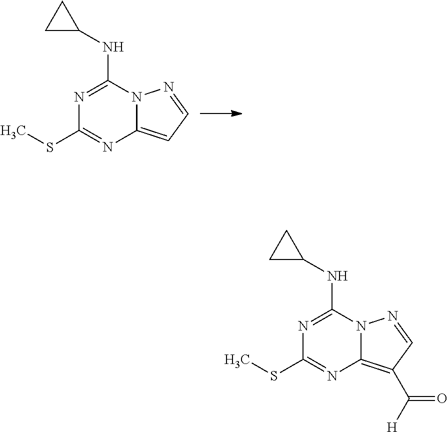

N-cyclopropyl-2-(methylthio)pyrazolo[1,5-a][1,3,5]triazin-4-amine (1.0 eq, 3.10 g, 14.00 mmol) was dissolved in anhydrous DMF (50 ml) under nitrogen atmosphere. Phosphorus oxychloride (5.0 eq, 6.4 ml, 69.9 mmol) was added dropwise over 5 minutes. Internal temperature rose to 45° C. The reaction was stirred in an oil bath at 70° C. for 4.5 hours. The mixture was cooled down and added dropwise into a solution of 6N NaOH (150 ml) chilled with an ice bath. The rate of addition was adjusted to maintain the internal temperature of the aqueous NaOH below 16° C. At the end of the addition, the mixture was neutralized by slow addition of 6N HCl to reach pH=5-6. The resulting solid was filtered, washed with water and dried in a vacuum oven overnight. 4-(cyclopropylamino)-2-(methylthio)pyrazolo[1,5-a][1,3,5]triazine-8-carbaldehyde was isolated as tan solid (9.26 g, 93%). LCMS (ES): >95% pure, m/z 250 [M+H]⁺.

The following compounds were prepared by using procedures similar to Example 132. Compounds were characterized by LCMS.

| | | LCMS m/z |
|---|---|---|
| Structure | MW | [M + 1]+ |
| 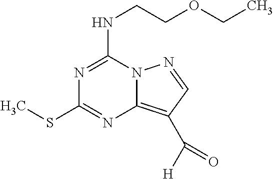 | 281.3341 | 282 |
| 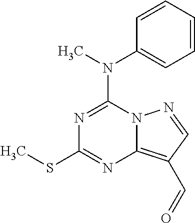 | 299.3509 | 300 |
| 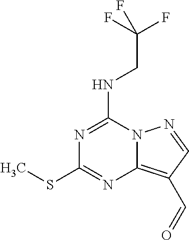 | 291.26 | 292 |
| 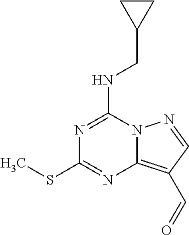 | 263.32 | 264 |
| 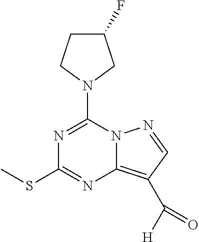 | 281.31 | 282 |
| 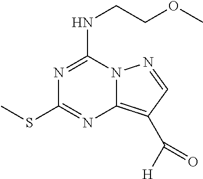 | 267.31 | 267 |
| 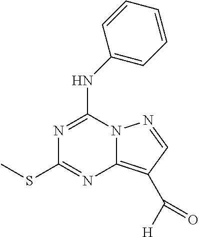 | 285.32 | 286 |
| 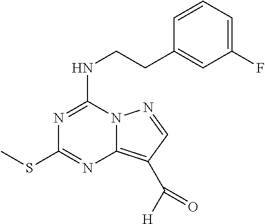 | 331.37 | 332 |

### Example 133

### Synthesis of (E)-3-((4-(cyclopropylamino)-2-(methylthio)pyrazolo[1,5-a][1,3,5]triazin-8-yl)methylene)pyrrolidine-2,5-dione

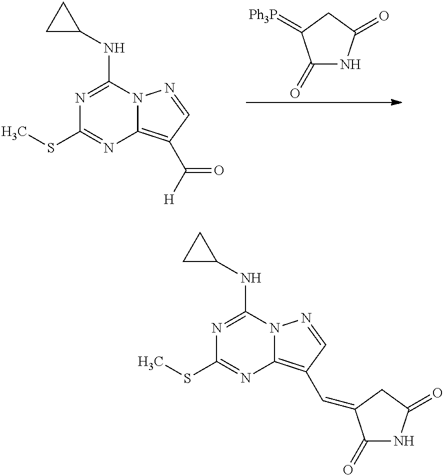

4-(cyclopropylamino)-2-(methylthio)pyrazolo[1,5-a][1,3,5]triazine-8-carbaldehyde (1.0 eq, 1.03 g, 4.120 mmol) was suspended in methanol (20 ml). 3-(triphenylphosphanylidene)-pyrrolidine-2,5-dione (1.0 eq, 1.48 g, 4.120 mmol) was added and the mixture was stirred at reflux for 4 hours, at which time LCMS of an aliquot indicated 82% conversion. An additional amount of phosphanylidene (0.5 g) was added and mixture refluxed for 2 hours. The reaction was cooled down and the solid filtered and washed with methanol. After drying in vacuo, (E)-3-((4-(cyclopropylamino)-2-(methylthio)pyrazolo[1,5-a][1,3,5]triazin-8-yl)methylene)pyrrolidine-2,5-dione was isolated as a yellow solid (1.26 g, 93% yield). LCMS (ES): >95% pure, m/z 331 [M+H]⁺.

### Example 134

### Synthesis of a mixture of (E)-3-((4-(cyclopropylamino)-2-(methylsulfinyl)pyrazolo[1,5-a][1,3,5]triazin-8-yl)methylene)pyrrolidine-2,5-dione and (E)-3-((4-(cyclopropylamino)-2-(methylsulfonyl)pyrazolo[1,5-a][1,3,5]-triazin-8-yl)methylene)pyrrolidine-2,5-dione

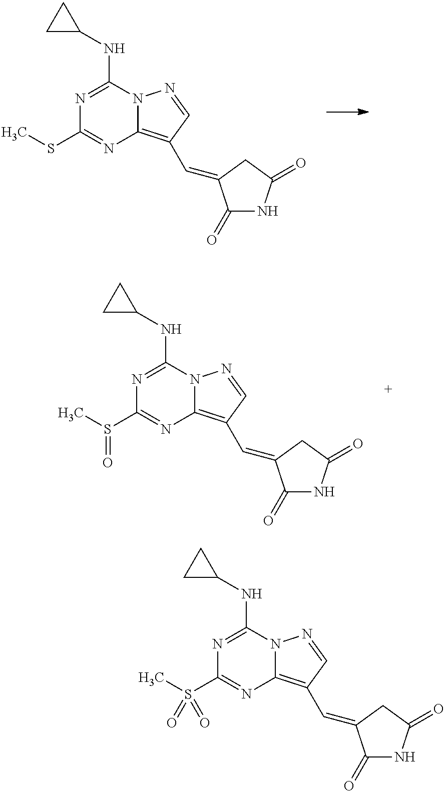

In a round bottom flask (E)-3-((4-(cyclopropylamino)-2-(methylthio)pyrazolo[1,5-a][1,3,5]triazin-8-yl)methylene)pyrrolidine-2,5-dione (1.0 eq, 1.242 g, 3.76 mmol) was suspended in methylene chloride (70 ml). m-chloroperoxybenzoic acid (70% pure grade, 5.0 eq, 4.63 g, 26.82 mmol) was added and the mixture stirred at room temperature for 8 hours. The mixture was diluted with methylene chloride and the solid was filtered. After drying in vacuo, the resulting yellow solid was characterized by LCMS as a mixture containing 6% of (E)-3-((4-(cyclopropylamino)-2-(methylsulfinyl)pyrazolo[1,5-a][1,3,5]triazin-8-yl)methylene)pyrrolidine-2,5-dione and 94% of (E)-3-((4-(cyclopropylamino)-2-(methylsulfonyl)pyrazolo[1,5-a][1,3,5]triazin-8-yl)methylene)pyrrolidine-2,5-dione (1.257 g, 92% yield). LCMS (ES): >95% pure, m/z 347 [M+H]⁺(sulfoxide), m/z 363 [M+H]⁺(sulfone).

The following two compounds and Examples 135 to 138 were prepared by using procedures similar to Examples 133 and 134. Compounds were characterized by LCMS.

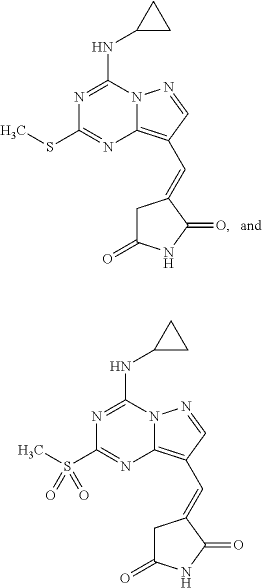

### Example 135

### Synthesis of a mixture of (E)-3-((4-((S)-3-fluoropyrrolidin-1-yl)-2-(methylsulfinyl)pyrazolo[1,5-a][1,3,5]triazin-8-yl)methylene)pyrrolidine-2,5-dione and (S,E)-3-((4-(3-fluoropyrrolidin-1-yl)-2-(methylsulfonyl)pyrazolo[1,5-a][1,3,5]triazin-8-yl)methylene)pyrrolidine-2,5-dione.

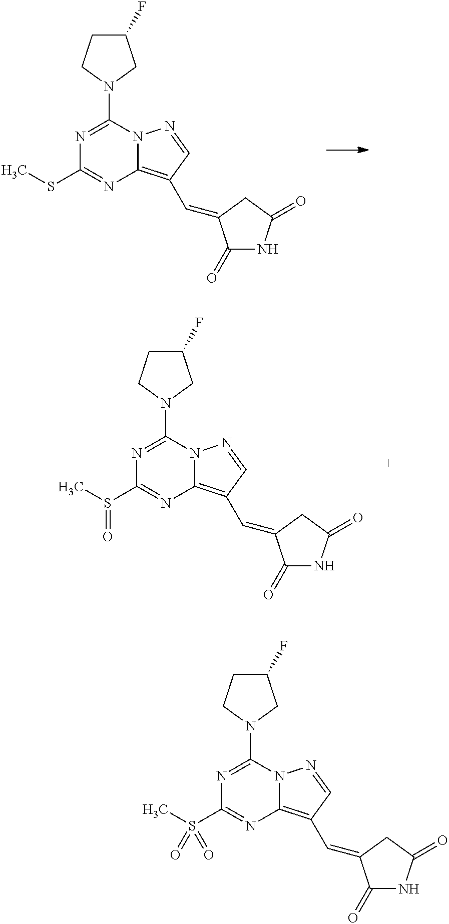

LCMS m/z 379 [M+H]⁺(sulfoxide), m/z 395 [M+H]⁺(sulfone).

### Example 136

### Synthesis of a mixture of (E)-3-((4-(2-methoxyethylamino)-2-(methylsulfinyl)pyrazolo[1,5-a][1,3,5]-triazin-8-yl)methylene)pyrrolidine-2,5-dione and (E)-3-((4-(2-methoxyethylamino)-2-(methylsulfonyl)pyrazolo[1,5-a][1,3,5]triazin-8-yl)methylene)pyrrolidine-2,5-dione

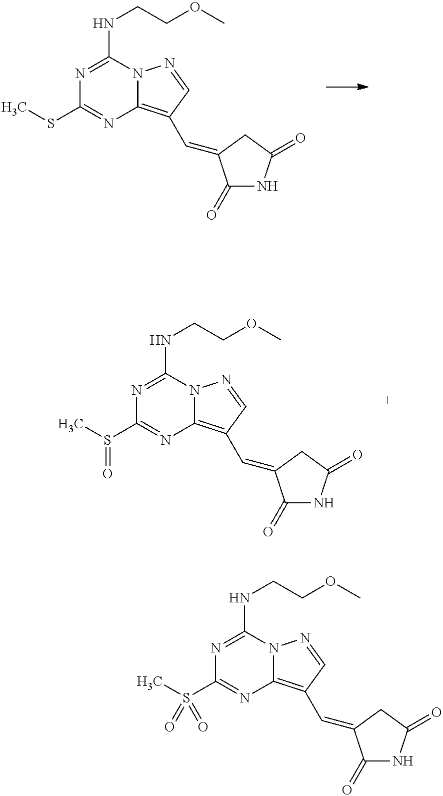

LCMS m/z 365 [M+H]⁺(sulfoxide), m/z 381 [M+H]⁺(sulfone).

### Example 137

### Synthesis of a mixture of (E)-3-((2-(methylsulfinyl)-4-(phenylamino)pyrazolo[1,5-a][1,3,5]-triazin-8-yl)methylene)pyrrolidine-2,5-dione and (E)-3-((2-(methylsulfonyl)-4-(phenylamino)pyrazolo[1,5-a][1,3,5]-triazin-8-yl)methylene)pyrrolidine-2,5-dione.

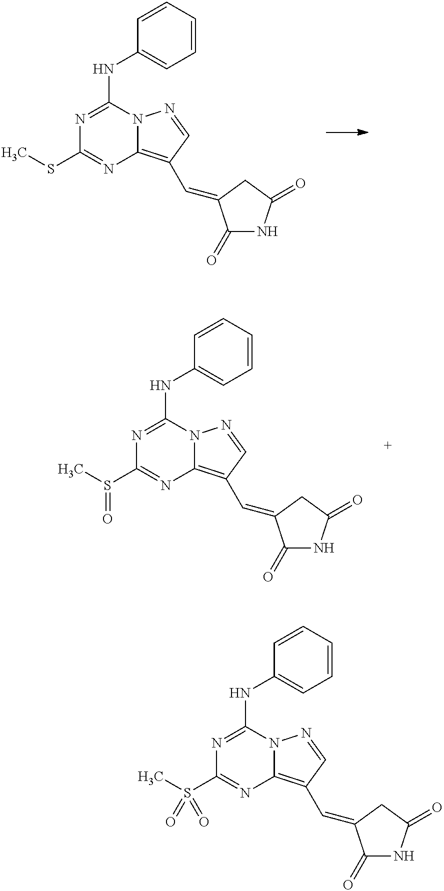

LCMS m/z 383 [M+H]⁺(sulfoxide), m/z 399 [M+H]⁺(sulfone).

### Example 138

### Synthesis of a mixture of (E)-3-((4-(3-fluorophenethylamino)-2-(methylsulfinyl)pyrazolo[1,5-a][1,3,5]triazin-8-yl)methylene)pyrrolidine-2,5-dione and (E)-3-(4-(3-fluorophenethylamino)-2-(methylsulfonyl)pyrazolo[1,5-a][1,3,5]-triazin-8-yl)methylene)pyrrolidine-2,5-dione.

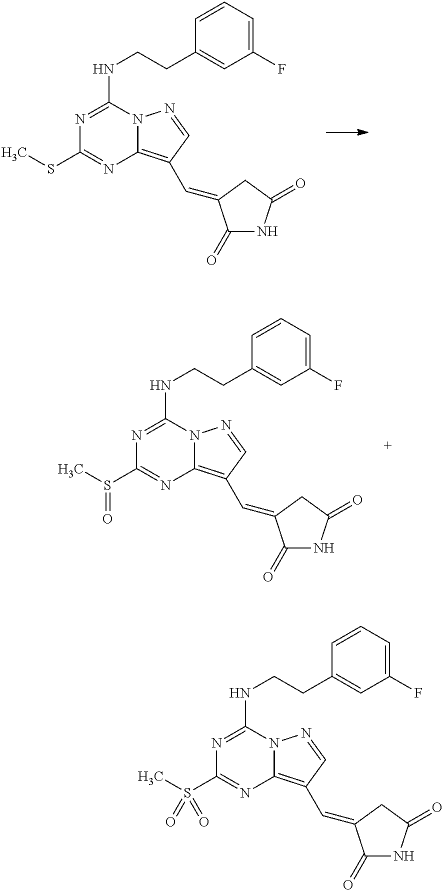

LCMS m/z 429 [M+H]⁺(sulfoxide), m/z 445 [M+H]⁺(sulforie).

The following four compounds were prepared by using the procedures described above. Compounds were characterized by LCMS.

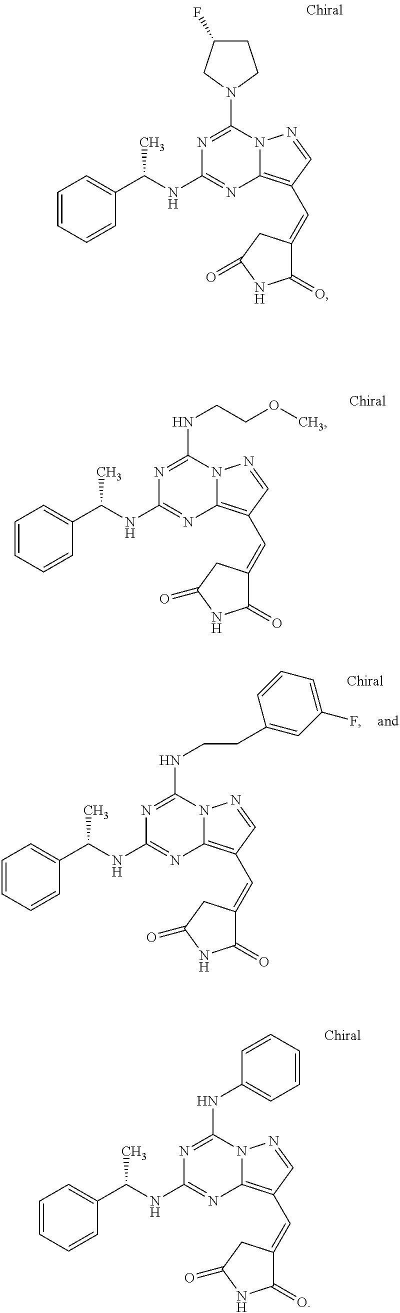

### Example 139

### Synthesis of (E)-3-((2-(3-chlorophenylamino)-4-(cyclopropylamino)pyrazolo[1,5-a][1,3,5]-triazin-8-yl)methylene)pyrrolidine-2,5-dione

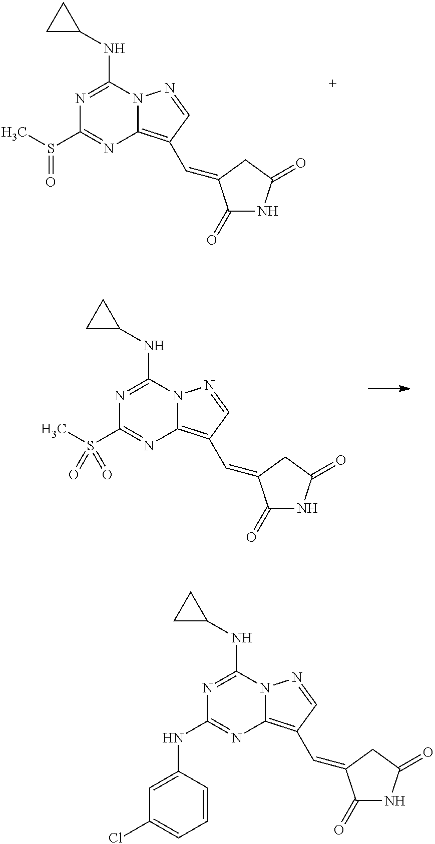

A mixture of (E)-3-((4-(cyclopropylamino)-2-(methylsulfinyl)pyrazolo[1,5-a][1,3,5]triazin-8-yl)methylene)pyrrolidine-2,5-dione and (E)-3-((4-(cyclopropylamino)-2-(methylsulfonyl)pyrazolo[1,5-a][1,3,5]triazin-8-yl)methylene)pyrrolidine-2,5-dione (9 mg) was mixed with 3-chloro-aniline (0.2 ml) and NMP (0.2 ml). The mixture was reacted in a microwave oven at 120° C. for 20 min. The reaction mixture was diluted and purified by preparative HPLC. (E)-3-((2-(3-chlorophenylamino)-4-(cyclopropylamino)pyrazolo[1,5-a][1,3,5]triazin-8-yl)methylene)pyrrolidine-2,5-dione was isolated a beige solid (5 mg). LCMS (ES): >95% pure, m/z 410 [M+H]⁺

### Example 140

### Synthesis of (S,E)-3-((4-(cyclopropylamino)-2-(1-cyclopropylethylamino)pyrazolo[1,5-a][1,3,5]triazin-8-yl)methylene)pyrrolidine-2,5-dione

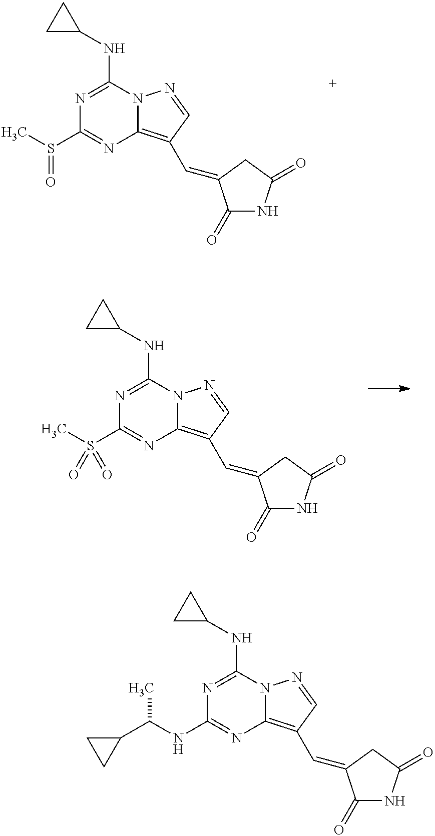

A mixture of (E)-3-((4-(cyclopropylamino)-2-(methylsulfinyl)pyrazolo[1,5-a][1,3,5]triazin-8-yl)methylene)pyrrolidine-2,5-dione and (E)-3-((4-(cyclopropylamino)-2-(methylsulfonyl)pyrazolo[1,5-a][1,3,5]triazin-8-yl)methylene)pyrrolidine-2,5-dione (8 mg, 0.022 mmol) in 0.4 mL NMP was reacted with (S)-1-cyclopropylethanamine (0.110 ml of 0.4M solution in NMP) at 70° C. for 2 h. The material was filtered and purified by mass-directed LC/MS to provide (S,E)-3-((4-(cyclopropylamino)-2-(1-cyclopropylethylamino)pyrazolo[1,5-a][1,3,5]triazin-8-yl)methylene)pyrrolidine-2,5-dione as the TFA salt. LCMS: m/z 368 [M+H]⁺

The following compounds were prepared by using procedures similar to Example 140. Compounds were characterized by LCMS.

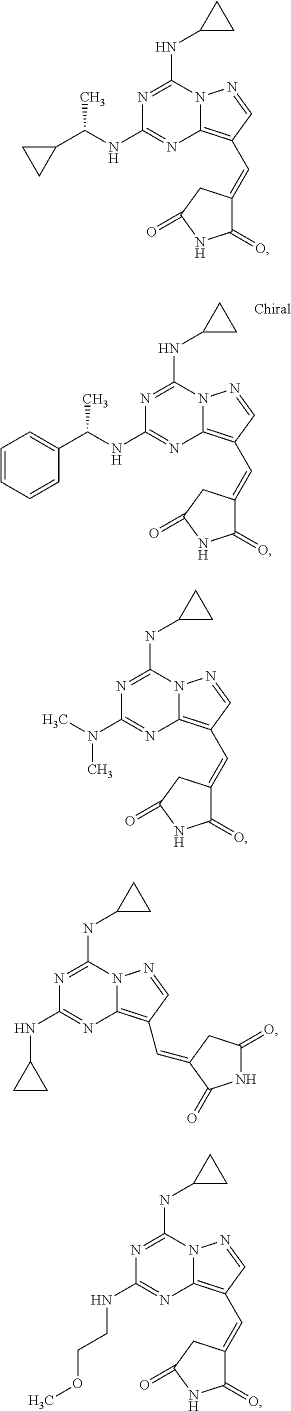

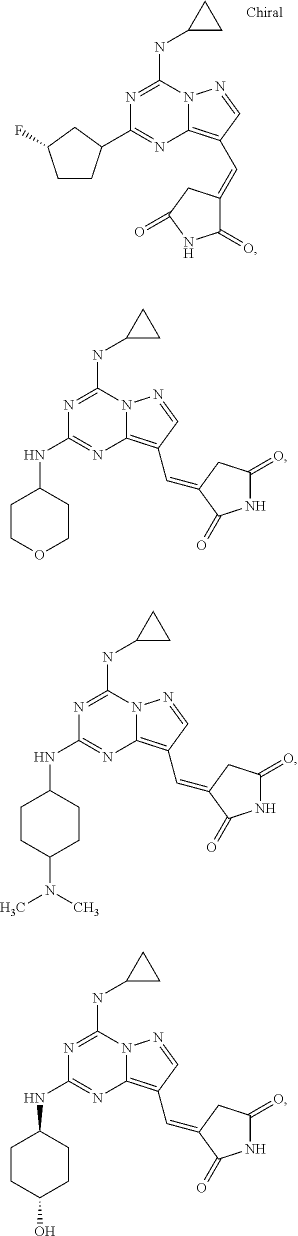

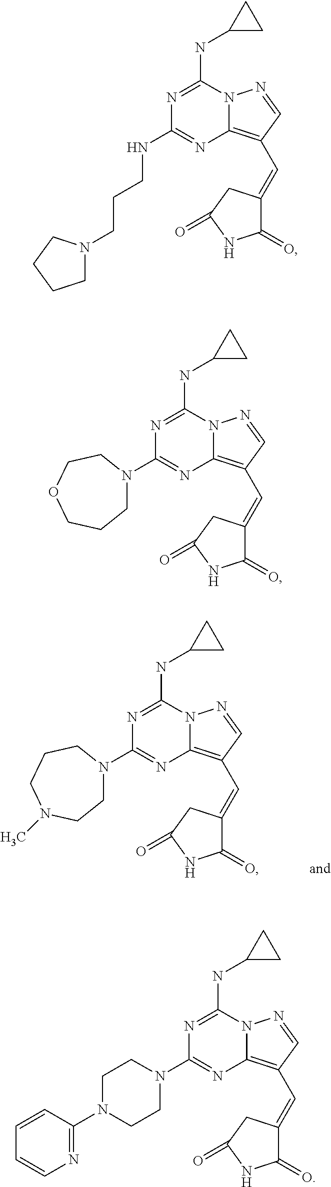

### Example 141

### Synthesis of (E)-3-((2-((S)-1-cyclopropylethylamino)-4-((S)-3-fluoropyrrolidin-1-yl)pyrazolo[1,5-a][1,3,5]-triazin-8-yl)methylene)pyrrolidine-2,5-dione

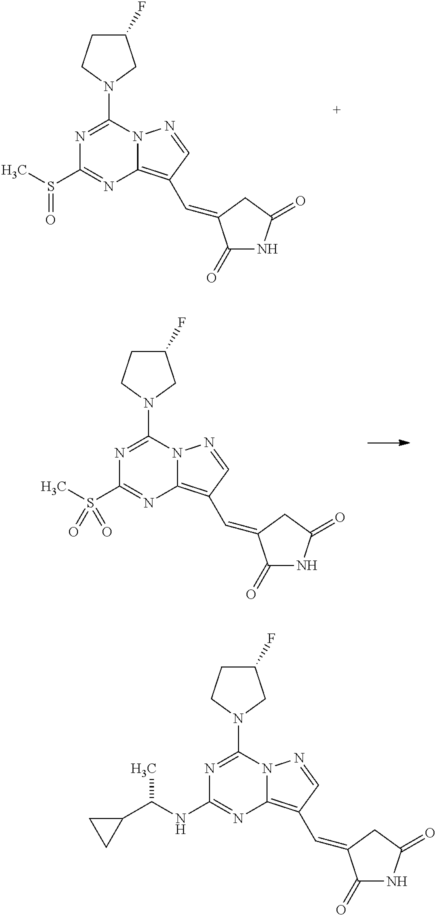

A mixture of (E)-3-((4-((S)-3-fluoropyrrolidin-1-yl)-2-(methylsulfinyl)pyrazolo[1,5-a][1,3,5]triazin-8-yl)methylene)pyrrolidine-2,5-dione and (S,E)-3-((4-(3-fluoropyrrolidin-1-yl)-2-(methylsulfonyl)pyrazolo[1,5-a][1,3,5]triazin-8-yl)methylene)pyrrolidine-2,5-dione (8 mg, 0.020 mmol) in 0.4 mL NMP was reacted with (S)-1-cyclopropylethanamine (0.101 ml of 0.4M solution in NMP) at 70° C. for 2 h. The material was filtered and purified by mass-directed LC/MS to provide (E)-3-((2-((S)-1-cyclopropylethylamino)-4-((S)-3-fluoropyrrolidin-1-yl)pyrazolo[1,5-a][1,3,5]triazin-8-yl)methylene)pyrrolidine-2,5-dione as the TFA salt. LCMS: m/z 400 [M+H]⁺

The following compounds were prepared by using procedures similar to Example 141. Compounds were characterized by LCMS.

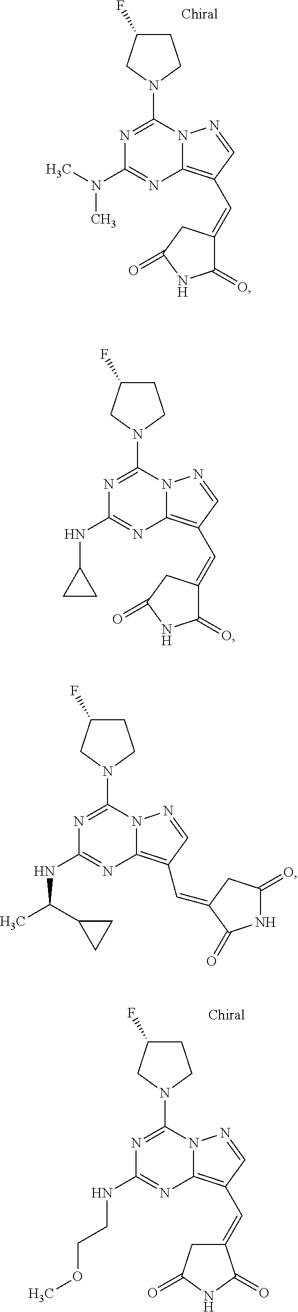

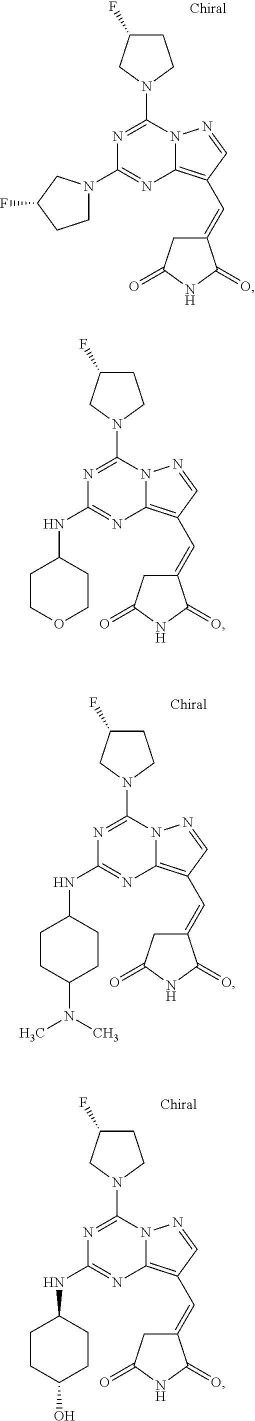

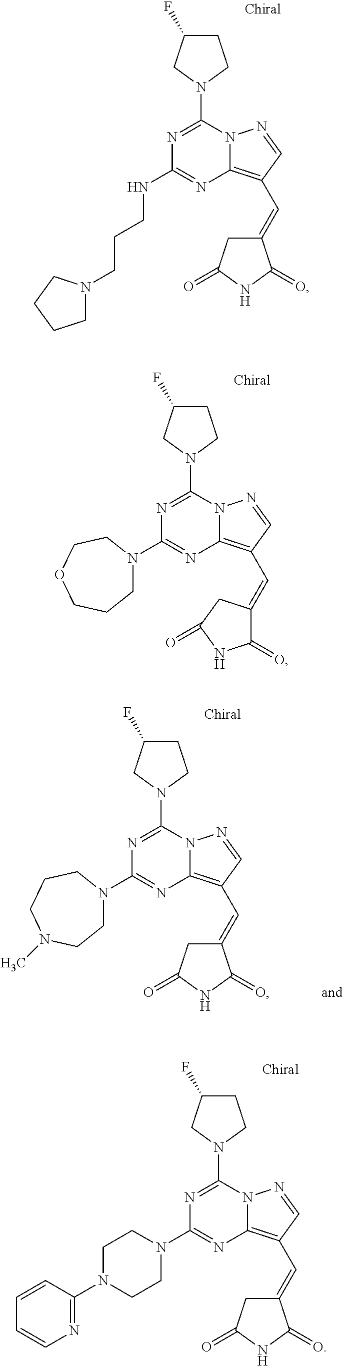

### Example 142

### Synthesis of (S,E)-3-((2-(1-cyclopropylethylamino)-4-(2-methoxyethylamino)pyrazolo[1,5-a][1,3,5]triazin-8-yl)methylene)pyrrolidine-2,5-dione

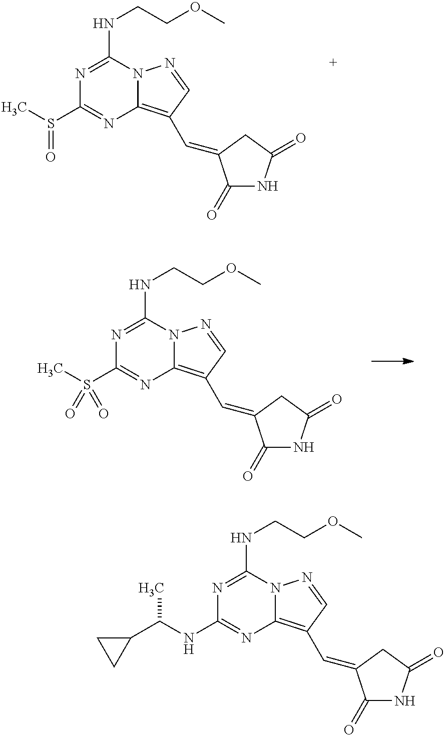

A mixture of (E)-3-((4-(2-methoxyethylamino)-2-(methylsulfinyl)pyrazolo[1,5-a][1,3,5]triazin-8-yl)methylene)pyrrolidine-2,5-dione and (E)-3-((4-(2-methoxyethylamino)-2-(methylsulfonyl)pyrazolo[1,5-a][1,3,5]triazin-8-yl)methylene)pyrrolidine-2,5-dione (8 mg, 0.021 mmol) in 0.4 mL NMP was reacted with (S)-1-cyclopropylethanamine (0.105 ml of 0.4M solution in NMP) at 70° C. for 2 h. The material was filtered and purified by mass-directed LC/MS to provide (S,E)-3-((2-(1-cyclopropylethylamino)-4-(2-methoxyethylamino)pyrazolo[1,5-a][1,3,5]triazin-8-yl)methylene)pyrrolidine-2,5-dione as the TFA salt. LCMS: m/z 386 [M+H]⁺

The following compounds were prepared by using procedures similar to Example 142. Compounds were characterized by LCMS.

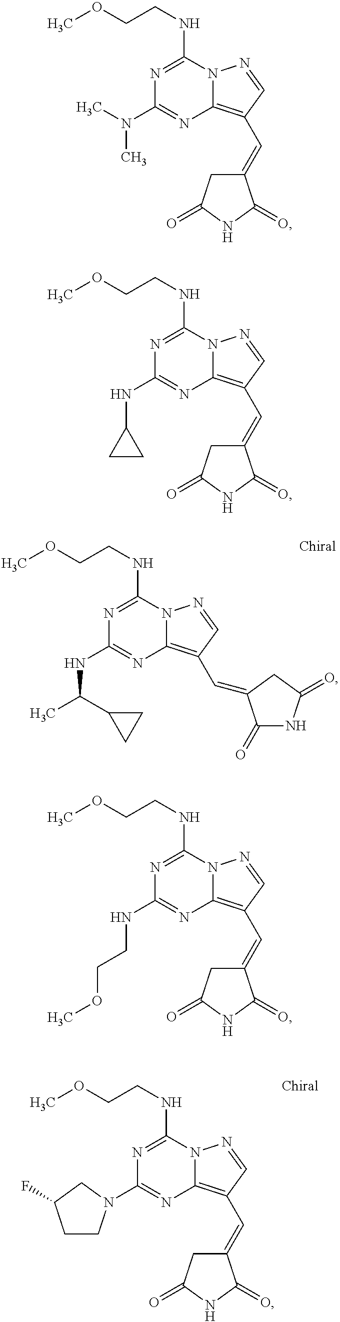

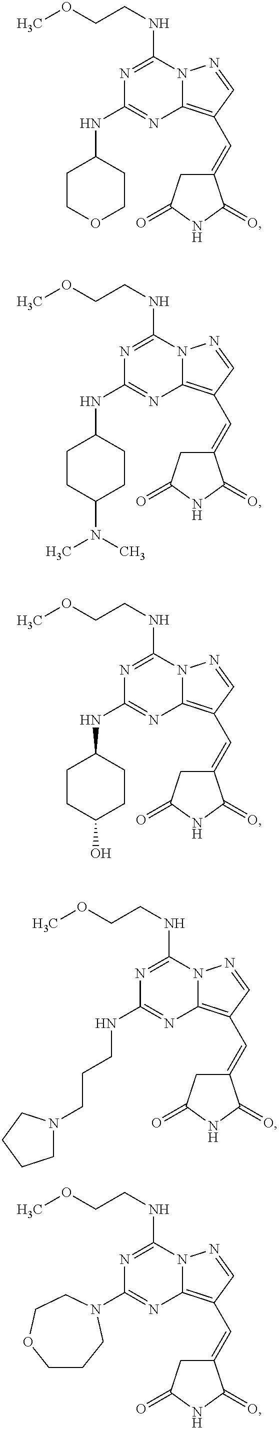

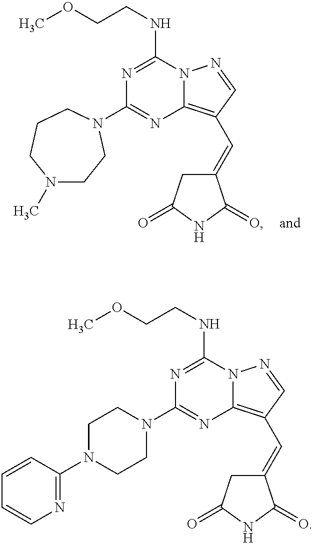

### Example 143

### Synthesis of (S,E)-3-((2-(1-cyclopropylethylamino)-4-(3-fluorophenethylamino)pyrazolo[1,5-a][1,3,5]triazin-8-yl)methylene)pyrrolidine-2,5-dione

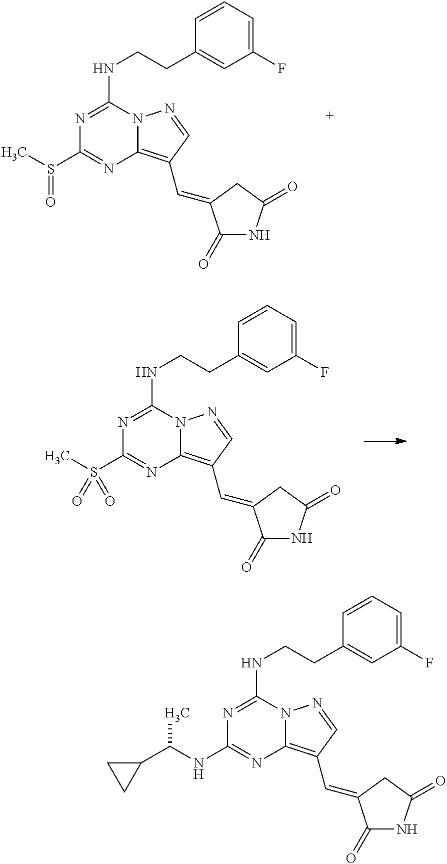

A mixture of (E)-3-((4-(3-fluorophenethylamino)-2-(methylsulfinyl)pyrazolo[1,5-a][1,3,5]triazin-8-yl)methylene)pyrrolidine-2,5-dione and (E)-3-((4-(3-fluorophenethylamino)-2-(methylsulfonyl)pyrazolo[1,5-a][1,3,5]triazin-8-yl)methylene)pyrrolidine-2,5-dione (8 mg, 0.018 mmol) in 0.4 mL NMP was reacted with (S)-1-cyclopropylethanamine (0.090 ml of 0.4M solution in NMP) at 70° C. for 2 h. The material was filtered and purified by mass-directed LC/MS to provide (S,E)-3-((2-(1-cyclopropylethylamino)-4-(3-fluorophenethylamino)pyrazolo[1,5-a][1,3,5]triazin-8-yl)methylene)pyrrolidine-2,5-dione as the TFA salt. LCMS: m/z 450 [M+H]+

The following compounds were prepared by using procedures similar to Example 143. Compounds were characterized by LCMS.

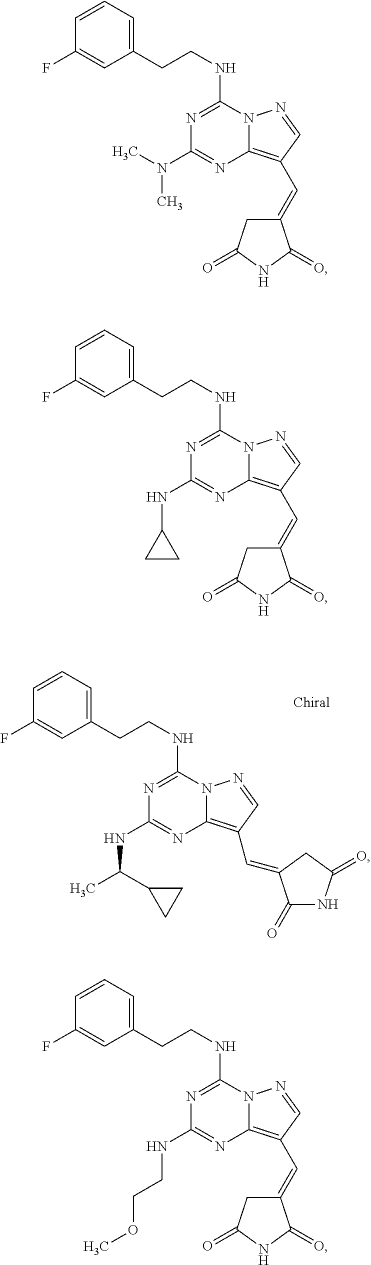

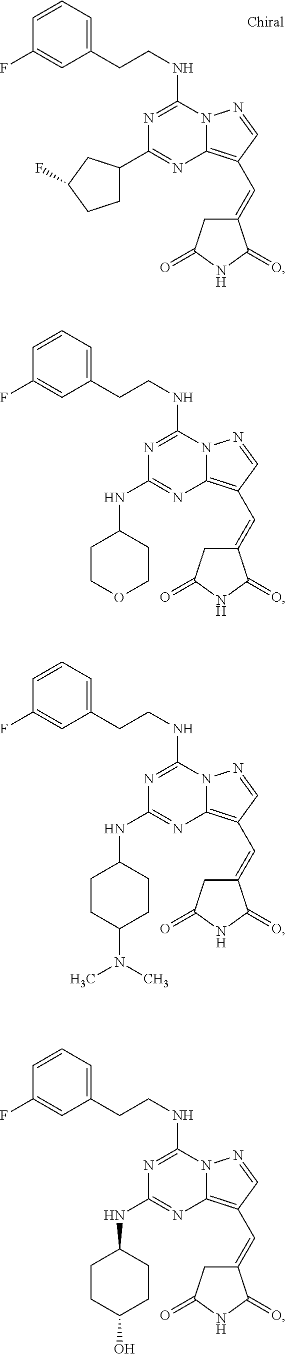

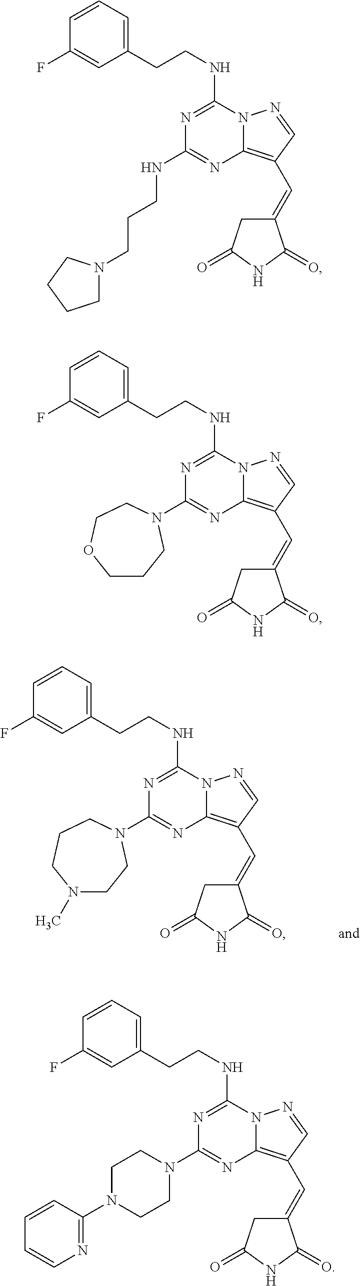

### Example 144

### Synthesis of (S,E)-3-((2-(1-cyclopropylethylamino)-4-(phenylamino)pyrazolo[1,5-a][1,3,5]-triazin-8-yl)methylene)pyrrolidine-2,5-dione

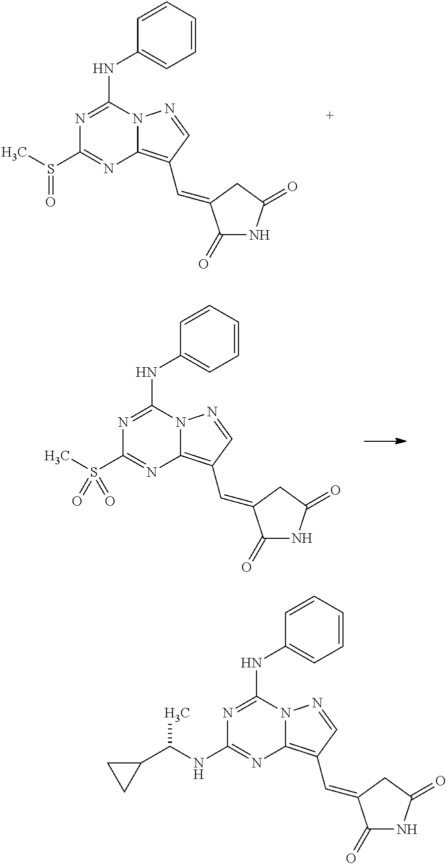

A mixture of (E)-3-((4-(3-fluorophenethylamino)-2-(methylsulfinyl)pyrazolo[1,5-a][1,3,5]triazin-8-yl)methylene)pyrrolidine-2,5-dione and (E)-3-((4-(3-fluorophenethylamino)-2-(methylsulfonyl)pyrazolo[1,5-a][1,3,5]triazin-8-yl)methylene)pyrrolidine-2,5-dione (8 mg, 0.018 mmol) in 0.4 mL NMP was reacted with (S)-1-cyclopropylethanamine (0.090 ml of 0.4M solution in NMP) at 70° C. for 2 h. The material was filtered and purified by mass-directed LC/MS to provide (S,E)-3-((2-(1-cyclopropylethylamino)-4-(phenylamino)pyrazolo[1,5-a][1,3,5]triazin-8-yl)methylene)pyrrolidine-2,5-dione as the TFA salt. LCMS: m/z 404 [M+H]⁺

The following compounds were prepared by using procedures similar to Example 144. Compounds were characterized by LCMS.

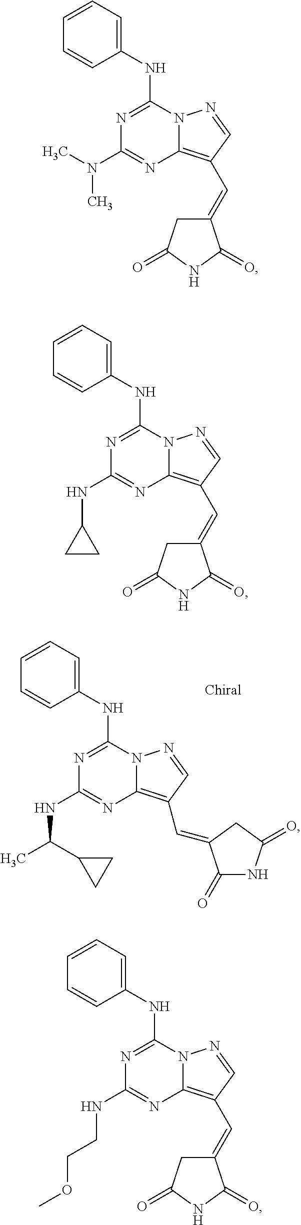

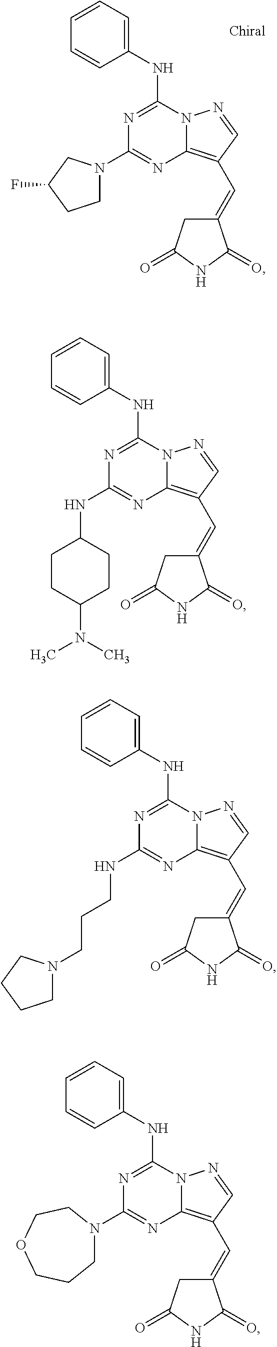

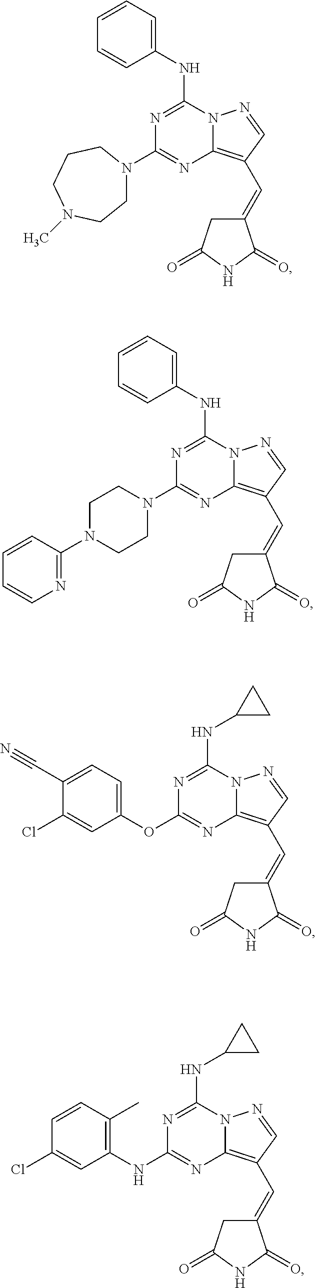

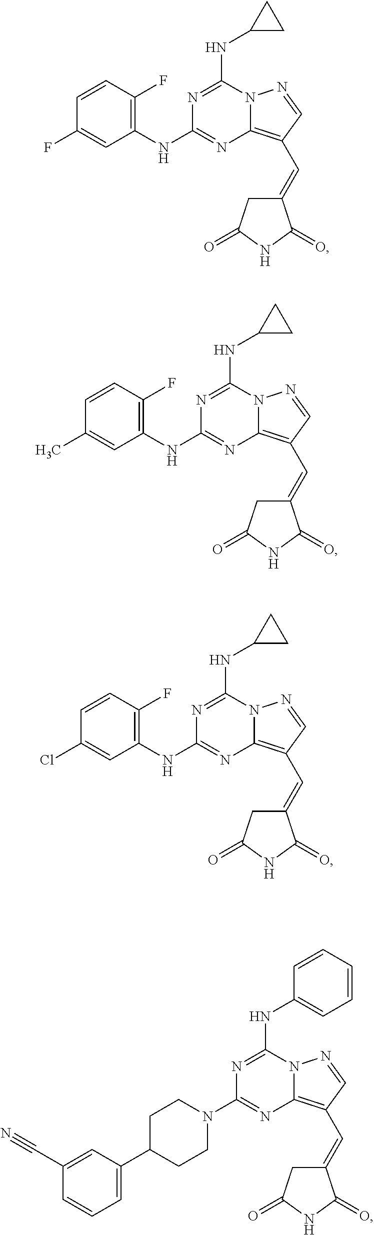

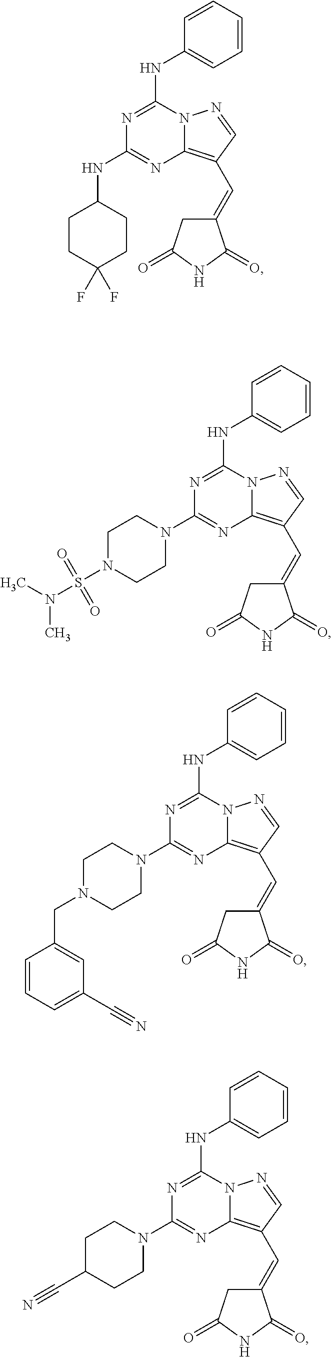

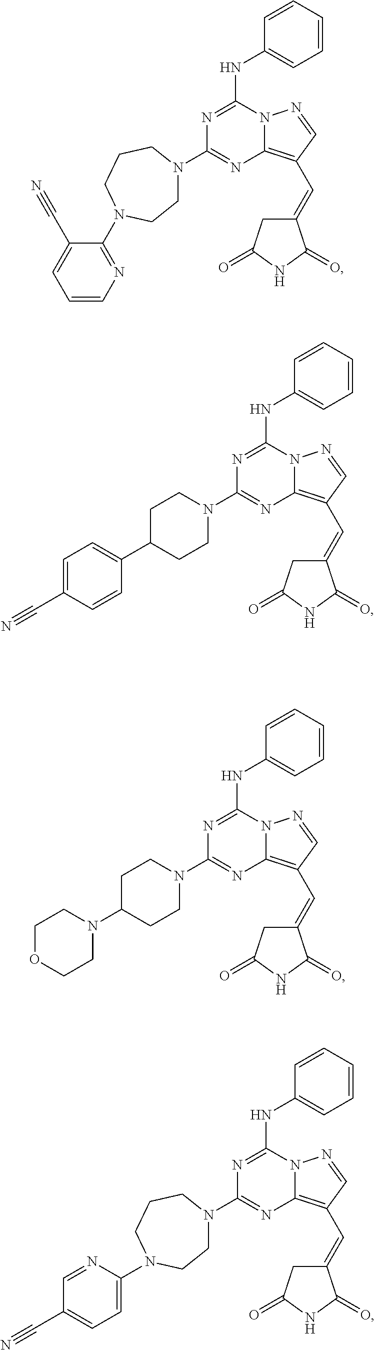

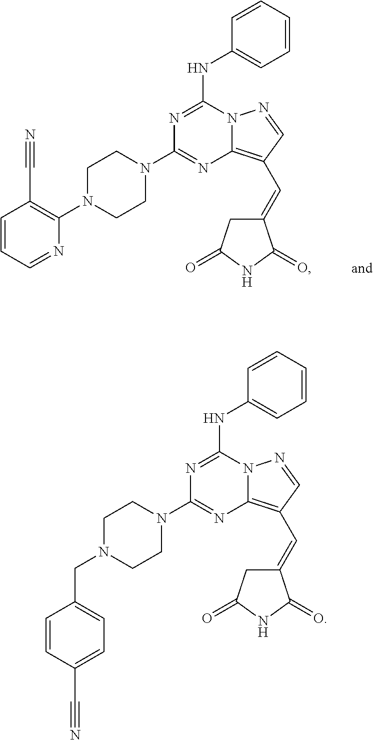

The synthetic methods described on Scheme G4 can be used to prepare analogs of formula 11. 4-bromo-6-chloropyridazin-3-amine 1 can be reacted with 2 using conditions analogous to the preparation described in the patent application WO2009/100375 to form compound 3. Compound 3 can react with amine R₈R₇NH to form compound 4. Compound 4 can be transformed to compound 5 by nucleophilic substitutions with amines, anilines, alcohols, phenols or thiophenols, in the presence of a base, or by transition metal catalyzed conversions such as Suzuki coupling with boronic acid or esters of formula WB(OR)₂. Compound 5 can be transformed to compound 6 by reduction with LiAlH₄. Alcohol 6 can be converted to aldehyde 7 by oxidation with DCC or under Swern conditions. Compound 5 can react with an organometallic reagent exemplified by Grignard reagent R⁴MgX to form secondary alcohol 8. This compound can be converted to alkylketone 9 under conditions analogous to the conditions used to convert 6 into 7. Compounds 7 and 9 can both be converted to compound 11 by reaction with 8a in a solvent such as ethanol.

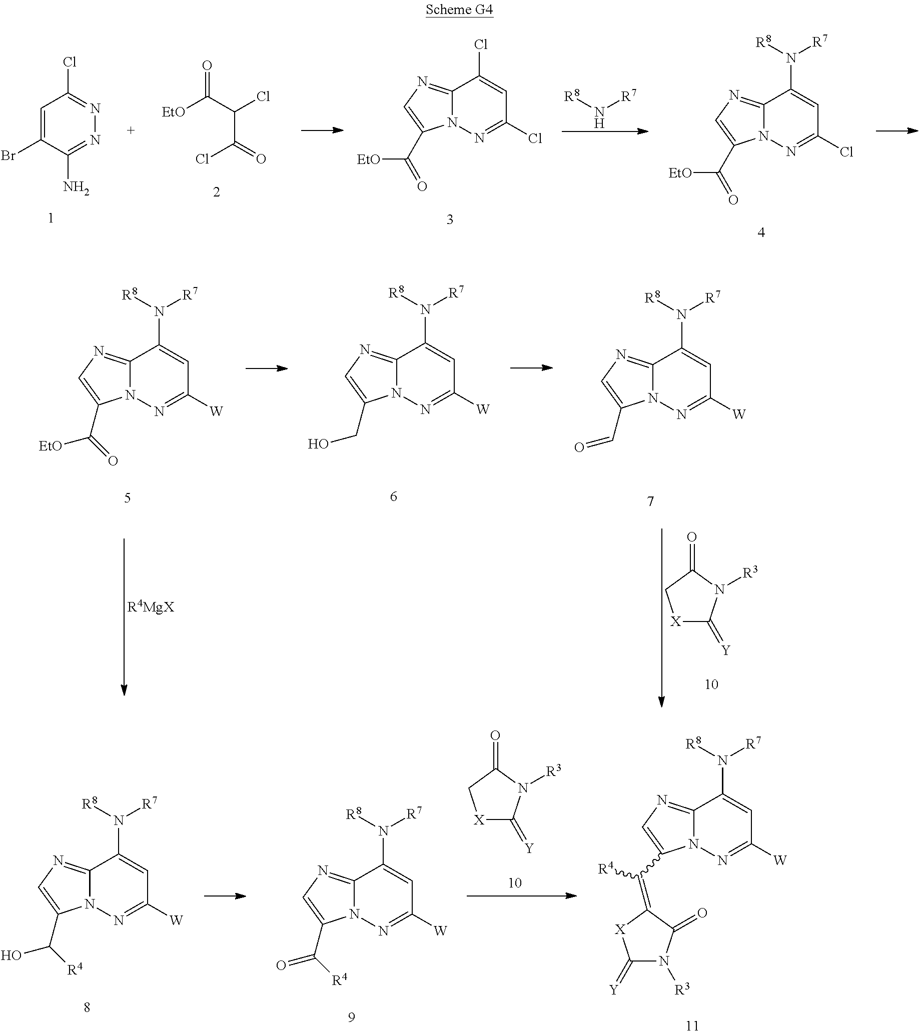

The compounds described in the following table were prepared by using procedures and methods described above including Scheme G4.

Structure

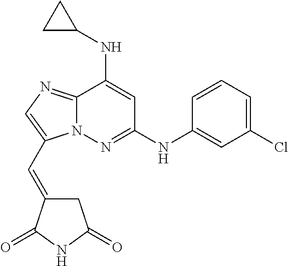

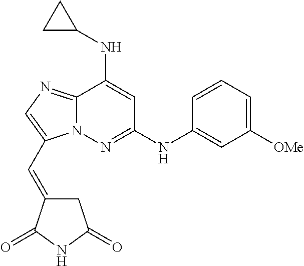

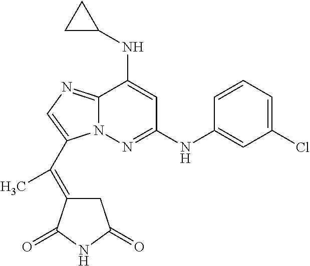

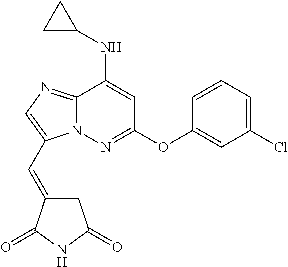

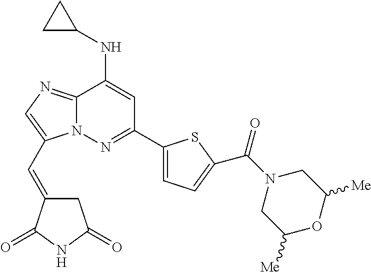

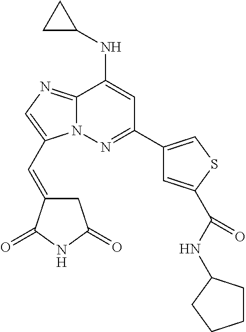

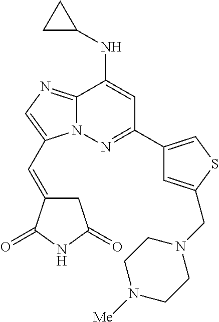

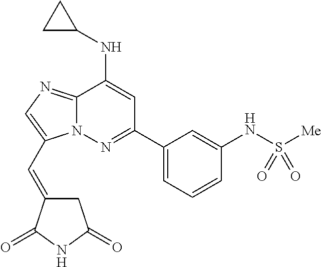

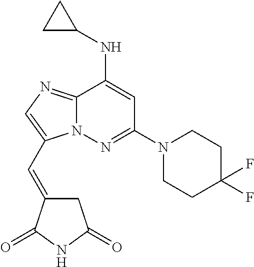

### Examples 145 to 165 were prepared by using the procedures as described above including Examples 56 and 67

### Example 145

### Synthesis of 3-((5-(3-chloro-4-methylphenylamino)-7-(cyclopropylamino)pyrazolo[1,5-a]pyrimidin-3-yl)methylene)pyrrolidine-2,5-dione

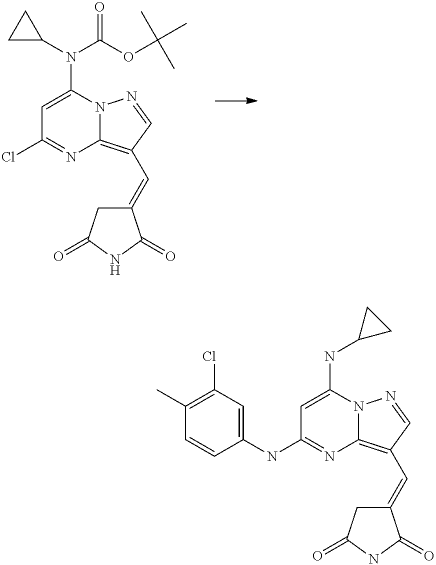

LCMS (M+1=423)

### Example 146

### Synthesis of (E)-3-((7-(cyclopropylamino)-5-(2-fluorophenylamino)pyrazolo[1,5-a]pyrimidin-3-yl)methylene)pyrrolidine-2,5-dione

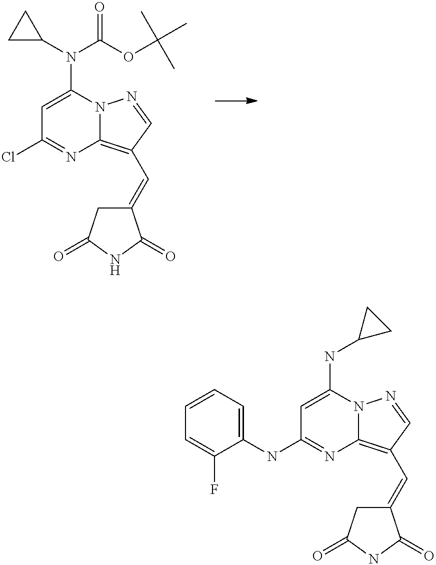

LCMS (M+1=393)

### Example 147

### Synthesis of (E)-3-((5-(2-chloro-3-fluorophenylamino)-7-(cyclopropylamino)pyrazolo[1,5-a]pyrimidin-3-yl)methylene)pyrrolidine-2,5-dione

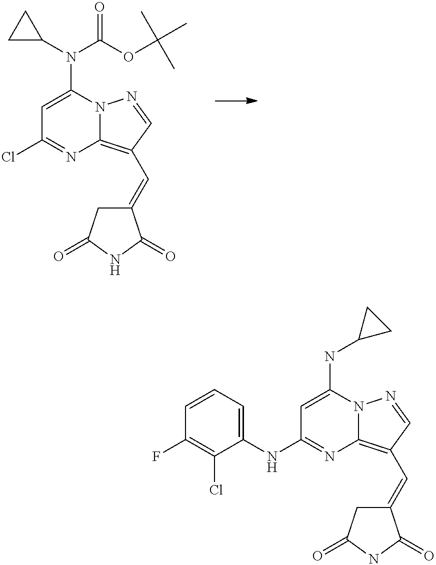

LCMS (M+1=427)

### Example 148

### Synthesis of (E)-3-((5-(3-chloro-4-methoxyphenylamino)-7-(cyclopropylamino)pyrazolo[1,5-a]pyrimidin-3-yl)methyl ene)pyrrolidine-2,5-dione

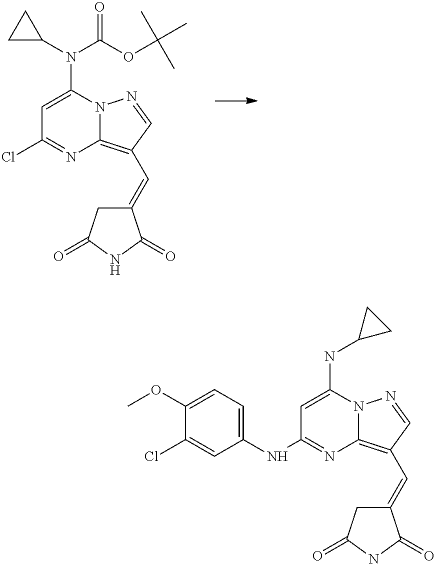

LCMS (M+1=439)

### Example 149

### Synthesis of (E)-3-((5-(3-chloro-2-fluorophenylamino)-7-(cyclopropylamino)pyrazolo[1,5-a]pyrimidin-3-yl)methylene)pyrrolidine-2,5-dione

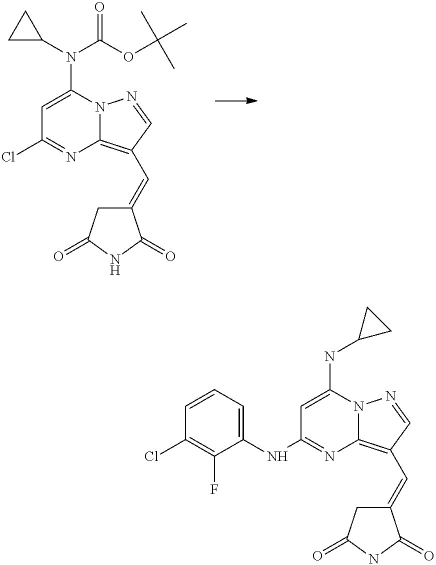

LCMS (M+1=427)

### Example 150

### Synthesis of (E)-3-((7-(cyclopropylamino)-5-(2-fluoro-4-(1H-imidazol-1-yl)phenylamino)pyrazolo[1,5-a]pyrimidin-3-yl)methylene)pyrrolidine-2,5-dione

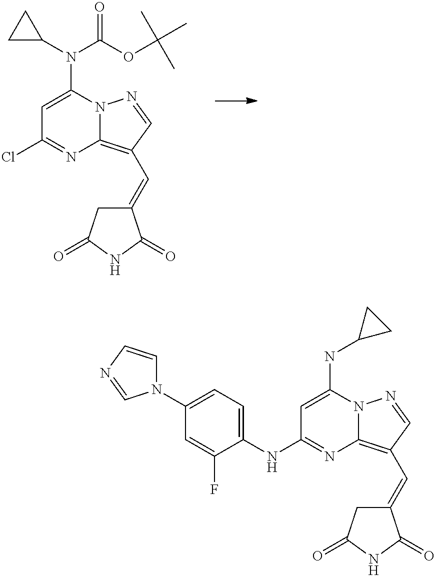

LCMS (M+1=459)

### Example 151

### Synthesis of (E)-3-((5-(2-chloro-5-fluorophenylamino)-7-(cyclopropylamino)pyrazolo[1,5-a]pyrimidin-3-yl)methylene)pyrrolidine-2,5-dione

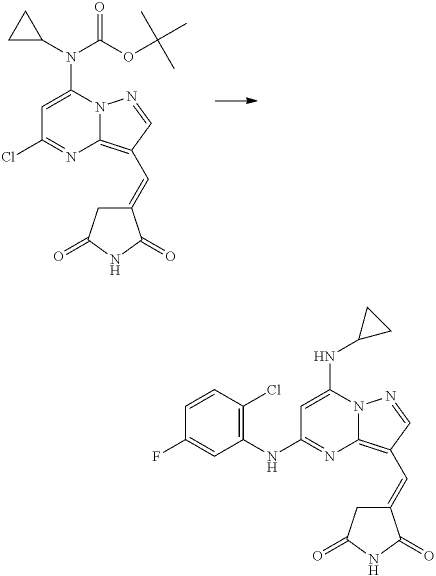

LCMS (M+1=427)

### Example 152

### Synthesis of (E)-3-((5-(2-chlorophenylamino)-7-(cyclopropylamino)pyrazolo[1,5-a]pyrimidin-3-yl)methylene)pyrrolidine-2,5-dione

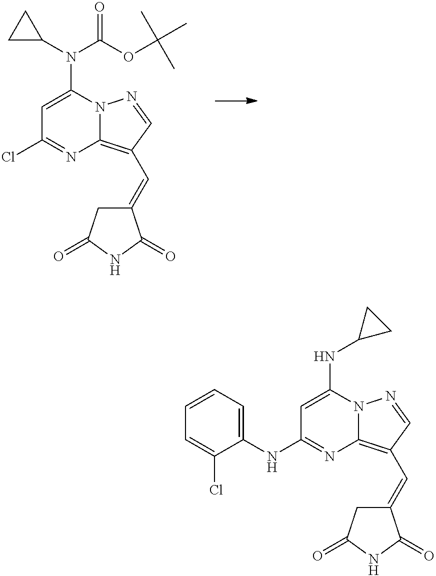

LCMS (M+1=409)

### Example 153

### Synthesis of (E)-2-(7-(cyclopropylamino)-3-((2,5-dioxopyrrolidin-3-ylidene)methyl)pyrazolo[1,5-a]pyrimidin-5-ylamino)benzonitrile

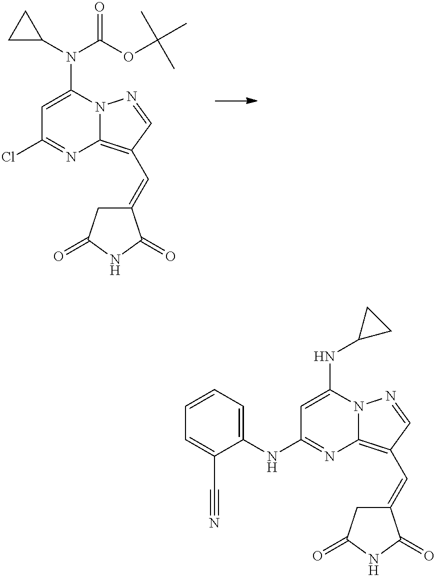

LCMS (M+1=400)

### Example 154

### Synthesis of (E)-2-chloro-4-(7-(cyclopropylamino)-3-((2,5-dioxopyrrolidin-3-ylidene)methyl)pyrazolo[1,5-a]pyrimidin-5-ylamino)benzonitrile

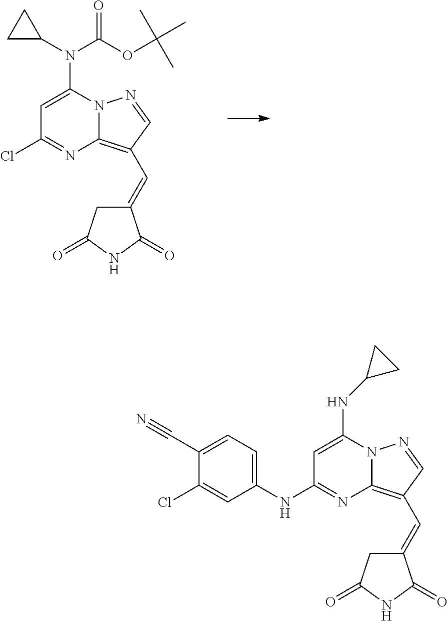

LCMS (M+1=434)

### Example 155

### Synthesis of (E)-3-((7-(cyclopropylamino)-5-(4-fluorophenylamino)pyrazolo[1,5-a]pyrimidin-3-yl)methylene)pyrrolidine-2,5-dione

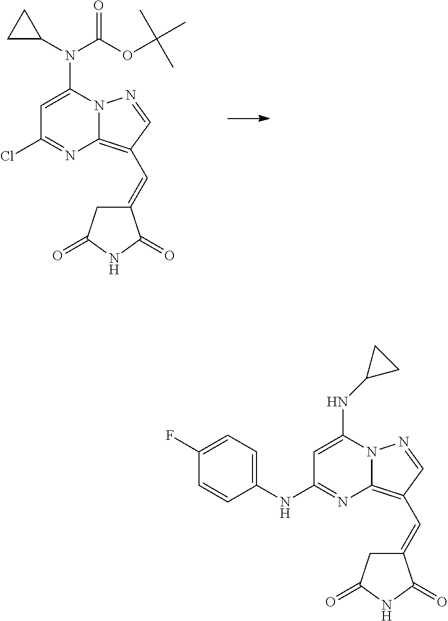

LCMS (M+1=393)

### Example 156

### Synthesis of (E)-3-((7-(cyclopropylamino)-5-(3-isopropoxyphenyl amino)pyrazolo[1,5-a]pyrimidin-3-yl)methylene)pyrrolidine-2,5-drone

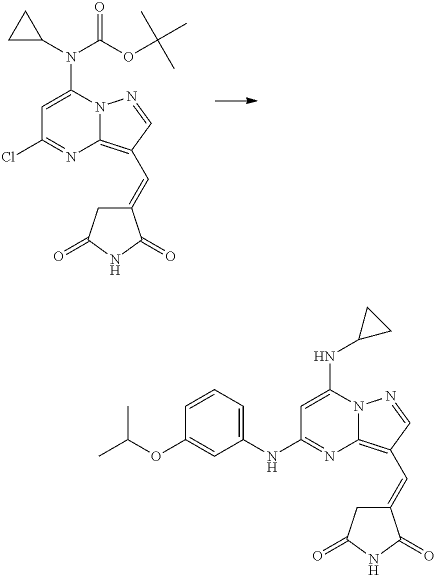

LCMS (M+1=433)

### Example 157

### Synthesis of (E)-3-((5-(2-chloro-5-methylphenylamino)-7-(cyclopropylamino)pyrazolo[1,5-a]pyrimidin-3-yl)methylene)pyrrolidine-2,5-dione

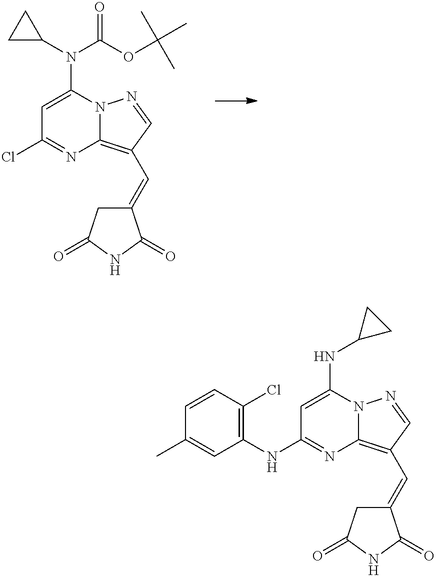

LCMS (M+1=423)

### Example 158

### Synthesis of (E)-4-(7-(cyclopropylamino)-3-((2,5-dioxopyrrolidin-3-ylidene)methyl)pyrazolo[1,5-a]pyrimidin-5-ylamino)benzonitrile

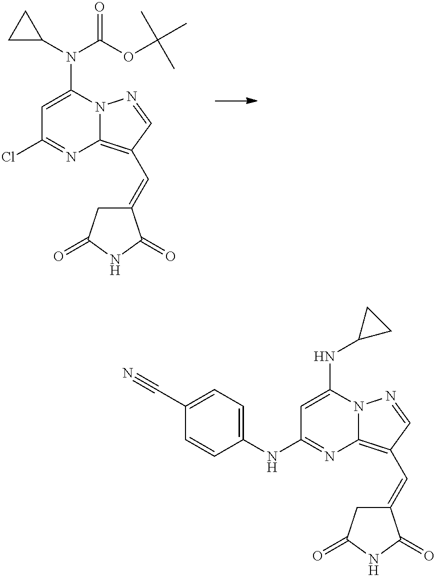

LCMS (M+1=400)

### Example 159

### Synthesis of (E)-3-((7-(cyclopropylamino)-5-(3-ethynylphenylamino)pyrazolo[1,5-a]pyrimidin-3-yl)methylene)pyrrolidine-2,5-dione

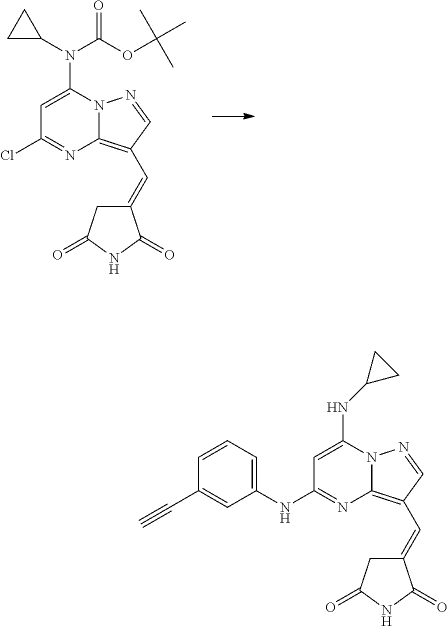

LCMS (M+1=399)

### Example 160

### Synthesis of (E)-3-((5-(3-((1H-imidazol-1-yl)methyl)phenylamino)-7-(cyclopropylamino)pyrazolo[1,5-a]pyrimidin-3-yl)methylene)pyrrolidine-2,5-dione

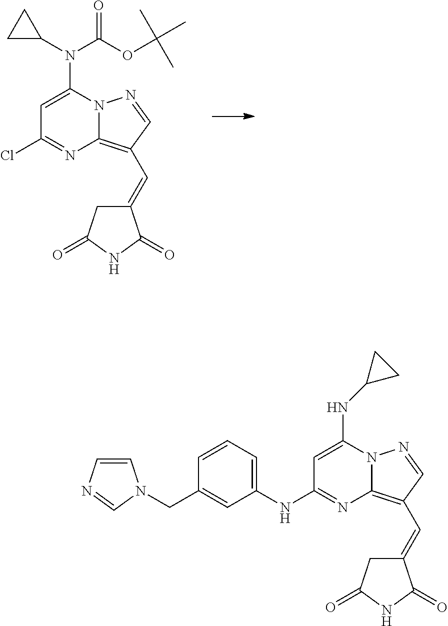

LCMS (M+1=455)

### Example 161

### Synthesis of (E)-3-((5-(3-chloro-4-hydroxyphenylamino)-7-(cyclopropylamino)pyrazolo[1,5-a]pyrimidin-3-yl)methylene)pyrrolidine-2,5-dione

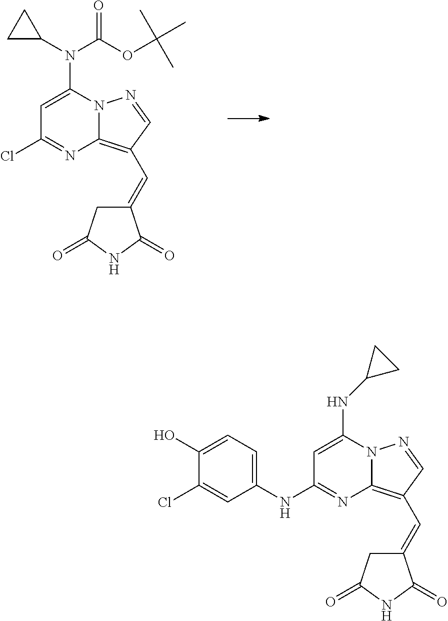

LCMS (M+1=425)

### Example 162

### Synthesis of (E)-3-((5-(5-chloro-2-hydroxyphenylamino)-7-(cyclopropylamino)pyrazolo[1,5-a]pyrimidin-3-yl)methylene)pyrrolidine-2,5-dione

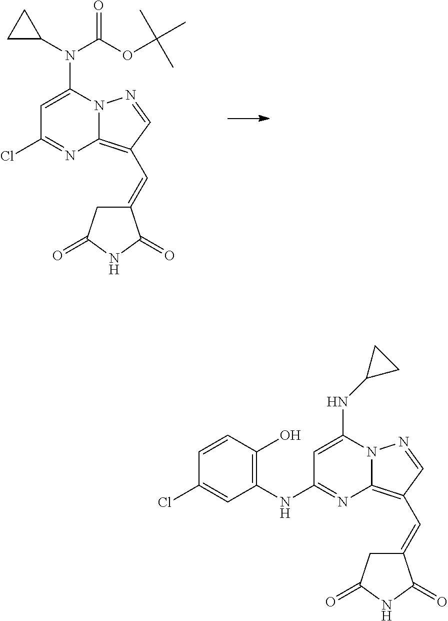

LCMS (M+1=425)

### Example 163

### Synthesis of (E)-3-((5-(1H-benzo[d]imidazol-1-yl)-7-(cyclopropylamino)pyrazolo[1,5-a]pyrimidin-3-yl)methylene)pyrrolidine-2,5-dione

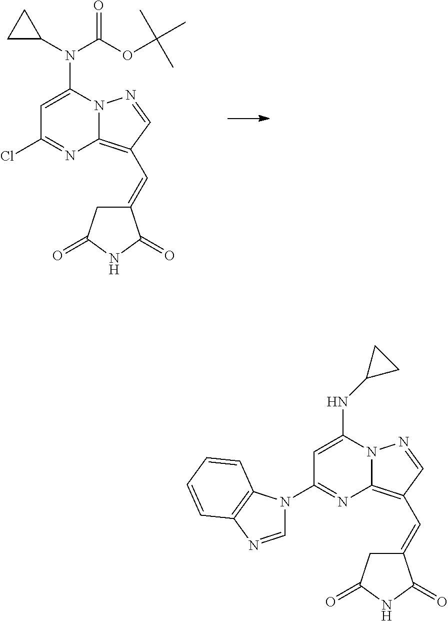

LCMS (M+1=400)

### Example 164

### Synthesis of 3-chloro-4-(2-(pyrrolidin-1-yl)ethoxy)aniline

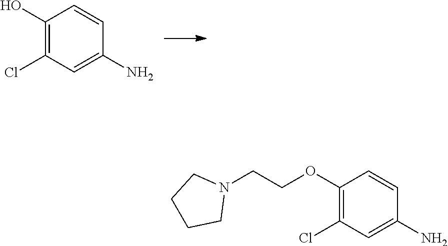

To 4-amino-2-chlorophenol (100 mg, 0.696 mmol) in 2 mL of DMF was added 1-(2-chloroethyl)pyrrolidine HCl (142 mg, 0.835 mmol) and NaOH (70 mg, 1.74 mmol). Stir at 50° C. overnight. Cool to rt and dilute with CH₂Cl₂. Wash 1×H₂O, 3×brine. Dry with MgSO₄, filter, and adsorb onto SiO₂. Purify by flash chromatography eluting with 10% MeOH/CH₂Cl₂ followed by 20% MeOH/CH₂Cl₂ to provide 78 mg of yellow oil. LCMS (M+1=241)

### Example 165

### Synthesis of (E)-3-((5-(3-chloro-4-(2-(pyrrolidin-1-yl)ethoxy)phenylamino)-7-(cyclopropylamino)pyrazolo[1,5-a]pyrimidin-3-yl)methylene)pyrrolidine-2,5-dione

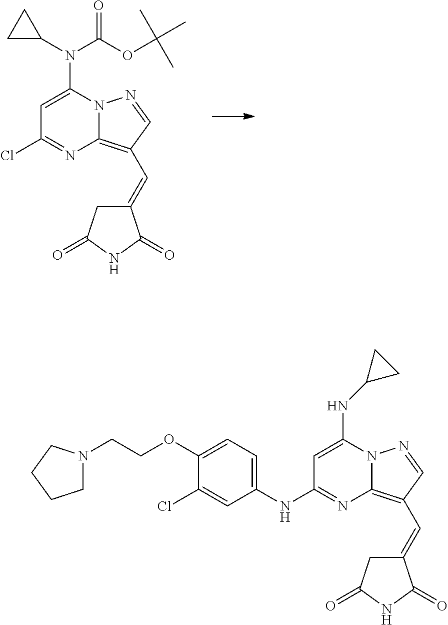

LCMS (M+1=522)

### Example 166

### Synthesis of (E)-3-((4-(cyclopropylamino)-2-(1H-imidazol-1-yl)pyrazolo[1,5-a][1,3,5]triazin-8-yl)methylene)pyrrolidine-2,5-dione

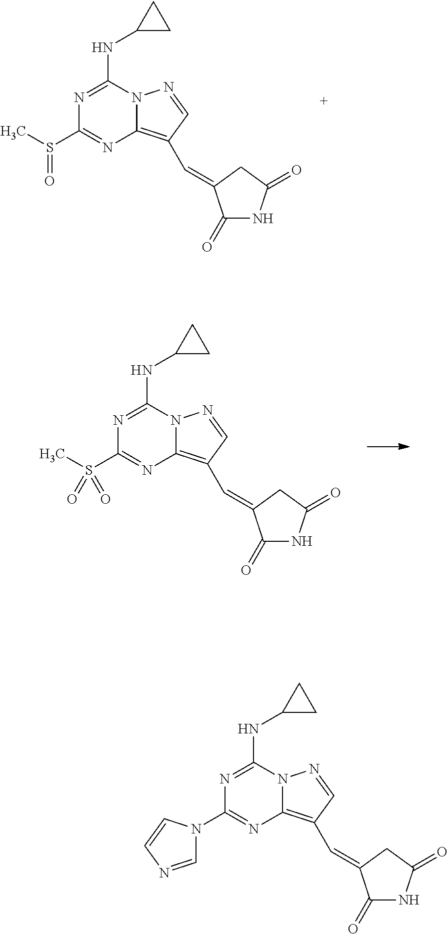

A mixture of (E)-3-((4-(cyclopropylamino)-2-(methylsulfinyl)pyrazolo[1,5-a][1,3,5]triazin-8-yl)methylene)pyrrolidine-2,5-dione and (E)-3-((4-(cyclopropylamino)-2-(methylsulfonyl)pyrazolo[1,5-a][1,3,5]triazin-8-yl)methylene)pyrrolidine-2,5-dione (10 mg, 0.028 mmol) in 1 mL of isopropanol was added imidazole (6 mg, 0.084 mmol). The reaction mixture was stirred at 80° C. for 3 h. Cooled to rt and filtered off resulting solid. Rinsed with water followed by isopropanol to provide (E)-3-(4-(cyclopropylamino)-2-(1H-imidazol-1-yl)pyrazolo[1,5-a][1,3,5]triazin-8-yl)methylene)pyrrolidine-2,5-dione. LCMS (M+1=352)

### Example 167

### Synthesis of (E)-3-((2-(1H-benzo[d]imidazol-1-yl)-4-(cyclopropylamino)pyrazolo[1,5-a][1,3,5]-triazin-8-yl)methylene)pyrrolidine-2,5-dione

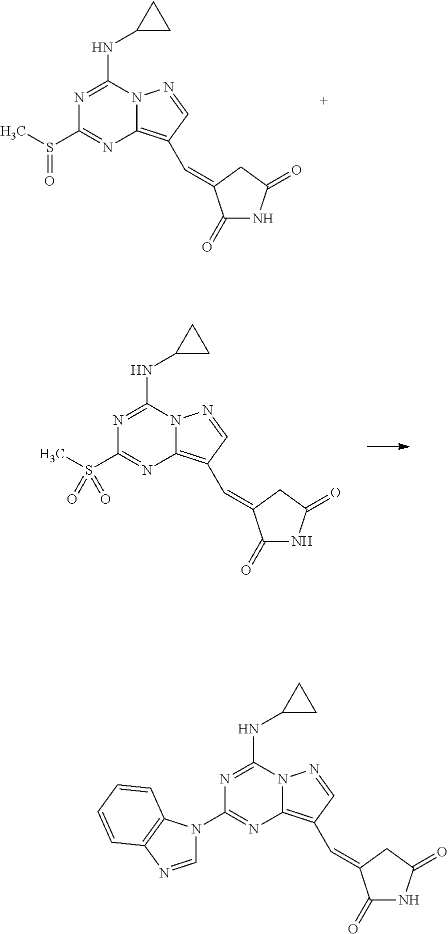

Same procedure as Example 166. LCMS (M+1=401)

### Example 168

### Synthesis of (E)-3-((4-(cyclopropylamino)-2-(5-methyl-1H-benzo[d]imidazol-1-yl)pyrazolo[1,5-a][1,3,5]-triazin-8-yl)methylene)pyrrolidine-2,5-dione

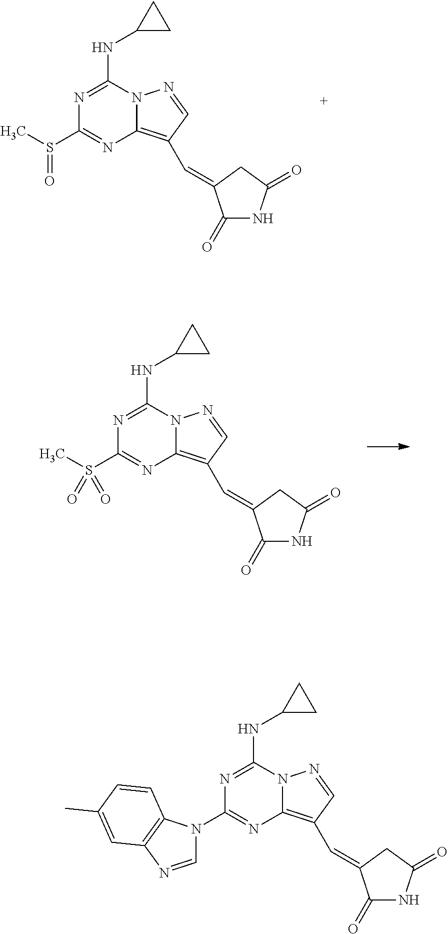

Same procedure as Example 166. LCMS (M+1=415)

### Example 169

### Synthesis of (E)-3-((7-(cyclopropylamino)-5-(1H-imidazol-1-yl)pyrazolo[1,5-a]pyrimidin-3-yl)methylene)pyrrolidine-2,5-dione

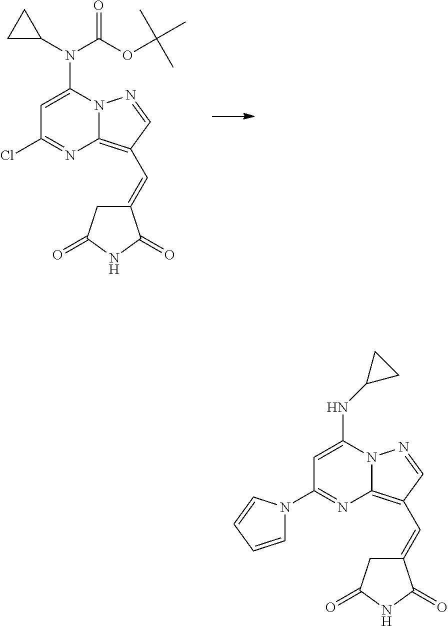

To a mixture of (E)-tert-butyl 5-chloro-3-((2,5-dioxopyrrolidin-3-ylidene)methyl)pyrazolo[1,5-a]pyrimidin-7-yl(cyclopropyl)carbamate (15 mg, 0.036 mmol) in 2 mL of isopropanol was added imidazole (7 mg, 0.108 mmol). The reaction mixture was stirred at reflux overnight. The solvent was removed by rotary evaporation and the residue was taken up in 1 mL of 4M HCl in dioxane and stirred at 50° C. for 1 hr. Excess HCl/dioxane was removed by rotary evaporation and added 2 mL of saturated NaHCO₃. Sonicate and filter the resulting solid. Rinse with H2O followed by 1:1 H2O/EtOH. Dry under vacuum to provide (E)-3-((7-(cyclopropylamino)-5-(1H-imidazol-1-yl)pyrazolo[1,5-a]pyrimidin-3-yl)methylene)pyrrolidine-2,5-dione. LCMS (M+1=350)

### Example 170

### Synthesis of (E)-1-(4-(cyclopropylamino)-8-((2,5-dioxopyrrolidin-3-ylidene)methyl)pyrazolo[1,5-a][1,3,5]triazin-2-yl)-1H-benzo[d]imidazole-5-carboxylic acid

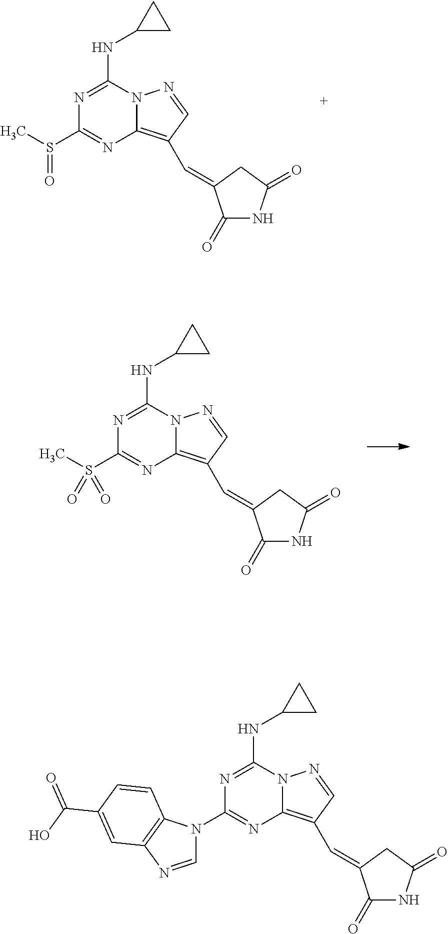

To a mixture of (E)-3-(4-(cyclopropylamino)-2-(methylsulfinyl)pyrazolo[1,5-a][1,3,5]triazin-8-yl)methylene)pyrrolidine-2,5-dione and (E)-3-(4-(cyclopropylamino)-2-(methylsulfonyl)pyrazolo[1,5-a][1,3,5]triazin-8-yl)methylene)pyrrolidine-2,5-dione (30 mg, 0.084 mmol) in 2.5 mL isopropanol was added 1H-benzo[d]imidazole-5-carboxylic acid (54 mg, 0.336 mmol) and the reaction mixture heated in MW at 140° C. for 20 minutes. Remove excess isopropanol on rotory evaporator and continue on to next step without further purification. LCMS (M+1=445)

### Example 171

### Synthesis of (E)-1-(4-(cyclopropylamino)-8-(2,5-dioxopyrrolidin-3-ylidene)methyl)pyrazolo[1,5-a][1,3,5]-triazin-2-yl)-N-(2-(dimethylamino)ethyl)-1H-benzo[d]imidazole-5-carboxamide

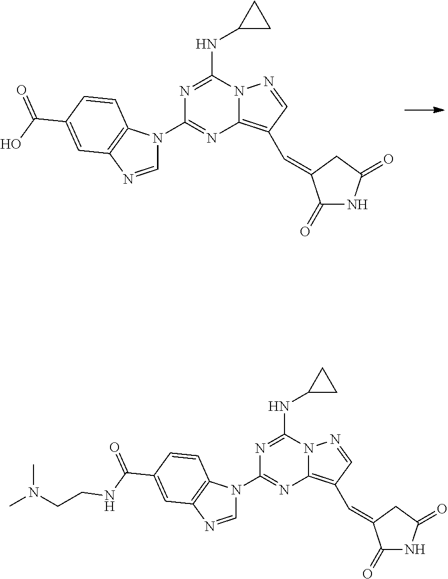

To (E)-1-(4-(cyclopropylamino)-8-(2,5-dioxopyrrolidin-3-ylidene)methyl)pyrazolo[1,5-a][1,3,5]triazin-2-yl)-1H-benzo[d]imidazole-5-carboxylic acid (7 mg, 0.016 mmol) in 1.5 mL DMF was added EDCI (64 mg, 0.334 mmol), HOBt (46 mg, 0.340 mmol), and N,N-dimethylethane-1,2-diamine (30 mg, 0.33 mmol). The reaction mixture was stirred at 50° C. for 16 h. Filtered through PTFE filter and purify by mass-directed prep LC/MS to provide (E)-1-(4-(cyclopropylamino)-8-(2,5-dioxopyrrolidin-3-ylidene)methyl)pyrazolo[1,5-a][1,3,5]triazin-2-yl)-N-(2-(dimethylamino)ethyl)-1H-benzo[d]imidazole-5-carboxamide as the TFA salt. LCMS (M+1=515)

### Example 172

### Synthesis of (E)-1-(4-(cyclopropylamino)-8-((2,5-dioxopyrrolidin-3-ylidene)methyl)pyrazolo[1,5-a][1,3,5]-triazin-2-yl)-N-(3-(pyrrolidin-1-yl)propyl)-1H-benzo[d]imidazole-5-carboxamide

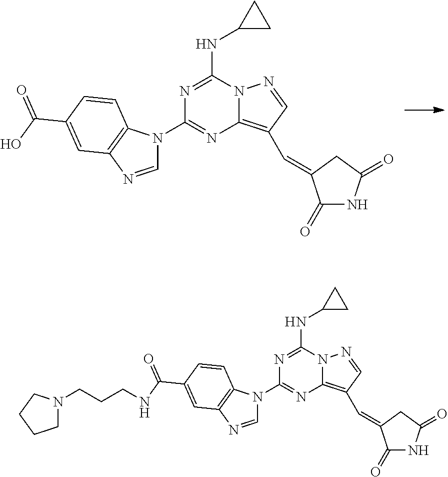

Same procedure as Example 166. LCMS (M+1=555)

### Example 173

### Synthesis of (E)-3-((4-(cyclopropylamino)-2-(5-(3-(dimethylamino)pyrrolidine-1-carbonyl)-1H-benzo[d]imidazol-1-yl)pyrazolo[1,5-a][1,3,5]triazin-8-yl)methylene)pyrrolidine-2,5-dione

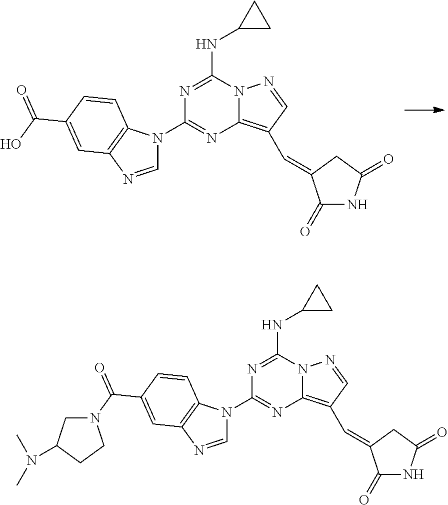

Same procedure as Example 166. LCMS (M+1=541)

### Example 174

### Synthesis of (E)-3-((4-(cyclopropylamino)-2-(5-(4-ethylpiperazine-1-carbonyl)-1H-benzo[d]imidazol-1-yl)pyrazolo[1,5-a][1,3,5]triazin-8-yl)methylene)pyrrolidine-2,5-dione

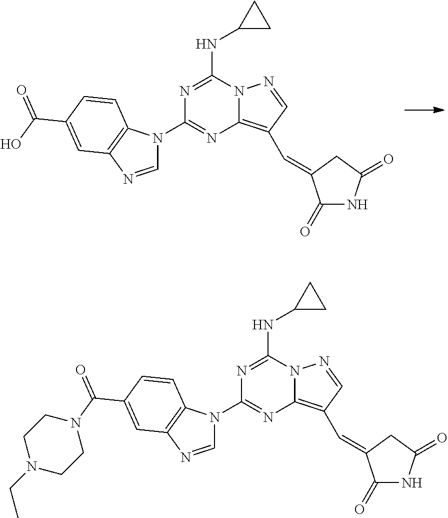

Same procedure as Example 166. LCMS (M+1=541)

### Example 175

### Synthesis of (E)-1-(4-(cyclopropylamino)-8-(2,5-dioxopyrrolidin-3-ylidene)methyl)pyrazolo[1,5-a][1,3,5]-triazin-2-yl)-N-(2-morpholinoethyl)-1H-benzo[d]imidazole-5-carboxamide

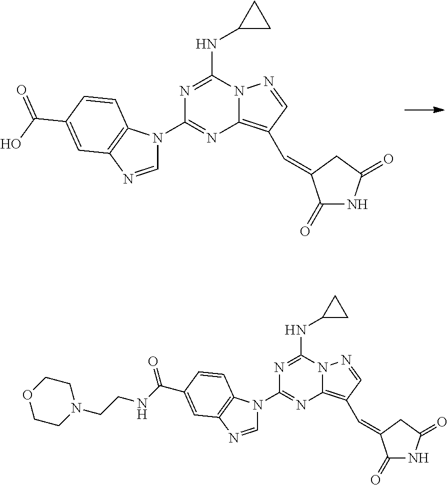

Same procedure as Example 166. LCMS (M+1=557)

### Example 176

### Synthesis of (E)-3-((4-(cyclopropylamino)-2-(dicyclopropylmethylamino)pyrazolo[1,5-a][1,3,5]triazin-8-yl)methylene)pyrrolidine-2,5-dione

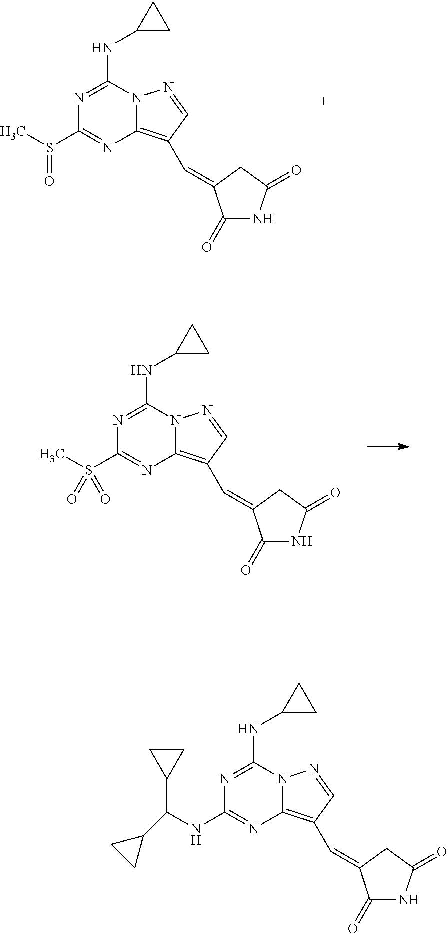

To a mixture of (E)-3((4-(cyclopropylamino)-2-(methylsulfinyl)pyrazolo[1,5-a][1,3,5]triazin-8-yl)methylene)pyrrolidine-2,5-dione and (E)-3-β4-(cyclopropylamino)-2-(methylsulfonyl)pyrazolo[1,5-a][1,3,5]triazin-8-yl)methylene)pyrrolidine-2,5-dione (10 mg, 0.028 mmol) in 1 mL of NMP was added dicyclopropylmethanamine (9 mg, 0.081 mmol) The reaction mixture was stirred at 70° C. for 3 h. Filtered and purified by mass-directed LC/MS to provide (E)-3-((4-(cyclopropylamino)-2-(dicyclopropylmethylamino)pyrazolo[1,5-a][1,3,5]triazin-8-yl)methylene)pyrrolidine-2,5-dione as the TFA salt. LCMS (M+1=394)

Examples 177 to 181 were prepared by using the procedures as described above including General Methods, Schems 1 to 3.

### Example 177

### Synthesis of (E)-2-(4-(cyclopropylamino)-8-((2,5-dioxopyrrolidin-3-ylidene)methyl)pyrazolo[1,5-a][1,3,5]triazin-2-ylamino)-2-phenylacetonitrile

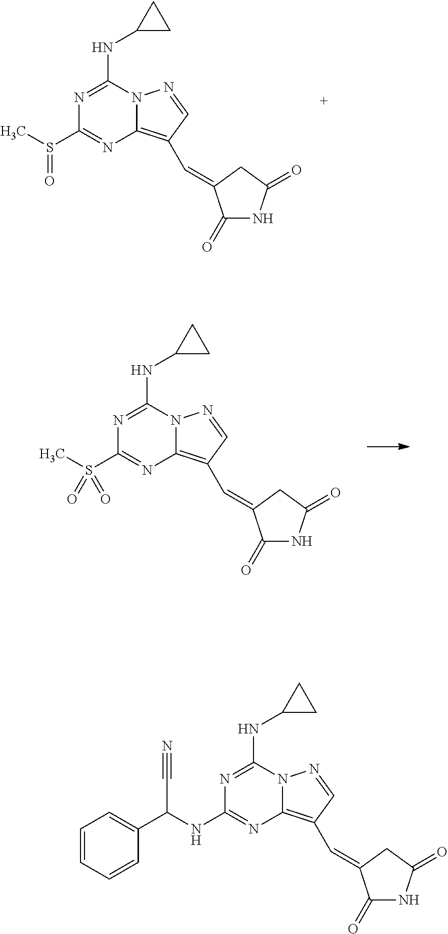

LCMS (M+1=415)

### Example 178

### Synthesis of (E)-3-((4-(cyclopropylamino)-2-(morpholino(phenyl)methylamino)pyrazolo[1,5-a][1,3,5]triazin-8-yl)methylene)pyrrolidine-2,5-dione

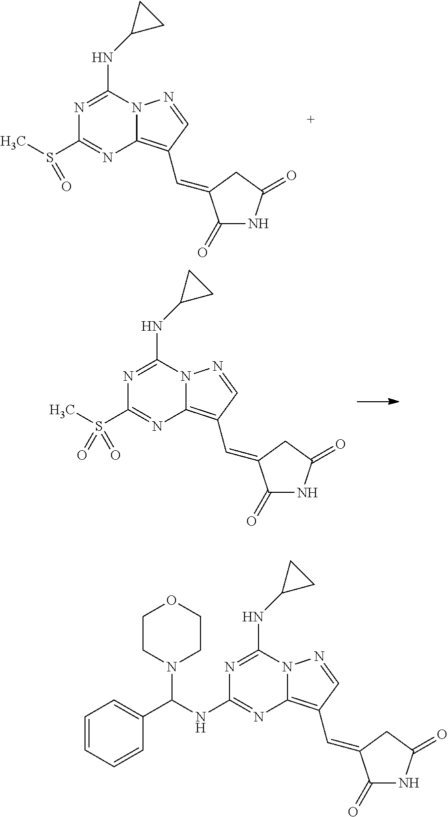

LCMS (M+1=475)

### Example 179

### Synthesis of (E)-3-((2-(dicyclopropylmethylamino)-4-(phenylamino)pyrazolo[1,5-a][1,3,5]triazin-8-yl)methylene)pyrrolidine-2,5-dione

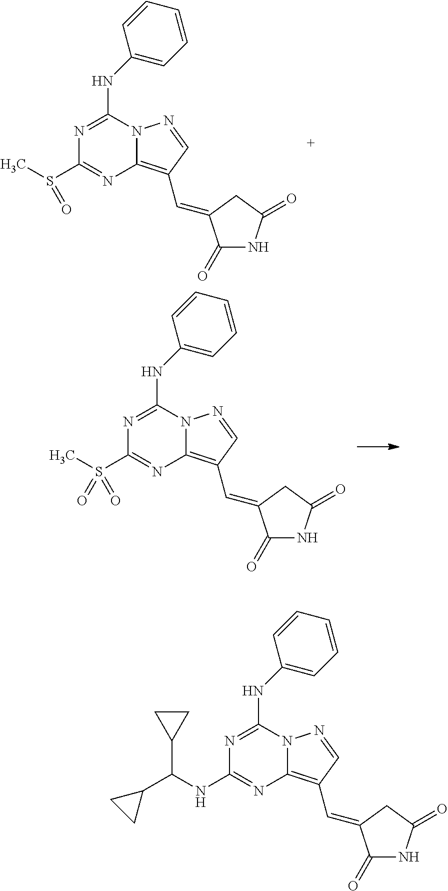

LCMS (M+1=430)

### Example 180

### Synthesis of (E)-2-(8-((2,5-dioxopyrrolidin-3-ylidene)methyl)-4-(phenylamino)pyrazolo[1,5-a][1,3,5]triazin-2-ylamino)-2-phenylacetonitrile

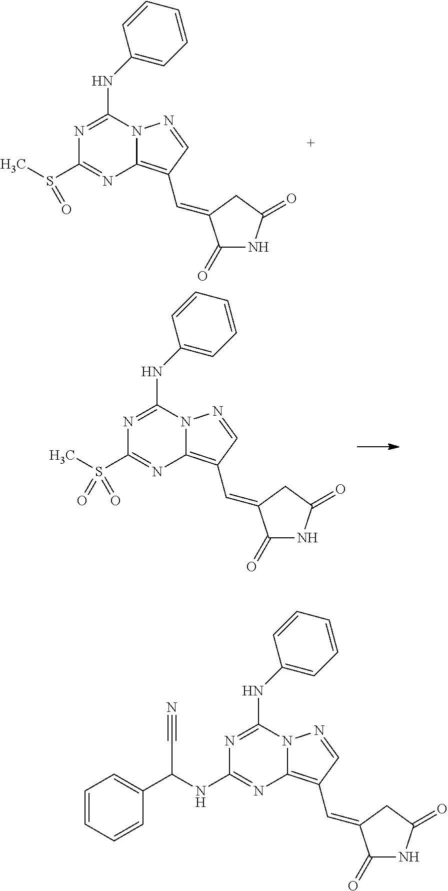

LCMS (M+1=451)

### Example 181

### Synthesis of (E)-3-((2-(morpholino(phenyl)methylamino)-4-(phenylamino)pyrazolo[1,5-a][1,3,5]triazin-8-yl)methylene)pyrrolidine-2,5-dione

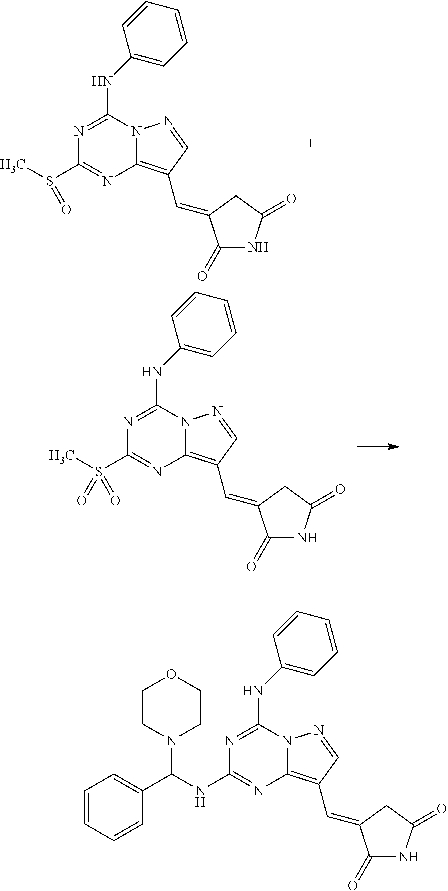

LCMS (M+1=511)

### Example 182

### Synthesis of 3-((5-(4-(1H-pyrazol-1-yl)phenylamino)-7-(cyclopropylamino)pyrazolo[1,5-a]pyrimidin-3-yl)methyl)pyrrolidine-2,5-dione

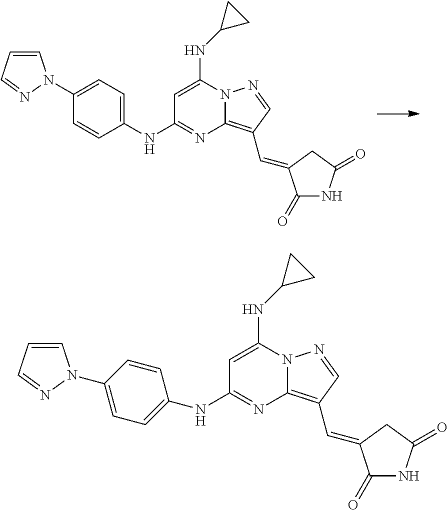

To a suspension of (E)-3-((5-(4-(1H-pyrazol-1-yl)phenylamino)-7-(cyclopropylamino)pyrazolo[1,5-a]pyrimidin-3-yl)methylene)pyrrolidine-2,5-dione (80 mg, 0.181 mmol) in acetic acid (8 mL) was added 40 mg of 10% Pd/C. Shake on Parr shaker at 60 psi for 7 days. Filter through a pad of celite and purify by mass-directed prep LC/MS to provide 3-((5-(4-(1H-pyrazol-1-yl)phenylamino)-7-(cyclopropylamino)pyrazolo[1,5-a]pyrimidin-3-yl)methyl)pyrrolidine-2,5-dione as the trifluoroacetic acid salt. LCMS (M+1=443)

### Example P1

### Synthesis of (E)-3-((5-(5-chloro-2-fluorophenylamino)-7-(cyclopropylamino)pyrazolo[1,5-a]pyrimidin-3-yl)methylene)-1-(hydroxymethyl)pyrrolidine-2,5-dione

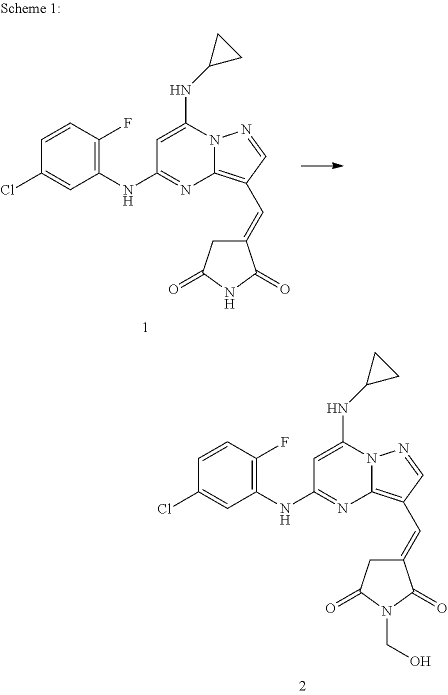

Compound 2 can be prepared from (E)-3-((5-(5-chloro-2-fluorophenylamino)-7-(cyclopropylamino)pyrazolo[1,5-a]pyrimidin-3-yl)methylene)pyrrolidine-2,5-dione and formaldehyde (Scheme 1) as described in U.S. Pat. No. 4,260,769. For example, 1 (2.0 g) can be treated with 8 mL of formalin in 70 mL of water and potassium carbonate (0.1 eq). The reaction can be stirred at room temperature for an appropriate amount of time between 2 hours and 24 hours. The product can be filtered off and washed with water.

### Example P2

### Synthesis of (E)-5-(3-((5-(5-chloro-2-fluorophenylamino)-7-(cyclopropylamino)pyrazolo[1,5-a]pyrimidin-3-yl)methylene)-2,5-dioxopyrrolidin-1-yl)methoxy)-5-oxopentanoic acid

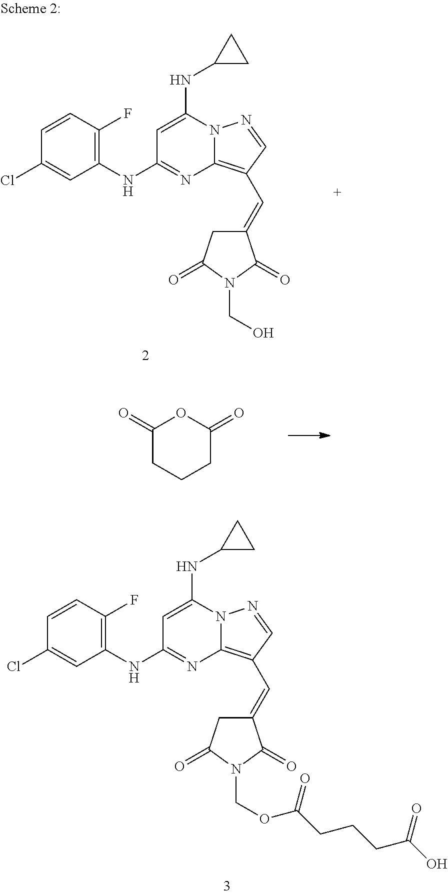

Compound 3 can be prepared from compound 2 and glutaric anhydride (Scheme 2) as described in U.S. Pat. No. 4,260,769. For example, compound 2 (1.0 g) in an appropriate solvent such as pyridine can be treated with glutaric anhydride (1.2 eq.) and stirred for an appropriate time between 2 hours and 5 days at room temperature, thus obtaining the desired compound.

### Example P3

### Synthesis of (E)-(3-((5-(5-chloro-2-fluorophenylamino)-7-(cyclopropylamino)pyrazolo[1,5-a]pyrimidin-3-yl)methylene)-2,5-dioxopyrrolidin-1-yl)methyl 3-(4-methylpiperazin-1-yl)propanoate

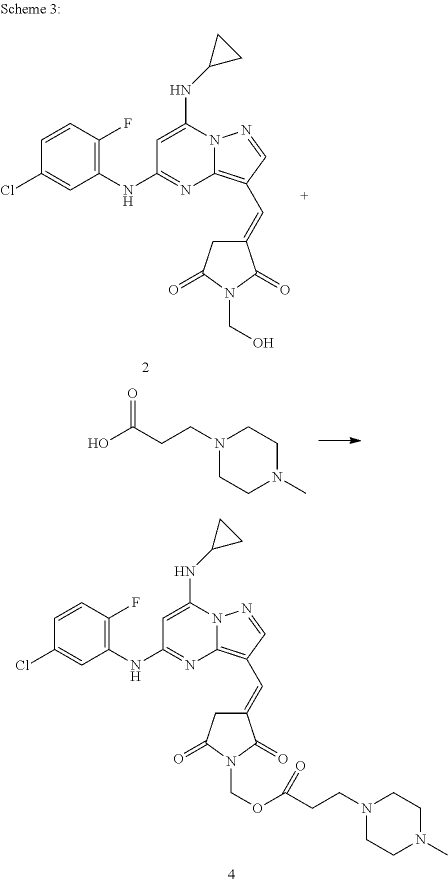

Compound 4 can be prepared from compound 2 and 3-(4-methylpiperazin-1-yl)propanoic acid as described in the literature (U.S. Pat. No. 4,260,769). For example, 2 (1.0 g) in an appropriate solvent such as pyridine can be treated with 3-(4-methylpiperazin-1-yl)propanoic acid (1.0 eq.) and dicyclohexylcarbodiimide (1.0 eq.) in the presence of DMAP and stirred for an appropriate amount of time between 2 hours and 24 hours after which the final product is obtained.

### Example P4

### Synthesis of (E)-(3-((5-(5-chloro-2-fluorophenylamino)-7-(cyclopropylamino)pyrazolo[1,5-a]pyrimidin-3-yl)methylene)-2,5-dioxopyrrolidin-1-yl)methyl ethyl carbonate

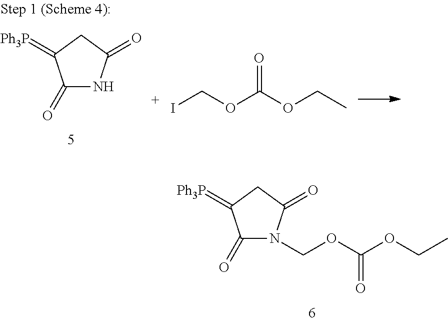

Compound 6 can be prepared by treating compound 5 (20 mg) with sodium hydride(1.2 eq.) in an appropriate solvent such as DMF and stirring at room temperature for 1 minute followed by treatment of ethyl iodomethyl carbonate (1.5 eq.). This can be stirred for an appropriate amount of time between 10 minutes and 24 hours after which the desired compound can be obtained.

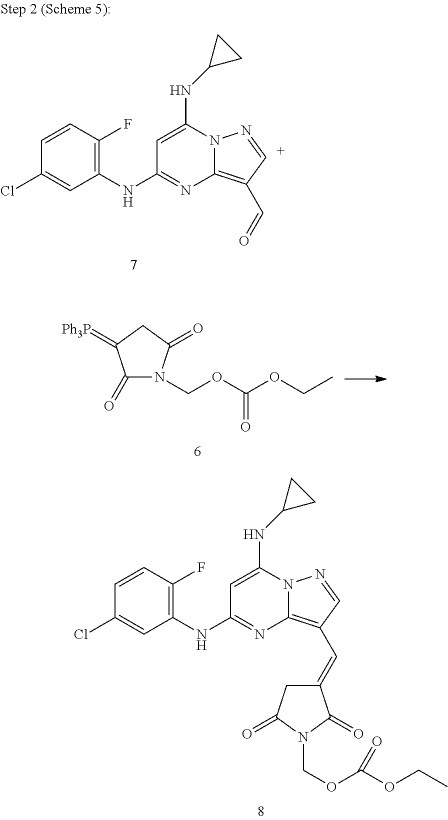

(E)-(3-((5-(5-chloro-2-fluorophenylamino)-7-(cyclopropylamino)pyrazolo[1,5-a]pyrimidin-3-yl)methylene)-2,5-dioxopyrrolidin-1-yl)methyl ethyl carbonate can be prepared by treating 5-(5-chloro-2-fluorophenylamino)-7-(cyclopropylamino)pyrazolo[1,5-a]pyrimidine-3-carbaldehyde (100 mg) with compound 6 (1.0 eq.) in an appropriate solvent such as ethanol and stirring at reflux temperature for an appropriate amount of time between 1 hour and 24 hours after which the resulting solid can be filtered off and washed with water and ethanol.

### Synthesis of (E)-(3-((5-(5-chloro-2-fluorophenylamino)-7-(cyclopropylamino)pyrazolo[1,5-a]pyrimidin-3-yl)methylene)-2,5-dioxopyrrolidin-1-yl)methyl dihydrogen phosphate

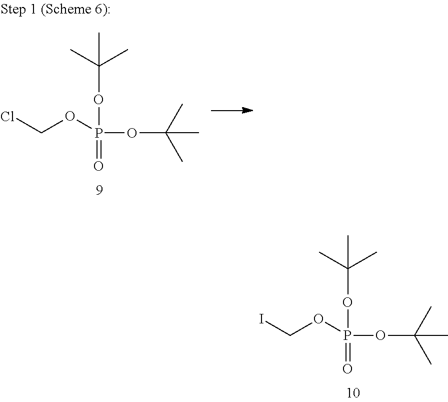

Di-tert-butyl iodomethyl phosphate can be prepared by treating di-tert-butyl chloromethyl phosphate (500 mg) with NaI (1.2 eq.) in an appropriate solvent such as acetone and stirring at reflux temperature for a period of between 4 hours and 24 hours after which the desired product can be isolated by removing excess acetone and performing an extraction from water and diethyl ether.

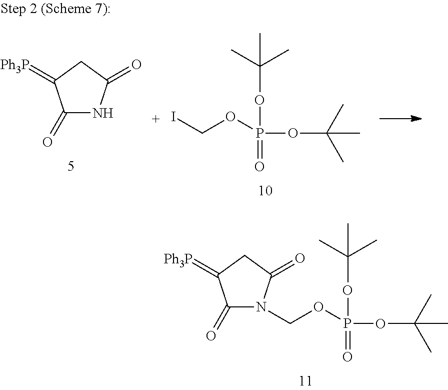

Compound 11 can be prepared by treating compound 5 (500 mg) with sodium hydride (1.1 eq.) in an appropriate solvent such as DMF at room temperature for a period of between 1 minute and 30 minutes followed by treatment of di-tert-butyl iodomethyl phosphate (1.2 eq.). The reaction mixture can be stirred at room temperature for a period of between 1 hour and 24 hours after which the desired compound is obtained.

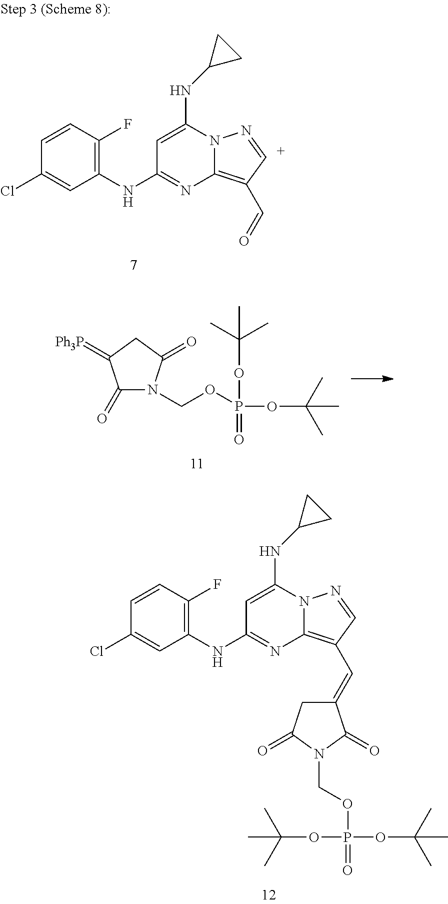

(E)-di-tert-butyl (3-((5-(5-chloro-2-fluorophenylamino)-7-(cyclopropylamino)pyrazolo[1,5-a]pyrimidin-3-yl)methylene)-2,5-dioxopyrrolidin-1-yl)methyl phosphate can be prepared by treating 5-(5-chloro-2-fluorophenylamino)-7-(cyclopropylamino)pyrazolo[1,5-a]pyrimidine-3-carbaldehyde (200 mg) with compound 11 (1.2 eq.) in an appropriate solvent such as ethanol and stirring at reflux temperature for a period of between 1 hour and 24 hours after which the resulting solid can be cooled to room temperature and filtered off and rinsed with water and ethanol.

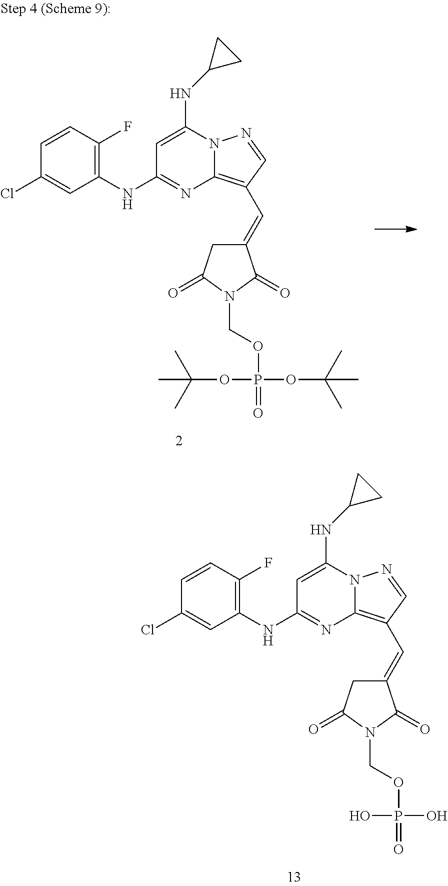

(E)-(3-((5-(5-chloro-2-fluorophenylamino)-7-(cyclopropylamino)pyrazolo[1,5-a]pyrimidin-3-yl)methylene)-2,5-dioxopyrrolidin-1-yl)methyl dihydrogen phosphate can be prepared by treating (E)-di-tert-butyl (3-((5-(5-chloro-2-fluorophenylamino)-7-(cyclopropylamino)pyrazolo[1,5-a]pyrimidin-3-yl)methylene)-2,5-dioxopyrrolidin-1-yl)methyl phosphate (100 mg) in 4M HCl/dioxane and stirring at room temperature for a period of between 1 hour and 24 hours after which the resulting precipitate formed can be filtered off and washed with water.

### Example P5

### Synthesis of (E)-4-((3-((5-(5-chloro-2-fluorophenylamino)-7-(cyclopropylamino)pyrazolo[1,5-a]pyrimidin-3-yl)methylene)-2,5-dioxopyrrolidin-1-yl)methoxy)-4-oxobutanoic acid

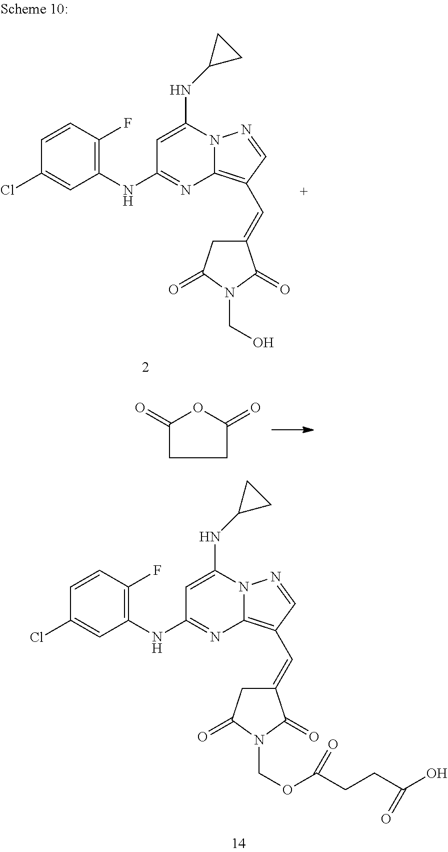

Compound 14 can be prepared from compound 2 and succinic anhydride (Scheme 10) as described in US 2004/0152905. For example, compound 2 (1.0 mmol) in an appropriate solvent such as pyridine can be treated with succinic anhydride (1.2 mmol) in the presence of 4-dimethylaminopyridine and stirred for an appropriate time between 2 hours and 5 days at room temperature, thus obtaining the desired compound.

### Example P6

### Synthesis of (E)-(3-((5-(5-chloro-2-fluorophenylamino)-7-(cyclopropylamino)pyrazolo[1,5-a]pyrimidin-3-yl)methylene)-2,5-dioxopyrrolidin-1-yl)methyl dihydrogen phosphate

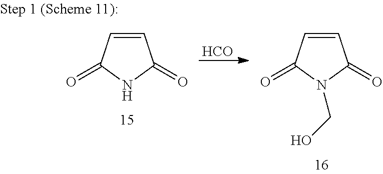

1-hydroxymethyl-pyrrole 16 can be prepared from maleimide 15 and fromaldehyde (Scheme 11) as described in the literature (e.g., U.S. Pat. No. 2,526,517, US 2006/128943 and US 2004/34011). For example, maleimide (2.0 g) can be treated with 10 percent formaline (6.8 g) at an appropriate temperature to give product 16.

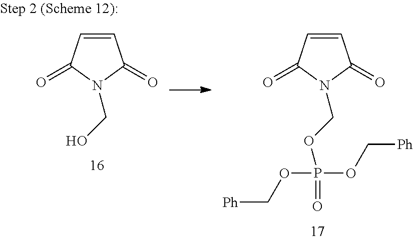

The synthesis of compound 17 can be achieved as described in WO 2006/086484. For example, compound 16 can be treated in an appropriate solvent such as anhydrous tetrahydrofuran with dibenzylphosphoramidate (3.5 equivalents) followed by the addition of tetrazole (3% solution in acetonitrile). The mixture can be stirred at an appropriate temperature. The workup can be done as described in WO 2006/086484.

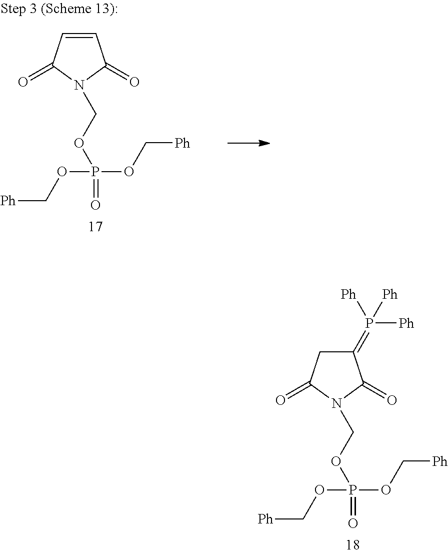

Compound 18 can be prepared by treating compound 17 with triphenylphosphine in an appropriate solvent such as acetone. The reaction can be heated at reflux to give product 18.

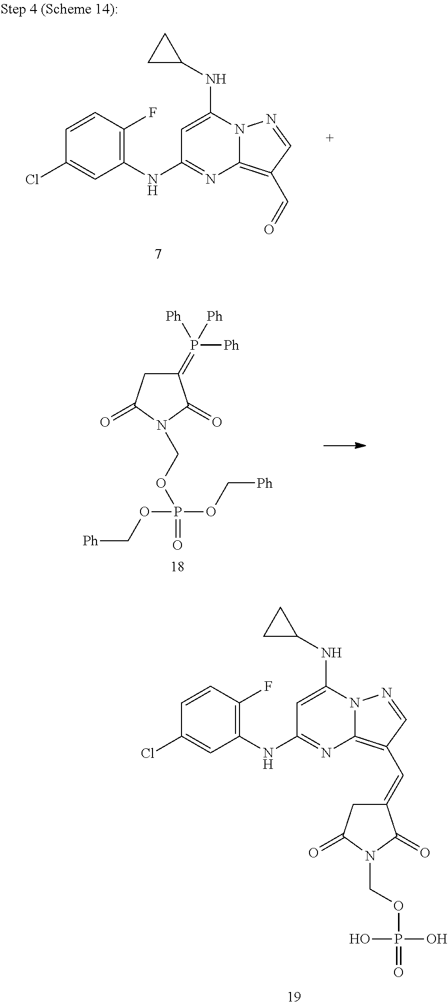

Compound 19 can be prepared by aldehyde 7 with compound 18 (1.2 eq.) in an appropriate solvent such as ethanol and stirring at reflux temperature for a period of between 1 hour and 24 hours after which the resulting intermediate can be treated with Pd/C (10%) in an appropriate solvent such methanol at room temperature under an appropriate pressure such as 1 atmosphere of hydrogen to give product 19.

### Example P7

### Synthesis of (E)-(3-((5-(5-chloro-2-fluorophenylamino)-7-(cyclopropylamino)pyrazolo[1,5-a]pyrimidin-3-yl)methylene)-2,5-dioxopyrrolidin-1-yl)methy 2-aminopropanoate

Compound 21 can be prepared from compound 2 in 2 steps as described in WO2006/086484.

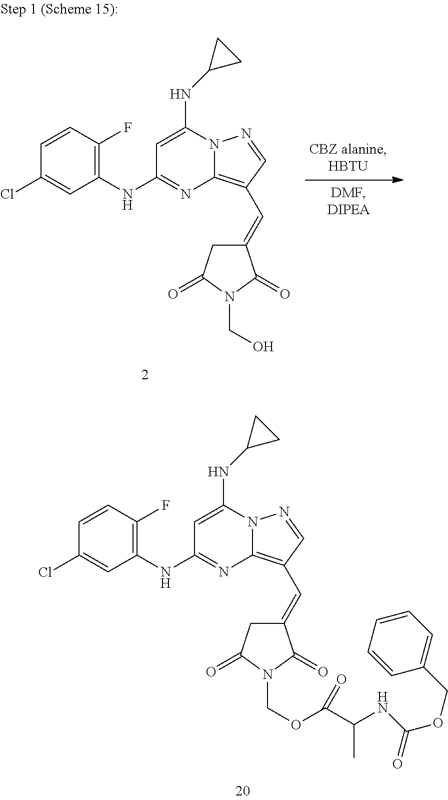

Compound 20 can be prepared by treating compound 2 with carbobenzyloxy aniline in the presence of HBTU and DIPEA in DMF. The mixture can be stirred at room temperature to give product after an appropriate workup.

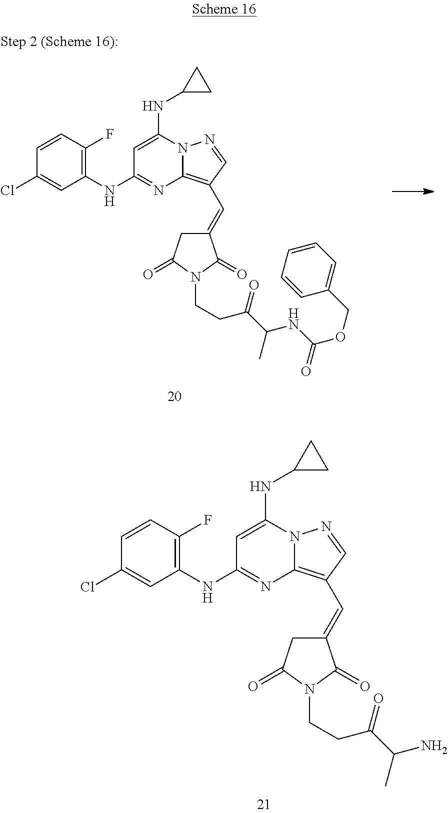

Compound 21 can be prepared by treating compound 20 with 10% Pd/C in an appropriate solvent such methanol at room temperature under an appropriate pressure such as 1 atmosphere of hydrogen to give product 21.

### Biological Test Methods

### Example 183

### CK2 Assay Method

Modulatory activity of compounds described herein was assessed in vitro in cell-free CK2 assays by the following method.

Test compounds in aqueous solution were added at a volume of 10 microliters, to a reaction mixture comprising 10 microliters Assay Dilution Buffer (ADB; 20 mM MOPS, pH 7.2, 25 mM beta-glycerolphosphate, 5 mM EGTA, 1 mM sodium orthovanadate and 1 mM dithiothreitol), 10 microliters of substrate peptide (RRRDDDSDDD, dissolved in ADB at a concentration of 1 mM), 10 microliters of recombinant human CK2 (25 ng dissolved in ADB; Upstate). Reactions were initiated by the addition of 10 microliters of ATP Solution (90% 75 mM MgCl₂, 75 micromolar ATP dissolved in ADB; 10% [γ-³³P]ATP (stock 1 mCi/100 μl; 3000 Ci/mmol (Perkin Elmer) and maintained for 10 minutes at 30° C. The reactions were quenched with 100 microliters of 0.75% phosphoric acid, then transferred to and filtered through a phosphocellulose filter plate (Millipore). After washing each well 5 times with 0.75% phosphoric acid, the plate was dried under vacuum for 5 min and, following the addition of 15 ul of scintilation fluid to each well, the residual radioactivity was measured using a Wallac luminescence counter.

### Example 184

### Pim-1 Assay Method

The following procedure was used to assay the Pim-1 kinase activity of compounds of the invention. Other methods for assaying Pim-1 and other Pim kinases, as well as methods to assay for activity against the various kinases disclosed herein are known in the art.

In a final reaction volume of 50 ul, recombinant Pim-1 (1 ng) was incubated with 12 mM MOPS pH 7.0, 0.4 mM EDTA, glycerol 1%, brij 35 0.002%, 2-mercaptoethanol 0.02%, BSA 0.2 mg/ml, 100 uM KKRNRTLTK, 10 mM MgAcetate, 15 uM ATP, [γ-³³P-ATP] (specific activity approx. 500 cpm/μmol), DMSO 4% and test inhibitor compound at the required concentration. The reaction was initiated by the addition of the magnesium ATP mixture. After 40 min incubation at 23° C., the reactions were quenched by the addition of 100 ul 0.75% Phosphoric acid, and the labeled peptide collected by filtration through a phosphocellulose filter plate. The plate was washed 4 times with 0.075% phosphoric acid (100 ul per well) and then, after the addition of scintillation fluid (20 ul per well), the counts were measured by a scintillation counter.

### Example 185

### PIM-2 Assay Method

Test compounds dissolved and diluted in DMSO (2 μl) were added to a reaction mixture comprising 10 μl of 5× Reaction Buffer (40 mM MOPS pH 7.0, 5 mM EDTA), 10 of recombinant human Pim-2 solution (4 ng Pim-2 dissolved in dilution buffer (20 mM MOPS pH 7.0; EDTA 1 mM; 5% Glycerol; 0.01% Brij 35; 0.1%; 0.1% 2-mercaptoethanol; 1 mg/ml BSA)) and 8 ul of water. Reactions were initiated by the addition of 10 ul of ATP Solution (49% (15 mM MgCl₂; 75 uM ATP) 1% ([γ-33P]ATP: Stock 1 mCi/100 μl; 3000 Ci/mmol (Perkin Elmer)) and 10 ul of substrate peptide solution (RSRSSYPAGT, dissolved in water at a concentration of 1 mM), Reactions were maintained for 10 mM at 30° C. The reactions were quenched with 100 ul of 0.75% phosphoric acid, then transferred to and filtered through a Phosphocellulose filter plate (Millipore, MSPH-N6B-50). After washing each well 4 times with 0.75% phosphoric acid, scintillation fluid (20 μL) was added to each well and the residual radioactivity was measured using a Wallac luminescence counter.

### Example 186

### Cell Proliferation Modulatory Activity

A representative cell-proliferation assay protocol using Alamar Blue dye (stored at 4° C., use 20 ul per well) is described hereafter.

### 96-Well Plate Setup and Compound Treatment

a. Split and trypsinize cells.

b. Count cells using hemocytometer.

c. Plate 4,000-5,000 cells per well in 100 μl of medium and seed into a 96-well plate according to the following plate layout. Add cell culture medium only to wells B10 to B12. Wells B1 to B9 have cells but no compound added.

| | 1 2 3 | 4 5 6 | 7 8 9 | 10 11 12 | |
|---|---|---|---|---|---|
| A | EMPTY | | | | |
| B | NO COMPOUND ADDED | | | Medium | |
| | | | | Only | |
| C | 10 nM | 100 nM | 1 uM | 10 uM | Control |
| D | 10 nM | 100 nM | 1 uM | 10 uM | Comp1 |
| E | 10 nM | 100 nM | 1 uM | 10 uM | Comp2 |
| F | 10 nM | 100 nM | 1 uM | 10 uM | Comp3 |
| G | 10 nM | 100 nM | 1 uM | 10 uM | Comp4 |

| | |
|---|---|
| H | EMPTY |

d. Add 100 μl of 2× drug dilution to each well in a concentration shown in the plate layout above. At the same time, add 100 μl of media into the control wells (wells B10 to B12). Total volume is 200 μl/well.

e. Incubate four (4) days at 37° C., 5% CO₂ in a humidified incubator.

f. Add 20 μl Alamar Blue reagent to each well.

g. Incubate for four (4) hours at 37° C., 5% CO₂ in a humidified incubator.

h. Record fluorescence at an excitation wavelength of 544 nm and emission wavelength of 590 nm using a microplate reader.

In the assays, cells are cultured with a test compound for approximately four days, the dye is then added to the cells and fluorescence of non-reduced dye is detected after approximately four hours. Different types of cells can be utilized in the assays (e.g., HCT-116 human colorectal carcinoma cells, PC-3 human prostatic cancer cells, MDA-MB231 human breast cancer cells, K-562 human chronic myelogenous leukemia (CML) cells, MiaPaca human pancreatic carcinoma cells, MV-4 human acute myeloid leukemia cells, and BxPC3 human pancreatic adenocarcinoma cells).

Various compounds of the invention were tested in bioassay for enzyme inhibition and cell growth inhibition. These tested compounds showed desirable biological activity to inhibit one or more of the following enzymes or cells: CK2 IC₅₀ (μM), PIM2 percent inhibition (2.5 μM), AB: MDAMB453 IC₅₀ (μM), and AB: BxPC3 IC₅₀ (μM). For example, all of the tested compounds showed CK2 IC50 of less than 50 uM; some of the tested compounds showed CK2 IC50 of less than 30 uM; some of the tested compounds showed CK2 IC50 of less than 20 uM; some of the tested compounds showed CK2 IC50 of less than 10 uM; some of the tested compounds showed CK2 IC50 of less than 5 uM; some of the tested compounds showed CK2 IC50 of less than 2.5 uM; some of the tested compounds showed CK2 IC50 of less than 1 uM; some of the tested compounds showed CK2 IC50 of less than 0.5 uM; and some of the tested compounds showed CK2 IC50 of less than 0.1 uM. Furthermore, all of the tested compounds showed PIM2 percent inhibition (2.5 μM) in a range from about −30% to about 99%; some of the tested compounds showed PIM2 percent inhibition (2.5 μM) in a range from about 5% to about 99%; some of the tested compounds showed PIM2 percent inhibition (2.5 μM) in a range from about 10% to about 99%; some of the tested compounds showed PIM2 percent inhibition (2.5 μM) in a range from about 20% to about 99%; some of the tested compounds showed PIM2 percent inhibition (2.5 μM) in a range from about 30% to about 99%; and some of the tested compounds showed PIM2 percent inhibition (2.5 μM) in a range from about 50% to about 99%. Moreover, all of the tested compounds showed AB: MDAMB453 IC50 (μM) and/or AB: BxPC3 IC50 (μM) of less than 100 uM; some of the tested compounds showed AB: MDAMB453 IC50 (μM) and/or AB: BxPC3 IC₅₀ (μM) of less than 75 uM; some of the tested compounds showed AB: MDAMB453 IC50 (μM) and/or AB: BxPC3 IC₅₀ (μM) of less than 50 uM; some of the tested compounds showed AB: MDAMB453 IC50 (μM) and/or AB: BxPC3 IC₅₀ (μM) of less than 40 uM; some of the tested compounds showed AB: MDAMB453 IC50 (μM) and/or AB: BxPC3 IC₅₀ (μM) of less than 30 uM; some of the tested compounds showed AB: MDAMB453 IC50 (μM) and/or AB: BxPC3 IC₅₀ (μM) of less than 20 uM; some of the tested compounds showed AB: MDAMB453 IC₅₀ (μM) and/or AB: BxPC3 IC₅₀ (μM) of less than 10 uM; some of the tested compounds showed AB: MDAMB453 IC₅₀ (μM) and/or AB: BxPC3 IC₅₀ (μM) of less than 5 uM; some of the tested compounds showed AB: MDAMB453 IC50 (μM) and/or AB: BxPC3 IC₅₀ (μM) of less than 2 uM; and some of the tested compounds showed AB: MDAMB453 IC50 (μM) and/or AB: BxPC3 IC₅₀ (μM) of less than 1 uM.

Biological activities for various compounds are summarized in the following tables, wherein Compounds A1 to T1 are Examples and specific compounds (i.e., species) as described herein above. For example, Compound A12 is described above as Example 25.

TABLE A

| | CK2: | PIM2: | AB: | AB: |
|---|---|---|---|---|
| | IC50 | % inh | MDAMB453: | BxPC3: |
| Compound | (μM) | 2.5 uM | IC50 (μM) | IC50 (μM) |
| A1 | <1 | | | |
| A2 | <1 | | >30 | >30 |
| A3 | >1 | | >30 | >30 |
| A4 | >1 | | >30 | >30 |
| A5 | >1 | | >30 | >30 |
| A6 | >1 | | 13.445 | 18.396 |
| A7 | >1 | | 14.089 | >30 |
| A8 | <1 | | >30 | >30 |
| A9 | <1 | | >30 | >30 |
| A10 | >1 | | >30 | >30 |
| A11 | >1 | | >30 | >30 |
| A12 | <1 | 6.72 | 6.09 | 11.335 |
| A13 | <1 | 14.975 | 2.268 | >30 |
| A14 | <1 | 48.772 | | |

TABLE B

| | CK2: | PIM2: | AB: | AB: |
|---|---|---|---|---|
| | IC50 | % inh | MDAMB453: | BxPC3: |
| Compound | (μM) | 2.5 uM | IC50 (μM) | IC50 (μM) |
| B1 | <1 | 35.971 | | |
| B2 | <1 | 41.48 | | |
| B3 | >1 | 7.979 | | |
| B4 | >1 | 25.31 | | |
| B5 | >1 | 17.288 | | |
| B6 | >1 | 19.107 | | |
| B7 | >1 | 71.946 | | |
| B8 | <1 | 55.844 | | |
| B9 | <1 | 37.964 | | |
| B10 | <0.1 | 42.771 | | |
| B11 | <1 | 70.751 | | |
| B12 | <0.1 | 42.346 | | |
| B13 | <0.1 | 46.19 | | |
| B14 | <1 | 51.629 | | |
| B15 | <0.1 | 55.895 | | |
| B16 | <0.1 | 34.817 | | |
| B17 | <1 | 54.704 | | |
| B18 | <1 | 56.592 | | |
| B19 | <0.1 | 49.988 | | |
| B20 | <1 | 52.641 | | |
| B21 | <1 | 50.973 | | |
| B22 | <0.1 | 18.079 | | |

TABLE C

| | CK2: | PIM2: | AB: | AB: |
|---|---|---|---|---|
| | IC50 | % inh | MDAMB453: | BxPC3: |
| Compound | (μM) | 2.5 uM | IC50 (μM) | IC50 (μM) |
| C1 | <1 | 38.074 | | |
| C2 | <1 | 59.301 | | |
| C3 | >1 | 37.241 | | |

TABLE D

| | | PIM2: | AB: | |
|---|---|---|---|---|
| | CK2: IC50 | % inh | MDAMB453: | AB: BxPC3: |
| Compound | (μM) | 2.5 uM | IC50 (μM) | IC50 (μM) |
| D1 | <0.01 | 37.032 | 2.887 | 3.221 |
| D2 | <1 | | | |
| D3 | <1 | | | |
| D4 | <0.1 | | 9.52 | 13.579 |
| D5 | <0.01 | 27.902 | 2.115 | 7.724 |
| D6 | <0.1 | | 6.459 | 16.608 |
| D7 | <0.1 | | | |
| D8 | <0.01 | | 7.523 | 11.764 |
| D9 | <0.01 | 27.619 | 1.368 | 17.281 |
| D10 | <0.1 | | | |
| D11 | <0.1 | | | |
| D12 | <0.01 | 29.583 | 3.319 | 5.556 |
| D13 | <0.01 | 1.584 | 6.725 | >30 |
| D14 | <0.01 | 31.009 | 0.321 | 0.688 |
| D15 | <0.01 | −4.691 | 4.935 | 14.253 |
| D16 | <0.01 | 0.199 | 6.388 | 22.598 |
| D17 | <0.1 | 8.599 | 11.421 | 23.361 |
| D18 | <0.01 | 4.543 | 3.203 | 16.637 |
| D19 | <0.1 | 23.924 | | |
| D20 | <0.01 | 2.683 | 8.96 | 11.429 |
| D21 | <0.01 | 9.774 | 9.686 | 11.332 |
| D22 | <0.01 | −28.402 | 1.33 | 7.414 |
| D23 | <0.1 | −11.021 | 6.602 | 12.821 |
| D24 | <0.01 | 30.077 | 1.546 | 4.626 |
| D25 | <0.01 | 50.581 | 19.005 | 2.483 |
| D26 | <0.01 | 53.327 | 7.834 | >30 |
| D27 | <0.01 | 67.463 | 2.705 | 15.312 |
| D28 | <0.01 | 44.314 | 0.834 | 3.209 |
| D29 | <0.01 | 55.771 | 3.005 | 6.944 |
| D30 | <0.01 | 55.373 | 26.604 | 27.487 |
| D31 | <0.01 | 60.552 | 3.062 | 8.099 |
| D32 | <0.01 | 75.167 | 3.137 | 9.075 |
| D33 | <0.1 | 52.087 | 2.806 | 7.145 |
| D34 | <0.01 | 59.325 | 1.6 | 3.866 |
| D35 | <0.01 | 66.642 | 2.567 | >30 |
| D36 | <0.01 | 58.41 | 1.996 | 4.008 |
| D37 | <0.01 | 71.56 | 1.719 | 3.141 |
| D38 | <0.1 | 70.229 | | |
| D39 | <0.01 | 90.309 | 0.512 | 1.189 |
| D40 | <0.01 | 79.929 | >30 | >30 |

TABLE E

| | | PIM2: | AB: | |
|---|---|---|---|---|
| | CK2: IC50 | % inh | MDAMB453: | AB: BxPC3: |
| Compound | (μM) | 2.5 uM | IC50 (μM) | IC50 (μM) |
| E1 | <0.1 | −20.388 | | |
| E2 | <0.1 | 11.175 | 2.52 | 1.334 |
| E3 | <0.1 | 12.763 | 5.373 | 3.008 |
| E4 | <0.01 | −4.586 | 5.068 | 11.617 |
| E5 | <1 | 8.499 | | |
| E6 | <1 | −13.991 | | |
| E7 | <1 | 40.129 | | |
| E8 | <0.01 | −11.032 | 2.722 | 2.485 |
| E9 | <0.01 | 22.775 | 1.466 | 0.964 |
| E10 | <1 | 16.923 | | |
| E11 | <1 | −8.359 | | |
| E12 | <0.01 | 24.166 | 4.952 | 16.275 |
| E13 | <0.01 | 11.528 | 18.881 | >30 |
| E14 | <0.1 | 13.078 | 1.81 | 1.535 |
| E15 | <0.1 | 26.539 | 1.075 | 1.352 |

TABLE F

| | | PIM2: | AB: | |
|---|---|---|---|---|
| | CK2: IC50 | % inh | MDAMB453: | AB: BxPC3: |
| Compound | (μM) | 2.5 uM | IC50 (μM) | IC50 (μM) |
| F1 | <0.1 | | 5.974 | 5.58 |
| F2 | <1 | | 12.863 | 11.976 |
| F3 | <1 | | >30 | >30 |
| F4 | <1 | | 20.828 | 18.017 |
| F5 | <0.1 | | | |
| F6 | <1 | | | |
| F7 | <0.1 | 25.503 | | |

TABLE G

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| G1 | <0.1 | 98.417 | | |
| G2 | <0.1 | 44.148 | | |

TABLE J

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| J1 | <1 | −18.512 | | |
| J2 | <1 | −2.957 | | |

TABLE K

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| K1 | >1 | 57.659 | 11.411 | 15.623 |
| K2 | <1 | 31.497 | 19.745 | 19.304 |
| K3 | >1 | 90.716 | 18.767 | 6.294 |
| K4 | <0.1 | 5.859 | 4.036 | 1.724 |

TABLE L

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| L1 | <0.1 | −0.747 | | |
| L2 | <0.1 | 67.732 | | 8.253 |
| L3 | <1 | 44.372 | | |
| L4 | < | 48.587 | | |
| L5 | <1 | 41.004 | | |
| L6 | <0.1 | 20.99 | 25.199 | 12.997 |
| L7 | <0.1 | 15.588 | | |
| L8 | <0.1 | 4.632 | | |
| L9 | <0.01 | 23.077 | 17.488 | 15.286 |
| L10 | >1 | 2.716 | | |
| L11 | <0.1 | 39.417 | | |
| L12 | <1 | 11.193 | | |
| L13 | <0.1 | 95.25 | | |

TABLE M

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| M1 | >1 | 36.106 | | |
| M2 | >1 | 56.248 | | |
| M3 | >1 | 41.976 | | |
| M4 | >1 | 6.146 | | |
| M5 | >1 | 39.415 | | |
| M6 | >1 | 21.558 | | |
| M7 | >1 | 17.634 | | |
| M8 | >1 | 19.872 | | |
| M9 | >1 | 14.32 | | |
| M10 | >1 | 40.644 | | |
| M11 | >1 | 37.758 | | |
| M12 | >1 | 51.723 | | |

TABLE N

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| N1 | >1 | 48.858 | | |
| N2 | >1 | 27.411 | | |
| N3 | >1 | −0.959 | | |
| N4 | >1 | 10.616 | | |
| N5 | <0.1 | 20.971 | | |
| N6 | <1 | 29.026 | | |
| N7 | <1 | 11.485 | | |
| N8 | <1 | 53.398 | | |
| N9 | >1 | 22.943 | | |
| N10 | <1 | 40.144 | | |
| N11 | >1 | 5.101 | | |
| N12 | >1 | 60.012 | | |

TABLE O

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| O1 | <1 | 73.415 | | |
| O2 | >1 | 69.198 | | |
| O3 | <1 | 74.643 | | |
| O4 | >1 | 71.237 | | |
| O5 | <1 | 77.266 | | |
| O6 | <1 | 76.327 | | |
| O7 | <1 | 55.752 | | |
| O8 | <0.1 | 86.115 | | |
| O9 | >1 | 44.027 | | |
| O10 | >1 | 93.821 | | |
| O11 | >1 | 73.255 | | |
| O12 | <1 | 98.219 | | |

TABLE P

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| P1 | <0.1 | 32.072 | | |
| P2 | <0.1 | 73.771 | >30 | >30 |
| P3 | <0.1 | 85.754 | 11.909 | 21.039 |
| P4 | <0.1 | 44.295 | >30 | >30 |
| P5 | <0.01 | 63.6 | 11.155 | 9.794 |
| P6 | <0.01 | 43.506 | 7.08 | 12.059 |
| P7 | <1 | 49.047 | 10.49 | 12.402 |
| P8 | <0.1 | 88.486 | >30 | >30 |
| P9 | <1 | 57.612 | 1.528 | 2.906 |
| P10 | <0.1 | 97.865 | 16.002 | 9.437 |
| P11 | <0.1 | 70.741 | | |
| P12 | <0.01 | 6.148 | 19.608 | 6.146 |
| P13 | <0.01 | 26.35 | | |
| P14 | <0.01 | 21.411 | | |
| P15 | <0.01 | 27.239 | | |
| P16 | <1 | 25.027 | | |
| P17 | <0.1 | 22.086 | | |
| P18 | <0.1 | −12.064 | | |
| P19 | <0.1 | 77.727 | | |
| P20 | <0.1 | 13.279 | | |
| P21 | <0.1 | 28.861 | | |
| P22 | <0.1 | 26.605 | | |
| P23 | <1 | 7.683 | | |
| P24 | <0.1 | 28.855 | | |
| P25 | <0.1 | 19.104 | | |
| P26 | <0.1 | 42.609 | | |

TABLE Q

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| Q1 | <0.01 | 59.325 | 1.6 | 3.866 |
| Q2 | <0.01 | | 5.149 | 2.544 |
| Q3 | <0.01 | | 17.396 | >30 |
| Q4 | <0.01 | | 0.606 | 2.005 |
| Q5 | <0.01 | | 6.457 | 13.826 |
| Q6 | <0.1 | | 26.79 | >30 |
| Q7 | <0.01 | | 14.521 | >30 |

TABLE R

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| R1 | <0.01 | | >30 | >30 |
| R2 | >1 | | | |
| R3 | <0.01 | | >30 | 22.202 |
| R4 | <0.01 | | 14.143 | 11.481 |
| R5 | <0.1 | | 4.873 | 4.694 |
| R6 | <0.01 | | 10.741 | >30 |
| R7 | <0.01 | | >30 | 8.086 |

TABLE S

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| S1 | <0.1 | | | |
| S2 | <0.01 | | 9.711 | 22.513 |
| S3 | <0.01 | | 8.342 | 9.757 |
| S4 | <0.01 | | 1.704 | 4.023 |
| S5 | <0.1 | | | |
| S6 | <0.01 | | | |

TABLE T

| | | | AB: | |
|---|---|---|---|---|
| | CK2: IC50 | PIM2: % inh | MDAMB453: | AB: BxPC3: |
| Compound | (μM) | 2.5 uM | IC50 (μM) | IC50 (μM) |
| T1 | <0.01 | | 11.963 | 18.538 |

Citation of the above patents, patent applications, publications and documents is not an admission that any of the foregoing is pertinent prior art, nor does it constitute any admission as to the contents or date of these publications or documents. Furthermore, the contents of the patents, patent applications, publications and documents cited herein are incorporated by reference in their entirety for all purposes to the same extent as each and everyone of them is incorporated by references specifically.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

1. A compound of Formula (I): 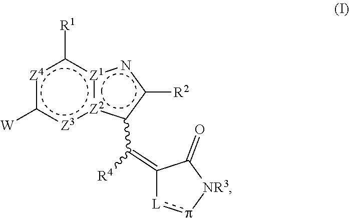
or a pharmaceutically acceptable salt, solvate, and/or prodrug thereof,
wherein:
the bicyclic ring system containing Z¹-Z⁴ is aromatic;
one of Z¹ and Z² is C, the other of Z¹ and Z² is N;
Z³ and Z⁴ are independently CR^{1a} or N,
R¹ and R^{1a} are independently H, halo, CN, optionally substituted C1-C4 alkyl, optionally substituted C2-C4 alkenyl, optionally substituted C2-C4 alkynyl, optionally substituted C1-C4 alkoxy, or —NR⁷R⁸;
R² is H, halo, CN, or an optionally substituted group selected from C1-C4 alkyl, C2-C4 alkenyl, and C2-C4 alkynyl;
R³ and R⁴ are independently selected from H and optionally substituted C1-C10 alkyl;
π is sp²-hybridized C or N;
the bond shown with a dotted line is a single bond if it is C═Y, where Y is O or S,
or the bond shown with a dotted line is a double bond if π is N or CR¹;
L is a one-carbon or two-carbon linker;
or L and π taken together form an additional 6-membered ring fused onto the ring containing the N of NR³, wherein the 6-membered ring optionally contains up to two heteroatoms selected from N, O and S as ring members;
W is halo, —OR⁷, —NR⁷R⁸, —S(O)ₙR⁷, —C(O)OR⁷, optionally substituted aryl, optionally substituted heteroaryl, optionally substituted heterocyclyl, optionally substituted C3-C8 cycloalkyl, or CR⁷R⁸R⁹,
wherein n is 0, 1 or 2,
each R⁷, R⁸, and R⁹ is independently selected from H, optionally substituted C1-C10 alkyl, optionally substituted aryl, optionally substituted arylalkyl, optionally substituted heteroaryl, optionally substituted heteroarylalkyl, and optionally substituted heterocyclyl; or alternatively, R⁷ and R⁸ in NR⁷R⁸, taken together with the nitrogen atom to which they are attached, form a 5 to 8 membered ring that is optionally substituted and optionally contain an additional heteroatom selected from N, O and S as a ring member.

2. The compound of claim 1, wherein Z¹ is N; and Z² is C.

3. The compound of claim 1, wherein Z³ is N.

4. The compound of claim 1, wherein Z⁴ is N or CR^{1a},wherein R^{1a} is H or C1-C4 alkyl.

5. The compound of claim 1, wherein R² is H.

6. The compound of claim 1, wherein R³ and R⁴ are both H.

7. The compound of claim 1, wherein R¹ is H or —NR⁷R⁸.

8. The compound of claim 1, wherein π is C═Y, where Y is O or S.

9. The compound of claim 8, wherein L is C(R⁶)₂.

10. The compound of claim 1, wherein L is CR⁶, where R⁶ is H or optionally substituted C1-C10 alkyl.

11. The compound of claim 10, wherein -L-π-N(R³)— is —CR⁶═N—N(R³)—.

12. The compound of claim 11, wherein R⁶ is H or optionally substituted C1-C4 alkyl.

13. The compound of claim 1, wherein -L-π-N(R³)— is 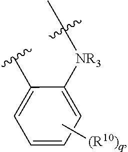
where R¹⁰ is selected from halogen, cyano, R″, OR″, NR″R″, CONR″R″, SO₂NR″R″, where each R″ is independently H or C1-C4 alkyl, and q is 0, 1, or 2.

14. The compound of claim 1, wherein W is —OR⁷ or —NR⁷R⁸.

15. The compound of claim 14, wherein R⁷ is optionally substituted aryl or optionally substituted heteroaryl; and R⁸ is H.

16. The compound of claim 15, wherein R⁸ is optionally substituted phenyl.

17. The compound of claim 14, wherein R⁷ and R⁸, taken together with the nitrogen atom, forms a 5 to 8 membered ring that is optionally substituted and optionally contains an additional heteroatom selected from N, O and S as a ring member.

18. The compound of claim 1, which is represented by Formula (Ia) or Formula (Ib): 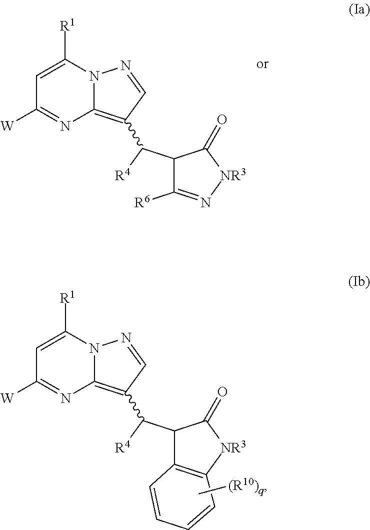
or a pharmaceutically acceptable salt, solvate, and/or prodrug thereof,
wherein
q is 0, 1, or 2;
each R¹⁰ is independently selected from halogen, cyano, R″, OR″, NR″R″, CONR″R″, and SO₂NR″R″, wherein each R″ is independently H or C1-C4 alkyl; and
R⁶ is H or an optionally substituted C1-C10 alkyl.

19. The compound of claim 1, which is represented by Formula (Ic) or Formula (Id): 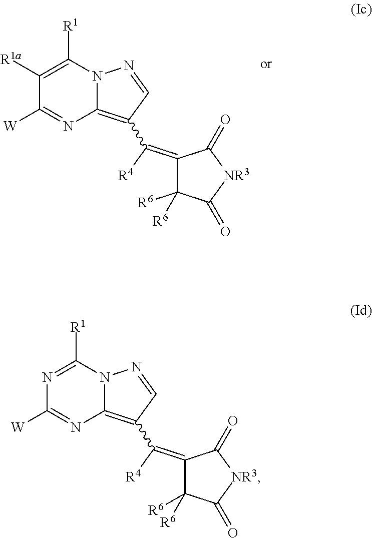
or a pharmaceutically acceptable salt, solvate, and/or prodrug thereof,
wherein
R^{1a} is H or C1-C4 alkyl;
R¹ is —NR⁷R⁸; and
each R⁶ is H or an optionally substituted C1-C10 alkyl.

20. The compound of claim 1, which is selected from the group consisting of 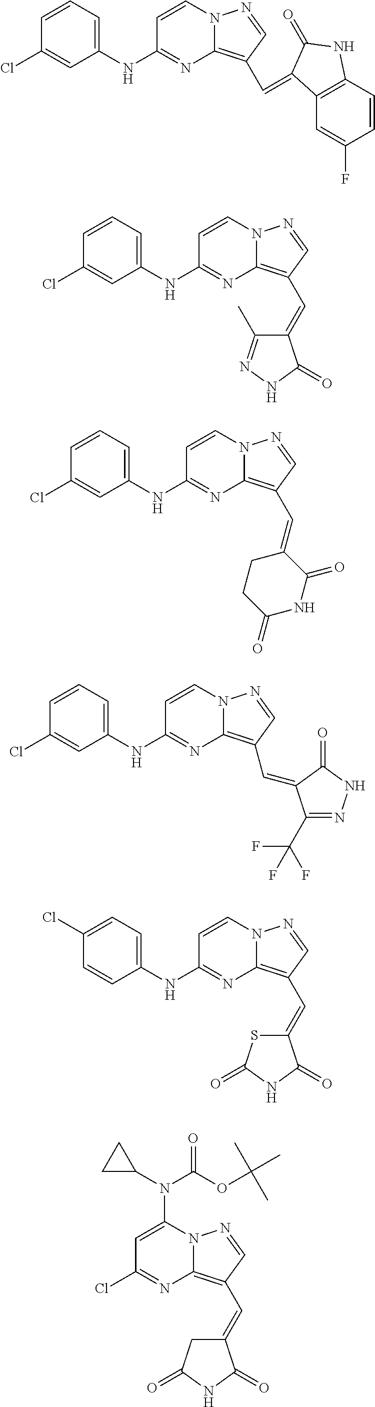 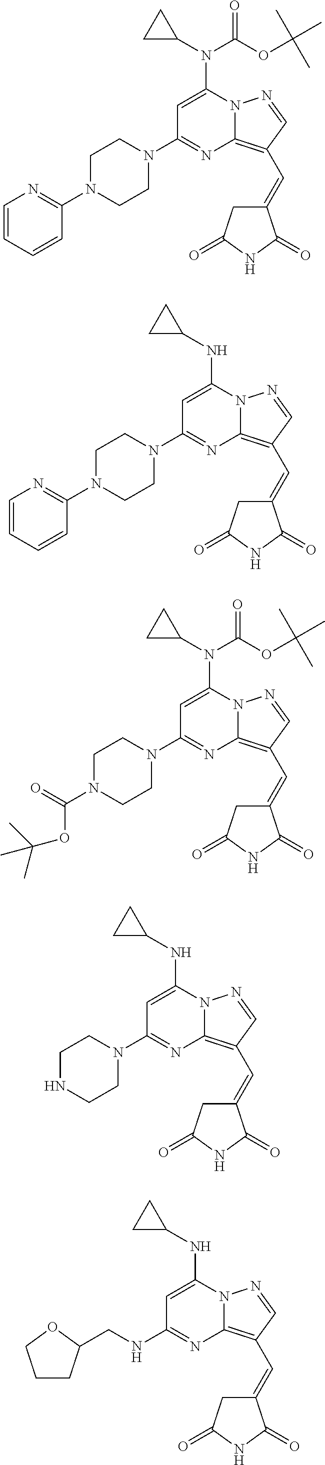 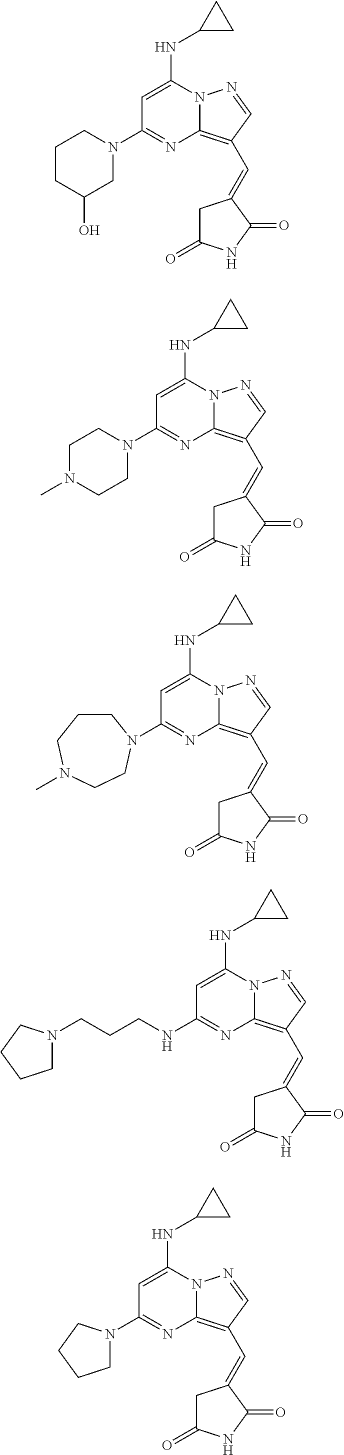 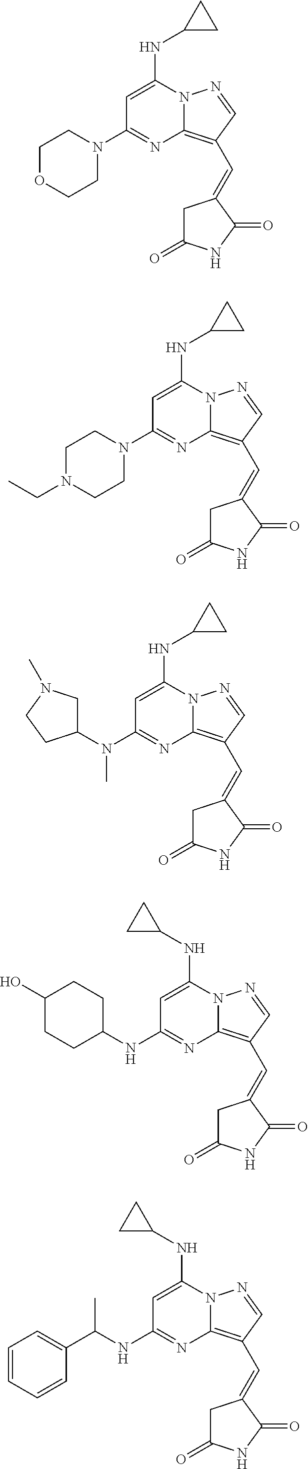 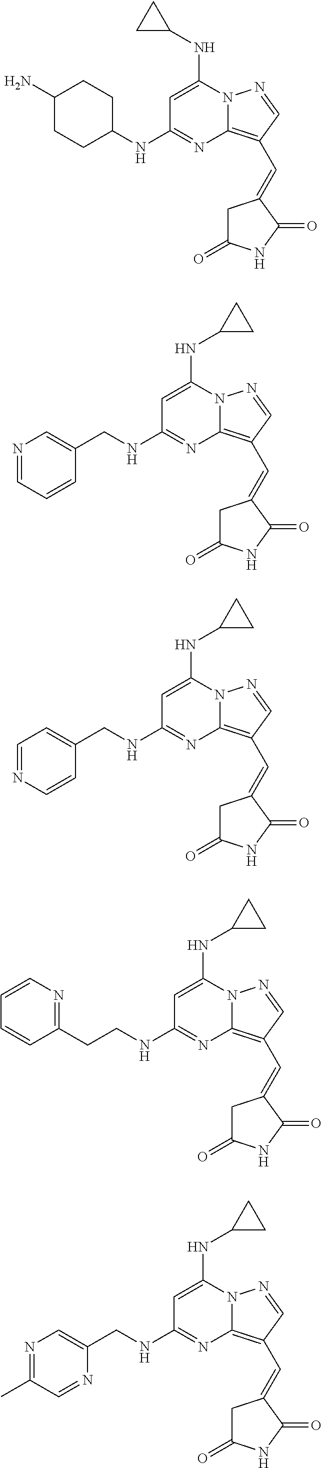 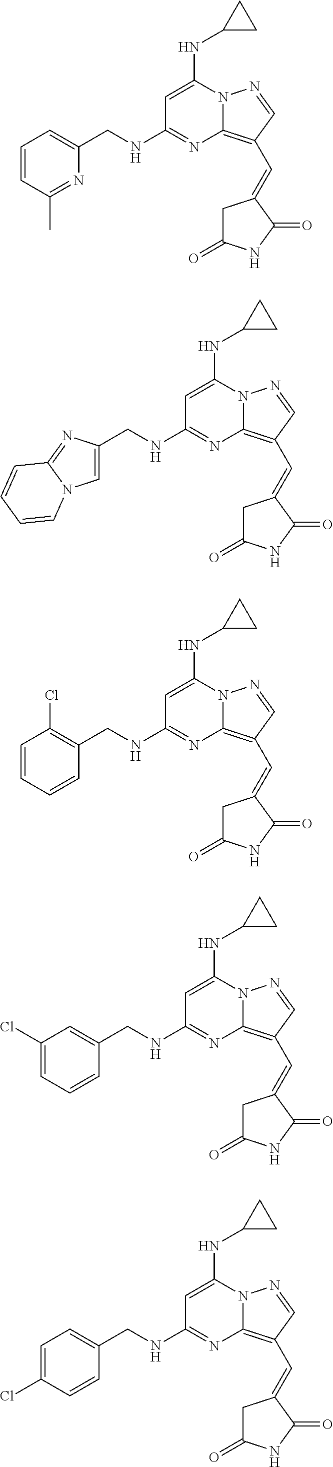 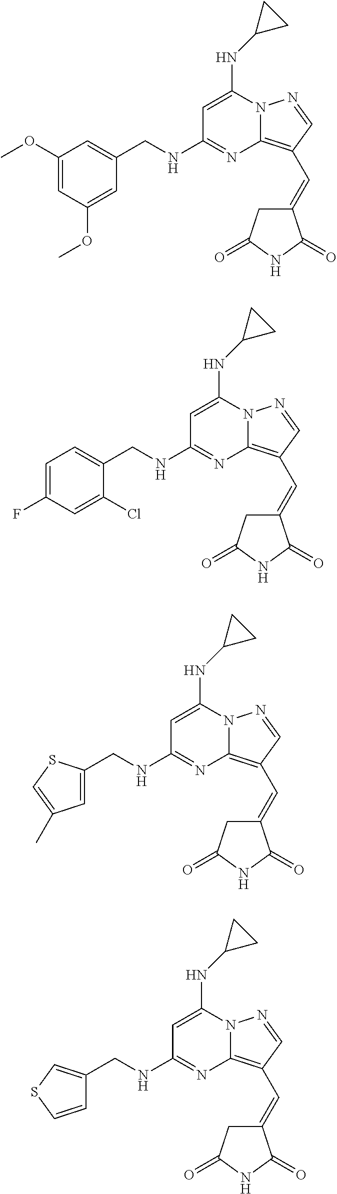 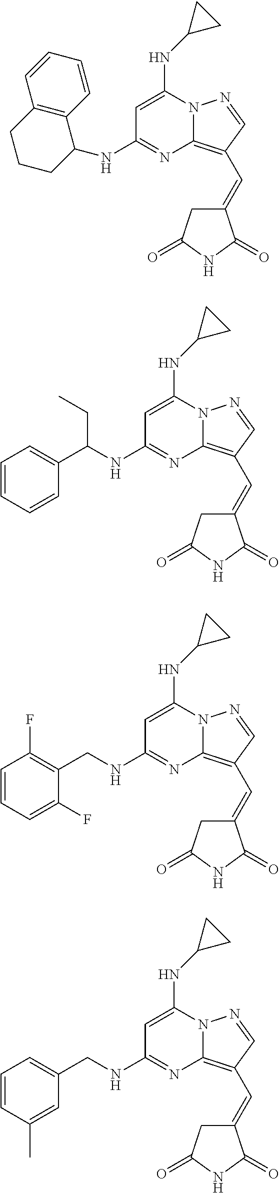 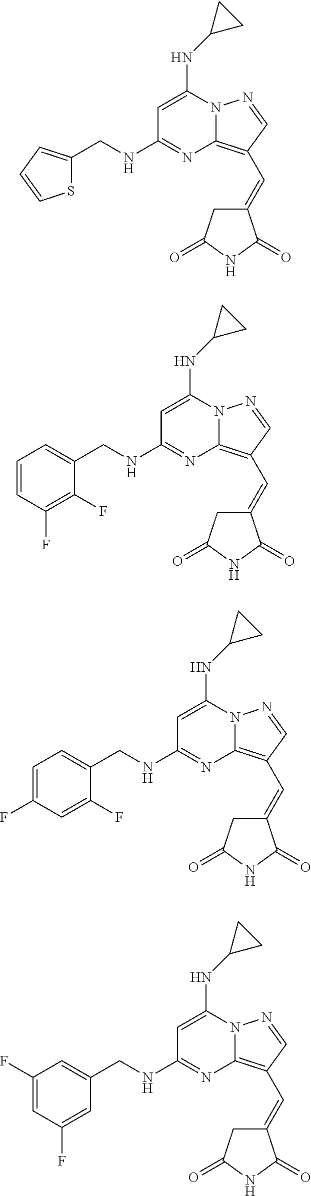 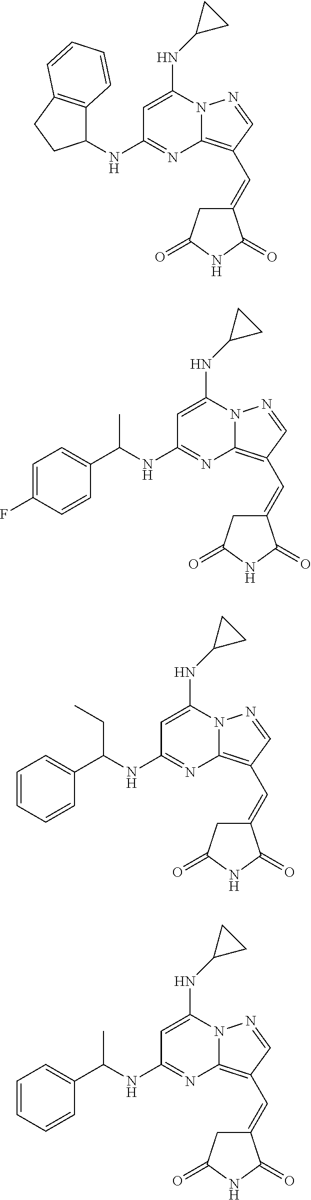 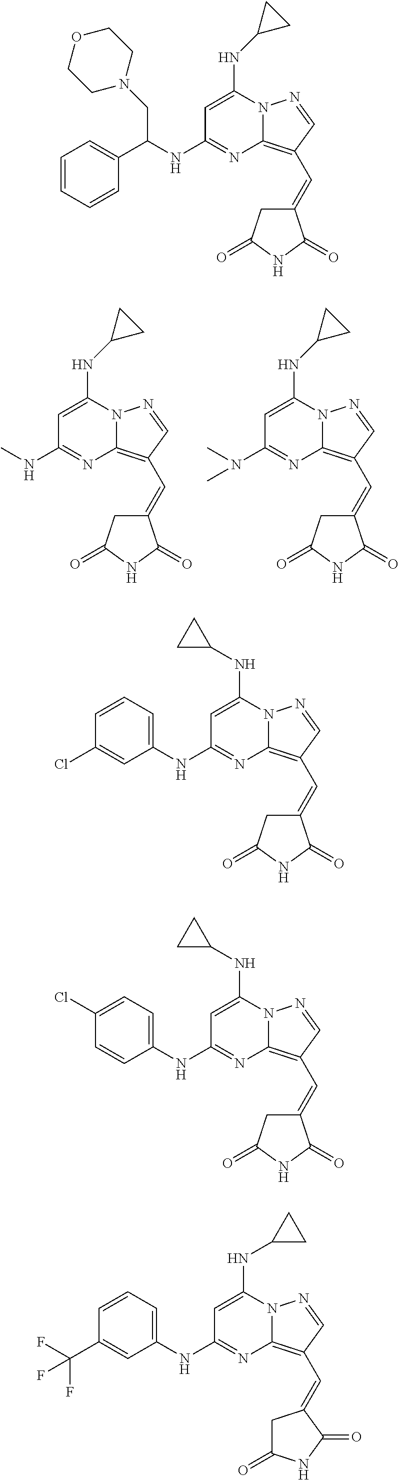 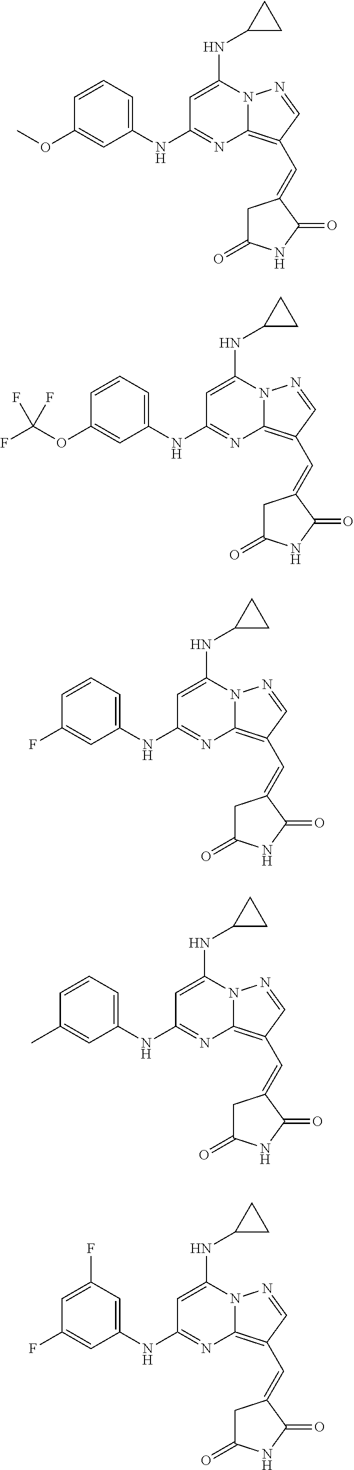 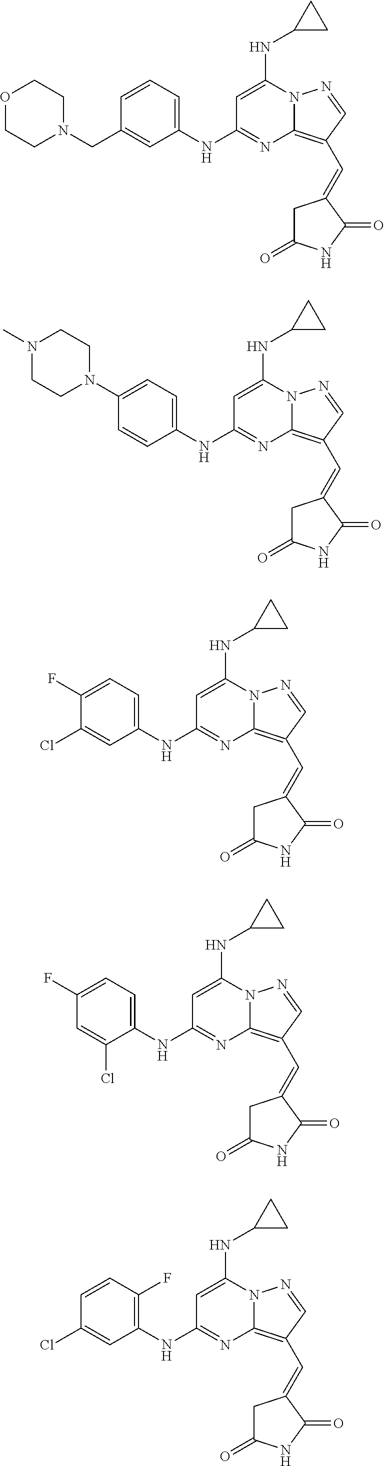 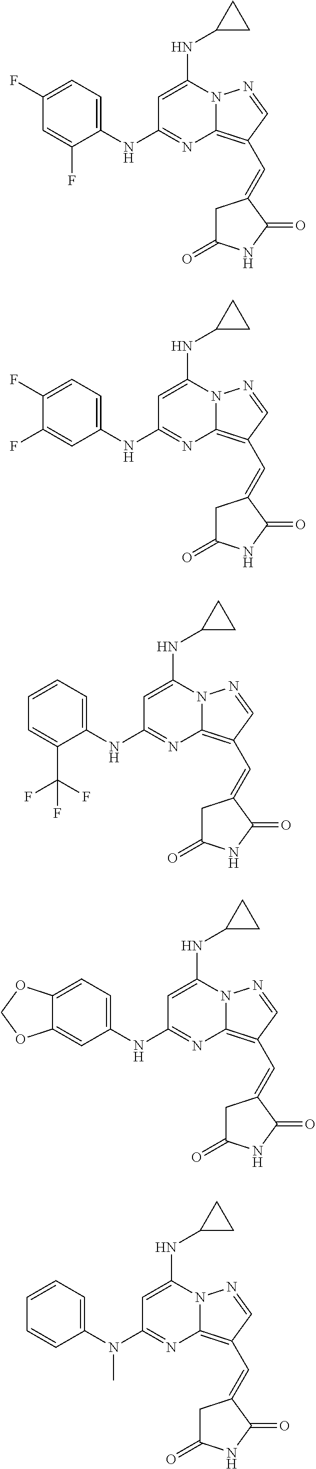 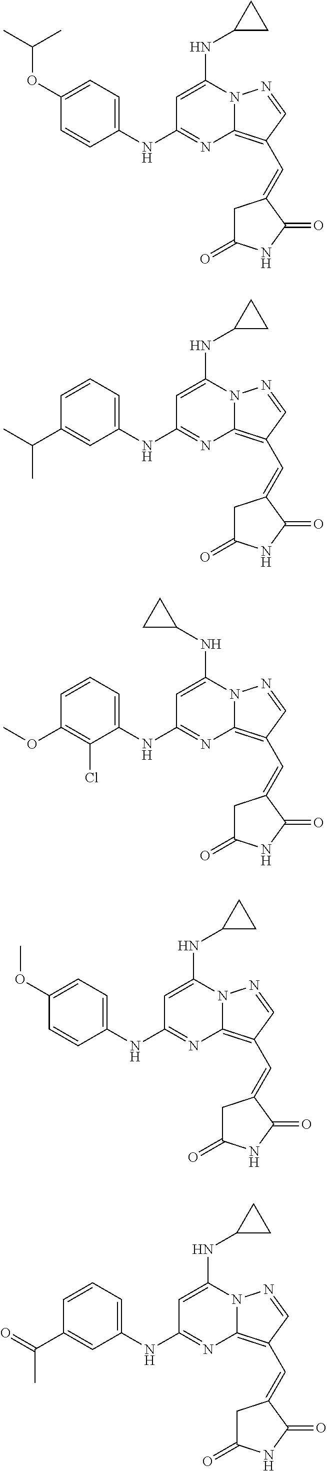 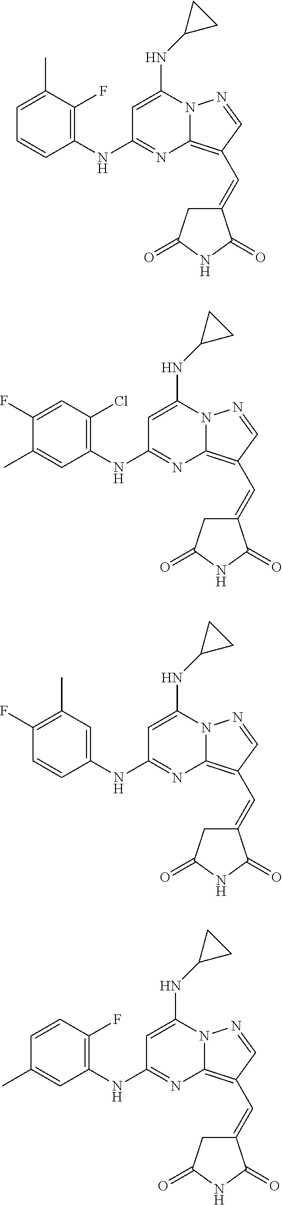 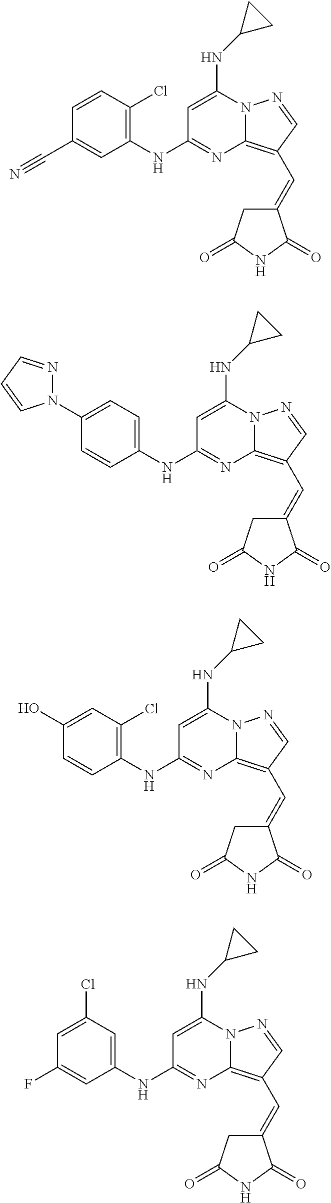 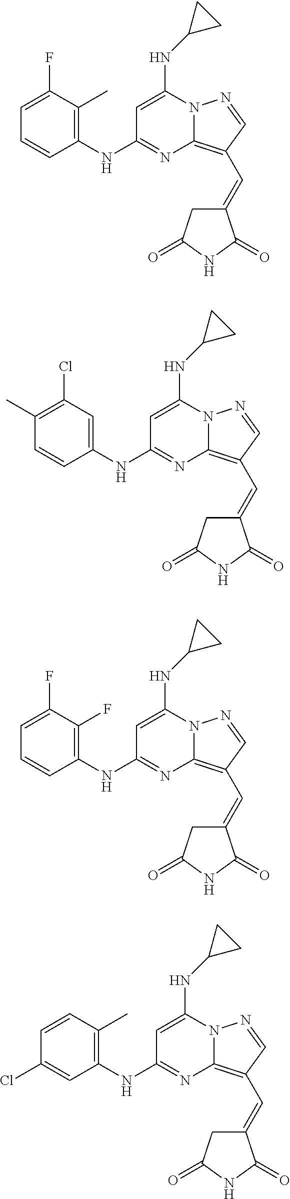 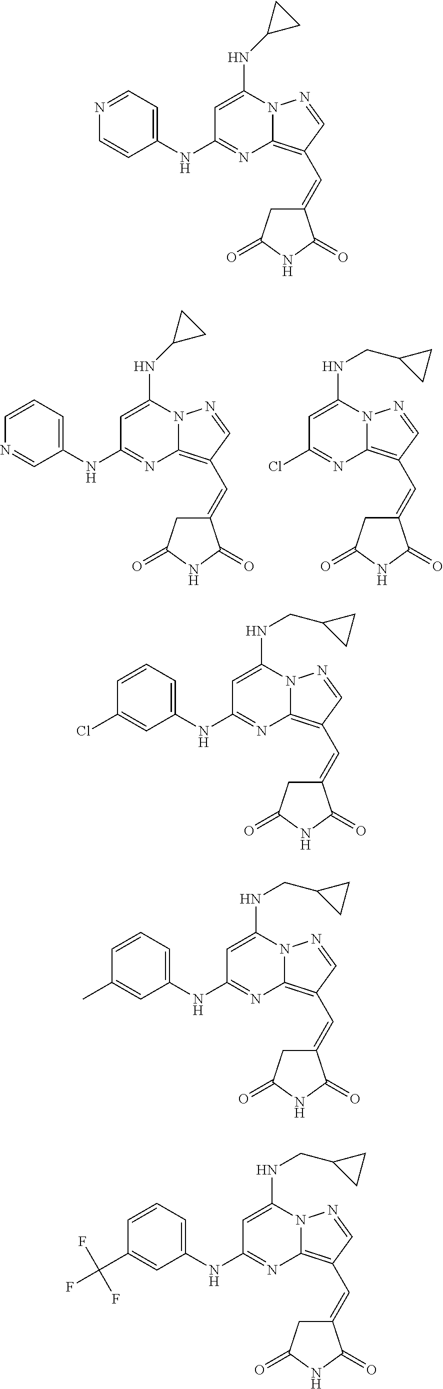 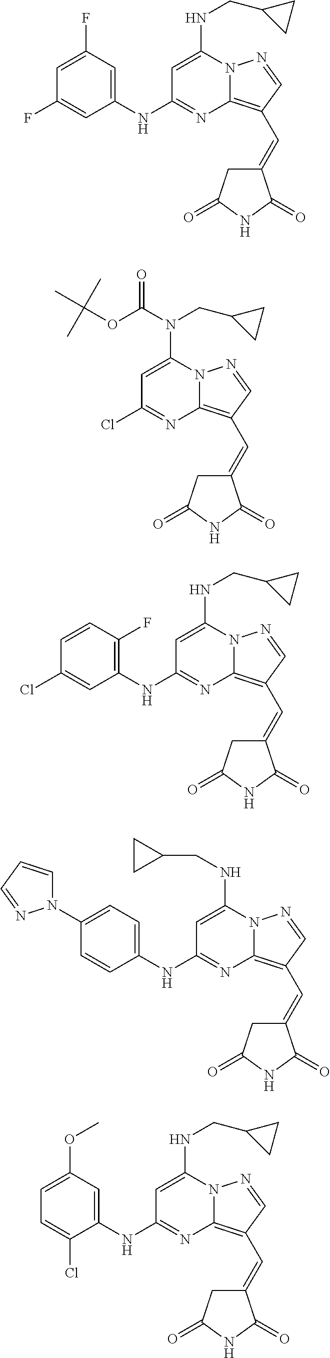 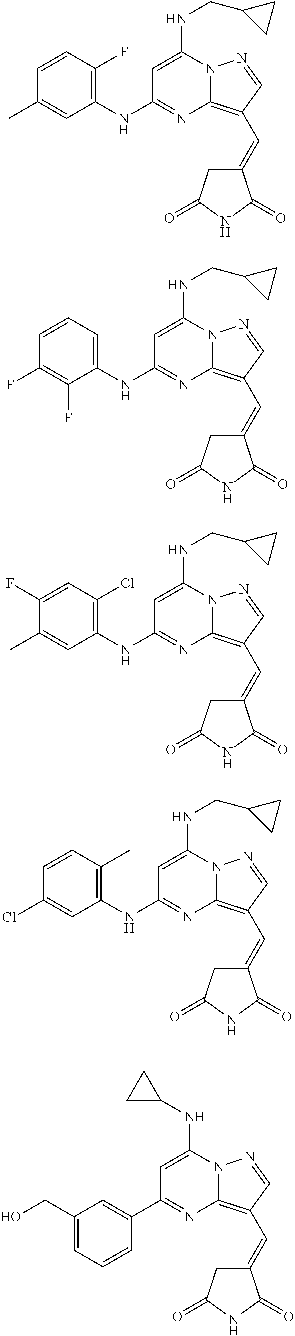 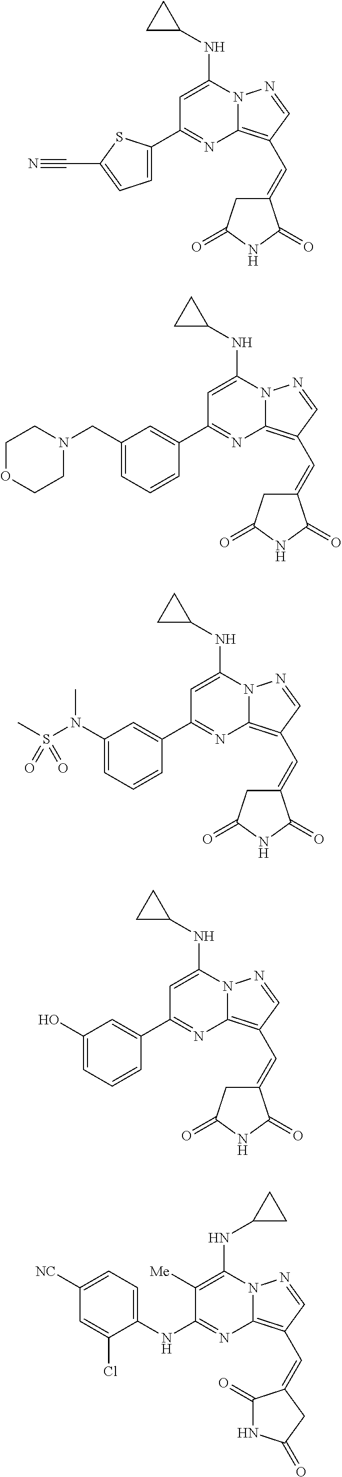 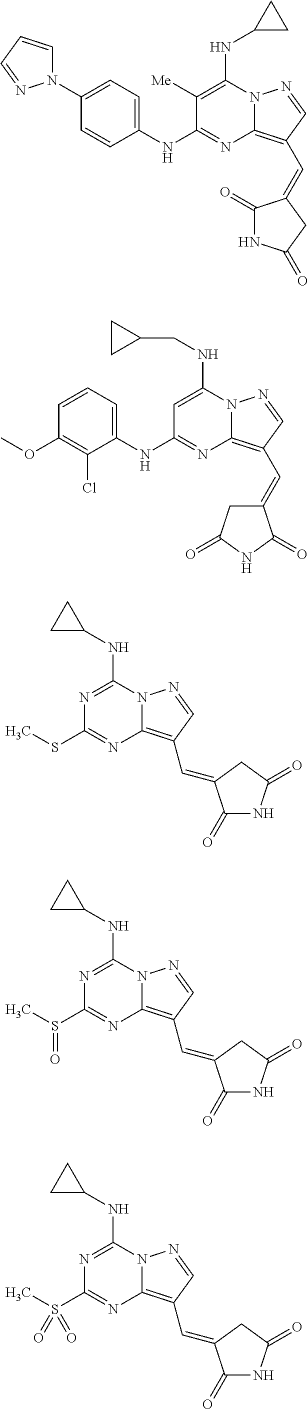 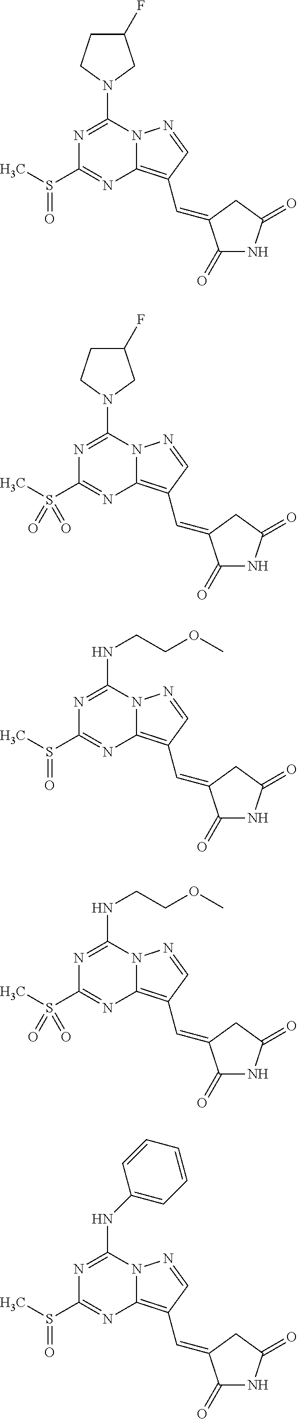 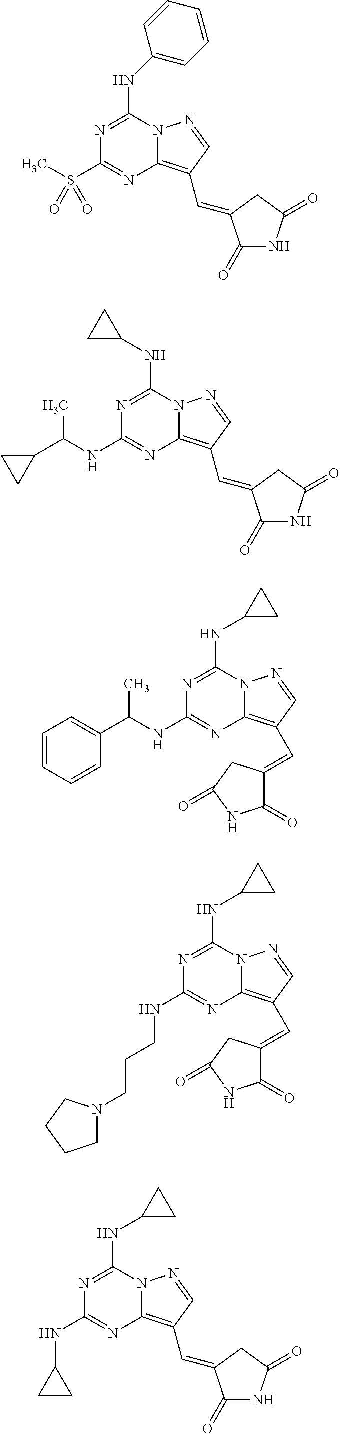 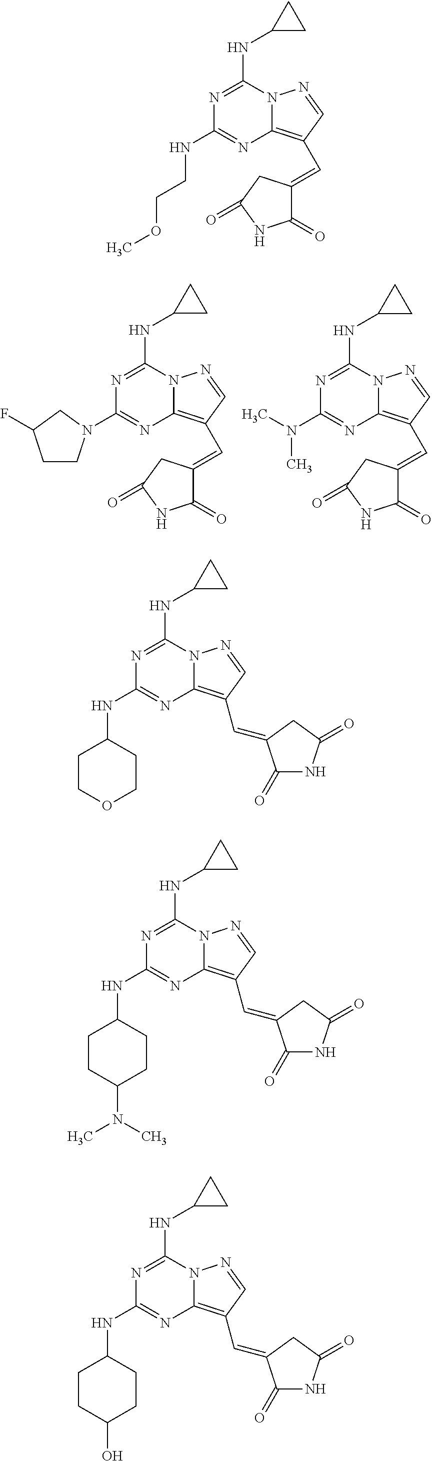 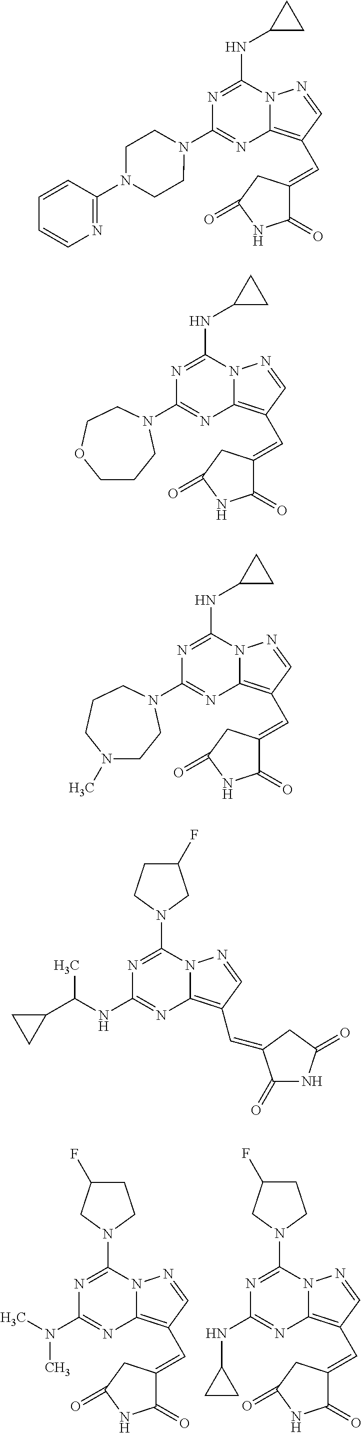 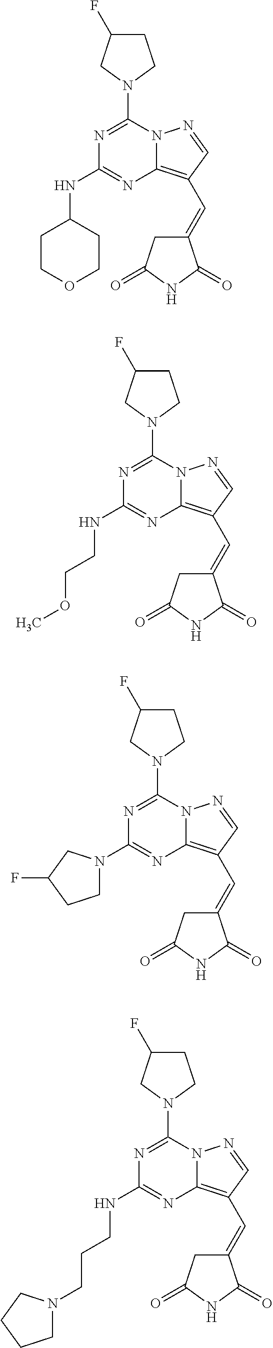 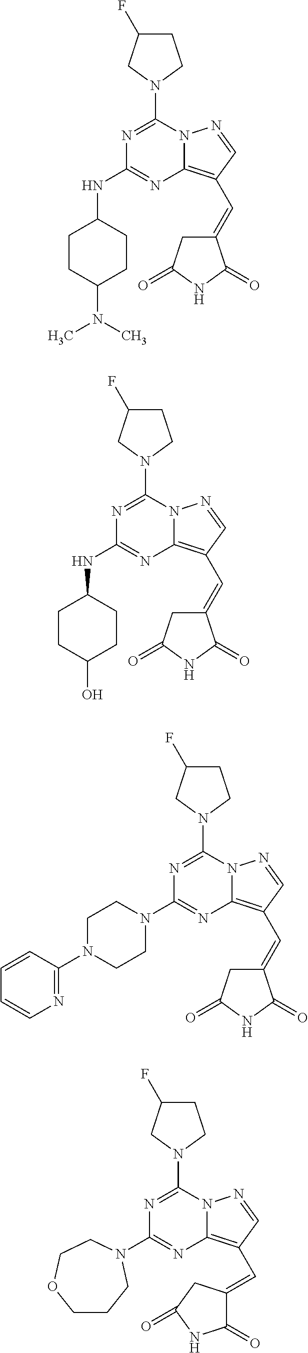 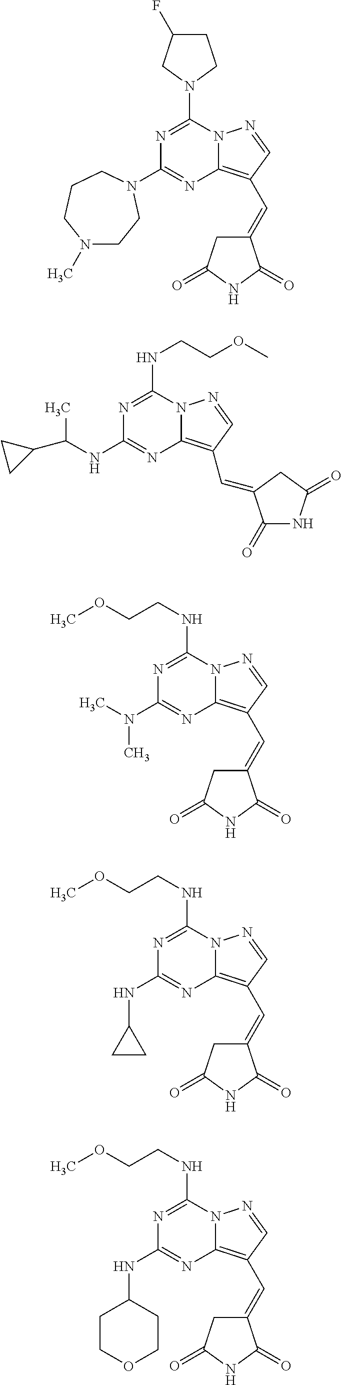 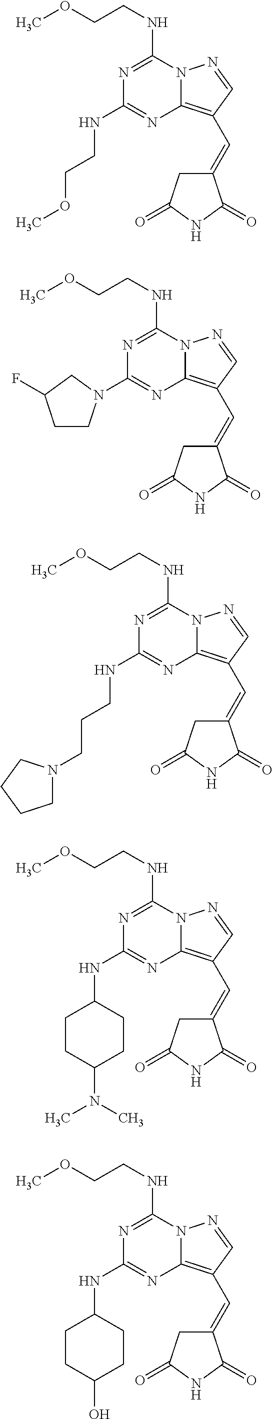 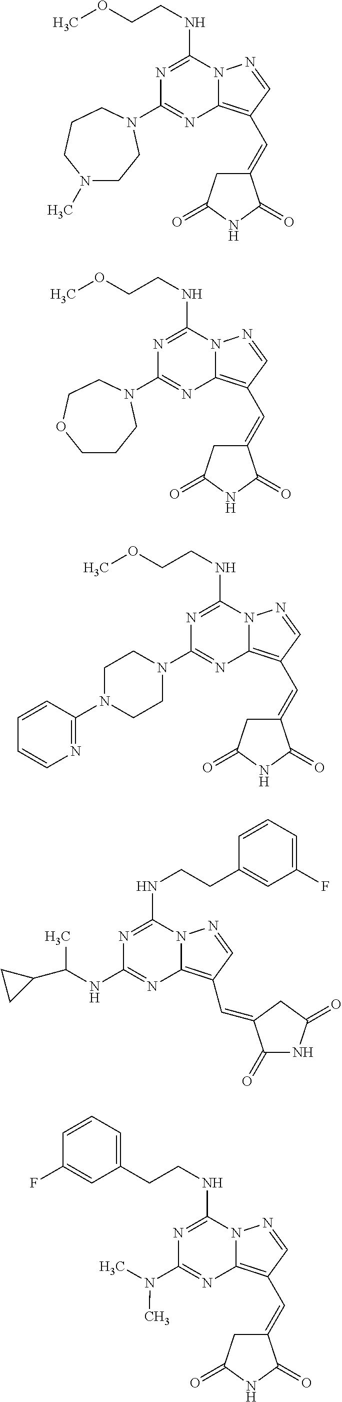 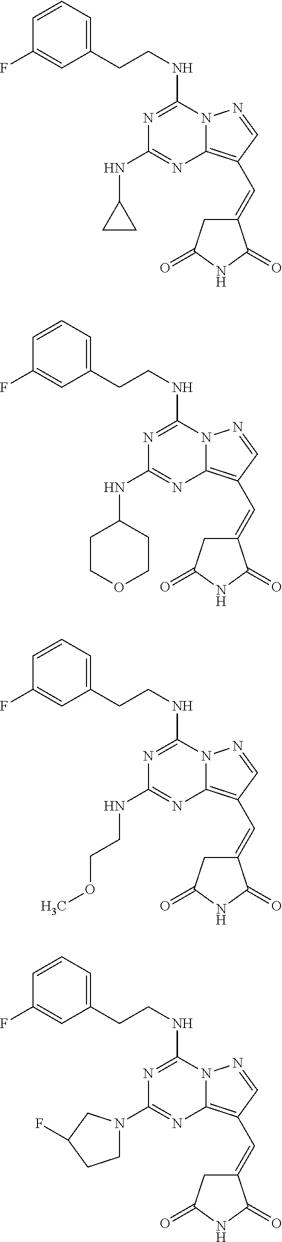 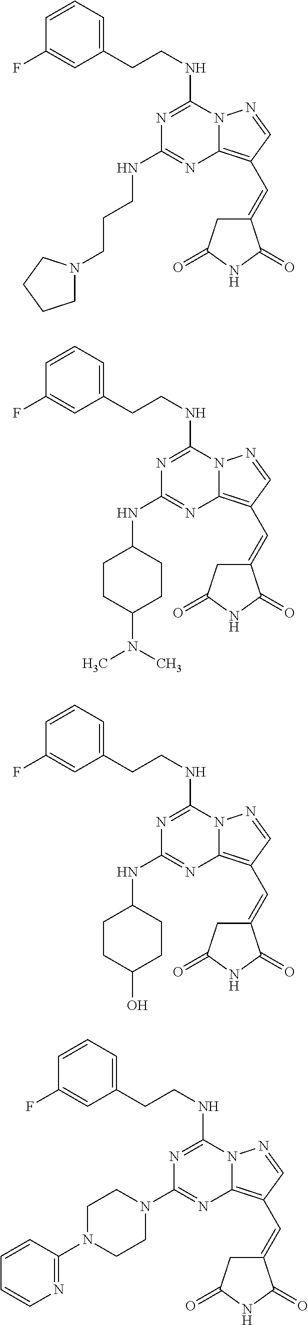 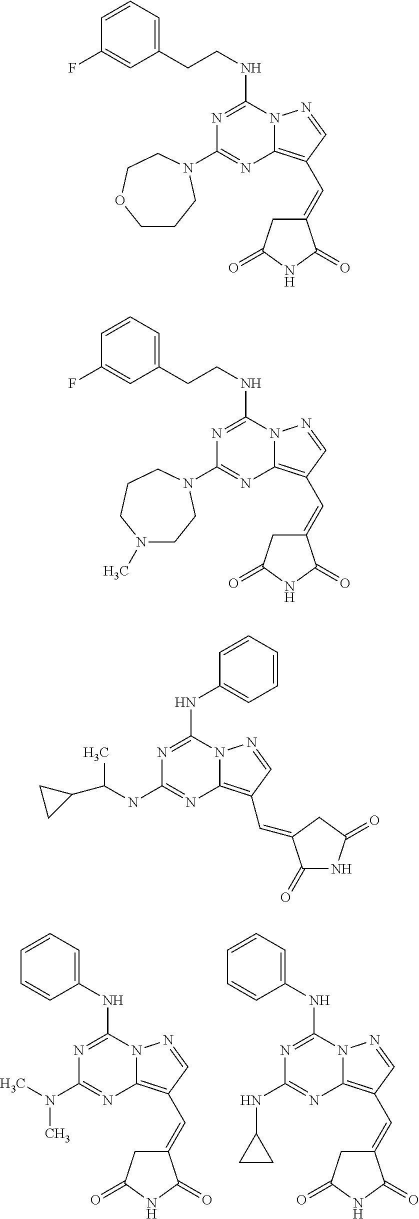 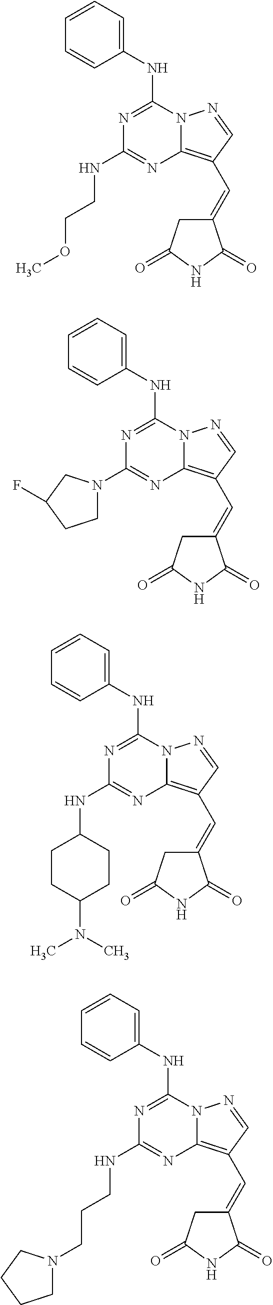 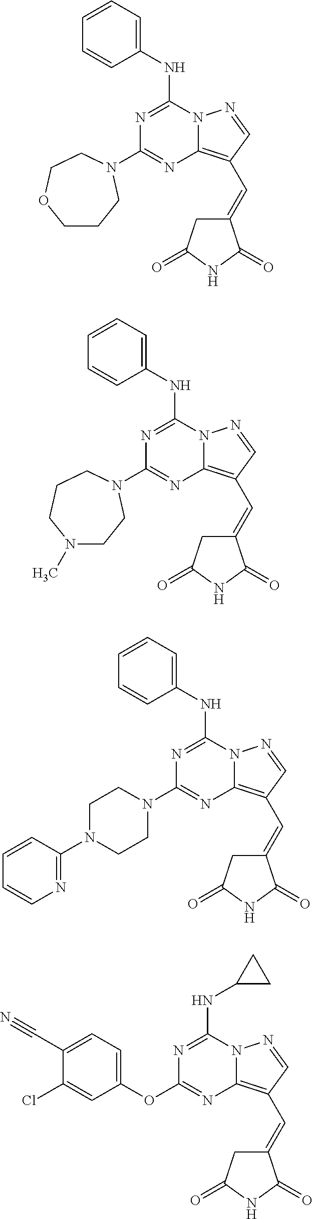 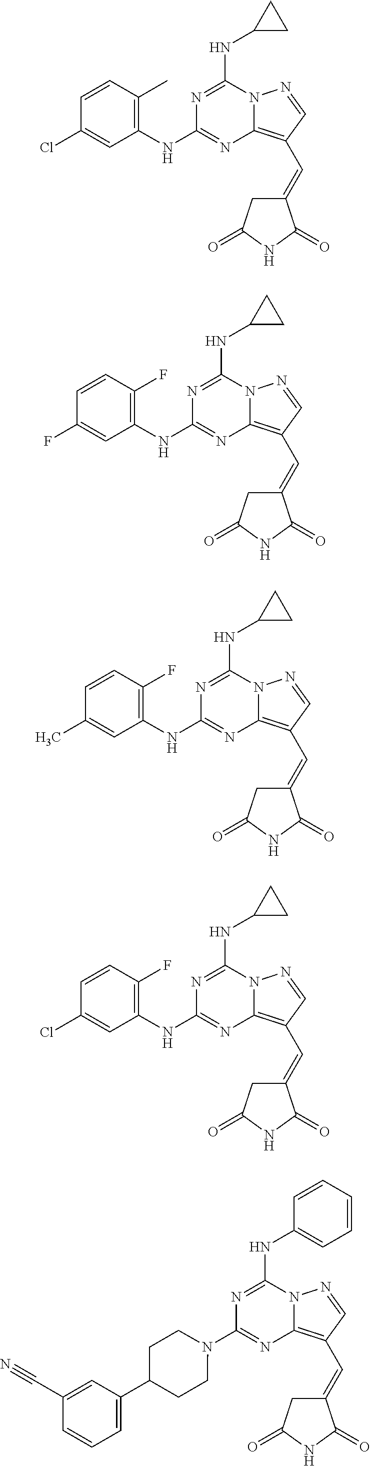 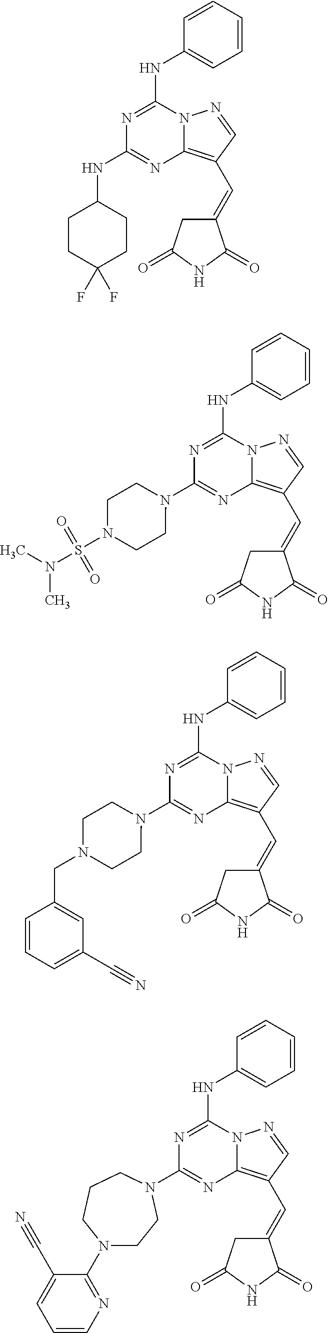 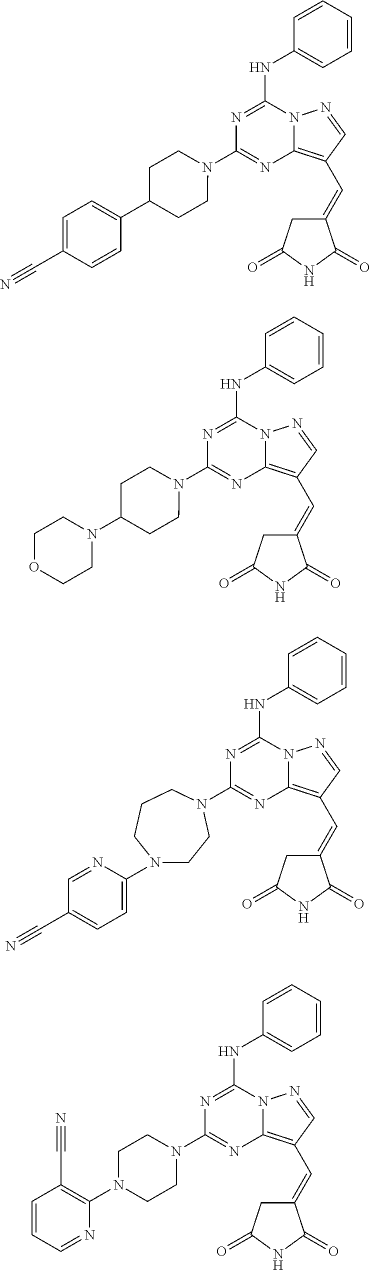 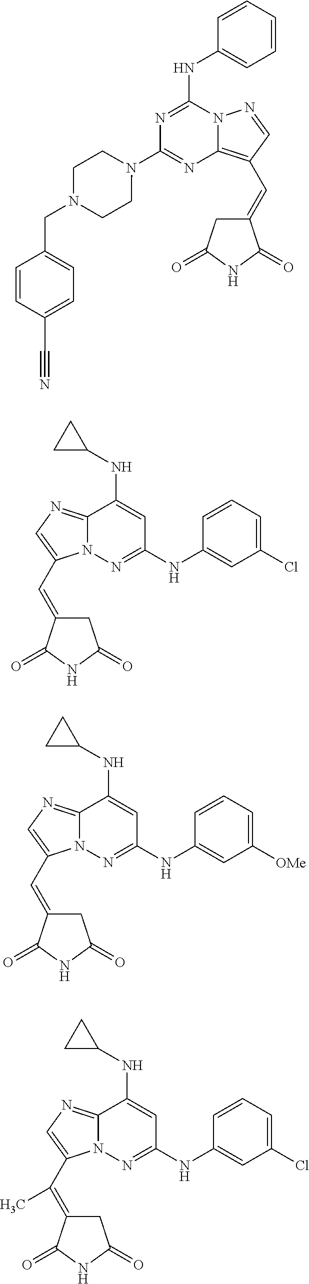 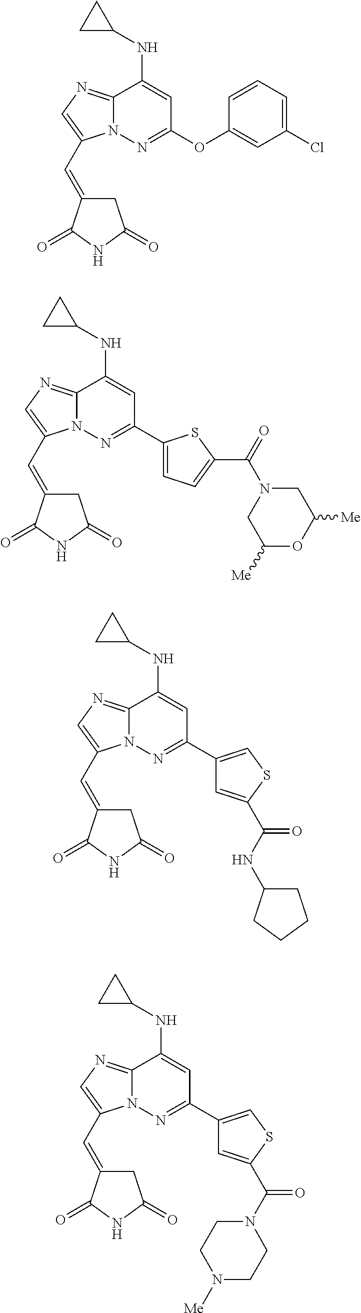 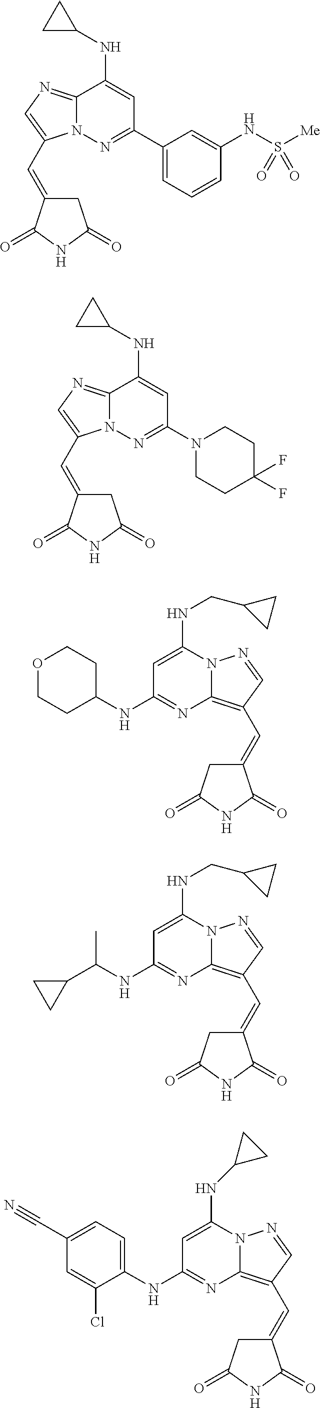 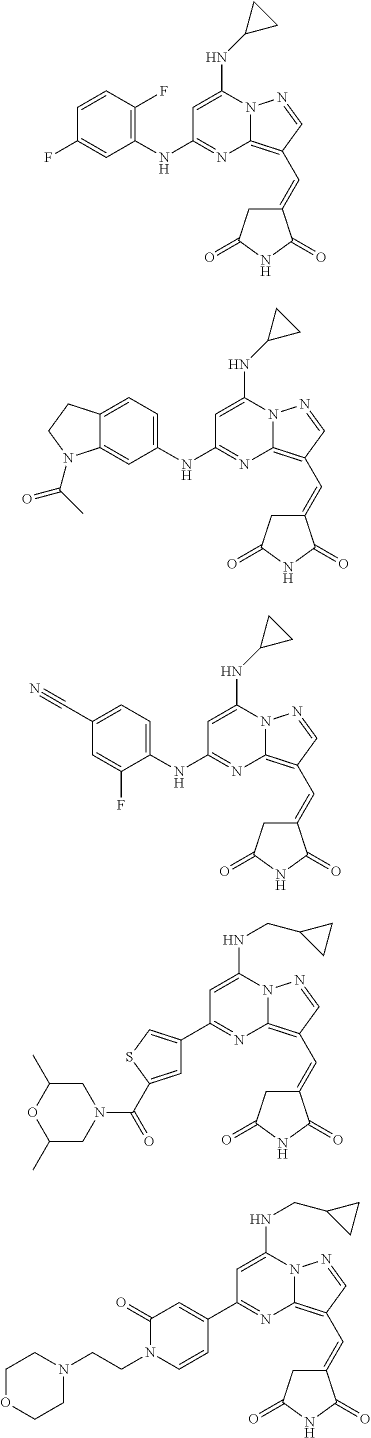 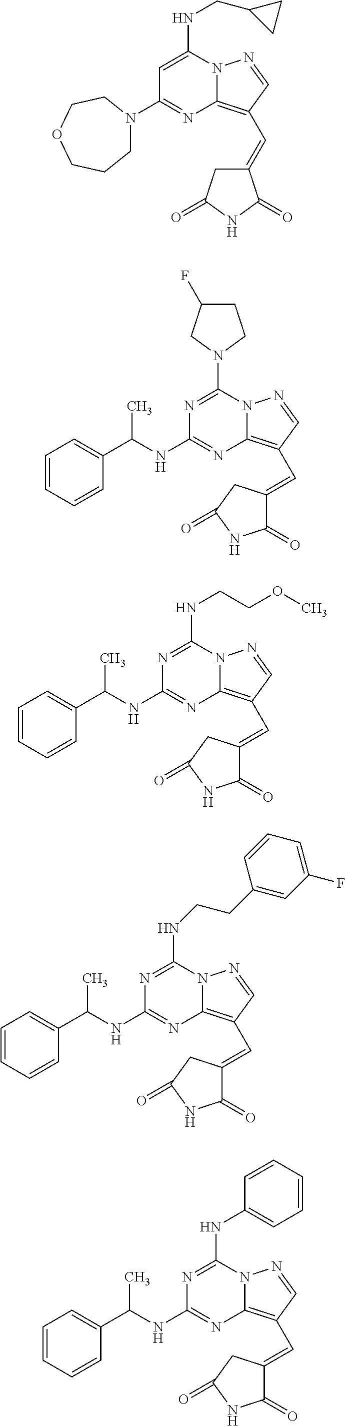 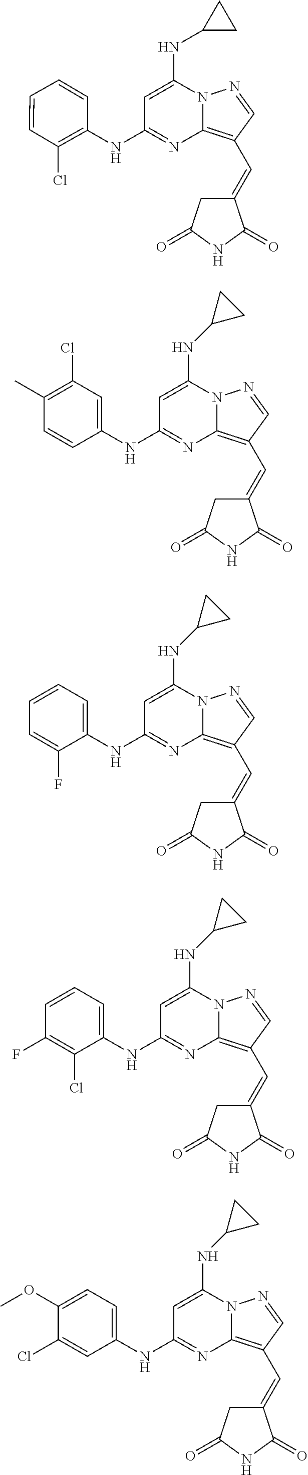 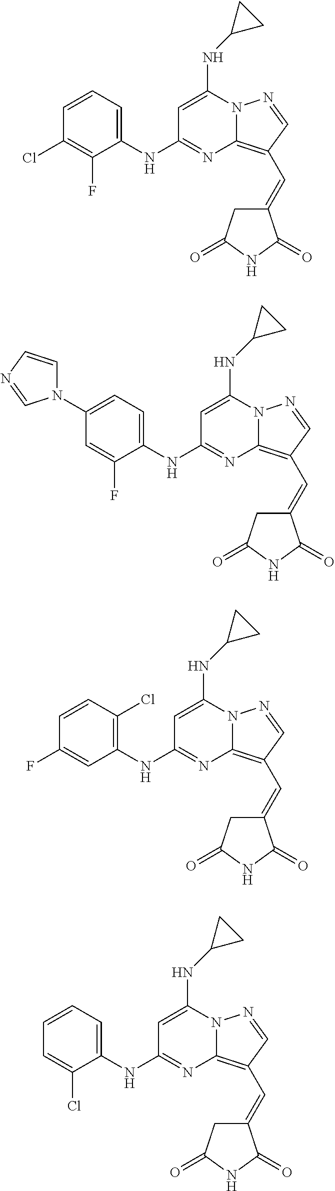 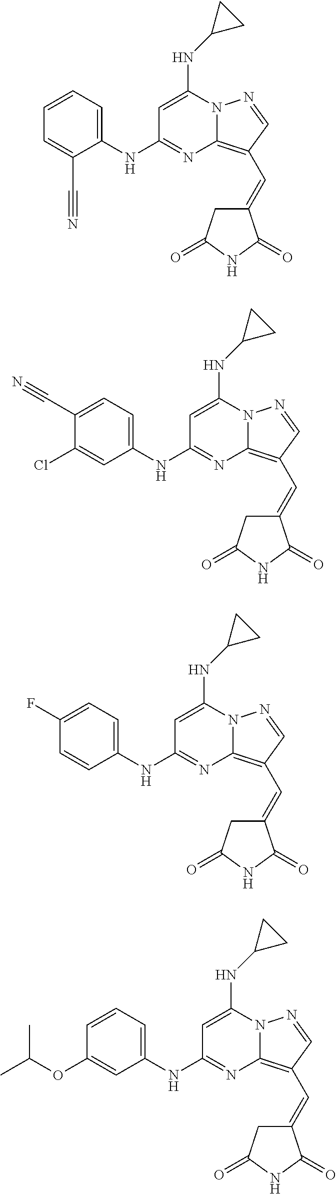 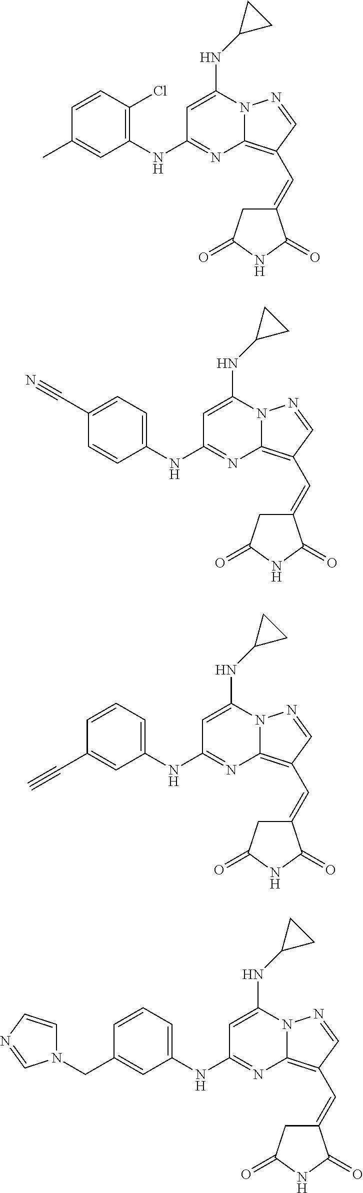 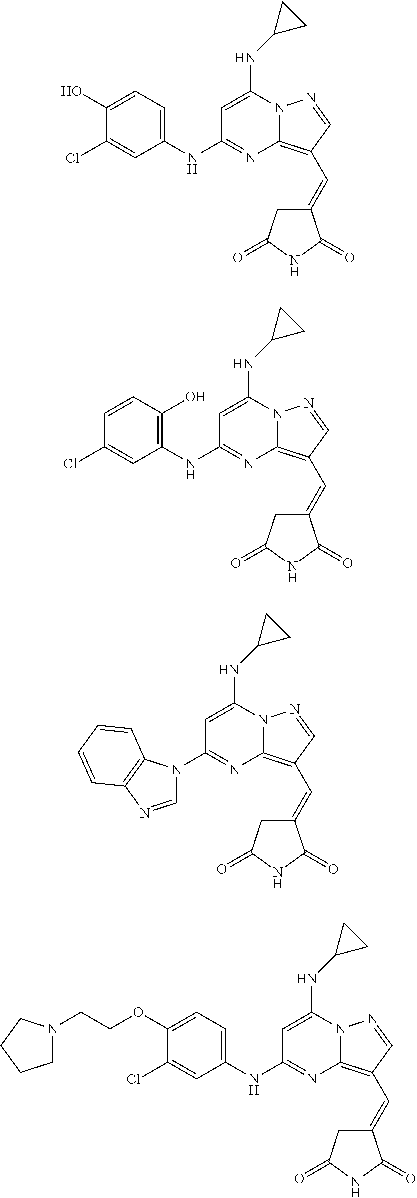 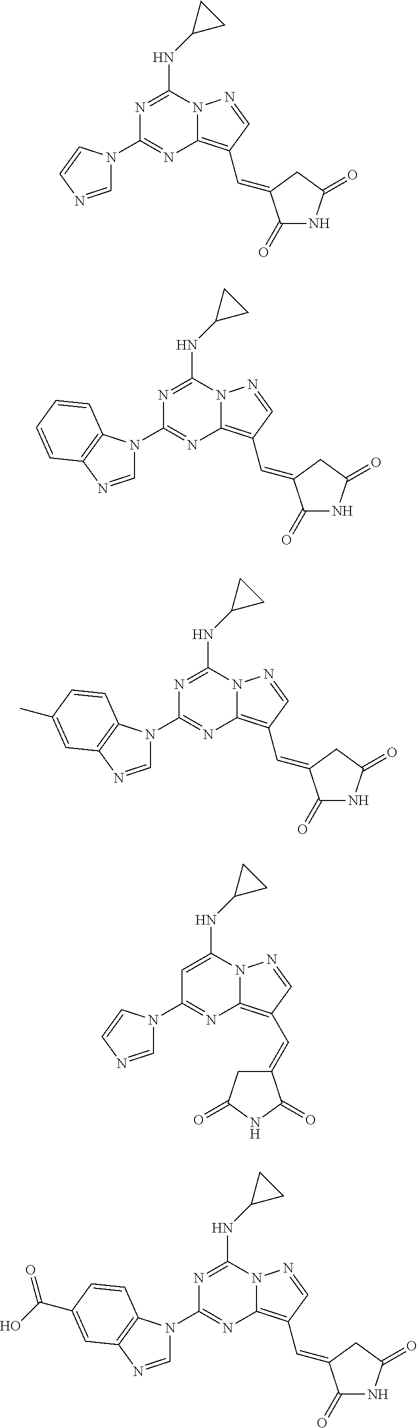 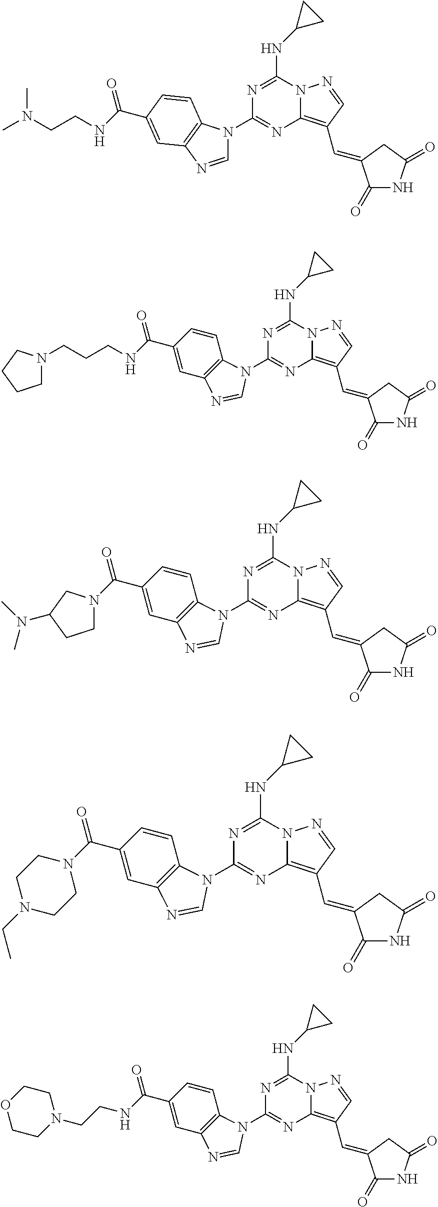 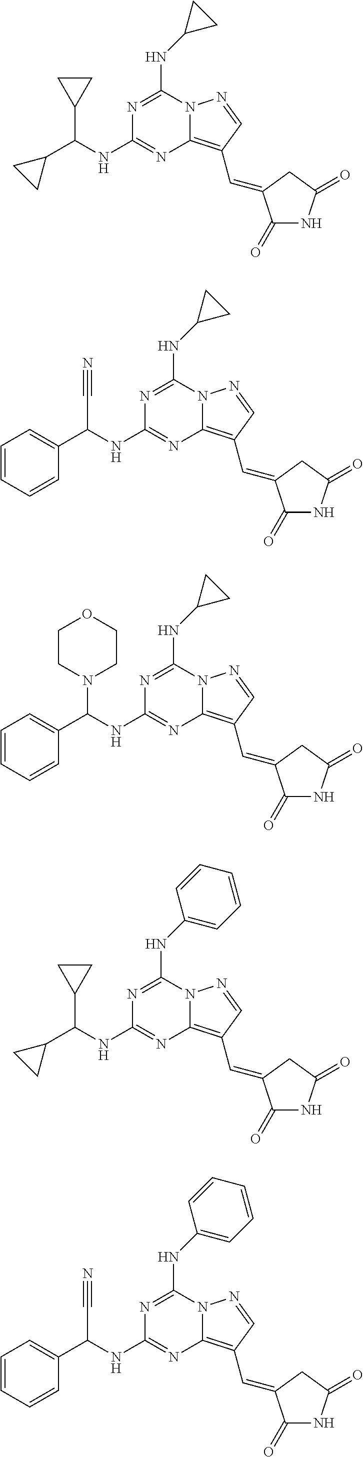 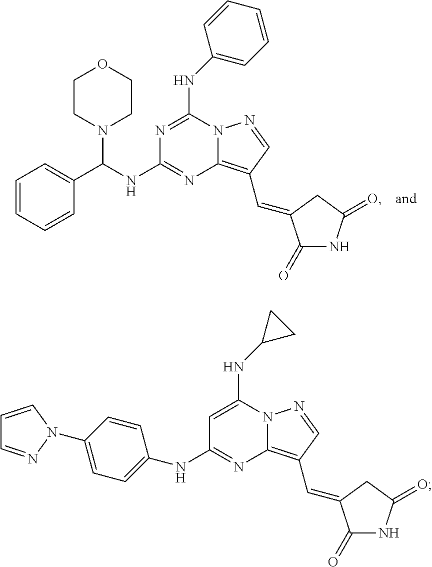
or a pharmaceutically acceptable salt, solvate, and/or prodrug thereof.

21. The compound of claim 1, which is represented by Formula (Ie): 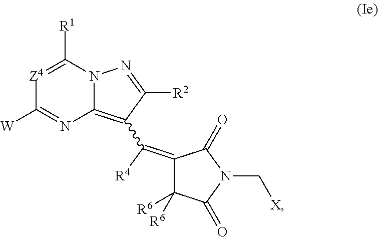
or a pharmaceutically acceptable salt and/or solvate thereof;
wherein,
Z⁴ are independently CR^{1a} or N,
R¹ and R^{1a} are independently H, halo, CN, optionally substituted C1-C4 alkyl, optionally substituted C2-C4 alkenyl, optionally substituted C2-C4 alkynyl, optionally substituted C1-C4 alkoxy, or —NR⁷R⁸;
R² is H, halo, CN, or an optionally substituted group selected from C1-C4 alkyl, C2-C4 alkenyl, and C2-C4 alkynyl;
R⁴ is H or optionally substituted C1-C10 alkyl;
each R⁶ is independently H or optionally substituted C1-C10 alkyl
W is halo, —OR⁷, —NR⁷R⁸, —S(O)ₙR⁷, —C(O)OR⁷, optionally substituted aryl, optionally substituted heteroaryl, optionally substituted heterocyclyl, optionally substituted C3-C8 cycloalkyl, or CR⁷R⁸R⁹,
wherein n is 0, 1 or 2,
each R⁷, R⁸, and R⁹ is independently selected from H, optionally substituted C1-C10 alkyl, optionally substituted aryl, optionally substituted arylalkyl, optionally substituted heteroaryl, optionally substituted heteroarylalkyl, and optionally substituted heterocyclyl; or alternatively, R⁷ and R⁸ in NR⁷R⁸, taken together with the nitrogen atom to which they are attached, form a 5 to 8 membered ring that is optionally substituted and optionally contain an additional heteroatom selected from N, O and S as a ring member;
X is hydroxyl or a group having structural formula (II), (III), (IV), or (V): 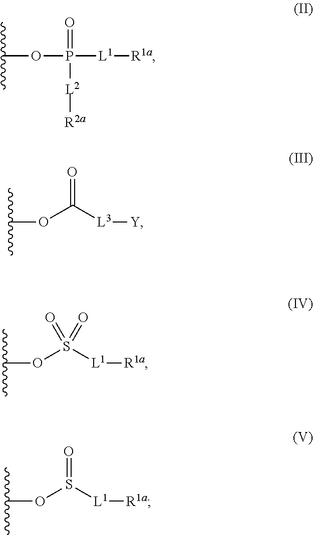
L¹ and L² are each independently a covalent bond, —O—, or —NR^{3a}—;
R^{1a} and R^{2a} are each independently hydrogen, alkyl, heteroalkyl, heteroaryl, heterocyclyl, alkenyl, alkynyl, arylalkyl, heteroarylalkyl, heterocyclylalkyl, -alkylene-C(O)—O—R^{4a}, or -alkylene-O—C(O)—O—R^{4a}; and
—R^{3a} and R^{4a} are each independently hydrogen, alkyl, heteroalkyl, cyclylalkyl, heterocyclyl, aryl, heteroaryl, alkenyl, alkynyl, arylalkyl, heterocyclylalkyl, or heteroarylalkyl;
L³ is a covalent bond or alkylene;
Y is OR^{5a}, NR^{5a}R^{6a}, or C(O)OR^{7a}, provided that when Y is C(O)OR^{7a}, then L³ is not a covalent bond; and
R^{5a}, R^{6a}, and R^{7a} are each independently hydrogen, alkyl, arylalkyl, aryl, heteroalkyl, alkylheteroaryl, heterocyclyl, or heteroaryl; or alternatively, R^{5a} and R^{6a}, taken together with the nitrogen atom to which they are attached, form a heterocyclyl ring optionally containing one or more additional heteroatom independently selected from N, O, and S.

22. The compound of claim 21, wherein R² is H.

23. The compound of claim 22, wherein R⁴ is H.

24. The compound of claim 21, wherein R¹ is —NR⁷R⁸.

25. The compound of claim 21, wherein W is —OR⁷ or —NR⁷R⁸.

26. The compound of claim 25, wherein R⁷ is optionally substituted aryl or optionally substituted heteroaryl; and R⁸ is H.

27. The compound of claim 26, wherein R⁸ is optionally substituted phenyl.

28. The compound of claim 21, wherein
L¹ and L² are —O—; and
R^{1a} and R^{2a} are each independently hydrogen or alkyl.

29. The compound of claim 21, wherein
L³ is alkylene; and
Y is C(O)OR^{7a} or NR^{5a}R^{6a}.

30. The compound of claim 21, wherein
L³ is a covalent bond; and
Y is OR^{5a} or NR^{5a}R^{6a}.

31. The compound of claim 21, which is selected from the group consisting of 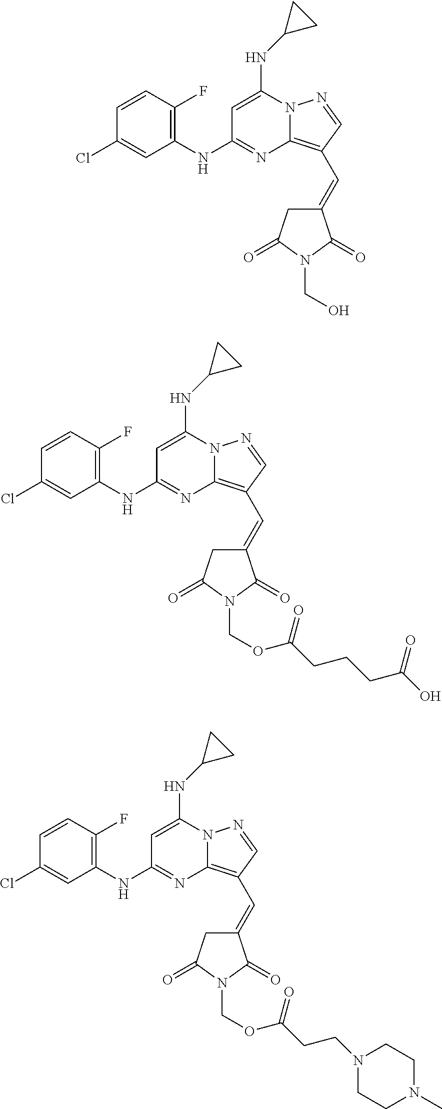 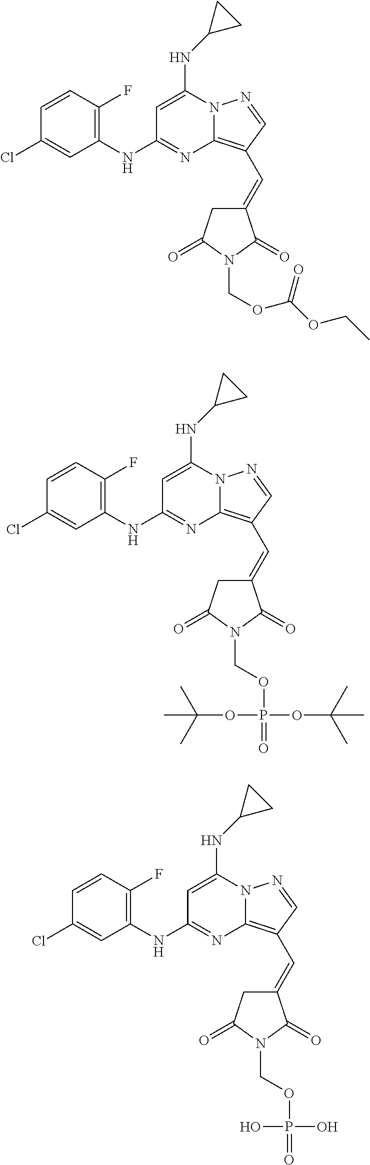 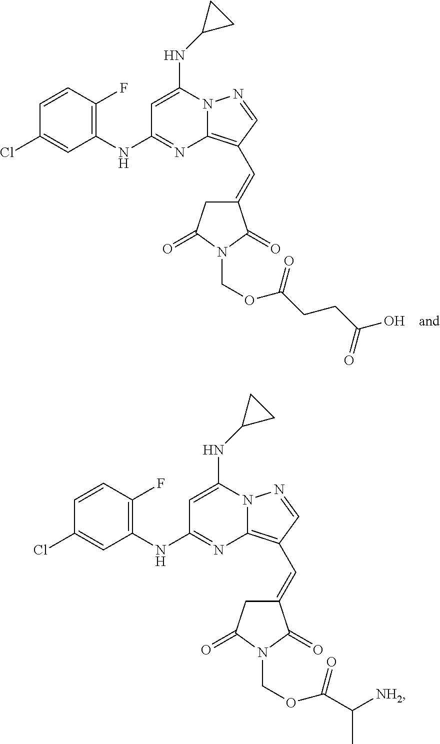
or a pharmaceutically acceptable salt and/or solvate thereof.

32. A pharmaceutical composition comprising
a compound of claim 1; and
a pharmaceutically acceptable excipient.

33. A method for modulating casein kinase 2 activity and/or Pim kinase activity in a cell comprising contacting the cell with a compound of claim 1.

34. A method of treating a condition or disease associated with casein kinase 2 activity and/or Pim kinase activity in a patient comprising administering to the patient a therapeutically effective amount of the compound of claim 1.

35. The method of claim 34, wherein the condition or disease is selected from a group consisting of a cancer, a vascular disorder, a inflammation, a pathogenic infection, a immunological disorder, and a combination thereof.

36. Ther method of claim 35, the cancer is of the colorectum, breast, lung, liver, pancreas, lymph node, colon, prostate, brain, head and neck, skin, liver, kidney, blood and heart.

37. A method for inhibiting cell proliferation, which comprises contacting cells with the compound of claim 1, in an amount effective to inhibit proliferation of the cells.

38. The method of claim 37, wherein the cells are in a cancer cell line or in a tumor in a subject.

39. The method of claim 38, wherein the cancer cell line is a breast cancer, prostate cancer, pancreatic cancer, lung cancer, hematopoietic cancer, colorectal cancer, skin cancer, ovary cancer cell line.

40. A method for inhibiting angiogenesis in a subject, which comprises administering to the subject the compound of claim 1 in an amount effective to inhibit the angiogenesis.

41. A method of treating a condition or disease associated with casein kinase 2 activity and/or Pim kinase activity in a patient comprising co-administering to the patient the compound of claim 1 and at least another therapeutic agent.

42. The method of claim 41, wherein the condition or disease is cancer.

43. The method of claim 41, wherein the at least another therapeutic agent is an anticancer agent.
